(19) United States
(12) Patent Application Publication
(10) Pub. No.: US 2023/0212177 A1
(43) Pub. Date: Jul. 6, 2023

(54) COMPOUND, LIGHT-EMITTING MATERIAL, AND LIGHT-EMITTING ELEMENT

(71) Applicant: KYULUX, INC., Fukuoka-shi, Fukuoka (JP)

(72) Inventors: Kaori FUJISAWA, Fukuoka-shi, Fukuoka (JP); Yong Joo CHO, Fukuoka-shi, Fukuoka (JP); Yoshitake SUZUKI, Fukuoka-shi, Fukuoka (JP); Hayato KAKIZOE, Fukuoka-shi, Fukuoka (JP); Yu INADA, Fukuoka-shi, Fukuoka (JP); YuSeok YANG, Fukuoka-shi, Fukuoka (JP)

(21) Appl. No.: 18/001,115

(22) PCT Filed: Jun. 10, 2021
(86) PCT No.: PCT/JP2021/022100

§ 371 Date: Dec. 8, 2022

(30) Foreign Application Priority Data
Jun. 11, 2020 (JP) 2020-101635

(51) Int. Cl. C07D 491/048; H10K 50/11; H10K 85/60; C07D 495/04

(52) U.S. Cl. CPC C07D 491/048; H10K 50/11; H10K 85/6572; H10K 85/657; C07D 495/04

(57) ABSTRACT

A compound represented by the following general formula is useful as a light emitting material. Two of R¹ to R⁵ each are an aromatic hydrocarbon cyclic group, or a nitrogen atom-containing aromatic heterocyclic group, three of R¹ to R⁵ each are a donor group, and at least one thereof is a benzofuran ring-condensed carbazolyl-9-yl group.

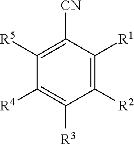

### TECHNICAL FIELD

The present invention relates to a compound useful as a light emitting material, and a light emitting device using the compound.

### BACKGROUND ART

Studies for enhancing the light emission efficiency of light emitting devices such as organic electroluminescent devices (organic EL devices) are being made actively. In particular, various kinds of efforts have been made for increasing light emission efficiency by newly developing and combining an electron transporting material, a hole transporting material, and a light emitting material to constitute an organic electroluminescent device. Among them, there are seen some reports relating to an organic electroluminescent device that utilizes a delayed fluorescent material.

A delayed fluorescent material is a material which, in an excited state, after having undergone reverse intersystem crossing from an excited triplet state to an excited singlet state, emits fluorescence when returning back from the excited singlet state to a ground state thereof. Fluorescence through the route is observed later than fluorescence from the excited singlet state directly occurring from the ground state (ordinary fluorescence), and is therefore referred to as delayed fluorescence. Here, for example, in the case where a light emitting compound is excited through carrier injection thereinto, the occurring probability of the excited singlet state to the excited triplet state is statistically 25%/75%, and therefore improvement of light emission efficiency by the fluorescence alone from the directly occurring excited singlet state is limited. On the other hand, in a delayed fluorescent material, not only the excited singlet state thereof but also the excited triplet state can be utilized for fluorescent emission through the route via the above-mentioned reverse intersystem crossing, and therefore as compared with an ordinary fluorescent material, a delayed fluorescent material can realize a higher emission efficiency.

Since such principles have been clarified, various delayed fluorescent materials have become discovered by various studies. However, every material capable of emitting delayed fluorescence is not always immediately useful as a light emitting material. Some delayed fluorescent materials are relatively less likely to undergo reverse intersystem crossing, and some delayed fluorescent materials have a long lifetime. In a high current density region, excitons may accumulate to lower emission efficiency, or in continuous long-time driving, some materials may rapidly worsen. Consequently, in fact, there are very many delayed fluorescent materials with room for improvement in point of practicability. Therefore, it is pointed out that benzonitrile compounds that are known as delayed fluorescent materials also have various problems. For example, 2CzPN having the following structure is a material that emits delayed fluorescence, but has a problem in that the emission efficiency is not high and, in addition, the emission efficiency greatly reduces in a high-current density region (see NPL 1).

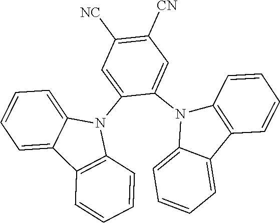 [SMILES: N#Cc1cc(-n2c3ccccc3c3ccccc32)c(-n2c3ccccc3c3ccccc32)cc1C#N]

### CITATION LIST

### Non-Patent Literature

NPL 1: Organic Electronics 14 (2013) 2721-2726

### SUMMARY OF INVENTION

### Technical Problem

Although it has been pointed out that such a problem has been encountered, it can be said that the relationship between the chemical structure and the characteristics of the delayed fluorescent material has been sufficiently elucidated. Consequently, at present, it is difficult to generalize the chemical structure of a compound useful as a light emitting material, and there are many unclear points.

Given the situation, the present inventors have made repeated studies for the purpose of providing a compound more useful as a light emitting material for light emitting devices. With that, the inventors have further made assiduous studies in order to derive and generalize a general formula of a compound more useful as a light emitting material.

### Solution to Problem

As a result of further promoting assiduous studies for attaining the above-mentioned object, the present inventors have found that, among benzonitrile derivatives, compounds having a structure that satisfies a specific requirement are useful as a light emitting material. The present invention has been proposed on the basis of these findings, and specifically has the following constitution.

A compound represented by the following general formula (1):

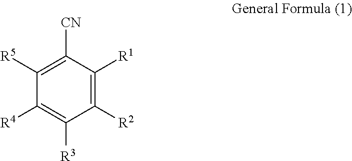

In the general formula (1);

two of R¹ to R⁵ are each independently a substituted or unsubstituted aromatic hydrocarbon cyclic group, or a substituted or unsubstituted aromatic heterocyclic group containing a nitrogen atom as a ring skeleton constituting atom,

three of R¹ to R⁵ are each independently a donor group (but excepting a substituted or unsubstituted aromatic hydrocarbon cyclic group, and a substituted or unsubstituted aromatic heterocyclic group containing a nitrogen atom as a ring skeleton constituting atom), all the three donor groups are not the same, and at least one of the three donor groups is a carbazolyl-9-yl group condensed with a benzofuran ring.

[2] The compound according to [1], wherein R¹, R² and R⁴ each are independently a donor group.
[3] The compound according to [1], wherein R¹, R³ and R⁴ each are independently a donor group.
[4] The compound according to any one of [1] to [3], wherein two of the three donor groups are the same as each other.
[5] The compound according to any one of [1] to [4], wherein the carbazolyl-9-yl group condensed with a benzofuran ring has a structure where the benzofuran ring is directly condensed with one benzene ring constituting the carbazolyl-9-yl group.
[6] The compound according to [5], wherein the benzofuran ring-condensed carbazolyl-9-yl group has a structure of any of the following:

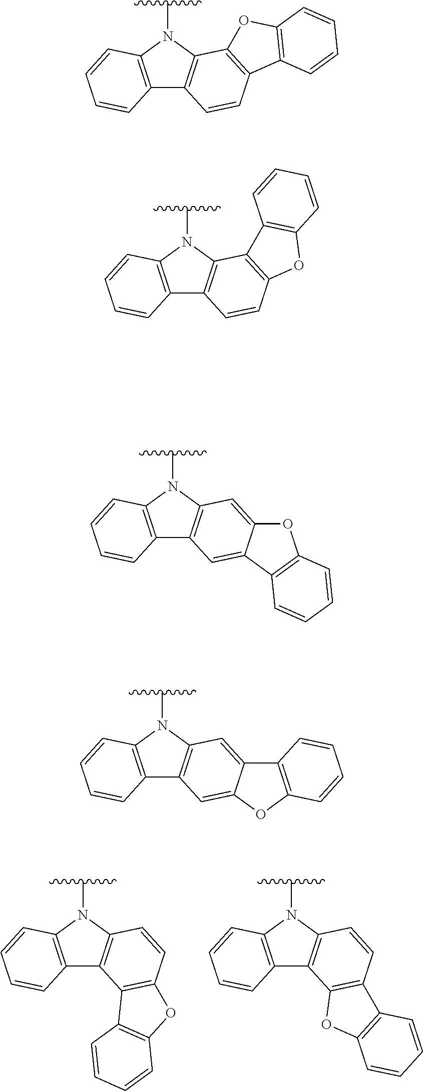

In the above structures, the hydrogen atom can be substituted.

[7] The compound according to [5] or [6], wherein two of R¹ to R⁵ are benzofuran ring-condensed carbazolyl-9-yl groups.
[8] The compound according to [7], wherein the two benzofuran ring-condensed carbazolyl-9-yl groups are the same as each other.
[9] The compound according to [5] or [6], wherein only one of R¹ to R⁵ is a benzofuran ring-condensed carbazolyl-9-yl group.
[10] The compound according to any one of [1] to [9], wherein two of R¹ to R⁵ each are independently a substituted or unsubstituted aromatic hydrocarbon cyclic group.

The compound according to any one of [1] to [10], wherein two of R¹ to R⁵ are each independently a substituted or unsubstituted aromatic heterocyclic group containing a nitrogen atom as a ring skeleton constituting atom.

The compound according to [10] or [11], wherein two of R¹ to R⁵ are the same as each other.

A light emitting material of a compound of any one of [1] to [12].

A light emitting device containing a compound of any one of [1] to [12].

The light emitting device according to [14], wherein the light emitting device has a light emitting layer and the light emitting layer contains the above compound and a host material.

The light emitting device according to [14], wherein the light emitting device has a light emitting layer, the light emitting layer contains the above compound and a light emitting material, and the light emitting material mainly emits light.

### Advantageous Effects of Invention

The compound of the present invention is useful as a light emitting material. Also, the compound of the present invention includes a compound that emits delayed fluorescence. Also, the compound of the present invention is useful as a material for organic light emitting devices.

### BRIEF DESCRIPTION OF DRAWING

FIG. 1 This is a schematic cross-sectional view showing an example of a layer configuration of an organic electroluminescent device.

### DESCRIPTION OF EMBODIMENTS

The contents of the invention will be described in detail below. The constitutional elements may be described below with reference to representative embodiments and specific examples of the invention, but the invention is not limited to the embodiments and the examples. In the description herein, a numerical range expressed as “to” means a range that includes the numerical values described before and after “to” as the upper limit and the lower limit. The isotopic species of the hydrogen atom existing in the molecule of the compound used in the present invention is not specifically limited, and for example, the hydrogen atoms in the molecule can be all ¹H, or a part or all thereof can be ²H (deuterium D).

### (Compound Represented by General Formula (1))

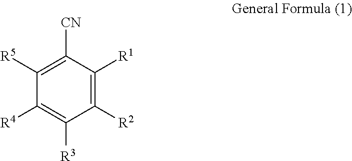

In the general formula (1), two of R¹ to R⁵ are each independently a substituted or unsubstituted aromatic hydrocarbon cyclic group, or a substituted or unsubstituted aromatic heterocyclic group containing a nitrogen atom as a ring skeleton constituting atom.

“Aromatic hydrocarbon cyclic group” as referred to in the present invention means a group in which the bonding ring (one ring) is an aromatic hydrocarbon ring. For example, it includes a phenyl group that bonds via one carbon atom constituting the ring skeleton of a benzene ring. The hydrogen atom constituting the bonding aromatic hydrocarbon ring can be substituted. Also one or more rings can be condensed with the bonding aromatic hydrocarbon ring. Also the condensed ring can be further condensed with any other ring. The condensing ring includes an aromatic hydrocarbon ring, an aromatic heteroring, an aliphatic hydrocarbon ring, and an aliphatic heteroring. The aromatic hydrocarbon ring includes a benzene ring. The aromatic heteroring includes a pyridine ring, a pyridazine ring, a pyrimidine ring, a pyrazine ring, a triazine ring, a pyrrole ring, a pyrazole ring, and an imidazole ring. The aliphatic hydrocarbon ring includes a cyclopentane ring, a cyclohexane ring, and a cycloheptane ring. The aliphatic heteroring includes a piperidine ring, a pyrrolidine ring, and an imidazoline ring. Specific examples of a condensed ring that constitutes an aromatic hydrocarbon ring includes a naphthalene ring, an anthracene ring, a phenanthrene ring, a pyran ring, and a tetracene ring. Also specific examples of a condensed ring that contains a hetero atom include an indole ring, an isoindole ring, a benzimidazole ring, a benzotriazole ring, a quinoline ring, an isoquinoline ring, a quinazoline ring, a quinoxaline ring and a cinnoline ring. The condensed ring of these specific examples bonds via the carbon atom that constitutes the benzene ring thereof.

The number of the carbon atoms of the substituted or unsubstituted aromatic hydrocarbon cyclic group that R¹ to R⁵ can represent is preferably 6 to 40, more preferably 6 to 30, even more preferably 6 to 20. The number of the ring skeleton constituting atoms of the bonding ring is preferably 6 to 14, more preferably 6 to 12, even more preferably 6.

“Aromatic heterocyclic group” as referred to in the present invention means a group such that the bonding ring (one ring) is an aromatic heteroring and that the group bonds via one carbon atom that constitutes the ring skeleton of the aromatic hetero ring. For example, it includes a pyridyl group bonding via one carbon atom that constitutes the ring skeleton of a pyridine ring. The aromatic heteroring that R₁ to R⁵ can represent contains a nitrogen atom as a ring skeleton constituting atom of the bonding ring (one ring). The bonding ring can contain any other hetero ring than a nitrogen atom as the ring skeleton constituting atom, but preferably contains a nitrogen atom alone as the ring skeleton constituting hetero atom. The number of the hetero atoms contained in the bonding ring is preferably 1 to 3, more preferably 1 or 2. The bonding ring includes a pyridine ring, a pyridazine ring, a pyrimidine ring, a pyrazine ring, a triazine ring, a pyrrole ring, a pyrazole ring, and an imidazole ring. The hydrogen atom constituting the bonding ring can be substituted. The bonding ring can be condensed with one or more rings. The condensed ring can be further condensed with any other rings. The bonding ring includes an aromatic hydrocarbon ring, an aromatic heteroring, an aliphatic hydrocarbon ring, and an aliphatic heteroring. For specific examples of the aromatic hydrocarbon ring, the aromatic heteroring, the aliphatic hydrocarbon ring, and the aliphatic heteroring, reference can be made to the corresponding description in the “aromatic hydrocarbon cyclic ring” mentioned hereinabove. Specific examples of the condensed ring to constitute the aromatic heteroring include a quinoline ring, an isoquinoline ring, a quinazoline ring, a quinoxaline ring, a cinnoline ring, and a pteridine ring. The condensed ring of these specific examples bonds via the carbon atom that constitutes the ring skeleton of the heteroring.

The number of the carbon atoms of the substituted or unsubstituted aromatic heterocyclic group that R¹ to R⁵ can represent is preferably 3 to 30, more preferably 3 to 20, even more preferably 4 to 15. The number of the ring skeleton constituting atoms of the bonding ring is preferably 6 to 14, more preferably 6 to 12, even more preferably 6.

The aromatic hydrocarbon cyclic group and the aromatic heterocyclic group that R¹ to R⁵ can represent can be substituted. The substituent includes an alkyl group, an alkenyl group, an aryl group, a heteroaryl group, an alkoxy group, an alkylthio group, an aryloxy group, an arylthio group, a heteroaryloxy group, a heteroarylthio group, and a cyano group. These substituents can be substituted with any other substituents. A preferred substituent group includes an alkyl group, an aryl group, an alkoxy group or an alkylthio group.

The “alkyl group” as referred to herein may be any of a linear, branched or cyclic one. The group may have two or more kinds of a linear moiety, a cyclic moiety and a branched moiety as combined. The carbon number of the alkyl group may be, for example, 1 or more, 2 or more, or 4 or more. The carbon number may be 30 or less, 20 or less, 10 or less, 6 or less, or 4 or less. Specific examples of the alkyl group include a methyl group, an ethyl group, an n-propyl group, an isopropyl group, an n-butyl group, an isobutyl group, a tert-butyl group, an n-pentyl group, an isopentyl group, an n-hexyl group, an isohexyl group, a 2-ethylhexyl group, an n-heptyl group, an isoheptyl group, an n-octyl group, an isooctyl group, an n-nonyl group, an isononyl group, an n-decanyl group, an isodecanyl group, a cyclopentyl group, a cyclohexyl group, and a cycloheptyl group. The alkyl group to be a substituent may be further substituted with an aryl group.

The “alkenyl group” as referred to herein may be any of a linear, branched or cyclic one. The group may have two or more kinds of a linear moiety, a cyclic moiety and a branched moiety as combined. The carbon number of the alkenyl group may be, for example, 2 or more, or 4 or more. The carbon number may be 30 or less, 20 or less, 10 or less, 6 or less, or 4 or less. Specific examples of the alkenyl group include an ethenyl group, an n-propenyl group, an isopropenyl group, an n-butenyl group, an isobutenyl group, an n-pentenyl group, an isopentenyl group, an n-hexenyl group, an isohexenyl group, and a 2-ethylhexenyl group. The alkenyl group to be a substituent may be further substituted.

The “aryl group” and the “heteroaryl group” each may be a single ring or may be a condensed ring of two or more kinds of rings. In the case of a condensed ring, the number of the rings that are condensed is preferably 2 to 6, and, for example, can be selected from 2 to 4. Specific examples of the ring include a benzene ring, a pyridine ring, a pyrimidine ring, a triazine ring, a naphthalene ring, an anthracene ring, a phenanthrene ring, a triphenylene ring, a quinoline ring, a pyrazine ring, a quinoxaline ring, and a naphthyridine ring. Specific examples of the arylene ring or the heteroarylene ring include a phenyl group, a 1-naphthyl group, a 2-naphthyl group, a 1-anthracenyl group, a 2-anthracenyl group, a 9-anthracenyl group, a 2-pyridyl group, a 3-pyridyl group, and a 4-pyridyl group.

Regarding the alkyl moiety in the “alkoxy group” and the “alkylthio group”, reference can be made to the description and the specific examples of the alkyl group mentioned above. Regarding the aryl moiety in the “aryloxy group” and the “arylthio group”, reference can be made to the description and the specific examples of the aryl group mentioned above. Regarding the heteroaryl moiety in the “heteroaryloxy group” and the “heteroarylthio group”, reference can be made to the description and the specific examples of the heteroaryl group mentioned above.

Of R¹ to R⁵ in the general formula (1), the substituted or unsubstituted aromatic hydrocarbon cyclic group, or the substituted or unsubstituted aromatic heterocyclic group containing a nitrogen atom as a ring skeleton constituting atom can be any arbitrary two. The two can be the same as or different from each other, but are preferably the same. Regarding the combination of the two, for example, preferred are combinations of R³ and R⁵, R² and R⁵, R¹ and R⁵, and R² and R⁴. More preferred are combinations of R³ and R⁵, and R² and R⁵.

In one preferred embodiment of the present invention, two of R¹ to R⁵ in the general formula (1) are each independently a substituted or unsubstituted aromatic hydrocarbon cyclic group. More preferably. R³ and R⁵, R² and R⁵, R¹ and R⁵, or R² and R⁴ are each independently a substituted or unsubstituted aromatic hydrocarbon cyclic group. For example, there are mentioned a group where R³ and R⁵ each are independently a substituted or unsubstituted aromatic hydrocarbon cyclic group, and a group where R² and R⁵ each are independently a substituted or unsubstituted aromatic hydrocarbon cyclic group.

Hereinunder shown are specific examples of a substituted or unsubstituted aromatic hydrocarbon cyclic group and a substituted or unsubstituted aromatic heterocyclic group containing a nitrogen atom as a ring skeleton constituting atom, which two of R¹ to R⁵ in the general formula (1) can represent.

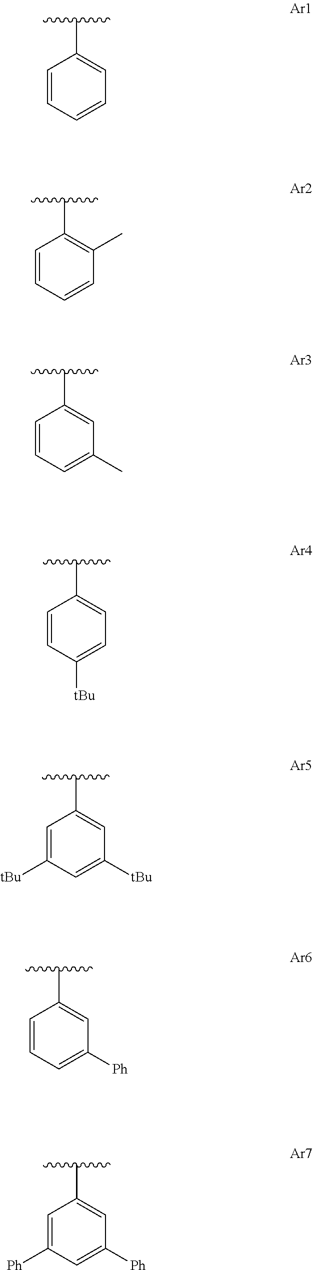

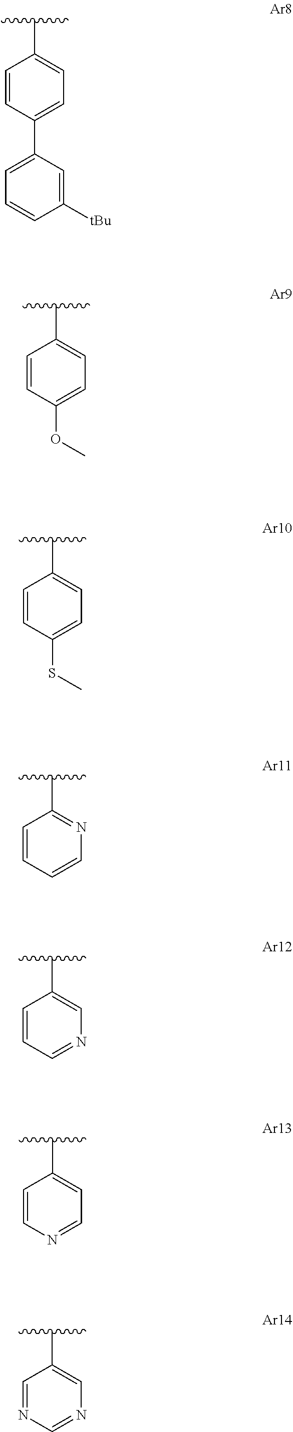

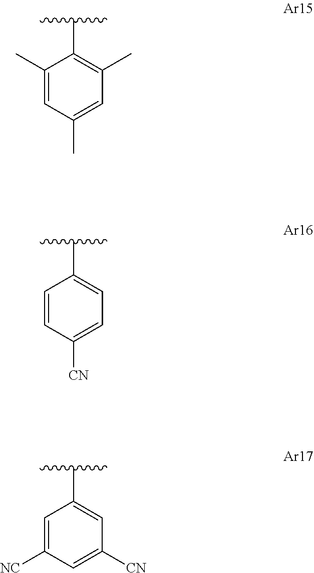

Three of R¹ to R⁵ in the general formula (1) are each independently a donor group. Here, the donor group is neither a substituted or unsubstituted aromatic hydrocarbon cyclic group, nor a substituted or unsubstituted aromatic heterocyclic group containing a nitrogen atom as a ring skeleton constituting atom.

The “donor group” in the present invention is a group having a negative Hammett's σp value. Here, “Hammett's σₚ value” is one propounded by L. P. Hammett, and is one to quantify the influence of a substituent on the reaction rate or the equilibrium of a para-substituted benzene derivative. Specifically, the value is a constant (σₚ) peculiar to the substituent in the following equation that is established between a substituent and a reaction rate constant or an equilibrium constant in a para-substituted benzene derivative:

log(k/k₀)=ρσₚ

or

log(K/K₀)=ρσₚ

In the above equations, k represents a rate constant of a benzene derivative not having a substituent; k₀ represents a rate constant of a benzene derivative substituted with a substituent; K represents an equilibrium constant of a benzene derivative not having a substituent; K₀ represents an equilibrium constant of a benzene derivative substituted with a substituent; ρ represents a reaction constant to be determined by the kind and the condition of reaction. Regarding the description relating to the “Hammett's σₚ value” and the numerical value of each substituent in the present invention, reference may be made to the description relating to 6p value in Hansch, C. et. al., Chem. Rev., 91, 165-195 (1991). A group having a negative Hammett's σₚ value tends to exhibit an electron donor property, and a group having a positive Hammett's σₚ value tends to exhibit an electron acceptor property.

The donor group in the present invention is preferably a group containing a substituted amino group. The substituent bonding to the nitrogen atom of the amino group is preferably a substituted or unsubstituted alkyl group, a substituted or unsubstituted alkenyl group, a substituted or unsubstituted aryl group or a substituted or unsubstituted heteroaryl group, more preferably a substituted or unsubstituted aryl group or a substituted or unsubstituted heteroaryl group. Especially, the substituted amino group is preferably a substituted or unsubstituted diarylamino group or a substituted or unsubstituted diheteroarylamino group. In the present invention, the donor group can be a group bonding at the nitrogen atom of a substituted amino group, or can be a group bonding to the substituted amino group-bonding group. The substituted amino group-bonding group is preferably a n-conjugated group. More preferred is a group bonding at the nitrogen atom of the substituted amino group. Regarding the alkyl group, the alkenyl group, the aryl group and the heteroaryl group as referred to herein as substituents, reference can be made to the corresponding description relating to the substituents for the aromatic hydrocarbon cyclic group and the aromatic heterocyclic group mentioned hereinabove.

The other donor group especially preferred in the present invention is a substituted or unsubstituted carbazol-9-yl group. Preferably, the three donor groups existing in the general formula (1) are each independently a substituted or unsubstituted carbazol-9-yl group. The substituent for the carbazol-9-yl group includes an alkyl group, an alkenyl group, an aryl group, a heteroaryl group, an alkoxy group, an alkylthio group, an aryloxy group, an arylthio group, a heteroaryloxy group, a heteroarylthio group, and a substituted amino group. Preferred substituents are an alkyl group, an aryl group and a substituted amino group. Regarding the description of the substituted amino group, reference can be made to the description in the preceding paragraph. The substituted amino group as referred to herein includes a substituted or unsubstituted carbazolyl group, and especially includes a substituted or unsubstituted carbazol-9-yl group.

The number of the atoms except hydrogen atoms constituting the donor group in the present invention is preferably 5 or more, more preferably 10 or more, even more preferably 13 or more, and is preferably 80 or less, more preferably 60 or less, even more preferably 40 or less.

At least one of the three donor groups existing in the general formula (1) is a benzofuran ring-condensed carbazol-9-yl group. The benzofuran ring can be condensed with the carbazol-9-yl group via the furan ring, or can be condensed with the carbazol-9-yl group via the benzene ring. The former is preferred. One benzofuran ring can be condensed with the carbazol-9-yl group, or two or more can be condensed. In the case where two or more are condensed, these benzofuran rings can be the same structure, or can be different structures. Also the kind of the condensed rings can be the same or different. The benzofuran ring-condensed carbazol-9-yl group can be substituted. For the substituent, preferably, reference can be made to the substituents mentioned hereinabove for the substituent for the carbazol-9-yl group. In the case where any other ring than the benzofuran ring is condensed with the benzofuran ring-condensed carbazol-9-yl group, the condensed ring is preferably a ring alone selected from the group consisting of an aromatic hydrocarbon ring and an aliphatic hydrocarbon ring, more preferably an aromatic hydrocarbon ring alone. Also preferably, the benzofuran ring-condensed carbazol-9-yl group is not condensed with any other ring than the benzofuran ring. Also preferably, the benzofuran ring-condensed carbazol-9-yl group is unsubstituted.

The three donor groups existing in the general formula (1) are not all the same. All the three can differ from each other, or two can be the same and one can differ. The latter is preferred. In one preferred embodiment of the present invention, two are benzofuran ring-condensed carbazol-9-yl groups, and the remaining one is a donor group excepting them. In another preferred embodiment of the present invention, one is a benzofuran ring-condensed carbazol-9-yl group and the other two are donor groups other than it. The other donor group is preferably a carbazol-9-yl group not condensed with a benzofuran ring.

Three of R₁ to R⁵ in the general formula (1) that are donor groups can be in any combination. A preferred combination is R¹ and R² and R⁴, and also exemplified is an embodiment where R¹ and R² are the same and R⁴ differs. Also exemplified is an embodiment where R¹ and R⁴ are the same and R² differs. Further exemplified is an embodiment where R² and R⁴ are the same and R¹ differs. Another preferred combination is R¹ and R³ and R⁴, and exemplified is an embodiment where R¹ and R³ are the same and R⁴ differs. Also exemplified is an embodiment where R¹ and R⁴ are the same and R³ differs. Further exemplified is an embodiment where R³ and R⁴ are the same and R¹ differs. Another preferred combination is R² and R³ and R⁴, and exemplified is an embodiment where R² and R³ are the same and R⁴ differs. Also exemplified is an embodiment where R² and R⁴ are the same and R³ differs. Further exemplified is an embodiment where R³ and R⁴ are the same and R² differs. Another preferred combination is R¹ and R³ and R⁵, and exemplified is an embodiment where R¹ and R³ are the same and R⁵ differs. Also exemplified is an embodiment where R¹ and R⁵ are the same and R³ differs. Further exemplified is an embodiment where R³ and R⁵ are the same and R¹ differs.

Hereinunder shown are specific examples of the donor group that three of R¹ to R⁵ in the general formula (1) can represent. D21 to D26 correspond to specific examples of a benzofuran ring-condensed carbazol-9-yl group.

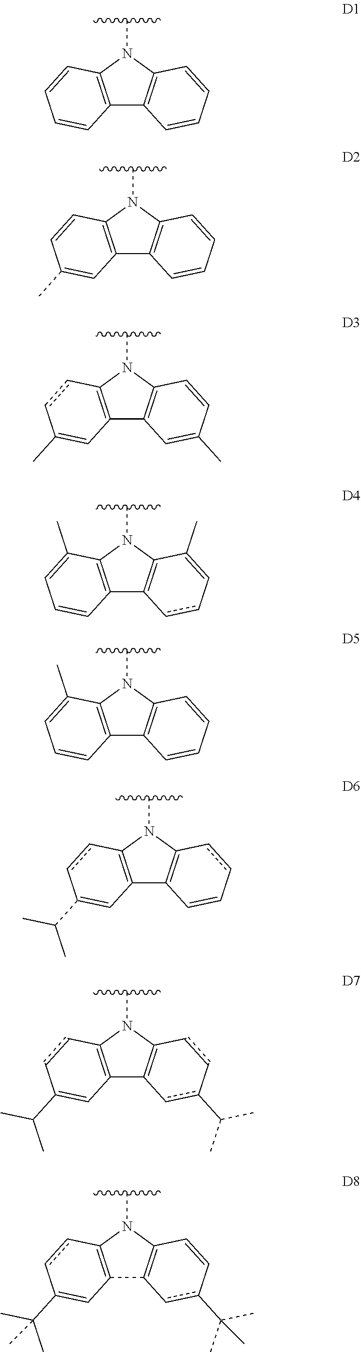

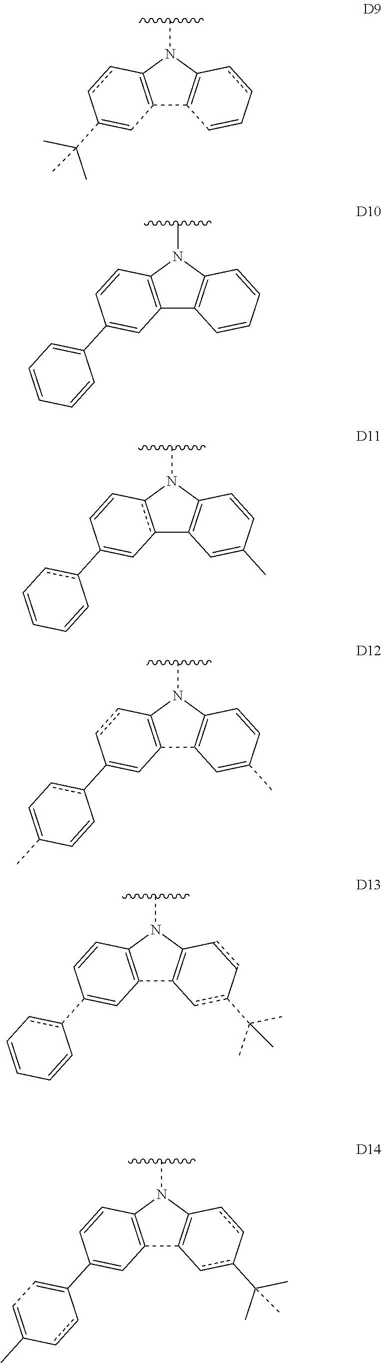

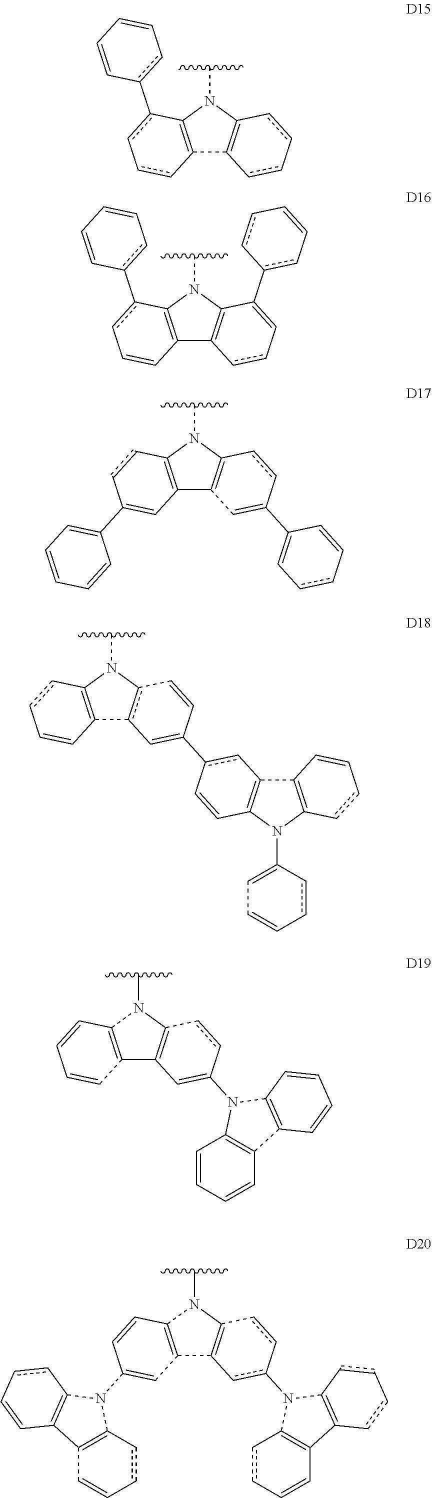

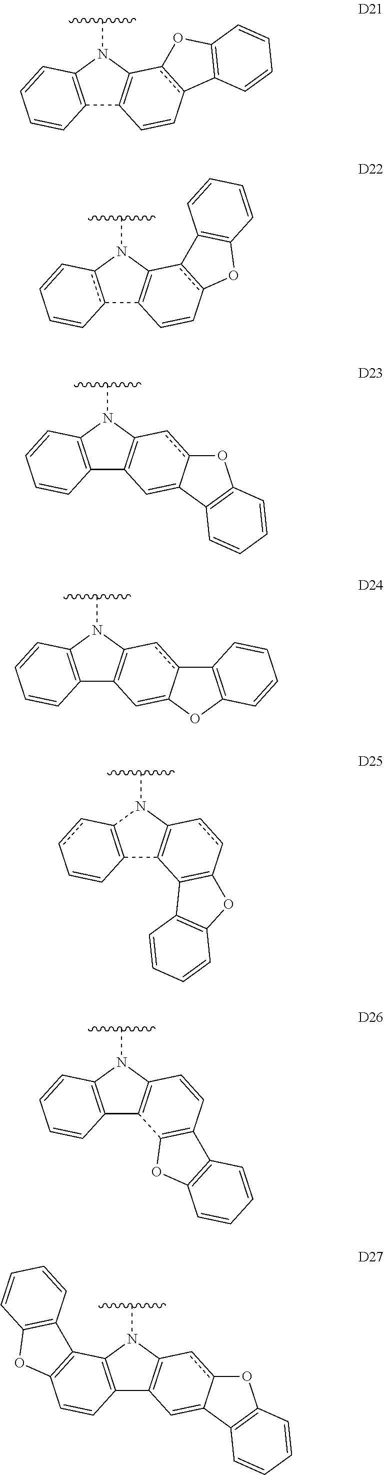

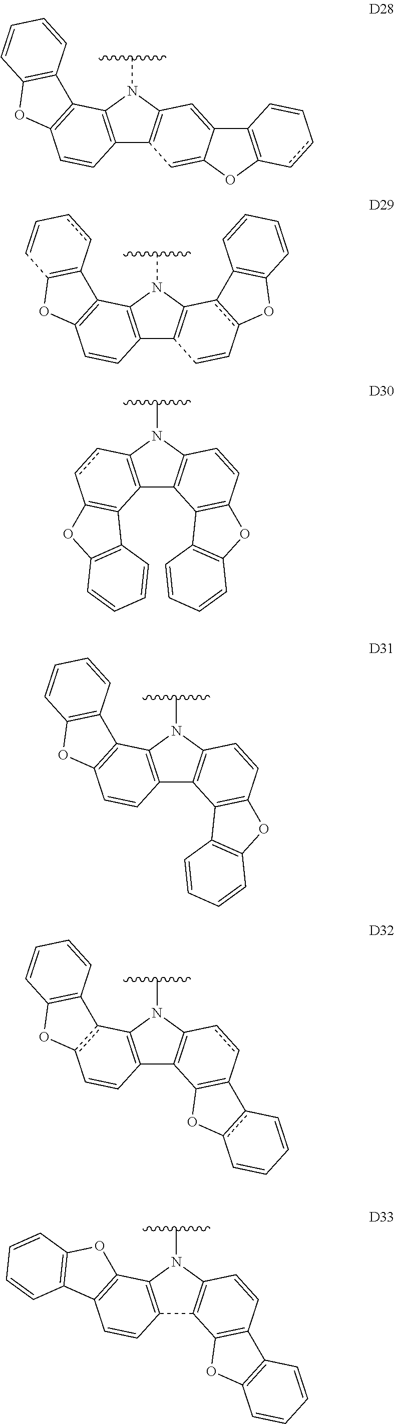

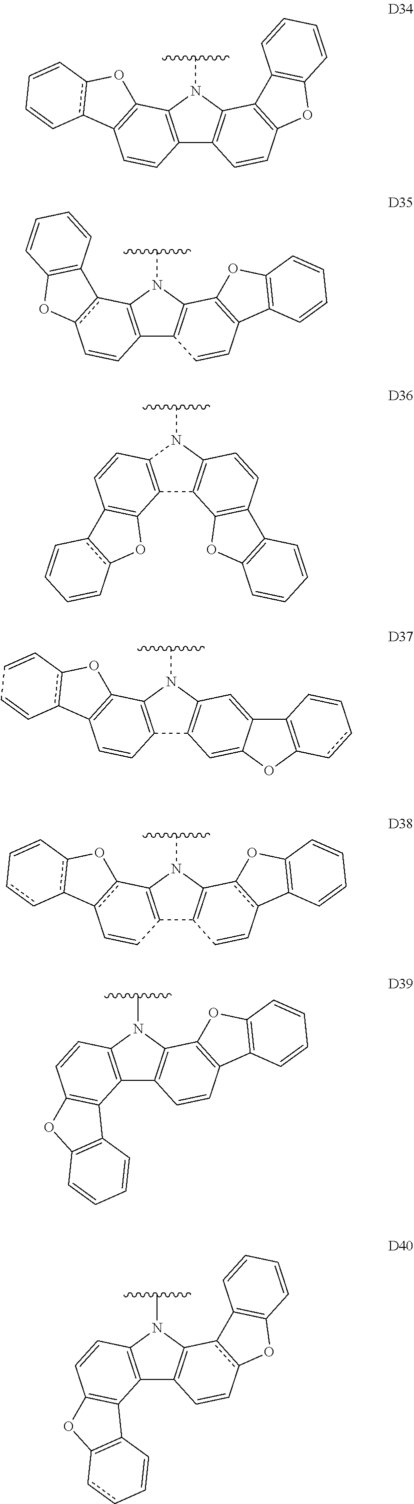

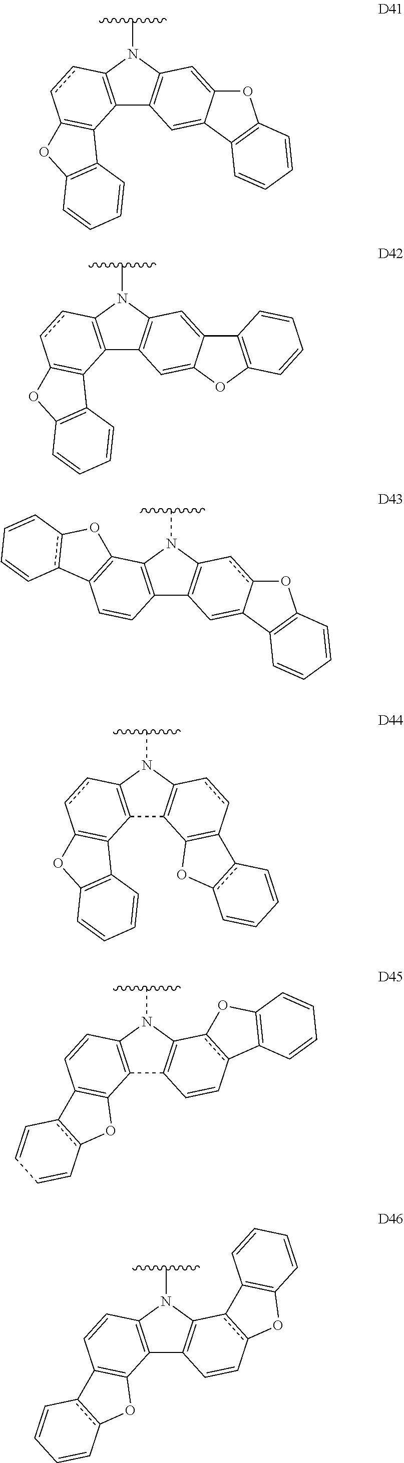

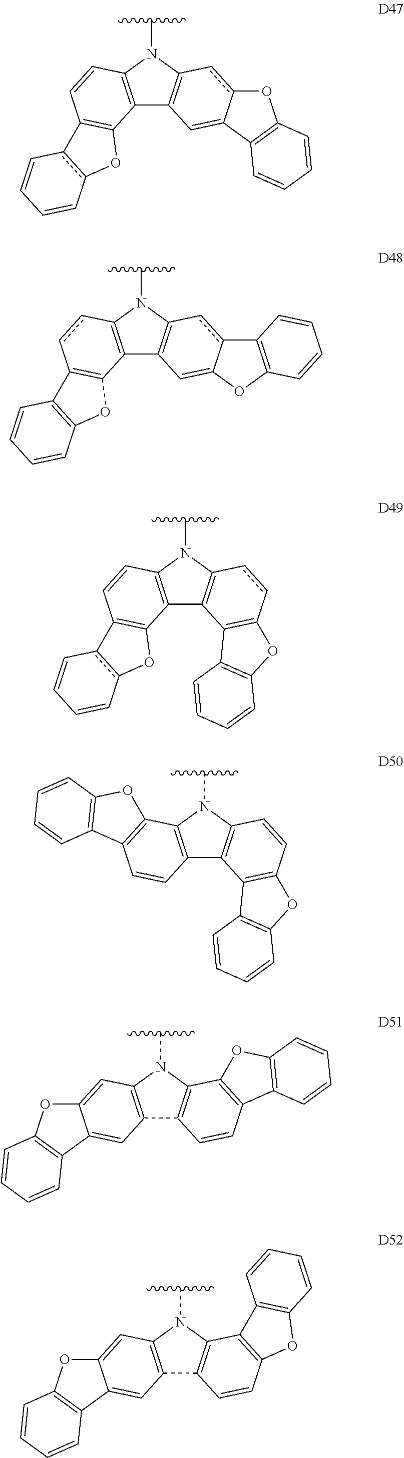

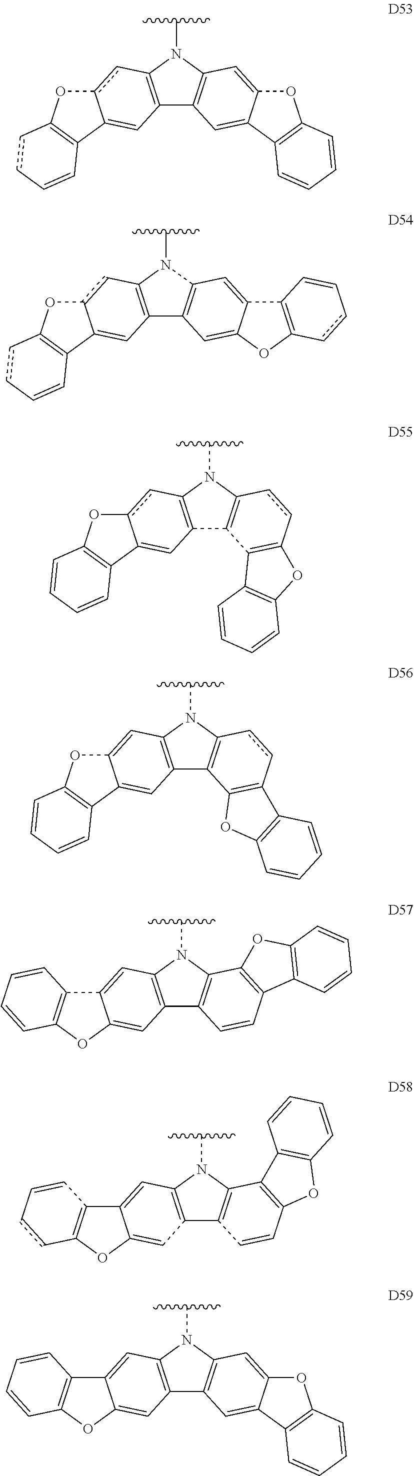

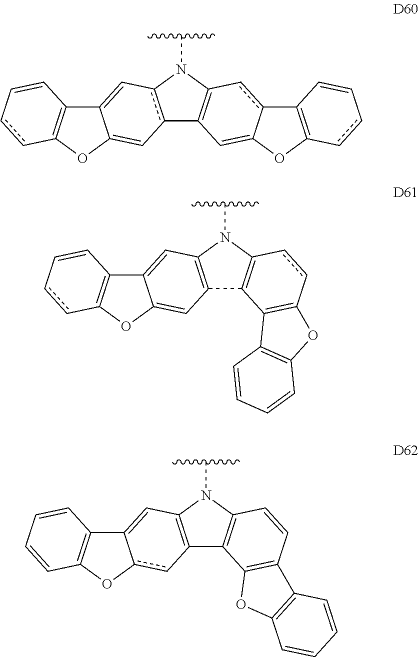

In one preferred embodiment of the present invention, the compound represented by the general formula (1) is composed of atoms alone selected from the group consisting of a carbon atom, a hydrogen atom, a nitrogen atom, an oxygen atom and a sulfur atom. In one preferred embodiment of the present invention, the compound represented by the general formula (1) is composed of a carbon atom, a hydrogen atom, a nitrogen atom and an oxygen atom alone.

In the following Table 1 and Table 2, specific examples of the compound represented by the general formula (1) are shown. In Table 1 and Table 2, R¹ to R⁵ are specified to show the structures of the compounds. In Table 2, plural compounds are collectively shown in every row. For example, in Compounds 4537 to 4572 in Table 2, R² to R⁵ each are fixed to D1, Ar1, D1 and Ar1, respectively. With that, those where R¹ is D27 to D62 are compounds 4537 to 4572 in that order. Of compounds 4573 to 5868, those where R¹ is D1, R³ and R⁵ are Ar1 and where R² is D27 and R⁴ is D27 to D62 are Compounds 4573 to 4608, and those where R² is D28 and where R⁴ is D27 to D62 are Compounds 4609 to 4644, and those where R³ is D29 and where R⁴ is D27 to D62 are Compounds 4645 to 4680 all in that order, and lastly, those where R² is D62 and where R⁴ is D27 to D62 are Compounds 5833 to 5868. In Table 1 and Table 2, Compounds 1 to 16452 are individually specified for their structures, and are specifically disclosed in the present specification. Those where all the hydrogen atoms existing in the molecules of Compounds 1 to 16452 are substituted with deuterium atoms are disclosed as Compounds 1d to 16452d. In the case where the following compounds have rotational isomers, mixtures of rotational isomers and each separated rotational isomer are considered to be disclosed in the present specification.

TABLE 1

| No. | R¹ | R² | R³ | R⁴ | R⁵ |
|---|---|---|---|---|---|
| 1 | D21 | D1 | Ar1 | D1 | Ar1 |
| 2 | D21 | D2 | Ar1 | D2 | Ar1 |
| 3 | D21 | D3 | Ar1 | D3 | Ar1 |
| 4 | D21 | D4 | Ar1 | D4 | Ar1 |
| 5 | D21 | D5 | Ar1 | D5 | Ar1 |
| 6 | D21 | D6 | Ar1 | D6 | Ar1 |
| 7 | D21 | D7 | Ar1 | D7 | Ar1 |
| 8 | D21 | D8 | Ar1 | D8 | Ar1 |
| 9 | D21 | D9 | Ar1 | D9 | Ar1 |
| 10 | D21 | D10 | Ar1 | D10 | Ar1 |
| 11 | D21 | D11 | Ar1 | D11 | Ar1 |
| 12 | D21 | D12 | Ar1 | D12 | Ar1 |
| 13 | D21 | D13 | Ar1 | D13 | Ar1 |
| 14 | D21 | D14 | Ar1 | D14 | Ar1 |
| 15 | D21 | D15 | Ar1 | D15 | Ar1 |
| 16 | D21 | D16 | Ar1 | D16 | Ar1 |
| 17 | D21 | D17 | Ar1 | D17 | Ar1 |
| 18 | D21 | D18 | Ar1 | D18 | Ar1 |
| 19 | D21 | D19 | Ar1 | D19 | Ar1 |
| 20 | D21 | D20 | Ar1 | D20 | Ar1 |
| 21 | D21 | D22 | Ar1 | D22 | Ar1 |
| 22 | D21 | D23 | Ar1 | D23 | Ar1 |
| 23 | D21 | D24 | Ar1 | D24 | Ar1 |
| 24 | D21 | D25 | Ar1 | D25 | Ar1 |
| 25 | D21 | D26 | Ar1 | D26 | Ar1 |
| 26 | D22 | D1 | Ar1 | D1 | Ar1 |
| 27 | D22 | D2 | Ar1 | D2 | Ar1 |
| 28 | D22 | D3 | Ar1 | D3 | Ar1 |
| 29 | D22 | D4 | Ar1 | D4 | Ar1 |
| 30 | D22 | D5 | Ar1 | D5 | Ar1 |
| 31 | D22 | D6 | Ar1 | D6 | Ar1 |
| 32 | D22 | D7 | Ar1 | D7 | Ar1 |
| 33 | D22 | D8 | Ar1 | D8 | Ar1 |
| 34 | D22 | D9 | Ar1 | D9 | Ar1 |
| 35 | D22 | D10 | Ar1 | D10 | Ar1 |
| 36 | D22 | D11 | Ar1 | D11 | Ar1 |
| 37 | D22 | D12 | Ar1 | D12 | Ar1 |
| 38 | D22 | D13 | Ar1 | D13 | Ar1 |
| 39 | D22 | D14 | Ar1 | D14 | Ar1 |
| 40 | D22 | D15 | Ar1 | D15 | Ar1 |
| 41 | D22 | D16 | Ar1 | D16 | Ar1 |
| 42 | D22 | D17 | Ar1 | D17 | Ar1 |
| 43 | D22 | D18 | Ar1 | D18 | Ar1 |
| 44 | D22 | D19 | Ar1 | D19 | Ar1 |
| 45 | D22 | D20 | Ar1 | D20 | Ar1 |
| 46 | D22 | D21 | Ar1 | D21 | Ar1 |
| 47 | D22 | D23 | Ar1 | D23 | Ar1 |
| 48 | D22 | D24 | Ar1 | D24 | Ar1 |
| 49 | D22 | D25 | Ar1 | D25 | Ar1 |
| 50 | D22 | D26 | Ar1 | D26 | Ar1 |
| 51 | D23 | D1 | Ar1 | D1 | Ar1 |
| 52 | D23 | D2 | Ar1 | D2 | Ar1 |
| 53 | D23 | D3 | Ar1 | D3 | Ar1 |
| 54 | D23 | D4 | Ar1 | D4 | Ar1 |
| 55 | D23 | D5 | Ar1 | D5 | Ar1 |
| 56 | D23 | D6 | Ar1 | D6 | Ar1 |
| 57 | D23 | D7 | Ar1 | D7 | Ar1 |
| 58 | D23 | D8 | Ar1 | D8 | Ar1 |
| 59 | D23 | D9 | Ar1 | D9 | Ar1 |
| 60 | D23 | D10 | Ar1 | D10 | Ar1 |
| 61 | D23 | D11 | Ar1 | D11 | Ar1 |
| 62 | D23 | D12 | Ar1 | D12 | Ar1 |
| 63 | D23 | D13 | Ar1 | D13 | Ar1 |
| 64 | D23 | D14 | Ar1 | D14 | Ar1 |
| 65 | D23 | D15 | Ar1 | D15 | Ar1 |
| 66 | D23 | D16 | Ar1 | D16 | Ar1 |
| 67 | D23 | D17 | Ar1 | D17 | Ar1 |
| 68 | D23 | D18 | Ar1 | D18 | Ar1 |
| 69 | D23 | D19 | Ar1 | D19 | Ar1 |
| 70 | D23 | D20 | Ar1 | D20 | Ar1 |
| 71 | D23 | D21 | Ar1 | D21 | Ar1 |
| 72 | D23 | D22 | Ar1 | D22 | Ar1 |
| 73 | D23 | D24 | Ar1 | D24 | Ar1 |
| 74 | D23 | D25 | Ar1 | D25 | Ar1 |
| 75 | D23 | D26 | Ar1 | D26 | Ar1 |
| 76 | D24 | D1 | Ar1 | D1 | Ar1 |
| 77 | D24 | D2 | Ar1 | D2 | Ar1 |
| 78 | D24 | D3 | Ar1 | D3 | Ar1 |
| 79 | D24 | D4 | Ar1 | D4 | Ar1 |
| 80 | D24 | D5 | Ar1 | D5 | Ar1 |
| 81 | D24 | D6 | Ar1 | D6 | Ar1 |
| 82 | D24 | D7 | Ar1 | D7 | Ar1 |
| 83 | D24 | D8 | Ar1 | D8 | Ar1 |
| 84 | D24 | D9 | Ar1 | D9 | Ar1 |
| 85 | D24 | D10 | Ar1 | D10 | Ar1 |
| 86 | D24 | D11 | Ar1 | D11 | Ar1 |
| 87 | D24 | D12 | Ar1 | D12 | Ar1 |
| 88 | D24 | D13 | Ar1 | D13 | Ar1 |
| 89 | D24 | D14 | Ar1 | D14 | Ar1 |
| 90 | D24 | D15 | Ar1 | D15 | Ar1 |
| 91 | D24 | D16 | Ar1 | D16 | Ar1 |
| 92 | D24 | D17 | Ar1 | D17 | Ar1 |
| 93 | D24 | D18 | Ar1 | D18 | Ar1 |
| 94 | D24 | D19 | Ar1 | D19 | Ar1 |
| 95 | D24 | D20 | Ar1 | D20 | Ar1 |
| 96 | D24 | D21 | Ar1 | D21 | Ar1 |
| 97 | D24 | D22 | Ar1 | D22 | Ar1 |
| 98 | D24 | D23 | Ar1 | D23 | Ar1 |
| 99 | D24 | D25 | Ar1 | D25 | Ar1 |
| 100 | D24 | D26 | Ar1 | D26 | Ar1 |
| 101 | D25 | D1 | Ar1 | D1 | Ar1 |
| 102 | D25 | D2 | Ar1 | D2 | Ar1 |
| 103 | D25 | D3 | Ar1 | D3 | Ar1 |
| 104 | D25 | D4 | Ar1 | D4 | Ar1 |
| 105 | D25 | D5 | Ar1 | D5 | Ar1 |
| 106 | D25 | D6 | Ar1 | D6 | Ar1 |
| 107 | D25 | D7 | Ar1 | D7 | Ar1 |
| 108 | D25 | D8 | Ar1 | D8 | Ar1 |
| 109 | D25 | D9 | Ar1 | D9 | Ar1 |
| 110 | D25 | D10 | Ar1 | D10 | Ar1 |
| 111 | D25 | D11 | Ar1 | D11 | Ar1 |
| 112 | D25 | D12 | Ar1 | D12 | Ar1 |
| 113 | D25 | D13 | Ar1 | D13 | Ar1 |
| 114 | D25 | D14 | Ar1 | D14 | Ar1 |
| 115 | D25 | D15 | Ar1 | D15 | Ar1 |
| 116 | D25 | D16 | Ar1 | D16 | Ar1 |
| 117 | D25 | D17 | Ar1 | D17 | Ar1 |
| 118 | D25 | D18 | Ar1 | D18 | Ar1 |
| 119 | D25 | D19 | Ar1 | D19 | Ar1 |
| 120 | D25 | D20 | Ar1 | D20 | Ar1 |
| 121 | D25 | D21 | Ar1 | D21 | Ar1 |
| 122 | D25 | D22 | Ar1 | D22 | Ar1 |
| 123 | D25 | D23 | Ar1 | D23 | Ar1 |
| 124 | D25 | D24 | Ar1 | D24 | Ar1 |
| 125 | D25 | D26 | Ar1 | D26 | Ar1 |
| 126 | D26 | D1 | Ar1 | D1 | Ar1 |
| 127 | D26 | D2 | Ar1 | D2 | Ar1 |
| 128 | D26 | D3 | Ar1 | D3 | Ar1 |
| 129 | D26 | D4 | Ar1 | D4 | Ar1 |
| 130 | D26 | D5 | Ar1 | D5 | Ar1 |
| 131 | D26 | D6 | Ar1 | D6 | Ar1 |
| 132 | D26 | D7 | Ar1 | D7 | Ar1 |
| 133 | D26 | D8 | Ar1 | D8 | Ar1 |
| 134 | D26 | D9 | Ar1 | D9 | Ar1 |
| 135 | D26 | D10 | Ar1 | D10 | Ar1 |
| 136 | D26 | D11 | Ar1 | D11 | Ar1 |
| 137 | D26 | D12 | Ar1 | D12 | Ar1 |
| 138 | D26 | D13 | Ar1 | D13 | Ar1 |
| 139 | D26 | D14 | Ar1 | D14 | Ar1 |
| 140 | D26 | D15 | Ar1 | D15 | Ar1 |
| 141 | D26 | D16 | Ar1 | D16 | Ar1 |
| 142 | D26 | D17 | Ar1 | D17 | Ar1 |
| 143 | D26 | D18 | Ar1 | D18 | Ar1 |
| 144 | D26 | D19 | Ar1 | D19 | Ar1 |
| 145 | D26 | D20 | Ar1 | D20 | Ar1 |
| 146 | D26 | D21 | Ar1 | D21 | Ar1 |
| 147 | D26 | D22 | Ar1 | D22 | Ar1 |
| 148 | D26 | D23 | Ar1 | D23 | Ar1 |
| 149 | D26 | D24 | Ar1 | D24 | Ar1 |
| 150 | D26 | D25 | Ar1 | D25 | Ar1 |
| 151 | D1 | D21 | Ar1 | D21 | Ar1 |
| 152 | D2 | D21 | Ar1 | D21 | Ar1 |
| 153 | D3 | D21 | Ar1 | D21 | Ar1 |
| 154 | D4 | D21 | Ar1 | D21 | Ar1 |
| 155 | D5 | D21 | Ar1 | D21 | Ar1 |
| 158 | D6 | D21 | Ar1 | D21 | Ar1 |
| 157 | D7 | D21 | Ar1 | D21 | Ar1 |
| 158 | D8 | D21 | Ar1 | D21 | Ar1 |
| 159 | D9 | D21 | Ar1 | D21 | Ar1 |
| 160 | D10 | D21 | Ar1 | D21 | Ar1 |
| 161 | D11 | D21 | Ar1 | D21 | Ar1 |
| 162 | D12 | D21 | Ar1 | D21 | Ar1 |
| 163 | D13 | D21 | Ar1 | D21 | Ar1 |
| 164 | D14 | D21 | Ar1 | D21 | Ar1 |
| 165 | D15 | D21 | Ar1 | D21 | Ar1 |
| 166 | D16 | D21 | Ar1 | D21 | Ar1 |
| 167 | D17 | D21 | Ar1 | D21 | Ar1 |
| 168 | D18 | D21 | Ar1 | D21 | Ar1 |
| 169 | D19 | D21 | Ar1 | D21 | Ar1 |
| 170 | D20 | D21 | Ar1 | D21 | Ar1 |
| 171 | D22 | D21 | Ar1 | D21 | Ar1 |
| 172 | D23 | D21 | Ar1 | D21 | Ar1 |
| 173 | D24 | D21 | Ar1 | D21 | Ar1 |
| 174 | D25 | D21 | Ar1 | D21 | Ar1 |
| 175 | D26 | D21 | Ar1 | D21 | Ar1 |
| 176 | D1 | D22 | Ar1 | D22 | Ar1 |
| 177 | D2 | D22 | Ar1 | D22 | Ar1 |
| 178 | D3 | D22 | Ar1 | D22 | Ar1 |
| 179 | D4 | D22 | Ar1 | D22 | Ar1 |
| 180 | D5 | D22 | Ar1 | D22 | Ar1 |
| 181 | D6 | D22 | Ar1 | D22 | Ar1 |
| 182 | D7 | D22 | Ar1 | D22 | Ar1 |
| 183 | D8 | D22 | Ar1 | D22 | Ar1 |
| 184 | D9 | D22 | Ar1 | D22 | Ar1 |
| 185 | D10 | D22 | Ar1 | D22 | Ar1 |
| 186 | D11 | D22 | Ar1 | D22 | Ar1 |
| 187 | D12 | D22 | Ar1 | D22 | Ar1 |
| 188 | D13 | D22 | Ar1 | D22 | Ar1 |
| 189 | D14 | D22 | Ar1 | D22 | Ar1 |
| 190 | D15 | D22 | Ar1 | D22 | Ar1 |
| 191 | D16 | D22 | Ar1 | D22 | Ar1 |
| 192 | D17 | D22 | Ar1 | D22 | Ar1 |
| 193 | D18 | D22 | Ar1 | D22 | Ar1 |
| 194 | D19 | D22 | Ar1 | D22 | Ar1 |
| 195 | D20 | D22 | Ar1 | D22 | Ar1 |
| 196 | D21 | D22 | Ar1 | D22 | Ar1 |
| 197 | D23 | D22 | Ar1 | D22 | Ar1 |
| 198 | D24 | D22 | Ar1 | D22 | Ar1 |
| 199 | D25 | D22 | Ar1 | D22 | Ar1 |
| 200 | D26 | D22 | Ar1 | D22 | Ar1 |
| 201 | D1 | D23 | Ar1 | D23 | Ar1 |
| 202 | D2 | D23 | Ar1 | D23 | Ar1 |
| 203 | D3 | D23 | Ar1 | D23 | Ar1 |
| 204 | D4 | D23 | Ar1 | D23 | Ar1 |
| 205 | D5 | D23 | Ar1 | D23 | Ar1 |
| 206 | D6 | D23 | Ar1 | D23 | Ar1 |
| 207 | D7 | D23 | Ar1 | D23 | Ar1 |
| 208 | D8 | D23 | Ar1 | D23 | Ar1 |
| 209 | D9 | D23 | Ar1 | D23 | Ar1 |
| 210 | D10 | D23 | Ar1 | D23 | Ar1 |
| 211 | D11 | D23 | Ar1 | D23 | Ar1 |
| 212 | D12 | D23 | Ar1 | D22 | Ar1 |
| 213 | D13 | D23 | Ar1 | D23 | Ar1 |
| 214 | D14 | D23 | Ar1 | D23 | Ar1 |
| 215 | D15 | D23 | Ar1 | D23 | Ar1 |
| 216 | D16 | D23 | Ar1 | D23 | Ar1 |
| 217 | D17 | D23 | Ar1 | D23 | Ar1 |
| 218 | D18 | D23 | Ar1 | D23 | Ar1 |
| 219 | D19 | D23 | Ar1 | D23 | Ar1 |
| 220 | D20 | D23 | Ar1 | D23 | Ar1 |
| 221 | D21 | D23 | Ar1 | D23 | Ar1 |
| 222 | D22 | D23 | Ar1 | D23 | Ar1 |
| 223 | D24 | D23 | Ar1 | D23 | Ar1 |
| 224 | D25 | D23 | Ar1 | D23 | Ar1 |
| 225 | D26 | D23 | Ar1 | D23 | Ar1 |
| 226 | D1 | D24 | Ar1 | D24 | Ar1 |
| 227 | D2 | D24 | Ar1 | D24 | Ar1 |
| 228 | D3 | D24 | Ar1 | D24 | Ar1 |
| 229 | D4 | D24 | Ar1 | D24 | Ar1 |
| 230 | D5 | D24 | Ar1 | D24 | Ar1 |
| 231 | D6 | D24 | Ar1 | D24 | Ar1 |
| 232 | D7 | D24 | Ar1 | D24 | Ar1 |
| 233 | D8 | D24 | Ar1 | D24 | Ar1 |
| 234 | D9 | D24 | Ar1 | D24 | Ar1 |
| 235 | D10 | D24 | Ar1 | D24 | Ar1 |
| 236 | D11 | D24 | Ar1 | D24 | Ar1 |
| 237 | D12 | D24 | Ar1 | D24 | Ar1 |
| 238 | D13 | D24 | Ar1 | D24 | Ar1 |
| 239 | D14 | D24 | Ar1 | D24 | Ar1 |
| 240 | D15 | D24 | Ar1 | D24 | Ar1 |
| 241 | D16 | D24 | Ar1 | D24 | Ar1 |
| 242 | D17 | D24 | Ar1 | D24 | Ar1 |
| 243 | D18 | D24 | Ar1 | D24 | Ar1 |
| 244 | D19 | D24 | Ar1 | D24 | Ar1 |
| 245 | D20 | D24 | Ar1 | D24 | Ar1 |
| 246 | D21 | D24 | Ar1 | D24 | Ar1 |
| 247 | D22 | D24 | Ar1 | D24 | Ar1 |
| 248 | D23 | D24 | Ar1 | D24 | Ar1 |
| 249 | D25 | D24 | Ar1 | D24 | Ar1 |
| 250 | D26 | D24 | Ar1 | D24 | Ar1 |
| 251 | D1 | D25 | Ar1 | D25 | Ar1 |
| 252 | D2 | D25 | Ar1 | D25 | Ar1 |
| 253 | D3 | D25 | Ar1 | D25 | Ar1 |
| 254 | D4 | D25 | Ar1 | D25 | Ar1 |
| 255 | D5 | D25 | Ar1 | D25 | Ar1 |
| 256 | D6 | D25 | Ar1 | D25 | Ar1 |
| 257 | D7 | D25 | Ar1 | D25 | Ar1 |
| 258 | D8 | D25 | Ar1 | D25 | Ar1 |
| 259 | D9 | D25 | Ar1 | D25 | Ar1 |
| 260 | D10 | D25 | Ar1 | D25 | Ar1 |
| 261 | D11 | D25 | Ar1 | D25 | Ar1 |
| 262 | D12 | D25 | Ar1 | D25 | Ar1 |
| 263 | D13 | D25 | Ar1 | D25 | Ar1 |
| 264 | D14 | D25 | Ar1 | D25 | Ar1 |
| 265 | D15 | D25 | Ar1 | D25 | Ar1 |
| 266 | D16 | D25 | Ar1 | D25 | Ar1 |
| 267 | D17 | D25 | Ar1 | D25 | Ar1 |
| 268 | D18 | D25 | Ar1 | D25 | Ar1 |
| 269 | D19 | D25 | Ar1 | D25 | Ar1 |
| 270 | D20 | D25 | Ar1 | D25 | Ar1 |
| 271 | D21 | D25 | Ar1 | D25 | Ar1 |
| 272 | D22 | D25 | Ar1 | D26 | Ar1 |
| 273 | D23 | D25 | Ar1 | D25 | Ar1 |
| 274 | D24 | D25 | Ar1 | D25 | Ar1 |
| 275 | D26 | D25 | Ar1 | D25 | Ar1 |
| 276 | D1 | D26 | Ar1 | D26 | Ar1 |
| 277 | D2 | D26 | Ar1 | D26 | Ar1 |
| 278 | D3 | D26 | Ar1 | D26 | Ar1 |
| 279 | D4 | D26 | Ar1 | D26 | Ar1 |
| 280 | D5 | D26 | Ar1 | D26 | Ar1 |
| 281 | D6 | D26 | Ar1 | D26 | Ar1 |
| 282 | D7 | D26 | Ar1 | D26 | Ar1 |
| 283 | D8 | D26 | Ar1 | D26 | Ar1 |
| 284 | D9 | D26 | Ar1 | D26 | Ar1 |
| 285 | D10 | D26 | Ar1 | D26 | Ar1 |
| 286 | D11 | D26 | Ar1 | D26 | Ar1 |
| 287 | D12 | D26 | Ar1 | D26 | Ar1 |
| 288 | D13 | D26 | Ar1 | D26 | Ar1 |
| 289 | D14 | D26 | Ar1 | D26 | Ar1 |
| 290 | D15 | D26 | Ar1 | D26 | Ar1 |
| 291 | D16 | D26 | Ar1 | D26 | Ar1 |
| 292 | D17 | D26 | Ar1 | D26 | Ar1 |
| 293 | D18 | D26 | Ar1 | D26 | Ar1 |
| 294 | D19 | D26 | Ar1 | D26 | Ar1 |
| 295 | D20 | D26 | Ar1 | D26 | Ar1 |
| 296 | D21 | D26 | Ar1 | D26 | Ar1 |
| 297 | D22 | D26 | Ar1 | D26 | Ar1 |
| 298 | D23 | D26 | Ar1 | D26 | Ar1 |
| 299 | D24 | D26 | Ar1 | D26 | Ar1 |
| 300 | D25 | D26 | Ar1 | D26 | Ar1 |
| 301 | D1 | D1 | Ar1 | D21 | Ar1 |
| 302 | D2 | D2 | Ar1 | D21 | Ar1 |
| 303 | D3 | D3 | Ar1 | D21 | Ar1 |
| 304 | D4 | D4 | Ar1 | D21 | Ar1 |
| 305 | D5 | D5 | Ar1 | D21 | Ar1 |
| 306 | D6 | D6 | Ar1 | D21 | Ar1 |
| 307 | D7 | D7 | Ar1 | D21 | Ar1 |
| 308 | D8 | D8 | Ar1 | D21 | Ar1 |
| 309 | D9 | D9 | Ar1 | D21 | Ar1 |
| 310 | D10 | D10 | Ar1 | D21 | Ar1 |
| 311 | D11 | D11 | Ar1 | D21 | Ar1 |
| 312 | D12 | D12 | Ar1 | D21 | Ar1 |
| 313 | D13 | D13 | Ar1 | D21 | Ar1 |
| 314 | D14 | D14 | Ar1 | D21 | Ar1 |
| 315 | D15 | D15 | Ar1 | D21 | Ar1 |
| 316 | D16 | D16 | Ar1 | D21 | Ar1 |
| 317 | D17 | D17 | Ar1 | D21 | Ar1 |
| 318 | D18 | D18 | Ar1 | D21 | Ar1 |
| 319 | D19 | D19 | Ar1 | D21 | Ar1 |
| 320 | D20 | D20 | Ar1 | D21 | Ar1 |
| 321 | D22 | D22 | Ar1 | D21 | Ar1 |
| 322 | D23 | D23 | Ar1 | D21 | Ar1 |
| 323 | D24 | D24 | Ar1 | D21 | Ar1 |
| 324 | D25 | D25 | Ar1 | D21 | Ar1 |
| 325 | D26 | D26 | Ar1 | D21 | Ar1 |
| 326 | D1 | D1 | Ar1 | D22 | Ar1 |
| 327 | D2 | D2 | Ar1 | D22 | Ar1 |
| 328 | D3 | D3 | Ar1 | D22 | Ar1 |
| 329 | D4 | D4 | Ar1 | D22 | Ar1 |
| 330 | D5 | D5 | Ar1 | D22 | Ar1 |
| 331 | D6 | D6 | Ar1 | D22 | Ar1 |
| 332 | D7 | D7 | Ar1 | D22 | Ar1 |
| 333 | D8 | D8 | Ar1 | D22 | Ar1 |
| 334 | D9 | D9 | Ar1 | D22 | Ar1 |
| 335 | D10 | D10 | Ar1 | D22 | Ar1 |
| 336 | D11 | D11 | Ar1 | D22 | Ar1 |
| 337 | D12 | D12 | Ar1 | D22 | Ar1 |
| 338 | D13 | D13 | Ar1 | D22 | Ar1 |
| 339 | D14 | D14 | Ar1 | D22 | Ar1 |
| 340 | D15 | D15 | Ar1 | D22 | Ar1 |
| 341 | D16 | D16 | Ar1 | D22 | Ar1 |
| 342 | D17 | D17 | Ar1 | D22 | Ar1 |
| 343 | D18 | D18 | Ar1 | D22 | Ar1 |
| 344 | D19 | D19 | Ar1 | D22 | Ar1 |
| 345 | D20 | D20 | Ar1 | D22 | Ar1 |
| 346 | D21 | D21 | Ar1 | D22 | Ar1 |
| 347 | D23 | D23 | Ar1 | D22 | Ar1 |
| 348 | D24 | D24 | Ar1 | D22 | Ar1 |
| 349 | D25 | D25 | Ar1 | D22 | Ar1 |
| 350 | D26 | D26 | Ar1 | D22 | Ar1 |
| 351 | D1 | D1 | Ar1 | D23 | Ar1 |
| 352 | D2 | D2 | Ar1 | D23 | Ar1 |
| 353 | D3 | D3 | Ar1 | D23 | Ar1 |
| 354 | D4 | D4 | Ar1 | D23 | Ar1 |
| 355 | D5 | D5 | Ar1 | D23 | Ar1 |
| 356 | D6 | D6 | Ar1 | D23 | Ar1 |
| 357 | D7 | D7 | Ar1 | D23 | Ar1 |
| 358 | D8 | D8 | Ar1 | D23 | Ar1 |
| 359 | D9 | D9 | Ar1 | D23 | Ar1 |
| 360 | D10 | D10 | Ar1 | D23 | Ar1 |
| 361 | D11 | D11 | Ar1 | D23 | Ar1 |
| 362 | D12 | D12 | Ar1 | D22 | Ar1 |
| 363 | D13 | D13 | Ar1 | D23 | Ar1 |
| 364 | D14 | D14 | Ar1 | D23 | Ar1 |
| 365 | D15 | D15 | Ar1 | D23 | Ar1 |
| 366 | D16 | D16 | Ar1 | D23 | Ar1 |
| 367 | D17 | D17 | Ar1 | D23 | Ar1 |
| 368 | D18 | D18 | Ar1 | D23 | Ar1 |
| 369 | D19 | D19 | Ar1 | D23 | Ar1 |
| 370 | D20 | D20 | Ar1 | D23 | Ar1 |
| 371 | D21 | D21 | Ar1 | D23 | Ar1 |
| 372 | D22 | D22 | Ar1 | D23 | Ar1 |
| 373 | D24 | D24 | Ar1 | D23 | Ar1 |
| 374 | D25 | D25 | Ar1 | D23 | Ar1 |
| 375 | D26 | D26 | Ar1 | D23 | Ar1 |
| 376 | D1 | D1 | Ar1 | D24 | Ar1 |
| 377 | D2 | D2 | Ar1 | D24 | Ar1 |
| 378 | D3 | D3 | Ar1 | D24 | Ar1 |
| 379 | D4 | D4 | Ar1 | D24 | Ar1 |
| 380 | D5 | D5 | Ar1 | D24 | Ar1 |
| 381 | D6 | D6 | Ar1 | D24 | Ar1 |
| 382 | D7 | D7 | Ar1 | D24 | Ar1 |
| 383 | D8 | D8 | Ar1 | D24 | Ar1 |
| 384 | D9 | D9 | Ar1 | D24 | Ar1 |
| 385 | D10 | D10 | Ar1 | D24 | Ar1 |
| 386 | D11 | D11 | Ar1 | D24 | Ar1 |
| 387 | D12 | D12 | Ar1 | D24 | Ar1 |
| 388 | D13 | D13 | Ar1 | D24 | Ar1 |
| 389 | D14 | D14 | Ar1 | D24 | Ar1 |
| 390 | D15 | D15 | Ar1 | D24 | Ar1 |
| 391 | D16 | D16 | Ar1 | D24 | Ar1 |
| 392 | D17 | D17 | Ar1 | D24 | Ar1 |
| 393 | D18 | D18 | Ar1 | D24 | Ar1 |
| 394 | D19 | D19 | Ar1 | D24 | Ar1 |
| 395 | D20 | D20 | Ar1 | D24 | Ar1 |
| 396 | D21 | D21 | Ar1 | D24 | Ar1 |
| 397 | D22 | D22 | Ar1 | D24 | Ar1 |
| 398 | D23 | D23 | Ar1 | D24 | Ar1 |
| 399 | D25 | D25 | Ar1 | D24 | Ar1 |
| 400 | D26 | D26 | Ar1 | D24 | Ar1 |
| 401 | D1 | D1 | Ar1 | D25 | Ar1 |
| 402 | D2 | D2 | Ar1 | D25 | Ar1 |
| 403 | D3 | D3 | Ar1 | D25 | Ar1 |
| 404 | D4 | D4 | Ar1 | D25 | Ar1 |
| 405 | D5 | D5 | Ar1 | D25 | Ar1 |
| 406 | D6 | D6 | Ar1 | D25 | Ar1 |
| 407 | D7 | D7 | Ar1 | D25 | Ar1 |
| 408 | D8 | D8 | Ar1 | D25 | Ar1 |
| 409 | D9 | D9 | Ar1 | D25 | Ar1 |
| 410 | D10 | D10 | Ar1 | D25 | Ar1 |
| 411 | D11 | D11 | Ar1 | D25 | Ar1 |
| 412 | D12 | D12 | Ar1 | D25 | Ar1 |
| 413 | D13 | D13 | Ar1 | D25 | Ar1 |
| 414 | D14 | D14 | Ar1 | D25 | Ar1 |
| 415 | D15 | D15 | Ar1 | D25 | Ar1 |
| 416 | D16 | D16 | Ar1 | D25 | Ar1 |
| 417 | D17 | D17 | Ar1 | D25 | Ar1 |
| 418 | D18 | D18 | Ar1 | D25 | Ar1 |
| 419 | D19 | D19 | Ar1 | D25 | Ar1 |
| 420 | D20 | D20 | Ar1 | D25 | Ar1 |
| 421 | D21 | D21 | Ar1 | D25 | Ar1 |
| 422 | D22 | D22 | Ar1 | D25 | Ar1 |
| 423 | D23 | D23 | Ar1 | D25 | Ar1 |
| 424 | D24 | D24 | Ar1 | D25 | Ar1 |
| 425 | D25 | D26 | Ar1 | D25 | Ar1 |
| 426 | D1 | D1 | Ar1 | D26 | Ar1 |
| 427 | D2 | D2 | Ar1 | D26 | Ar1 |
| 428 | D3 | D3 | Ar1 | D26 | Ar1 |
| 429 | D4 | D4 | Ar1 | D26 | Ar1 |
| 430 | D5 | D5 | Ar1 | D26 | Ar1 |
| 431 | D6 | D6 | Ar1 | D26 | Ar1 |
| 432 | D7 | D7 | Ar1 | D26 | Ar1 |
| 433 | D8 | D8 | Ar1 | D26 | Ar1 |
| 434 | D9 | D9 | Ar1 | D26 | Ar1 |
| 435 | D10 | D10 | Ar1 | D26 | Ar1 |
| 436 | D11 | D11 | Ar1 | D26 | Ar1 |
| 437 | D12 | D12 | Ar1 | D26 | Ar1 |
| 438 | D13 | D13 | Ar1 | D26 | Ar1 |
| 439 | D14 | D14 | Ar1 | D26 | Ar1 |
| 440 | D15 | D15 | Ar1 | D26 | Ar1 |
| 441 | D16 | D16 | Ar1 | D26 | Ar1 |
| 442 | D17 | D17 | Ar1 | D26 | Ar1 |
| 443 | D18 | D18 | Ar1 | D26 | Ar1 |
| 444 | D19 | D19 | Ar1 | D26 | Ar1 |
| 445 | D20 | D20 | Ar1 | D26 | Ar1 |
| 446 | D21 | D21 | Ar1 | D26 | Ar1 |
| 447 | D22 | D22 | Ar1 | D26 | Ar1 |
| 448 | D23 | D23 | Ar1 | D26 | Ar1 |
| 449 | D24 | D24 | Ar1 | D26 | Ar1 |
| 450 | D25 | D25 | Ar1 | D26 | Ar1 |
| 451 | D21 | D21 | Ar1 | D1 | Ar1 |
| 452 | D21 | D21 | Ar1 | D2 | Ar1 |
| 453 | D21 | D21 | Ar1 | D3 | Ar1 |
| 454 | D21 | D21 | Ar1 | D4 | Ar1 |
| 455 | D21 | D21 | Ar1 | D5 | Ar1 |
| 456 | D21 | D21 | Ar1 | D6 | Ar1 |
| 457 | D21 | D21 | Ar1 | D7 | Ar1 |
| 458 | D21 | D21 | Ar1 | D8 | Ar1 |
| 459 | D21 | D21 | Ar1 | D9 | Ar1 |
| 460 | D21 | D21 | Ar1 | D10 | Ar1 |
| 461 | D21 | D21 | Ar1 | D11 | Ar1 |
| 462 | D21 | D21 | Ar1 | D12 | Ar1 |
| 463 | D21 | D21 | Ar1 | D13 | Ar1 |
| 464 | D21 | D21 | Ar1 | D14 | Ar1 |
| 465 | D21 | D21 | Ar1 | D15 | Ar1 |
| 466 | D21 | D21 | Ar1 | D16 | Ar1 |
| 467 | D21 | D21 | Ar1 | D17 | Ar1 |
| 468 | D21 | D21 | Ar1 | D18 | Ar1 |
| 469 | D21 | D21 | Ar1 | D19 | Ar1 |
| 470 | D21 | D21 | Ar1 | D20 | Ar1 |
| 471 | D21 | D21 | Ar1 | D22 | Ar1 |
| 472 | D21 | D21 | Ar1 | D23 | Ar1 |
| 473 | D21 | D21 | Ar1 | D24 | Ar1 |
| 474 | D21 | D21 | Ar1 | D25 | Ar1 |
| 475 | D21 | D21 | Ar1 | D26 | Ar1 |
| 476 | D22 | D22 | Ar1 | D1 | Ar1 |
| 477 | D22 | D22 | Ar1 | D2 | Ar1 |
| 478 | D22 | D22 | Ar1 | D3 | Ar1 |
| 479 | D22 | D22 | Ar1 | D4 | Ar1 |
| 480 | D22 | D22 | Ar1 | D5 | Ar1 |
| 481 | D22 | D22 | Ar1 | D6 | Ar1 |
| 482 | D22 | D22 | Ar1 | D7 | Ar1 |
| 483 | D22 | D22 | Ar1 | D8 | Ar1 |
| 484 | D22 | D22 | Ar1 | D9 | Ar1 |
| 485 | D22 | D22 | Ar1 | D10 | Ar1 |
| 486 | D22 | D22 | Ar1 | D11 | Ar1 |
| 487 | D22 | D22 | Ar1 | D12 | Ar1 |
| 488 | D22 | D22 | Ar1 | D13 | Ar1 |
| 489 | D22 | D22 | Ar1 | D14 | Ar1 |
| 490 | D22 | D22 | Ar1 | D15 | Ar1 |
| 491 | D22 | D22 | Ar1 | D16 | Ar1 |
| 492 | D22 | D22 | Ar1 | D17 | Ar1 |
| 493 | D22 | D22 | Ar1 | D18 | Ar1 |
| 494 | D22 | D22 | Ar1 | D19 | Ar1 |
| 495 | D22 | D22 | Ar1 | D20 | Ar1 |
| 496 | D22 | D22 | Ar1 | D21 | Ar1 |
| 497 | D22 | D22 | Ar1 | D23 | Ar1 |
| 498 | D22 | D22 | Ar1 | D24 | Ar1 |
| 499 | D22 | D22 | Ar1 | D25 | Ar1 |
| 500 | D22 | D22 | Ar1 | D26 | Ar1 |
| 501 | D23 | D23 | Ar1 | D1 | Ar1 |
| 502 | D23 | D23 | Ar1 | D2 | Ar1 |
| 503 | D23 | D23 | Ar1 | D3 | Ar1 |
| 504 | D23 | D23 | Ar1 | D4 | Ar1 |
| 505 | D23 | D23 | Ar1 | D5 | Ar1 |
| 506 | D23 | D23 | Ar1 | D6 | Ar1 |
| 507 | D23 | D23 | Ar1 | D7 | Ar1 |
| 508 | D23 | D23 | Ar1 | D8 | Ar1 |
| 509 | D23 | D23 | Ar1 | D9 | Ar1 |
| 510 | D23 | D23 | Ar1 | D10 | Ar1 |
| 511 | D23 | D23 | Ar1 | D11 | Ar1 |
| 512 | D23 | D23 | Ar1 | D12 | Ar1 |
| 513 | D23 | D23 | Ar1 | D13 | Ar1 |
| 514 | D23 | D23 | Ar1 | D14 | Ar1 |
| 515 | D23 | D23 | Ar1 | D15 | Ar1 |
| 516 | D23 | D23 | Ar1 | D16 | Ar1 |
| 517 | D23 | D23 | Ar1 | D17 | Ar1 |
| 518 | D23 | D23 | Ar1 | D18 | Ar1 |
| 519 | D23 | D23 | Ar1 | D19 | Ar1 |
| 520 | D23 | D23 | Ar1 | D20 | Ar1 |
| 521 | D23 | D23 | Ar1 | D21 | Ar1 |
| 522 | D23 | D23 | Ar1 | D22 | Ar1 |
| 523 | D23 | D23 | Ar1 | D24 | Ar1 |
| 524 | D23 | D23 | Ar1 | D25 | Ar1 |
| 525 | D23 | D23 | Ar1 | D26 | Ar1 |
| 526 | D24 | D24 | Ar1 | D1 | Ar1 |
| 527 | D24 | D24 | Ar1 | D2 | Ar1 |
| 528 | D24 | D24 | Ar1 | D3 | Ar1 |
| 529 | D24 | D24 | Ar1 | D4 | Ar1 |
| 530 | D24 | D24 | Ar1 | D5 | Ar1 |
| 531 | D24 | D24 | Ar1 | D6 | Ar1 |
| 532 | D24 | D24 | Ar1 | D7 | Ar1 |
| 533 | D24 | D24 | Ar1 | D8 | Ar1 |
| 534 | D24 | D24 | Ar1 | D9 | Ar1 |
| 535 | D24 | D24 | Ar1 | D10 | Ar1 |
| 536 | D24 | D24 | Ar1 | D11 | Ar1 |
| 537 | D24 | D24 | Ar1 | D12 | Ar1 |
| 538 | D24 | D24 | Ar1 | D13 | Ar1 |
| 539 | D24 | D24 | Ar1 | D14 | Ar1 |
| 540 | D24 | D24 | Ar1 | D15 | Ar1 |
| 541 | D24 | D24 | Ar1 | D16 | Ar1 |
| 542 | D24 | D24 | Ar1 | D17 | Ar1 |
| 543 | D24 | D24 | Ar1 | D18 | Ar1 |
| 544 | D24 | D24 | Ar1 | D19 | Ar1 |
| 545 | D24 | D24 | Ar1 | D20 | Ar1 |
| 546 | D24 | D24 | Ar1 | D21 | Ar1 |
| 547 | D24 | D24 | Ar1 | D22 | Ar1 |
| 548 | D24 | D24 | Ar1 | D23 | Ar1 |
| 549 | D24 | D24 | Ar1 | D25 | Ar1 |
| 550 | D24 | D24 | Ar1 | D26 | Ar1 |
| 551 | D25 | D25 | Ar1 | D1 | Ar1 |
| 552 | D25 | D25 | Ar1 | D2 | Ar1 |
| 553 | D25 | D25 | Ar1 | D3 | Ar1 |
| 554 | D25 | D25 | Ar1 | D4 | Ar1 |
| 555 | D25 | D25 | Ar1 | D5 | Ar1 |
| 556 | D25 | D25 | Ar1 | D6 | Ar1 |
| 557 | D25 | D25 | Ar1 | D7 | Ar1 |
| 558 | D25 | D25 | Ar1 | D8 | Ar1 |
| 559 | D25 | D25 | Ar1 | D9 | Ar1 |
| 560 | D25 | D25 | Ar1 | D10 | Ar1 |
| 561 | D25 | D25 | Ar1 | D11 | Ar1 |
| 562 | D25 | D25 | Ar1 | D12 | Ar1 |
| 563 | D25 | D25 | Ar1 | D13 | Ar1 |
| 564 | D25 | D25 | Ar1 | D14 | Ar1 |
| 565 | D25 | D25 | Ar1 | D15 | Ar1 |
| 566 | D25 | D25 | Ar1 | D16 | Ar1 |
| 567 | D25 | D25 | Ar1 | D17 | Ar1 |
| 568 | D25 | D25 | Ar1 | D18 | Ar1 |
| 569 | D25 | D25 | Ar1 | D19 | Ar1 |
| 570 | D25 | D25 | Ar1 | D20 | Ar1 |
| 571 | D25 | D25 | Ar1 | D21 | Ar1 |
| 572 | D25 | D25 | Ar1 | D22 | Ar1 |
| 573 | D25 | D25 | Ar1 | D23 | Ar1 |
| 574 | D25 | D25 | Ar1 | D24 | Ar1 |
| 575 | D25 | D25 | Ar1 | D26 | Ar1 |
| 576 | D26 | D26 | Ar1 | D1 | Ar1 |
| 577 | D26 | D26 | Ar1 | D2 | Ar1 |
| 578 | D26 | D26 | Ar1 | D3 | Ar1 |
| 579 | D26 | D26 | Ar1 | D4 | Ar1 |
| 580 | D26 | D26 | Ar1 | D5 | Ar1 |
| 581 | D26 | D26 | Ar1 | D6 | Ar1 |
| 582 | D26 | D26 | Ar1 | D7 | Ar1 |
| 583 | D26 | D26 | Ar1 | D8 | Ar1 |
| 584 | D26 | D26 | Ar1 | D9 | Ar1 |
| 585 | D26 | D26 | Ar1 | D10 | Ar1 |
| 586 | D26 | D26 | Ar1 | D11 | Ar1 |
| 587 | D26 | D26 | Ar1 | D12 | Ar1 |
| 588 | D26 | D26 | Ar1 | D13 | Ar1 |
| 589 | D26 | D26 | Ar1 | D14 | Ar1 |
| 590 | D26 | D26 | Ar1 | D15 | Ar1 |
| 591 | D26 | D26 | Ar1 | D16 | Ar1 |
| 592 | D26 | D26 | Ar1 | D17 | Ar1 |
| 593 | D26 | D26 | Ar1 | D18 | Ar1 |
| 594 | D26 | D26 | Ar1 | D19 | Ar1 |
| 595 | D26 | D26 | Ar1 | D20 | Ar1 |
| 596 | D26 | D26 | Ar1 | D21 | Ar1 |
| 597 | D26 | D26 | Ar1 | D22 | Ar1 |
| 598 | D26 | D26 | Ar1 | D23 | Ar1 |
| 599 | D26 | D26 | Ar1 | D24 | Ar1 |
| 600 | D26 | D26 | Ar1 | D25 | Ar1 |
| 601 | D1 | D21 | Ar1 | D1 | Ar1 |
| 602 | D2 | D21 | Ar1 | D2 | Ar1 |
| 603 | D3 | D21 | Ar1 | D3 | Ar1 |
| 604 | D4 | D21 | Ar1 | D4 | Ar1 |
| 605 | D5 | D21 | Ar1 | D5 | Ar1 |
| 606 | D6 | D21 | Ar1 | D6 | Ar1 |
| 607 | D7 | D21 | Ar1 | D7 | Ar1 |
| 608 | D8 | D21 | Ar1 | D8 | Ar1 |
| 609 | D9 | D21 | Ar1 | D9 | Ar1 |
| 610 | D10 | D21 | Ar1 | D10 | Ar1 |
| 611 | D11 | D21 | Ar1 | D11 | Ar1 |
| 612 | D12 | D21 | Ar1 | D12 | Ar1 |
| 613 | D13 | D21 | Ar1 | D13 | Ar1 |
| 614 | D14 | D21 | Ar1 | D14 | Ar1 |
| 615 | D15 | D21 | Ar1 | D15 | Ar1 |
| 616 | D16 | D21 | Ar1 | D16 | Ar1 |
| 617 | D17 | D21 | Ar1 | D17 | Ar1 |
| 618 | D18 | D21 | Ar1 | D18 | Ar1 |
| 619 | D19 | D21 | Ar1 | D19 | Ar1 |
| 620 | D20 | D21 | Ar1 | D20 | Ar1 |
| 621 | D22 | D21 | Ar1 | D22 | Ar1 |
| 622 | D23 | D21 | Ar1 | D23 | Ar1 |
| 623 | D24 | D21 | Ar1 | D24 | Ar1 |
| 624 | D25 | D21 | Ar1 | D25 | Ar1 |
| 625 | D26 | D21 | Ar1 | D26 | Ar1 |
| 626 | D1 | D22 | Ar1 | D1 | Ar1 |
| 627 | D2 | D22 | Ar1 | D2 | Ar1 |
| 628 | D3 | D22 | Ar1 | D3 | Ar1 |
| 629 | D4 | D22 | Ar1 | D4 | Ar1 |
| 630 | D5 | D22 | Ar1 | D5 | Ar1 |
| 631 | D6 | D22 | Ar1 | D6 | Ar1 |
| 632 | D7 | D22 | Ar1 | D7 | Ar1 |
| 633 | D8 | D22 | Ar1 | D8 | Ar1 |
| 634 | D9 | D22 | Ar1 | D9 | Ar1 |
| 635 | D10 | D22 | Ar1 | D10 | Ar1 |
| 636 | D11 | D22 | Ar1 | D11 | Ar1 |
| 637 | D12 | D22 | Ar1 | D12 | Ar1 |
| 638 | D13 | D22 | Ar1 | D13 | Ar1 |
| 639 | D14 | D22 | Ar1 | D14 | Ar1 |
| 640 | D15 | D22 | Ar1 | D15 | Ar1 |
| 641 | D18 | D22 | Ar1 | D16 | Ar1 |
| 642 | D17 | D22 | Ar1 | D17 | Ar1 |
| 643 | D16 | D22 | Ar1 | D18 | Ar1 |
| 644 | D19 | D22 | Ar1 | D19 | Ar1 |
| 645 | D20 | D22 | Ar1 | D20 | Ar1 |
| 646 | D21 | D22 | Ar1 | D21 | Ar1 |
| 647 | D23 | D22 | Ar1 | D23 | Ar1 |
| 648 | D24 | D22 | Ar1 | D24 | Ar1 |
| 649 | D25 | D22 | Ar1 | D25 | Ar1 |
| 650 | D26 | D22 | Ar1 | D26 | Ar1 |
| 651 | D1 | D23 | Ar1 | D1 | Ar1 |
| 652 | D2 | D23 | Ar1 | D2 | Ar1 |
| 653 | D3 | D23 | Ar1 | D3 | Ar1 |
| 654 | D4 | D23 | Ar1 | D4 | Ar1 |
| 655 | D5 | D23 | Ar1 | D5 | Ar1 |
| 656 | D8 | D23 | Ar1 | D6 | Ar1 |
| 657 | D7 | D23 | Ar1 | D7 | Ar1 |
| 658 | D8 | D23 | Ar1 | D8 | Ar1 |
| 659 | D9 | D23 | Ar1 | D9 | Ar1 |
| 660 | D10 | D23 | Ar1 | D10 | Ar1 |
| 661 | D11 | D23 | Ar1 | D11 | Ar1 |
| 662 | D12 | D23 | Ar1 | D12 | Ar1 |
| 663 | D13 | D23 | Ar1 | D13 | Ar1 |
| 664 | D14 | D23 | Ar1 | D14 | Ar1 |
| 665 | D15 | D23 | Ar1 | D15 | Ar1 |
| 666 | D16 | D23 | Ar1 | D16 | Ar1 |
| 667 | D17 | D23 | Ar1 | D17 | Ar1 |
| 668 | D18 | D23 | Ar1 | D18 | Ar1 |
| 669 | D19 | D23 | Ar1 | D19 | Ar1 |
| 670 | D20 | D23 | Ar1 | D20 | Ar1 |
| 671 | D21 | D23 | Ar1 | D21 | Ar1 |
| 672 | D22 | D23 | Ar1 | D22 | Ar1 |
| 673 | D24 | D23 | Ar1 | D24 | Ar1 |
| 674 | D25 | D23 | Ar1 | D25 | Ar1 |
| 675 | D26 | D23 | Ar1 | D26 | Ar1 |
| 676 | D1 | D24 | Ar1 | D1 | Ar1 |
| 677 | D2 | D24 | Ar1 | D2 | Ar1 |
| 678 | D3 | D24 | Ar1 | D3 | Ar1 |
| 679 | D4 | D24 | Ar1 | D4 | Ar1 |
| 680 | D5 | D24 | Ar1 | D5 | Ar1 |
| 681 | D6 | D24 | Ar1 | D6 | Ar1 |
| 682 | D7 | D24 | Ar1 | D7 | Ar1 |
| 683 | D8 | D24 | Ar1 | D8 | Ar1 |
| 684 | D9 | D24 | Ar1 | D9 | Ar1 |
| 685 | D10 | D24 | Ar1 | D10 | Ar1 |
| 686 | D11 | D24 | Ar1 | D11 | Ar1 |
| 687 | D12 | D24 | Ar1 | D12 | Ar1 |
| 688 | D13 | D24 | Ar1 | D13 | Ar1 |
| 689 | D14 | D24 | Ar1 | D14 | Ar1 |
| 690 | D15 | D24 | Ar1 | D15 | Ar1 |
| 691 | D16 | D24 | Ar1 | D16 | Ar1 |
| 692 | D17 | D24 | Ar1 | D17 | Ar1 |
| 693 | D18 | D24 | Ar1 | D18 | Ar1 |
| 694 | D19 | D24 | Ar1 | D19 | Ar1 |
| 695 | D20 | D24 | Ar1 | D20 | Ar1 |
| 696 | D21 | D24 | Ar1 | D21 | Ar1 |
| 697 | D22 | D24 | Ar1 | D22 | Ar1 |
| 698 | D23 | D24 | Ar1 | D23 | Ar1 |
| 699 | D25 | D24 | Ar1 | D25 | Ar1 |
| 700 | D26 | D24 | Ar1 | D26 | Ar1 |
| 701 | D1 | D25 | Ar1 | D1 | Ar1 |
| 702 | D2 | D25 | Ar1 | D2 | Ar1 |
| 703 | D3 | D26 | Ar1 | D3 | Ar1 |
| 704 | D4 | D25 | Ar1 | D4 | Ar1 |
| 705 | D5 | D25 | Ar1 | D5 | Ar1 |
| 706 | D6 | D25 | Ar1 | D6 | Ar1 |
| 707 | D7 | D25 | Ar1 | D7 | Ar1 |
| 708 | D8 | D25 | Ar1 | D8 | Ar1 |
| 709 | D9 | D25 | Ar1 | D9 | Ar1 |
| 710 | D10 | D25 | Ar1 | D10 | Ar1 |
| 711 | D11 | D25 | Ar1 | D11 | Ar1 |
| 712 | D12 | D25 | Ar1 | D12 | Ar1 |
| 713 | D13 | D25 | Ar1 | D13 | Ar1 |
| 714 | D14 | D25 | Ar1 | D14 | Ar1 |
| 715 | D15 | D25 | Ar1 | D15 | Ar1 |
| 716 | D16 | D25 | Ar1 | D16 | Ar1 |
| 717 | D17 | D25 | Ar1 | D17 | Ar1 |
| 718 | D18 | D25 | Ar1 | D18 | Ar1 |
| 719 | D19 | D25 | Ar1 | D19 | Ar1 |
| 720 | D20 | D25 | Ar1 | D20 | Ar1 |
| 721 | D21 | D25 | Ar1 | D21 | Ar1 |
| 722 | D22 | D25 | Ar1 | D22 | Ar1 |
| 723 | D23 | D25 | Ar1 | D23 | Ar1 |
| 724 | D24 | D25 | Ar1 | D24 | Ar1 |
| 725 | D26 | D25 | Ar1 | D26 | Ar1 |
| 726 | D1 | D26 | Ar1 | D1 | Ar1 |
| 727 | D2 | D26 | Ar1 | D2 | Ar1 |
| 728 | D3 | D26 | Ar1 | D3 | Ar1 |
| 729 | D4 | D26 | Ar1 | D4 | Ar1 |
| 730 | D5 | D26 | Ar1 | D5 | Ar1 |
| 731 | D6 | D26 | Ar1 | D6 | Ar1 |
| 732 | D7 | D26 | Ar1 | D7 | Ar1 |
| 733 | D8 | D26 | Ar1 | D8 | Ar1 |
| 734 | D9 | D26 | Ar1 | D9 | Ar1 |
| 735 | D10 | D26 | Ar1 | D10 | Ar1 |
| 736 | D11 | D26 | Ar1 | D11 | Ar1 |
| 737 | D12 | D26 | Ar1 | D12 | Ar1 |
| 738 | D13 | D26 | Ar1 | D13 | Ar1 |
| 739 | D14 | D26 | Ar1 | D14 | Ar1 |
| 740 | D15 | D26 | Ar1 | D15 | Ar1 |
| 741 | D16 | D26 | Ar1 | D16 | Ar1 |
| 742 | D17 | D26 | Ar1 | D17 | Ar1 |
| 743 | D18 | D26 | Ar1 | D18 | Ar1 |
| 744 | D19 | D26 | Ar1 | D19 | Ar1 |
| 745 | D20 | D26 | Ar1 | D20 | Ar1 |
| 746 | D21 | D26 | Ar1 | D21 | Ar1 |
| 747 | D22 | D26 | Ar1 | D22 | Ar1 |
| 748 | D23 | D26 | Ar1 | D23 | Ar1 |
| 749 | D24 | D26 | Ar1 | D24 | Ar1 |
| 750 | D25 | D26 | Ar1 | D25 | Ar1 |
| 751 | D21 | D1 | Ar1 | D21 | Ar1 |
| 752 | D21 | D2 | Ar1 | D21 | Ar1 |
| 753 | D21 | D3 | Ar1 | D21 | Ar1 |
| 754 | D21 | D4 | Ar1 | D21 | Ar1 |
| 755 | D21 | D5 | Ar1 | D21 | Ar1 |
| 756 | D21 | D6 | Ar1 | D21 | Ar1 |
| 757 | D21 | D7 | Ar1 | D21 | Ar1 |
| 758 | D21 | D8 | Ar1 | D21 | Ar1 |
| 759 | D21 | D9 | Ar1 | D21 | Ar1 |
| 760 | D21 | D10 | Ar1 | D21 | Ar1 |
| 761 | D21 | D11 | Ar1 | D21 | Ar1 |
| 762 | D21 | D12 | Ar1 | D21 | Ar1 |
| 763 | D21 | D13 | Ar1 | D21 | Ar1 |
| 764 | D21 | D14 | Ar1 | D21 | Ar1 |
| 765 | D21 | D15 | Ar1 | D21 | Ar1 |
| 766 | D21 | D16 | Ar1 | D21 | Ar1 |
| 767 | D21 | D17 | Ar1 | D21 | Ar1 |
| 768 | D21 | D18 | Ar1 | D21 | Ar1 |
| 769 | D21 | D19 | Ar1 | D21 | Ar1 |
| 770 | D21 | D20 | Ar1 | D21 | Ar1 |
| 771 | D21 | D22 | Ar1 | D21 | Ar1 |
| 772 | D21 | D23 | Ar1 | D21 | Ar1 |
| 773 | D21 | D24 | Ar1 | D21 | Ar1 |
| 774 | D21 | D25 | Ar1 | D21 | Ar1 |
| 775 | D21 | D26 | Ar1 | D21 | Ar1 |
| 776 | D22 | D1 | Ar1 | D22 | Ar1 |
| 777 | D22 | D2 | Ar1 | D22 | Ar1 |
| 778 | D22 | D3 | Ar1 | D22 | Ar1 |
| 779 | D22 | D4 | Ar1 | D22 | Ar1 |
| 780 | D22 | D5 | Ar1 | D22 | Ar1 |
| 781 | D22 | D6 | Ar1 | D22 | Ar1 |
| 782 | D22 | D7 | Ar1 | D22 | Ar1 |
| 783 | D22 | D8 | Ar1 | D22 | Ar1 |
| 784 | D22 | D9 | Ar1 | D22 | Ar1 |
| 785 | D22 | D10 | Ar1 | D22 | Ar1 |
| 786 | D22 | D11 | Ar1 | D22 | Ar1 |
| 787 | D22 | D12 | Ar1 | D22 | Ar1 |
| 788 | D22 | D13 | Ar1 | D22 | Ar1 |
| 789 | D22 | D14 | Ar1 | D22 | Ar1 |
| 790 | D22 | D15 | Ar1 | D22 | Ar1 |
| 791 | D22 | D16 | Ar1 | D22 | Ar1 |
| 792 | D22 | D17 | Ar1 | D22 | Ar1 |
| 793 | D22 | D18 | Ar1 | D22 | Ar1 |
| 794 | D22 | D19 | Ar1 | D22 | Ar1 |
| 795 | D22 | D20 | Ar1 | D22 | Ar1 |
| 796 | D22 | D21 | Ar1 | D22 | Ar1 |
| 797 | D22 | D23 | Ar1 | D22 | Ar1 |
| 798 | D22 | D24 | Ar1 | D22 | Ar1 |
| 799 | D22 | D25 | Ar1 | D22 | Ar1 |
| 800 | D22 | D26 | Ar1 | D22 | Ar1 |
| 801 | D23 | D1 | Ar1 | D23 | Ar1 |
| 802 | D23 | D2 | Ar1 | D23 | Ar1 |
| 803 | D23 | D3 | Ar1 | D23 | Ar1 |
| 804 | D23 | D4 | Ar1 | D23 | Ar1 |
| 805 | D23 | D5 | Ar1 | D23 | Ar1 |
| 806 | D23 | D6 | Ar1 | D23 | Ar1 |
| 807 | D23 | D7 | Ar1 | D23 | Ar1 |
| 808 | D23 | D8 | Ar1 | D23 | Ar1 |
| 809 | D23 | D9 | Ar1 | D23 | Ar1 |
| 810 | D23 | D10 | Ar1 | D23 | Ar1 |
| 811 | D23 | D11 | Ar1 | D23 | Ar1 |
| 812 | D23 | D12 | Ar1 | D23 | Ar1 |
| 813 | D23 | D13 | Ar1 | D23 | Ar1 |
| 814 | D23 | D14 | Ar1 | D23 | Ar1 |
| 815 | D23 | D15 | Ar1 | D23 | Ar1 |
| 816 | D23 | D16 | Ar1 | D23 | Ar1 |
| 817 | D23 | D17 | Ar1 | D23 | Ar1 |
| 818 | D23 | D18 | Ar1 | D23 | Ar1 |
| 819 | D23 | D19 | Ar1 | D23 | Ar1 |
| 820 | D23 | D20 | Ar1 | D23 | Ar1 |
| 821 | D23 | D21 | Ar1 | D23 | Ar1 |
| 822 | D23 | D22 | Ar1 | D23 | Ar1 |
| 823 | D23 | D24 | Ar1 | D23 | Ar1 |
| 824 | D23 | D25 | Ar1 | D23 | Ar1 |
| 825 | D23 | D26 | Ar1 | D23 | Ar1 |
| 826 | D24 | D1 | Ar1 | D24 | Ar1 |
| 827 | D24 | D2 | Ar1 | D24 | Ar1 |
| 828 | D24 | D3 | Ar1 | D24 | Ar1 |
| 829 | D24 | D4 | Ar1 | D24 | Ar1 |
| 830 | D24 | D5 | Ar1 | D24 | Ar1 |
| 831 | D24 | D6 | Ar1 | D24 | Ar1 |
| 832 | D24 | D7 | Ar1 | D24 | Ar1 |
| 833 | D24 | D8 | Ar1 | D24 | Ar1 |
| 834 | D24 | D9 | Ar1 | D24 | Ar1 |
| 835 | D24 | D10 | Ar1 | D24 | Ar1 |
| 836 | D24 | D11 | Ar1 | D24 | Ar1 |
| 837 | D24 | D12 | Ar1 | D24 | Ar1 |
| 838 | D24 | D13 | Ar1 | D24 | Ar1 |
| 839 | D24 | D14 | Ar1 | D24 | Ar1 |
| 840 | D24 | D15 | Ar1 | D24 | Ar1 |
| 841 | D24 | D16 | Ar1 | D24 | Ar1 |
| 842 | D24 | D17 | Ar1 | D24 | Ar1 |
| 843 | D24 | D18 | Ar1 | D24 | Ar1 |
| 844 | D24 | D19 | Ar1 | D24 | Ar1 |
| 845 | D24 | D20 | Ar1 | D24 | Ar1 |
| 846 | D24 | D21 | Ar1 | D24 | Ar1 |
| 847 | D24 | D22 | Ar1 | D24 | Ar1 |
| 848 | D24 | D23 | Ar1 | D24 | Ar1 |
| 849 | D24 | D25 | Ar1 | D24 | Ar1 |
| 850 | D24 | D26 | Ar1 | D24 | Ar1 |
| 851 | D25 | D1 | Ar1 | D25 | Ar1 |
| 852 | D25 | D2 | Ar1 | D25 | Ar1 |
| 853 | D25 | D3 | Ar1 | D25 | Ar1 |
| 854 | D25 | D4 | Ar1 | D25 | Ar1 |
| 855 | D25 | D5 | Ar1 | D25 | Ar1 |
| 856 | D25 | D6 | Ar1 | D25 | Ar1 |
| 857 | D25 | D7 | Ar1 | D25 | Ar1 |
| 858 | D25 | D8 | Ar1 | D25 | Ar1 |
| 859 | D25 | D9 | Ar1 | D25 | Ar1 |
| 860 | D25 | D10 | Ar1 | D25 | Ar1 |
| 861 | D25 | D11 | Ar1 | D25 | Ar1 |
| 862 | D25 | D12 | Ar1 | D25 | Ar1 |
| 863 | D25 | D13 | Ar1 | D25 | Ar1 |
| 864 | D25 | D14 | Ar1 | D25 | Ar1 |
| 865 | D25 | D15 | Ar1 | D25 | Ar1 |
| 866 | D25 | D16 | Ar1 | D25 | Ar1 |
| 867 | D25 | D17 | Ar1 | D25 | Ar1 |
| 868 | D25 | D18 | Ar1 | D25 | Ar1 |
| 869 | D25 | D19 | Ar1 | D25 | Ar1 |
| 870 | D25 | D20 | Ar1 | D25 | Ar1 |
| 871 | D25 | D21 | Ar1 | D25 | Ar1 |
| 872 | D25 | D22 | Ar1 | D25 | Ar1 |
| 873 | D25 | D23 | Ar1 | D25 | Ar1 |
| 874 | D25 | D24 | Ar1 | D25 | Ar1 |
| 875 | D25 | D26 | Ar1 | D25 | Ar1 |
| 876 | D26 | D1 | Ar1 | D26 | Ar1 |
| 877 | D26 | D2 | Ar1 | D26 | Ar1 |
| 878 | D26 | D3 | Ar1 | D26 | Ar1 |
| 879 | D26 | D4 | Ar1 | D26 | Ar1 |
| 880 | D26 | D5 | Ar1 | D26 | Ar1 |
| 881 | D26 | D6 | Ar1 | D26 | Ar1 |
| 882 | D26 | D7 | Ar1 | D26 | Ar1 |
| 883 | D26 | D8 | Ar1 | D26 | Ar1 |
| 884 | D26 | D9 | Ar1 | D26 | Ar1 |
| 885 | D26 | D10 | Ar1 | D26 | Ar1 |
| 886 | D26 | D11 | Ar1 | D26 | Ar1 |
| 887 | D26 | D12 | Ar1 | D26 | Ar1 |
| 888 | D26 | D13 | Ar1 | D26 | Ar1 |
| 889 | D26 | D14 | Ar1 | D26 | Ar1 |
| 890 | D26 | D15 | Ar1 | D26 | Ar1 |
| 891 | D26 | D16 | Ar1 | D26 | Ar1 |
| 892 | D26 | D17 | Ar1 | D26 | Ar1 |
| 893 | D26 | D18 | Ar1 | D26 | Ar1 |
| 894 | D26 | D19 | Ar1 | D26 | Ar1 |
| 895 | D26 | D20 | Ar1 | D26 | Ar1 |
| 896 | D26 | D21 | Ar1 | D26 | Ar1 |
| 897 | D26 | D22 | Ar1 | D26 | Ar1 |
| 898 | D26 | D23 | Ar1 | D26 | Ar1 |
| 899 | D26 | D24 | Ar1 | D26 | Ar1 |
| 900 | D26 | D25 | Ar1 | D26 | Ar1 |
| 901 | D1 | Ar1 | D21 | D1 | Ar1 |
| 902 | D2 | Ar1 | D21 | D2 | Ar1 |
| 903 | D3 | Ar1 | D21 | D3 | Ar1 |
| 904 | D4 | Ar1 | D21 | D4 | Ar1 |
| 905 | D5 | Ar1 | D21 | D5 | Ar1 |
| 908 | D6 | Ar1 | D21 | D6 | Ar1 |
| 907 | D7 | Ar1 | D21 | D7 | Ar1 |
| 908 | D8 | Ar1 | D21 | D8 | Ar1 |
| 909 | D9 | Ar1 | D21 | D9 | Ar1 |
| 910 | D10 | Ar1 | D21 | D10 | Ar1 |
| 911 | D11 | Ar1 | D21 | D11 | Ar1 |
| 912 | D12 | Ar1 | D21 | D12 | Ar1 |
| 913 | D13 | Ar1 | D21 | D13 | Ar1 |
| 914 | D14 | Ar1 | D21 | D14 | Ar1 |
| 915 | D15 | Ar1 | D21 | D15 | Ar1 |
| 916 | D16 | Ar1 | D21 | D16 | Ar1 |
| 917 | D17 | Ar1 | D21 | D17 | Ar1 |
| 918 | D18 | Ar1 | D21 | D18 | Ar1 |
| 919 | D19 | Ar1 | D21 | D19 | Ar1 |
| 920 | D20 | Ar1 | D21 | D20 | Ar1 |
| 921 | D22 | Ar1 | D21 | D22 | Ar1 |
| 922 | D23 | Ar1 | D21 | D23 | Ar1 |
| 923 | D24 | Ar1 | D21 | D24 | Ar1 |
| 924 | D25 | Ar1 | D21 | D25 | Ar1 |
| 925 | D26 | Ar1 | D21 | D26 | Ar1 |
| 926 | D1 | Ar1 | D22 | D1 | Ar1 |
| 927 | D2 | Ar1 | D22 | D2 | Ar1 |
| 928 | D3 | Ar1 | D22 | D3 | Ar1 |
| 929 | D4 | Ar1 | D22 | D4 | Ar1 |
| 930 | D5 | Ar1 | D22 | D5 | Ar1 |
| 931 | D6 | Ar1 | D22 | D6 | Ar1 |
| 932 | D7 | Ar1 | D22 | D7 | Ar1 |
| 933 | D8 | Ar1 | D22 | D8 | Ar1 |
| 934 | D9 | Ar1 | D22 | D9 | Ar1 |
| 935 | D10 | Ar1 | D22 | D10 | Ar1 |
| 936 | D11 | Ar1 | D22 | D11 | Ar1 |
| 937 | D12 | Ar1 | D22 | D12 | Ar1 |
| 938 | D13 | Ar1 | D22 | D13 | Ar1 |
| 939 | D14 | Ar1 | D22 | D14 | Ar1 |
| 940 | D15 | Ar1 | D22 | D15 | Ar1 |
| 941 | D16 | Ar1 | D22 | D16 | Ar1 |
| 942 | D17 | Ar1 | D22 | D17 | Ar1 |
| 943 | D18 | Ar1 | D22 | D18 | Ar1 |
| 944 | D19 | Ar1 | D22 | D19 | Ar1 |
| 945 | D20 | Ar1 | D22 | D20 | Ar1 |
| 946 | D21 | Ar1 | D22 | D21 | Ar1 |
| 947 | D23 | Ar1 | D22 | D23 | Ar1 |
| 948 | D24 | Ar1 | D22 | D24 | Ar1 |
| 949 | D25 | Ar1 | D22 | D25 | Ar1 |
| 950 | D26 | Ar1 | D22 | D26 | Ar1 |
| 951 | D1 | Ar1 | D23 | D1 | Ar1 |
| 952 | D2 | Ar1 | D23 | D2 | Ar1 |
| 953 | D3 | Ar1 | D23 | D3 | Ar1 |
| 954 | D4 | Ar1 | D23 | D4 | Ar1 |
| 955 | D5 | Ar1 | D23 | D5 | Ar1 |
| 956 | D6 | Ar1 | D23 | D6 | Ar1 |
| 957 | D7 | Ar1 | D23 | D7 | Ar1 |
| 958 | D8 | Ar1 | D23 | D8 | Ar1 |
| 959 | D9 | Ar1 | D23 | D9 | Ar1 |
| 960 | D10 | Ar1 | D23 | D10 | Ar1 |
| 961 | D11 | Ar1 | D23 | D11 | Ar1 |
| 962 | D12 | Ar1 | D22 | D12 | Ar1 |
| 963 | D13 | Ar1 | D23 | D13 | Ar1 |
| 964 | D14 | Ar1 | D23 | D14 | Ar1 |
| 965 | D15 | Ar1 | D23 | D15 | Ar1 |
| 966 | D16 | Ar1 | D23 | D16 | Ar1 |
| 967 | D17 | Ar1 | D23 | D17 | Ar1 |
| 968 | D18 | Ar1 | D23 | D18 | Ar1 |
| 969 | D19 | Ar1 | D23 | D19 | Ar1 |
| 970 | D20 | Ar1 | D23 | D20 | Ar1 |
| 971 | D21 | Ar1 | D23 | D21 | Ar1 |
| 972 | D22 | Ar1 | D23 | D22 | Ar1 |
| 973 | D24 | Ar1 | D23 | D24 | Ar1 |
| 974 | D25 | Ar1 | D23 | D25 | Ar1 |
| 975 | D26 | Ar1 | D23 | D26 | Ar1 |
| 976 | D1 | Ar1 | D24 | D1 | Ar1 |
| 977 | D2 | Ar1 | D24 | D2 | Ar1 |
| 978 | D3 | Ar1 | D24 | D3 | Ar1 |
| 979 | D4 | Ar1 | D24 | D4 | Ar1 |
| 980 | D5 | Ar1 | D24 | D5 | Ar1 |
| 981 | D6 | Ar1 | D24 | D6 | Ar1 |
| 982 | D7 | Ar1 | D24 | D7 | Ar1 |
| 983 | D8 | Ar1 | D24 | D8 | Ar1 |
| 984 | D9 | Ar1 | D24 | D9 | Ar1 |
| 985 | D10 | Ar1 | D24 | D10 | Ar1 |
| 986 | D11 | Ar1 | D24 | D11 | Ar1 |
| 987 | D12 | Ar1 | D24 | D12 | Ar1 |
| 988 | D13 | Ar1 | D24 | D13 | Ar1 |
| 989 | D14 | Ar1 | D24 | D14 | Ar1 |
| 990 | D15 | Ar1 | D24 | D15 | Ar1 |
| 991 | D16 | Ar1 | D24 | D16 | Ar1 |
| 992 | D17 | Ar1 | D24 | D17 | Ar1 |
| 993 | D18 | Ar1 | D24 | D18 | Ar1 |
| 994 | D19 | Ar1 | D24 | D19 | Ar1 |
| 995 | D20 | Ar1 | D24 | D20 | Ar1 |
| 996 | D21 | Ar1 | D24 | D21 | Ar1 |
| 997 | D22 | Ar1 | D24 | D22 | Ar1 |
| 998 | D23 | Ar1 | D24 | D23 | Ar1 |
| 999 | D25 | Ar1 | D24 | D25 | Ar1 |
| 1000 | D26 | Ar1 | D24 | D26 | Ar1 |
| 1001 | D1 | Ar1 | D25 | D1 | Ar1 |
| 1002 | D2 | Ar1 | D25 | D2 | Ar1 |
| 1003 | D3 | Ar1 | D25 | D3 | Ar1 |
| 1004 | D4 | Ar1 | D25 | D4 | Ar1 |
| 1005 | D5 | Ar1 | D25 | D5 | Ar1 |
| 1006 | D6 | Ar1 | D25 | D6 | Ar1 |
| 1007 | D7 | Ar1 | D25 | D7 | Ar1 |
| 1008 | D6 | Ar1 | D25 | D8 | Ar1 |
| 1009 | D9 | Ar1 | D25 | D9 | Ar1 |
| 1010 | D10 | Ar1 | D25 | D10 | Ar1 |
| 1011 | D11 | Ar1 | D25 | D11 | Ar1 |
| 1012 | D12 | Ar1 | D25 | D12 | Ar1 |
| 1013 | D13 | Ar1 | D25 | D13 | Ar1 |
| 1014 | D14 | Ar1 | D25 | D14 | Ar1 |
| 1015 | D15 | Ar1 | D25 | D15 | Ar1 |
| 1016 | D16 | Ar1 | D25 | D16 | Ar1 |
| 1017 | D17 | Ar1 | D25 | D17 | Ar1 |
| 1018 | D18 | Ar1 | D25 | D18 | Ar1 |
| 1019 | D19 | Ar1 | D25 | D19 | Ar1 |
| 1020 | D20 | Ar1 | D25 | D20 | Ar1 |
| 1021 | D21 | Ar1 | D25 | D21 | Ar1 |
| 1022 | D22 | Ar1 | D25 | D22 | Ar1 |
| 1023 | D23 | Ar1 | D25 | D23 | Ar1 |
| 1024 | D24 | Ar1 | D25 | D24 | Ar1 |
| 1025 | D26 | Ar1 | D25 | D26 | Ar1 |
| 1026 | D1 | Ar1 | D26 | D1 | Ar1 |
| 1027 | D2 | Ar1 | D26 | D2 | Ar1 |
| 1028 | D3 | Ar1 | D26 | D3 | Ar1 |
| 1029 | D4 | Ar1 | D26 | D4 | Ar1 |
| 1030 | D5 | Ar1 | D26 | D5 | Ar1 |
| 1031 | D6 | Ar1 | D26 | D6 | Ar1 |
| 1032 | D7 | Ar1 | D26 | D7 | Ar1 |
| 1033 | D8 | Ar1 | D26 | D8 | Ar1 |
| 1034 | D9 | Ar1 | D26 | D9 | Ar1 |
| 1035 | D10 | Ar1 | D26 | D10 | Ar1 |
| 1036 | D11 | Ar1 | D26 | D11 | Ar1 |
| 1037 | D12 | Ar1 | D26 | D12 | Ar1 |
| 1038 | D13 | Ar1 | D26 | D13 | Ar1 |
| 1039 | D14 | Ar1 | D26 | D14 | Ar1 |
| 1040 | D15 | Ar1 | D26 | D15 | Ar1 |
| 1041 | D16 | Ar1 | D26 | D16 | Ar1 |
| 1042 | D17 | Ar1 | D26 | D17 | Ar1 |
| 1043 | D18 | Ar1 | D26 | D18 | Ar1 |
| 1044 | D19 | Ar1 | D26 | D19 | Ar1 |
| 1045 | D20 | Ar1 | D26 | D20 | Ar1 |
| 1046 | D21 | Ar1 | D26 | D21 | Ar1 |
| 1047 | D22 | Ar1 | D26 | D22 | Ar1 |
| 1048 | D23 | Ar1 | D26 | D23 | Ar1 |
| 1049 | D24 | Ar1 | D26 | D24 | Ar1 |
| 1050 | D25 | Ar1 | D26 | D25 | Ar1 |
| 1051 | D21 | Ar1 | D1 | D21 | Ar1 |
| 1052 | D21 | Ar1 | D2 | D21 | Ar1 |
| 1053 | D21 | Ar1 | D3 | D21 | Ar1 |
| 1054 | D21 | Ar1 | D4 | D21 | Ar1 |
| 1055 | D21 | Ar1 | D5 | D21 | Ar1 |
| 1056 | D21 | Ar1 | D6 | D21 | Ar1 |
| 1057 | D21 | Ar1 | D7 | D21 | Ar1 |
| 1058 | D21 | Ar1 | D8 | D21 | Ar1 |
| 1059 | D21 | Ar1 | D9 | D21 | Ar1 |
| 1060 | D21 | Ar1 | D10 | D21 | Ar1 |
| 1061 | D21 | Ar1 | D11 | D21 | Ar1 |
| 1062 | D21 | Ar1 | D12 | D21 | Ar1 |
| 1063 | D21 | Ar1 | D13 | D21 | Ar1 |
| 1064 | D21 | Ar1 | D14 | D21 | Ar1 |
| 1065 | D21 | Ar1 | D15 | D21 | Ar1 |
| 1066 | D21 | Ar1 | D16 | D21 | Ar1 |
| 1067 | D21 | Ar1 | D17 | D21 | Ar1 |
| 1068 | D21 | Ar1 | D18 | D21 | Ar1 |
| 1069 | D21 | Ar1 | D19 | D21 | Ar1 |
| 1070 | D21 | Ar1 | D20 | D21 | Ar1 |
| 1071 | D21 | Ar1 | D22 | D21 | Ar1 |
| 1072 | D21 | Ar1 | D23 | D21 | Ar1 |
| 1073 | D21 | Ar1 | D24 | D21 | Ar1 |
| 1074 | D21 | Ar1 | D25 | D21 | Ar1 |
| 1075 | D21 | Ar1 | D26 | D21 | Ar1 |
| 1076 | D22 | Ar1 | D1 | D22 | Ar1 |
| 1077 | D22 | Ar1 | D2 | D22 | Ar1 |
| 1078 | D22 | Ar1 | D3 | D22 | Ar1 |
| 1079 | D22 | Ar1 | D4 | D22 | Ar1 |
| 1080 | D22 | Ar1 | D5 | D22 | Ar1 |
| 1081 | D22 | Ar1 | D6 | D22 | Ar1 |
| 1082 | D22 | Ar1 | D7 | D22 | Ar1 |
| 1083 | D22 | Ar1 | D8 | D22 | Ar1 |
| 1084 | D22 | Ar1 | D9 | D22 | Ar1 |
| 1085 | D22 | Ar1 | D10 | D22 | Ar1 |
| 1088 | D22 | Ar1 | D11 | D22 | Ar1 |
| 1087 | D22 | Ar1 | D12 | D22 | Ar1 |
| 1088 | D22 | Ar1 | D13 | D22 | Ar1 |
| 1089 | D22 | Ar1 | D14 | D22 | Ar1 |
| 1090 | D22 | Ar1 | D15 | D22 | Ar1 |
| 1091 | D22 | Ar1 | D16 | D22 | Ar1 |
| 1092 | D22 | Ar1 | D17 | D22 | Ar1 |
| 1093 | D22 | Ar1 | D18 | D22 | Ar1 |
| 1094 | D22 | Ar1 | D19 | D22 | Ar1 |
| 1095 | D22 | Ar1 | D20 | D22 | Ar1 |
| 1096 | D22 | Ar1 | D21 | D22 | Ar1 |
| 1097 | D22 | Ar1 | D23 | D22 | Ar1 |
| 1098 | D22 | Ar1 | D24 | D22 | Ar1 |
| 1099 | D22 | Ar1 | D25 | D22 | Ar1 |
| 1100 | D22 | Ar1 | D26 | D22 | Ar1 |
| 1101 | D23 | Ar1 | D1 | D23 | Ar1 |
| 1102 | D23 | Ar1 | D2 | D23 | Ar1 |
| 1103 | D23 | Ar1 | D3 | D23 | Ar1 |
| 1104 | D23 | Ar1 | D4 | D23 | Ar1 |
| 1105 | D23 | Ar1 | D5 | D23 | Ar1 |
| 1106 | D23 | Ar1 | D6 | D23 | Ar1 |
| 1107 | D23 | Ar1 | D7 | D23 | Ar1 |
| 1108 | D23 | Ar1 | D8 | D23 | Ar1 |
| 1109 | D23 | Ar1 | D9 | D23 | Ar1 |
| 1110 | D23 | Ar1 | D10 | D23 | Ar1 |
| 1111 | D23 | Ar1 | D11 | D23 | Ar1 |
| 1112 | D23 | Ar1 | D12 | D23 | Ar1 |
| 1113 | D23 | Ar1 | D13 | D23 | Ar1 |
| 1114 | D23 | Ar1 | D14 | D23 | Ar1 |
| 1115 | D23 | Ar1 | D15 | D23 | Ar1 |
| 1116 | D23 | Ar1 | D16 | D23 | Ar1 |
| 1117 | D23 | Ar1 | D17 | D23 | Ar1 |
| 1118 | D23 | Ar1 | D18 | D23 | Ar1 |
| 1119 | D23 | Ar1 | D19 | D23 | Ar1 |
| 1120 | D23 | Ar1 | D20 | D23 | Ar1 |
| 1121 | D23 | Ar1 | D21 | D23 | Ar1 |
| 1122 | D23 | Ar1 | D22 | D23 | Ar1 |
| 1123 | D23 | Ar1 | D24 | D23 | Ar1 |
| 1124 | D23 | Ar1 | D25 | D23 | Ar1 |
| 1125 | D23 | Ar1 | D26 | D23 | Ar1 |
| 1126 | D24 | Ar1 | D1 | D24 | Ar1 |
| 1127 | D24 | Ar1 | D2 | D24 | Ar1 |
| 1128 | D24 | Ar1 | D3 | D24 | Ar1 |
| 1129 | D24 | Ar1 | D4 | D24 | Ar1 |
| 1130 | D24 | Ar1 | D5 | D24 | Ar1 |
| 1131 | D24 | Ar1 | D6 | D24 | Ar1 |
| 1132 | D24 | Ar1 | D7 | D24 | Ar1 |
| 1133 | D24 | Ar1 | D8 | D24 | Ar1 |
| 1134 | D24 | Ar1 | D9 | D24 | Ar1 |
| 1135 | D24 | Ar1 | D10 | D24 | Ar1 |
| 1136 | D24 | Ar1 | D11 | D24 | Ar1 |
| 1137 | D24 | Ar1 | D12 | D24 | Ar1 |
| 1138 | D24 | Ar1 | D13 | D24 | Ar1 |
| 1139 | D24 | Ar1 | D14 | D24 | Ar1 |
| 1140 | D24 | Ar1 | D15 | D24 | Ar1 |
| 1141 | D24 | Ar1 | D16 | D24 | Ar1 |
| 1142 | D24 | Ar1 | D17 | D24 | Ar1 |
| 1143 | D24 | Ar1 | D18 | D24 | Ar1 |
| 1144 | D24 | Ar1 | D19 | D24 | Ar1 |
| 1145 | D24 | Ar1 | D20 | D24 | Ar1 |
| 1146 | D24 | Ar1 | D21 | D24 | Ar1 |
| 1147 | D24 | Ar1 | D22 | D24 | Ar1 |
| 1148 | D24 | Ar1 | D23 | D24 | Ar1 |
| 1149 | D24 | Ar1 | D25 | D24 | Ar1 |
| 1150 | D24 | Ar1 | D26 | D24 | Ar1 |
| 1151 | D25 | Ar1 | D1 | D25 | Ar1 |
| 1152 | D25 | Ar1 | D2 | D25 | Ar1 |
| 1153 | D25 | Ar1 | D3 | D25 | Ar1 |
| 1154 | D25 | Ar1 | D4 | D25 | Ar1 |
| 1155 | D25 | Ar1 | D5 | D25 | Ar1 |
| 1156 | D25 | Ar1 | D6 | D25 | Ar1 |
| 1157 | D25 | Ar1 | D7 | D25 | Ar1 |
| 1158 | D25 | Ar1 | D8 | D25 | Ar1 |
| 1159 | D25 | Ar1 | D9 | D25 | Ar1 |
| 1160 | D25 | Ar1 | D10 | D25 | Ar1 |
| 1161 | D25 | Ar1 | D11 | D25 | Ar1 |
| 1162 | D25 | Ar1 | D12 | D25 | Ar1 |
| 1163 | D25 | Ar1 | D13 | D25 | Ar1 |
| 1164 | D25 | Ar1 | D14 | D25 | Ar1 |
| 1165 | D25 | Ar1 | D15 | D25 | Ar1 |
| 1166 | D25 | Ar1 | D16 | D25 | Ar1 |
| 1167 | D25 | Ar1 | D17 | D25 | Ar1 |
| 1168 | D25 | Ar1 | D18 | D25 | Ar1 |
| 1169 | D25 | Ar1 | D19 | D25 | Ar1 |
| 1170 | D25 | Ar1 | D20 | D25 | Ar1 |
| 1171 | D25 | Ar1 | D21 | D25 | Ar1 |
| 1172 | D25 | Ar1 | D22 | D25 | Ar1 |
| 1173 | D25 | Ar1 | D23 | D25 | Ar1 |
| 1174 | D25 | Ar1 | D24 | D25 | Ar1 |
| 1175 | D25 | Ar1 | D26 | D25 | Ar1 |
| 1176 | D26 | Ar1 | D1 | D26 | Ar1 |
| 1177 | D26 | Ar1 | D2 | D26 | Ar1 |
| 1178 | D26 | Ar1 | D3 | D26 | Ar1 |
| 1179 | D26 | Ar1 | D4 | D26 | Ar1 |
| 1180 | D26 | Ar1 | D5 | D26 | Ar1 |
| 1181 | D26 | Ar1 | D6 | D26 | Ar1 |
| 1182 | D26 | Ar1 | D7 | D26 | Ar1 |
| 1183 | D26 | Ar1 | D8 | D26 | Ar1 |
| 1184 | D26 | Ar1 | D9 | D26 | Ar1 |
| 1185 | D26 | Ar1 | D10 | D26 | Ar1 |
| 1186 | D26 | Ar1 | D11 | D26 | Ar1 |
| 1187 | D26 | Ar1 | D12 | D26 | Ar1 |
| 1188 | D26 | Ar1 | D13 | D26 | Ar1 |
| 1189 | D26 | Ar1 | D14 | D26 | Ar1 |
| 1190 | D26 | Ar1 | D15 | D26 | Ar1 |
| 1191 | D26 | Ar1 | D16 | D26 | Ar1 |
| 1192 | D26 | Ar1 | D17 | D26 | Ar1 |
| 1193 | D26 | Ar1 | D18 | D26 | Ar1 |
| 1194 | D26 | Ar1 | D19 | D26 | Ar1 |
| 1195 | D26 | Ar1 | D20 | D26 | Ar1 |
| 1196 | D26 | Ar1 | D21 | D26 | Ar1 |
| 1197 | D26 | Ar1 | D22 | D26 | Ar1 |
| 1198 | D26 | Ar1 | D23 | D26 | Ar1 |
| 1199 | D26 | Ar1 | D24 | D26 | Ar1 |
| 1200 | D26 | Ar1 | D25 | D26 | Ar1 |
| 1201 | D21 | Ar1 | D1 | D1 | Ar1 |
| 1202 | D21 | Ar1 | D2 | D2 | Ar1 |
| 1203 | D21 | Ar1 | D3 | D3 | Ar1 |
| 1204 | D21 | Ar1 | D4 | D4 | Ar1 |
| 1205 | D21 | Ar1 | D5 | D5 | Ar1 |
| 1206 | D21 | Ar1 | D6 | D6 | Ar1 |
| 1207 | D21 | Ar1 | D7 | D7 | Ar1 |
| 1208 | D21 | Ar1 | D8 | D8 | Ar1 |
| 1209 | D21 | Ar1 | D9 | D9 | Ar1 |
| 1210 | D21 | Ar1 | D10 | D10 | Ar1 |
| 1211 | D21 | Ar1 | D11 | D11 | Ar1 |
| 1212 | D21 | Ar1 | D12 | D12 | Ar1 |
| 1213 | D21 | Ar1 | D13 | D13 | Ar1 |
| 1214 | D21 | Ar1 | D14 | D14 | Ar1 |
| 1215 | D21 | Ar1 | D15 | D15 | Ar1 |
| 1216 | D21 | Ar1 | D16 | D16 | Ar1 |
| 1217 | D21 | Ar1 | D17 | D17 | Ar1 |
| 1218 | D21 | Ar1 | D18 | D18 | Ar1 |
| 1219 | D21 | Ar1 | D19 | D19 | Ar1 |
| 1220 | D21 | Ar1 | D20 | D20 | Ar1 |
| 1221 | D21 | Ar1 | D22 | D22 | Ar1 |
| 1222 | D21 | Ar1 | D23 | D23 | Ar1 |
| 1223 | D21 | Ar1 | D24 | D24 | Ar1 |
| 1224 | D21 | Ar1 | D25 | D25 | Ar1 |
| 1225 | D21 | Ar1 | D26 | D26 | Ar1 |
| 1226 | D22 | Ar1 | D1 | D1 | Ar1 |
| 1227 | D22 | Ar1 | D2 | D2 | Ar1 |
| 1228 | D22 | Ar1 | D3 | D3 | Ar1 |
| 1229 | D22 | Ar1 | D4 | D4 | Ar1 |
| 1230 | D22 | Ar1 | D5 | D5 | Ar1 |
| 1231 | D22 | Ar1 | D6 | D6 | Ar1 |
| 1232 | D22 | Ar1 | D7 | D7 | Ar1 |
| 1233 | D22 | Ar1 | D8 | D8 | Ar1 |
| 1234 | D22 | Ar1 | D9 | D9 | Ar1 |
| 1235 | D22 | Ar1 | D10 | D10 | Ar1 |
| 1236 | D22 | Ar1 | D11 | D11 | Ar1 |
| 1237 | D22 | Ar1 | D12 | D12 | Ar1 |
| 1238 | D22 | Ar1 | D13 | D13 | Ar1 |
| 1239 | D22 | Ar1 | D14 | D14 | Ar1 |
| 1240 | D22 | Ar1 | D15 | D15 | Ar1 |
| 1241 | D22 | Ar1 | D16 | D16 | Ar1 |
| 1242 | D22 | Ar1 | D17 | D17 | Ar1 |
| 1243 | D22 | Ar1 | D18 | D18 | Ar1 |
| 1244 | D22 | Ar1 | D19 | D19 | Ar1 |
| 1245 | D22 | Ar1 | D20 | D20 | Ar1 |
| 1246 | D22 | Ar1 | D21 | D21 | Ar1 |
| 1247 | D22 | Ar1 | D23 | D23 | Ar1 |
| 1248 | D22 | Ar1 | D24 | D24 | Ar1 |
| 1249 | D22 | Ar1 | D25 | D25 | Ar1 |
| 1250 | D22 | Ar1 | D26 | D26 | Ar1 |
| 1251 | D23 | Ar1 | D1 | D1 | Ar1 |
| 1252 | D23 | Ar1 | D2 | D2 | Ar1 |
| 1253 | D23 | Ar1 | D3 | D3 | Ar1 |
| 1254 | D23 | Ar1 | D4 | D4 | Ar1 |
| 1255 | D23 | Ar1 | D5 | D5 | Ar1 |
| 1256 | D23 | Ar1 | D6 | D6 | Ar1 |
| 1257 | D23 | Ar1 | D7 | D7 | Ar1 |
| 1258 | D23 | Ar1 | D8 | D8 | Ar1 |
| 1259 | D23 | Ar1 | D9 | D9 | Ar1 |
| 1260 | D23 | Ar1 | D10 | D10 | Ar1 |
| 1261 | D23 | Ar1 | D11 | D11 | Ar1 |
| 1262 | D23 | Ar1 | D12 | D12 | Ar1 |
| 1263 | D23 | Ar1 | D13 | D13 | Ar1 |
| 1264 | D23 | Ar1 | D14 | D14 | Ar1 |
| 1265 | D23 | Ar1 | D15 | D15 | Ar1 |
| 1266 | D23 | Ar1 | D16 | D16 | Ar1 |
| 1267 | D23 | Ar1 | D17 | D17 | Ar1 |
| 1268 | D23 | Ar1 | D18 | D18 | Ar1 |
| 1269 | D23 | Ar1 | D19 | D19 | Ar1 |
| 1270 | D23 | Ar1 | D20 | D20 | Ar1 |
| 1271 | D23 | Ar1 | D21 | D21 | Ar1 |
| 1272 | D23 | Ar1 | D22 | D22 | Ar1 |
| 1273 | D23 | Ar1 | D24 | D24 | Ar1 |
| 1274 | D23 | Ar1 | D25 | D25 | Ar1 |
| 1275 | D23 | Ar1 | D26 | D26 | Ar1 |
| 1276 | D24 | Ar1 | D1 | D1 | Ar1 |
| 1277 | D24 | Ar1 | D2 | D2 | Ar1 |
| 1278 | D24 | Ar1 | D3 | D3 | Ar1 |
| 1279 | D24 | Ar1 | D4 | D4 | Ar1 |
| 1280 | D24 | Ar1 | D5 | D5 | Ar1 |
| 1281 | D24 | Ar1 | D6 | D6 | Ar1 |
| 1282 | D24 | Ar1 | D7 | D7 | Ar1 |
| 1283 | D24 | Ar1 | D8 | D8 | Ar1 |
| 1284 | D24 | Ar1 | D9 | D9 | Ar1 |
| 1285 | D24 | Ar1 | D10 | D10 | Ar1 |
| 1286 | D24 | Ar1 | D11 | D11 | Ar1 |
| 1287 | D24 | Ar1 | D12 | D12 | Ar1 |
| 1288 | D24 | Ar1 | D13 | D13 | Ar1 |
| 1289 | D24 | Ar1 | D14 | D14 | Ar1 |
| 1290 | D24 | Ar1 | D15 | D15 | Ar1 |
| 1291 | D24 | Ar1 | D16 | D16 | Ar1 |
| 1292 | D24 | Ar1 | D17 | D17 | Ar1 |
| 1293 | D24 | Ar1 | D18 | D18 | Ar1 |
| 1294 | D24 | Ar1 | D19 | D19 | Ar1 |
| 1295 | D24 | Ar1 | D20 | D20 | Ar1 |
| 1296 | D24 | Ar1 | D21 | D21 | Ar1 |
| 1297 | D24 | Ar1 | D22 | D22 | Ar1 |
| 1298 | D24 | Ar1 | D23 | D23 | Ar1 |
| 1299 | D24 | Ar1 | D25 | D25 | Ar1 |
| 1300 | D24 | Ar1 | D26 | D26 | Ar1 |
| 1301 | D25 | Ar1 | D1 | D1 | Ar1 |
| 1302 | D25 | Ar1 | D2 | D2 | Ar1 |
| 1303 | D25 | Ar1 | D3 | D3 | Ar1 |
| 1304 | D25 | Ar1 | D4 | D4 | Ar1 |
| 1305 | D25 | Ar1 | D5 | D5 | Ar1 |
| 1306 | D25 | Ar1 | D6 | D6 | Ar1 |
| 1307 | D25 | Ar1 | D7 | D7 | Ar1 |
| 1308 | D25 | Ar1 | D8 | D8 | Ar1 |
| 1309 | D25 | Ar1 | D9 | D9 | Ar1 |
| 1310 | D25 | Ar1 | D10 | D10 | Ar1 |
| 1311 | D25 | Ar1 | D11 | D11 | Ar1 |
| 1312 | D25 | Ar1 | D12 | D12 | Ar1 |
| 1313 | D25 | Ar1 | D13 | D13 | Ar1 |
| 1314 | D25 | Ar1 | D14 | D14 | Ar1 |
| 1315 | D25 | Ar1 | D15 | D15 | Ar1 |
| 1316 | D25 | Ar1 | D16 | D16 | Ar1 |
| 1317 | D25 | Ar1 | D17 | D17 | Ar1 |
| 1318 | D25 | Ar1 | D18 | D18 | Ar1 |
| 1319 | D25 | Ar1 | D19 | D19 | Ar1 |
| 1320 | D25 | Ar1 | D20 | D20 | Ar1 |
| 1321 | D25 | Ar1 | D21 | D21 | Ar1 |
| 1322 | D25 | Ar1 | D22 | D22 | Ar1 |
| 1323 | D25 | Ar1 | D23 | D23 | Ar1 |
| 1324 | D25 | Ar1 | D24 | D24 | Ar1 |
| 1325 | D25 | Ar1 | D26 | D26 | Ar1 |
| 1326 | D26 | Ar1 | D1 | D1 | Ar1 |
| 1327 | D26 | Ar1 | D2 | D2 | Ar1 |
| 1328 | D26 | Ar1 | D3 | D3 | Ar1 |
| 1329 | D26 | Ar1 | D4 | D4 | Ar1 |
| 1330 | D26 | Ar1 | D5 | D5 | Ar1 |
| 1331 | D26 | Ar1 | D6 | D6 | Ar1 |
| 1332 | D26 | Ar1 | D7 | D7 | Ar1 |
| 1333 | D26 | Ar1 | D8 | D8 | Ar1 |
| 1334 | D26 | Ar1 | D9 | D9 | Ar1 |
| 1335 | D26 | Ar1 | D10 | D10 | Ar1 |
| 1336 | D26 | Ar1 | D11 | D11 | Ar1 |
| 1337 | D26 | Ar1 | D12 | D12 | Ar1 |
| 1338 | D26 | Ar1 | D13 | D13 | Ar1 |
| 1339 | D26 | Ar1 | D14 | D14 | Ar1 |
| 1340 | D26 | Ar1 | D15 | D15 | Ar1 |
| 1341 | D26 | Ar1 | D16 | D16 | Ar1 |
| 1342 | D26 | Ar1 | D17 | D17 | Ar1 |
| 1343 | D26 | Ar1 | D18 | D18 | Ar1 |
| 1344 | D26 | Ar1 | D19 | D19 | Ar1 |
| 1345 | D26 | Ar1 | D20 | D20 | Ar1 |
| 1346 | D26 | Ar1 | D21 | D21 | Ar1 |
| 1347 | D26 | Ar1 | D22 | D22 | Ar1 |
| 1348 | D26 | Ar1 | D23 | D23 | Ar1 |
| 1349 | D26 | Ar1 | D24 | D24 | Ar1 |
| 1350 | D26 | Ar1 | D25 | D25 | Ar1 |
| 1351 | D1 | Ar1 | D21 | D21 | Ar1 |
| 1352 | D2 | Ar1 | D21 | D21 | Ar1 |
| 1353 | D3 | Ar1 | D21 | D21 | Ar1 |
| 1354 | D4 | Ar1 | D21 | D21 | Ar1 |
| 1355 | D5 | Ar1 | D21 | D21 | Ar1 |
| 1356 | D6 | Ar1 | D21 | D21 | Ar1 |
| 1357 | D7 | Ar1 | D21 | D21 | Ar1 |
| 1358 | D8 | Ar1 | D21 | D21 | Ar1 |
| 1359 | D9 | Ar1 | D21 | D21 | Ar1 |
| 1360 | D10 | Ar1 | D21 | D21 | Ar1 |
| 1361 | D11 | Ar1 | D21 | D21 | Ar1 |
| 1362 | D12 | Ar1 | D21 | D21 | Ar1 |
| 1363 | D13 | Ar1 | D21 | D21 | Ar1 |
| 1364 | D14 | Ar1 | D21 | D21 | Ar1 |
| 1365 | D15 | Ar1 | D21 | D21 | Ar1 |
| 1366 | D16 | Ar1 | D21 | D21 | Ar1 |
| 1367 | D17 | Ar1 | D21 | D21 | Ar1 |
| 1368 | D18 | Ar1 | D21 | D21 | Ar1 |
| 1369 | D19 | Ar1 | D21 | D21 | Ar1 |
| 1370 | D20 | Ar1 | D21 | D21 | Ar1 |
| 1371 | D22 | Ar1 | D21 | D21 | Ar1 |
| 1372 | D23 | Ar1 | D21 | D21 | Ar1 |
| 1373 | D24 | Ar1 | D21 | D21 | Ar1 |
| 1374 | D25 | Ar1 | D21 | D21 | Ar1 |
| 1375 | D26 | Ar1 | D21 | D21 | Ar1 |
| 1376 | D1 | Ar1 | D22 | D21 | Ar1 |
| 1377 | D2 | Ar1 | D22 | D22 | Ar1 |
| 1378 | D3 | Ar1 | D22 | D22 | Ar1 |
| 1379 | D4 | Ar1 | D22 | D22 | Ar1 |
| 1380 | D5 | Ar1 | D22 | D21 | Ar1 |
| 1381 | D6 | Ar1 | D22 | D21 | Ar1 |
| 1382 | D7 | Ar1 | D22 | D22 | Ar1 |
| 1383 | D8 | Ar1 | D22 | D22 | Ar1 |
| 1384 | D9 | Ar1 | D22 | D22 | Ar1 |
| 1385 | D10 | Ar1 | D22 | D22 | Ar1 |
| 1386 | D11 | Ar1 | D22 | D22 | Ar1 |
| 1387 | D12 | Ar1 | D22 | D22 | Ar1 |
| 1388 | D13 | Ar1 | D22 | D22 | Ar1 |
| 1389 | D14 | Ar1 | D22 | D22 | Ar1 |
| 1390 | D15 | Ar1 | D22 | D22 | Ar1 |
| 1391 | D16 | Ar1 | D22 | D22 | Ar1 |
| 1392 | D17 | Ar1 | D22 | D22 | Ar1 |
| 1393 | D18 | Ar1 | D22 | D22 | Ar1 |
| 1394 | D19 | Ar1 | D22 | D22 | Ar1 |
| 1395 | D20 | Ar1 | D22 | D22 | Ar1 |
| 1396 | D21 | Ar1 | D22 | D22 | Ar1 |
| 1397 | D23 | Ar1 | D22 | D22 | Ar1 |
| 1398 | D24 | Ar1 | D22 | D22 | Ar1 |
| 1399 | D25 | Ar1 | D22 | D22 | Ar1 |
| 1400 | D26 | Ar1 | D22 | D22 | Ar1 |
| 1401 | D1 | Ar1 | D23 | D23 | Ar1 |
| 1402 | D2 | Ar1 | D23 | D23 | Ar1 |
| 1403 | D3 | Ar1 | D23 | D23 | Ar1 |
| 1404 | D4 | Ar1 | D23 | D23 | Ar1 |
| 1405 | D5 | Ar1 | D23 | D23 | Ar1 |
| 1406 | D6 | Ar1 | D23 | D23 | Ar1 |
| 1407 | D7 | Ar1 | D23 | D23 | Ar1 |
| 1408 | D8 | Ar1 | D23 | D23 | Ar1 |
| 1409 | D9 | Ar1 | D23 | D23 | Ar1 |
| 1410 | D10 | Ar1 | D23 | D23 | Ar1 |
| 1411 | D11 | Ar1 | D23 | D23 | Ar1 |
| 1412 | D12 | Ar1 | D23 | D23 | Ar1 |
| 1413 | D13 | Ar1 | D23 | D23 | Ar1 |
| 1414 | D14 | Ar1 | D23 | D23 | Ar1 |
| 1415 | D15 | Ar1 | D23 | D23 | Ar1 |
| 1416 | D16 | Ar1 | D23 | D23 | Ar1 |
| 1417 | D17 | Ar1 | D23 | D23 | Ar1 |
| 1418 | D18 | Ar1 | D23 | D23 | Ar1 |
| 1419 | D19 | Ar1 | D23 | D23 | Ar1 |
| 1420 | D20 | Ar1 | D23 | D23 | Ar1 |
| 1421 | D21 | Ar1 | D23 | D23 | Ar1 |
| 1422 | D22 | Ar1 | D23 | D23 | Ar1 |
| 1423 | D24 | Ar1 | D23 | D23 | Ar1 |
| 1424 | D25 | Ar1 | D23 | D23 | Ar1 |
| 1425 | D26 | Ar1 | D23 | D23 | Ar1 |
| 1426 | D1 | Ar1 | D24 | D24 | Ar1 |
| 1427 | D2 | Ar1 | D24 | D24 | Ar1 |
| 1428 | D3 | Ar1 | D24 | D24 | Ar1 |
| 1429 | D4 | Ar1 | D24 | D24 | Ar1 |
| 1430 | D5 | Ar1 | D24 | D24 | Ar1 |
| 1431 | D6 | Ar1 | D24 | D24 | Ar1 |
| 1432 | D7 | Ar1 | D24 | D24 | Ar1 |
| 1433 | D8 | Ar1 | D24 | D24 | Ar1 |
| 1434 | D9 | Ar1 | D24 | D24 | Ar1 |
| 1435 | D10 | Ar1 | D24 | D24 | Ar1 |
| 1436 | D11 | Ar1 | D24 | D24 | Ar1 |
| 1437 | D12 | Ar1 | D24 | D24 | Ar1 |
| 1438 | D13 | Ar1 | D24 | D24 | Ar1 |
| 1439 | D14 | Ar1 | D24 | D24 | Ar1 |
| 1440 | D15 | Ar1 | D24 | D24 | Ar1 |
| 1441 | D16 | Ar1 | D24 | D24 | Ar1 |
| 1442 | D17 | Ar1 | D24 | D24 | Ar1 |
| 1443 | D18 | Ar1 | D24 | D24 | Ar1 |
| 1444 | D19 | Ar1 | D24 | D24 | Ar1 |
| 1445 | D20 | Ar1 | D24 | D24 | Ar1 |
| 1446 | D21 | Ar1 | D24 | D24 | Ar1 |
| 1447 | D22 | Ar1 | D24 | D24 | Ar1 |
| 1448 | D23 | Ar1 | D24 | D24 | Ar1 |
| 1449 | D25 | Ar1 | D24 | D24 | Ar1 |
| 1450 | D26 | Ar1 | D24 | D24 | Ar1 |
| 1451 | D1 | Ar1 | D25 | D25 | Ar1 |
| 1452 | D2 | Ar1 | D25 | D25 | Ar1 |
| 1453 | D3 | Ar1 | D25 | D25 | Ar1 |
| 1454 | D4 | Ar1 | D25 | D25 | Ar1 |
| 1455 | D5 | Ar1 | D25 | D25 | Ar1 |
| 1456 | D6 | Ar1 | D25 | D25 | Ar1 |
| 1457 | D7 | Ar1 | D25 | D25 | Ar1 |
| 1458 | D8 | Ar1 | D25 | D25 | Ar1 |
| 1459 | D9 | Ar1 | D25 | D25 | Ar1 |
| 1460 | D10 | Ar1 | D25 | D25 | Ar1 |
| 1461 | D11 | Ar1 | D25 | D25 | Ar1 |
| 1462 | D12 | Ar1 | D25 | D25 | Ar1 |
| 1463 | D13 | Ar1 | D25 | D25 | Ar1 |
| 1464 | D14 | Ar1 | D25 | D25 | Ar1 |
| 1465 | D15 | Ar1 | D25 | D25 | Ar1 |
| 1466 | D16 | Ar1 | D25 | D25 | Ar1 |
| 1467 | D17 | Ar1 | D25 | D25 | Ar1 |
| 1468 | D18 | Ar1 | D25 | D25 | Ar1 |
| 1469 | D19 | Ar1 | D25 | D25 | Ar1 |
| 1470 | D20 | Ar1 | D25 | D25 | Ar1 |
| 1471 | D21 | Ar1 | D25 | D25 | Ar1 |
| 1472 | D22 | Ar1 | D25 | D25 | Ar1 |
| 1473 | D23 | Ar1 | D25 | D25 | Ar1 |
| 1474 | D24 | Ar1 | D25 | D25 | Ar1 |
| 1475 | D26 | Ar1 | D25 | D25 | Ar1 |
| 1476 | D1 | Ar1 | D26 | D26 | Ar1 |
| 1477 | D2 | Ar1 | D26 | D26 | Ar1 |
| 1478 | D3 | Ar1 | D26 | D26 | Ar1 |
| 1479 | D4 | Ar1 | D26 | D26 | Ar1 |
| 1480 | D5 | Ar1 | D26 | D26 | Ar1 |
| 1481 | D6 | Ar1 | D26 | D26 | Ar1 |
| 1482 | D7 | Ar1 | D26 | D26 | Ar1 |
| 1483 | D8 | Ar1 | D26 | D26 | Ar1 |
| 1484 | D9 | Ar1 | D26 | D26 | Ar1 |
| 1485 | D10 | Ar1 | D26 | D26 | Ar1 |
| 1486 | D11 | Ar1 | D26 | D26 | Ar1 |
| 1487 | D12 | Ar1 | D26 | D26 | Ar1 |
| 1488 | D13 | Ar1 | D26 | D26 | Ar1 |
| 1489 | D14 | Ar1 | D26 | D26 | Ar1 |
| 1490 | D15 | Ar1 | D26 | D26 | Ar1 |
| 1491 | D16 | Ar1 | D26 | D26 | Ar1 |
| 1492 | D17 | Ar1 | D26 | D26 | Ar1 |
| 1493 | D18 | Ar1 | D26 | D26 | Ar1 |
| 1494 | D19 | Ar1 | D26 | D26 | Ar1 |
| 1495 | D20 | Ar1 | D26 | D26 | Ar1 |
| 1496 | D21 | Ar1 | D26 | D26 | Ar1 |
| 1497 | D22 | Ar1 | D26 | D26 | Ar1 |
| 1498 | D23 | Ar1 | D26 | D26 | Ar1 |
| 1499 | D24 | Ar1 | D26 | D26 | Ar1 |
| 1500 | D25 | Ar1 | D26 | D26 | Ar1 |
| 1501 | D1 | Ar1 | D1 | D21 | Ar1 |
| 1502 | D2 | Ar1 | D2 | D21 | Ar1 |
| 1503 | D3 | Ar1 | D3 | D21 | Ar1 |
| 1504 | D4 | Ar1 | D4 | D21 | Ar1 |
| 1505 | D5 | Ar1 | D5 | D21 | Ar1 |
| 1506 | D6 | Ar1 | D6 | D21 | Ar1 |
| 1507 | D7 | Ar1 | D7 | D21 | Ar1 |
| 1508 | D8 | Ar1 | D8 | D21 | Ar1 |
| 1509 | D9 | Ar1 | D9 | D21 | Ar1 |
| 1510 | D10 | Ar1 | D10 | D21 | Ar1 |
| 1511 | D11 | Ar1 | D11 | D21 | Ar1 |
| 1512 | D12 | Ar1 | D12 | D21 | Ar1 |
| 1513 | D13 | Ar1 | D13 | D21 | Ar1 |
| 1514 | D14 | Ar1 | D14 | D21 | Ar1 |
| 1515 | D15 | Ar1 | D15 | D21 | Ar1 |
| 1516 | D16 | Ar1 | D16 | D21 | Ar1 |
| 1517 | D17 | Ar1 | D17 | D21 | Ar1 |
| 1518 | D18 | Ar1 | D18 | D21 | Ar1 |
| 1519 | D19 | Ar1 | D19 | D21 | Ar1 |
| 1520 | D20 | Ar1 | D20 | D21 | Ar1 |
| 1521 | D22 | Ar1 | D22 | D21 | Ar1 |
| 1522 | D23 | Ar1 | D23 | D21 | Ar1 |
| 1523 | D24 | Ar1 | D24 | D21 | Ar1 |
| 1524 | D25 | Ar1 | D25 | D21 | Ar1 |
| 1525 | D26 | Ar1 | D26 | D21 | Ar1 |
| 1526 | D1 | Ar1 | D1 | D22 | Ar1 |
| 1527 | D2 | Ar1 | D2 | D22 | Ar1 |
| 1528 | D3 | Ar1 | D3 | D22 | Ar1 |
| 1529 | D4 | Ar1 | D4 | D22 | Ar1 |
| 1530 | D5 | Ar1 | D5 | D22 | Ar1 |
| 1531 | D6 | Ar1 | D6 | D22 | Ar1 |
| 1532 | D7 | Ar1 | D7 | D22 | Ar1 |
| 1533 | D8 | Ar1 | D8 | D22 | Ar1 |
| 1534 | D9 | Ar1 | D9 | D22 | Ar1 |
| 1535 | D10 | Ar1 | D10 | D22 | Ar1 |
| 1536 | D11 | Ar1 | D11 | D22 | Ar1 |
| 1537 | D12 | Ar1 | D12 | D22 | Ar1 |
| 1538 | D13 | Ar1 | D13 | D22 | Ar1 |
| 1539 | D14 | Ar1 | D14 | D22 | Ar1 |
| 1540 | D15 | Ar1 | D15 | D22 | Ar1 |
| 1541 | D16 | Ar1 | D16 | D22 | Ar1 |
| 1542 | D17 | Ar1 | D17 | D22 | Ar1 |
| 1543 | D18 | Ar1 | D18 | D22 | Ar1 |
| 1544 | D19 | Ar1 | D19 | D22 | Ar1 |
| 1545 | D20 | Ar1 | D20 | D22 | Ar1 |
| 1546 | D21 | Ar1 | D21 | D22 | Ar1 |
| 1547 | D23 | Ar1 | D23 | D22 | Ar1 |
| 1548 | D24 | Ar1 | D24 | D22 | Ar1 |
| 1549 | D25 | Ar1 | D25 | D22 | Ar1 |
| 1550 | D26 | Ar1 | D26 | D22 | Ar1 |
| 1551 | D1 | Ar1 | D1 | D23 | Ar1 |
| 1552 | D2 | Ar1 | D2 | D23 | Ar1 |
| 1553 | D3 | Ar1 | D3 | D23 | Ar1 |
| 1554 | D4 | Ar1 | D4 | D23 | Ar1 |
| 1555 | D5 | Ar1 | D5 | D23 | Ar1 |
| 1556 | D6 | Ar1 | D6 | D23 | Ar1 |
| 1557 | D7 | Ar1 | D7 | D23 | Ar1 |
| 1558 | D8 | Ar1 | D8 | D23 | Ar1 |
| 1559 | D9 | Ar1 | D9 | D23 | Ar1 |
| 1560 | D10 | Ar1 | D10 | D23 | Ar1 |
| 1561 | D11 | Ar1 | D11 | D23 | Ar1 |
| 1562 | D12 | Ar1 | D12 | D23 | Ar1 |
| 1563 | D13 | Ar1 | D13 | D23 | Ar1 |
| 1564 | D14 | Ar1 | D14 | D23 | Ar1 |
| 1565 | D15 | Ar1 | D15 | D23 | Ar1 |
| 1566 | D16 | Ar1 | D16 | D23 | Ar1 |
| 1567 | D17 | Ar1 | D17 | D23 | Ar1 |
| 1568 | D18 | Ar1 | D18 | D23 | Ar1 |
| 1569 | D19 | Ar1 | D19 | D23 | Ar1 |
| 1570 | D20 | Ar1 | D20 | D23 | Ar1 |
| 1571 | D21 | Ar1 | D21 | D23 | Ar1 |
| 1572 | D22 | Ar1 | D22 | D23 | Ar1 |
| 1573 | D24 | Ar1 | D24 | D23 | Ar1 |
| 1574 | D25 | Ar1 | D25 | D23 | Ar1 |
| 1575 | D26 | Ar1 | D26 | D23 | Ar1 |
| 1576 | D1 | Ar1 | D1 | D24 | Ar1 |
| 1577 | D2 | Ar1 | D2 | D24 | Ar1 |
| 1578 | D3 | Ar1 | D3 | D24 | Ar1 |
| 1579 | D4 | Ar1 | D4 | D24 | Ar1 |
| 1580 | D5 | Ar1 | D5 | D24 | Ar1 |
| 1581 | D6 | Ar1 | D6 | D24 | Ar1 |
| 1582 | D7 | Ar1 | D7 | D24 | Ar1 |
| 1583 | D8 | Ar1 | D8 | D24 | Ar1 |
| 1584 | D9 | Ar1 | D9 | D24 | Ar1 |
| 1585 | D10 | Ar1 | D10 | D24 | Ar1 |
| 1586 | D11 | Ar1 | D11 | D24 | Ar1 |
| 1587 | D12 | Ar1 | D12 | D24 | Ar1 |
| 1588 | D13 | Ar1 | D13 | D24 | Ar1 |
| 1589 | D14 | Ar1 | D14 | D24 | Ar1 |
| 1590 | D15 | Ar1 | D15 | D24 | Ar1 |
| 1591 | D18 | Ar1 | D16 | D24 | Ar1 |
| 1592 | D17 | Ar1 | D17 | D24 | Ar1 |
| 1593 | D18 | Ar1 | D18 | D24 | Ar1 |
| 1594 | D19 | Ar1 | D19 | D24 | Ar1 |
| 1595 | D20 | Ar1 | D20 | D24 | Ar1 |
| 1596 | D21 | Ar1 | D21 | D24 | Ar1 |
| 1597 | D22 | Ar1 | D22 | D24 | Ar1 |
| 1598 | D23 | Ar1 | D23 | D24 | Ar1 |
| 1599 | D25 | Ar1 | D25 | D24 | Ar1 |
| 1600 | D26 | Ar1 | D26 | D24 | Ar1 |
| 1601 | D1 | Ar1 | D1 | D25 | Ar1 |
| 1602 | D2 | Ar1 | D2 | D25 | Ar1 |
| 1603 | D3 | Ar1 | D3 | D25 | Ar1 |
| 1604 | D4 | Ar1 | D4 | D25 | Ar1 |
| 1605 | D5 | Ar1 | D5 | D25 | Ar1 |
| 1606 | D6 | Ar1 | D6 | D25 | Ar1 |
| 1607 | D7 | Ar1 | D7 | D25 | Ar1 |
| 1608 | D8 | Ar1 | D8 | D25 | Ar1 |
| 1609 | D9 | Ar1 | D9 | D25 | Ar1 |
| 1610 | D10 | Ar1 | D10 | D25 | Ar1 |
| 1611 | D11 | Ar1 | D11 | D25 | Ar1 |
| 1612 | D12 | Ar1 | D12 | D25 | Ar1 |
| 1613 | D13 | Ar1 | D13 | D25 | Ar1 |
| 1614 | D14 | Ar1 | D14 | D25 | Ar1 |
| 1615 | D15 | Ar1 | D15 | D25 | Ar1 |
| 1616 | D16 | Ar1 | D16 | D25 | Ar1 |
| 1617 | D17 | Ar1 | D17 | D25 | Ar1 |
| 1618 | D18 | Ar1 | D18 | D25 | Ar1 |
| 1619 | D19 | Ar1 | D19 | D25 | Ar1 |
| 1620 | D20 | Ar1 | D20 | D25 | Ar1 |
| 1621 | D21 | Ar1 | D21 | D25 | Ar1 |
| 1622 | D22 | Ar1 | D22 | D25 | Ar1 |
| 1623 | D23 | Ar1 | D23 | D25 | Ar1 |
| 1624 | D24 | Ar1 | D24 | D25 | Ar1 |
| 1625 | D26 | Ar1 | D26 | D25 | Ar1 |
| 1626 | D1 | Ar1 | D1 | D26 | Ar1 |
| 1627 | D2 | Ar1 | D2 | D26 | Ar1 |
| 1628 | D3 | Ar1 | D3 | D26 | Ar1 |
| 1629 | D4 | Ar1 | D4 | D26 | Ar1 |
| 1630 | D5 | Ar1 | D5 | D26 | Ar1 |
| 1631 | D6 | Ar1 | D6 | D26 | Ar1 |
| 1632 | D7 | Ar1 | D7 | D26 | Ar1 |
| 1633 | D8 | Ar1 | D8 | D26 | Ar1 |
| 1634 | D9 | Ar1 | D9 | D26 | Ar1 |
| 1635 | D10 | Ar1 | D10 | D26 | Ar1 |
| 1636 | D11 | Ar1 | D11 | D26 | Ar1 |
| 1637 | D12 | Ar1 | D12 | D26 | Ar1 |
| 1638 | D13 | Ar1 | D13 | D26 | Ar1 |
| 1639 | D14 | Ar1 | D14 | D26 | Ar1 |
| 1640 | D15 | Ar1 | D15 | D26 | Ar1 |
| 1641 | D16 | Ar1 | D18 | D26 | Ar1 |
| 1642 | D17 | Ar1 | D17 | D26 | Ar1 |
| 1643 | D18 | Ar1 | D18 | D26 | Ar1 |
| 1644 | D19 | Ar1 | D19 | D26 | Ar1 |
| 1645 | D20 | Ar1 | D20 | D26 | Ar1 |
| 1646 | D21 | Ar1 | D21 | D26 | Ar1 |
| 1647 | D22 | Ar1 | D22 | D26 | Ar1 |
| 1648 | D23 | Ar1 | D23 | D26 | Ar1 |
| 1649 | D24 | Ar1 | D24 | D26 | Ar1 |
| 1650 | D25 | Ar1 | D25 | D26 | Ar1 |
| 1651 | D21 | Ar1 | D21 | D1 | Ar1 |
| 1652 | D21 | Ar1 | D21 | D2 | Ar1 |
| 1653 | D21 | Ar1 | D21 | D3 | Ar1 |
| 1654 | D21 | Ar1 | D21 | D4 | Ar1 |
| 1655 | D21 | Ar1 | D21 | D5 | Ar1 |
| 1656 | D21 | Ar1 | D21 | D6 | Ar1 |
| 1657 | D21 | Ar1 | D21 | D7 | Ar1 |
| 1658 | D21 | Ar1 | D21 | D8 | Ar1 |
| 1659 | D21 | Ar1 | D21 | D9 | Ar1 |
| 1660 | D21 | Ar1 | D21 | D10 | Ar1 |
| 1661 | D21 | Ar1 | D21 | D11 | Ar1 |
| 1662 | D21 | Ar1 | D21 | D12 | Ar1 |
| 1663 | D21 | Ar1 | D21 | D13 | Ar1 |
| 1664 | D21 | Ar1 | D21 | D14 | Ar1 |
| 1665 | D21 | Ar1 | D21 | D15 | Ar1 |
| 1666 | D21 | Ar1 | D21 | D16 | Ar1 |
| 1667 | D21 | Ar1 | D21 | D17 | Ar1 |
| 1668 | D21 | Ar1 | D21 | D18 | Ar1 |
| 1669 | D21 | Ar1 | D21 | D19 | Ar1 |
| 1670 | D21 | Ar1 | D21 | D20 | Ar1 |
| 1671 | D21 | Ar1 | D21 | D22 | Ar1 |
| 1672 | D21 | Ar1 | D21 | D23 | Ar1 |
| 1673 | D21 | Ar1 | D21 | D24 | Ar1 |
| 1674 | D21 | Ar1 | D21 | D25 | Ar1 |
| 1675 | D21 | Ar1 | D21 | D26 | Ar1 |
| 1676 | D22 | Ar1 | D22 | D1 | Ar1 |
| 1677 | D22 | Ar1 | D22 | D2 | Ar1 |
| 1678 | D22 | Ar1 | D22 | D3 | Ar1 |
| 1679 | D22 | Ar1 | D22 | D4 | Ar1 |
| 1680 | D22 | Ar1 | D22 | D5 | Ar1 |
| 1681 | D22 | Ar1 | D22 | D6 | Ar1 |
| 1682 | D22 | Ar1 | D22 | D7 | Ar1 |
| 1683 | D22 | Ar1 | D22 | D8 | Ar1 |
| 1684 | D22 | Ar1 | D22 | D9 | Ar1 |
| 1685 | D22 | Ar1 | D22 | D10 | Ar1 |
| 1686 | D22 | Ar1 | D22 | D11 | Ar1 |
| 1687 | D22 | Ar1 | D22 | D12 | Ar1 |
| 1688 | D22 | Ar1 | D22 | D13 | Ar1 |
| 1689 | D22 | Ar1 | D22 | D14 | Ar1 |
| 1690 | D22 | Ar1 | D22 | D15 | Ar1 |
| 1691 | D22 | Ar1 | D22 | D16 | Ar1 |
| 1692 | D22 | Ar1 | D22 | D17 | Ar1 |
| 1693 | D22 | Ar1 | D22 | D18 | Ar1 |
| 1694 | D22 | Ar1 | D22 | D19 | Ar1 |
| 1695 | D22 | Ar1 | D22 | D20 | Ar1 |
| 1696 | D22 | Ar1 | D22 | D21 | Ar1 |
| 1697 | D22 | Ar1 | D22 | D23 | Ar1 |
| 1698 | D22 | Ar1 | D22 | D24 | Ar1 |
| 1699 | D22 | Ar1 | D22 | D25 | Ar1 |
| 1700 | D22 | Ar1 | D22 | D26 | Ar1 |
| 1701 | D23 | Ar1 | D23 | D1 | Ar1 |
| 1702 | D23 | Ar1 | D23 | D2 | Ar1 |
| 1703 | D23 | Ar1 | D23 | D3 | Ar1 |
| 1704 | D23 | Ar1 | D23 | D4 | Ar1 |
| 1705 | D23 | Ar1 | D23 | D5 | Ar1 |
| 1706 | D23 | Ar1 | D23 | D6 | Ar1 |
| 1707 | D23 | Ar1 | D23 | D7 | Ar1 |
| 1708 | D23 | Ar1 | D23 | D8 | Ar1 |
| 1709 | D23 | Ar1 | D23 | D9 | Ar1 |
| 1710 | D23 | Ar1 | D23 | D10 | Ar1 |
| 1711 | D23 | Ar1 | D23 | D11 | Ar1 |
| 1712 | D23 | Ar1 | D23 | D12 | Ar1 |
| 1713 | D23 | Ar1 | D22 | D13 | Ar1 |
| 1714 | D23 | Ar1 | D23 | D14 | Ar1 |
| 1715 | D23 | Ar1 | D23 | D15 | Ar1 |
| 1716 | D23 | Ar1 | D23 | D16 | Ar1 |
| 1717 | D23 | Ar1 | D23 | D17 | Ar1 |
| 1718 | D23 | Ar1 | D23 | D18 | Ar1 |
| 1719 | D23 | Ar1 | D23 | D19 | Ar1 |
| 1720 | D23 | Ar1 | D23 | D20 | Ar1 |
| 1721 | D23 | Ar1 | D23 | D21 | Ar1 |
| 1722 | D23 | Ar1 | D23 | D22 | Ar1 |
| 1723 | D23 | Ar1 | D23 | D24 | Ar1 |
| 1724 | D23 | Ar1 | D23 | D25 | Ar1 |
| 1725 | D23 | Ar1 | D23 | D26 | Ar1 |
| 1726 | D24 | Ar1 | D24 | D1 | Ar1 |
| 1727 | D24 | Ar1 | D24 | D2 | Ar1 |
| 1728 | D24 | Ar1 | D24 | D3 | Ar1 |
| 1729 | D24 | Ar1 | D24 | D4 | Ar1 |
| 1730 | D24 | Ar1 | D24 | D5 | Ar1 |
| 1731 | D24 | Ar1 | D24 | D6 | Ar1 |
| 1732 | D24 | Ar1 | D24 | D7 | Ar1 |
| 1733 | D24 | Ar1 | D24 | D8 | Ar1 |
| 1734 | D24 | Ar1 | D24 | D9 | Ar1 |
| 1735 | D24 | Ar1 | D24 | D10 | Ar1 |
| 1736 | D24 | Ar1 | D24 | D11 | Ar1 |
| 1737 | D24 | Ar1 | D24 | D12 | Ar1 |
| 1738 | D24 | Ar1 | D24 | D13 | Ar1 |
| 1739 | D24 | Ar1 | D24 | D14 | Ar1 |
| 1740 | D24 | Ar1 | D24 | D15 | Ar1 |
| 1741 | D24 | Ar1 | D24 | D16 | Ar1 |
| 1742 | D24 | Ar1 | D24 | D17 | Ar1 |
| 1743 | D24 | Ar1 | D24 | D18 | Ar1 |
| 1744 | D24 | Ar1 | D24 | D19 | Ar1 |
| 1745 | D24 | Ar1 | D24 | D20 | Ar1 |
| 1746 | D24 | Ar1 | D24 | D21 | Ar1 |
| 1747 | D24 | Ar1 | D24 | D22 | Ar1 |
| 1748 | D24 | Ar1 | D24 | D23 | Ar1 |
| 1749 | D24 | Ar1 | D24 | D25 | Ar1 |
| 1750 | D24 | Ar1 | D24 | D26 | Ar1 |
| 1751 | D25 | Ar1 | D25 | D1 | Ar1 |
| 1752 | D25 | Ar1 | D25 | D2 | Ar1 |
| 1753 | D25 | Ar1 | D25 | D3 | Ar1 |
| 1754 | D25 | Ar1 | D25 | D4 | Ar1 |
| 1755 | D25 | Ar1 | D25 | D5 | Ar1 |
| 1756 | D25 | Ar1 | D25 | D6 | Ar1 |
| 1757 | D25 | Ar1 | D25 | D7 | Ar1 |
| 1758 | D25 | Ar1 | D25 | D8 | Ar1 |
| 1759 | D25 | Ar1 | D25 | D9 | Ar1 |
| 1760 | D25 | Ar1 | D25 | D10 | Ar1 |
| 1761 | D25 | Ar1 | D25 | D11 | Ar1 |
| 1762 | D25 | Ar1 | D25 | D12 | Ar1 |
| 1763 | D25 | Ar1 | D25 | D13 | Ar1 |
| 1764 | D25 | Ar1 | D25 | D14 | Ar1 |
| 1765 | D25 | Ar1 | D25 | D15 | Ar1 |
| 1766 | D25 | Ar1 | D25 | D16 | Ar1 |
| 1767 | D25 | Ar1 | D25 | D17 | Ar1 |
| 1768 | D25 | Ar1 | D25 | D18 | Ar1 |
| 1769 | D25 | Ar1 | D25 | D19 | Ar1 |
| 1770 | D25 | Ar1 | D25 | D20 | Ar1 |
| 1771 | D25 | Ar1 | D25 | D21 | Ar1 |
| 1772 | D25 | Ar1 | D25 | D22 | Ar1 |
| 1773 | D25 | Ar1 | D25 | D23 | Ar1 |
| 1774 | D25 | Ar1 | D25 | D24 | Ar1 |
| 1775 | D25 | Ar1 | D25 | D26 | Ar1 |
| 1776 | D26 | Ar1 | D26 | D1 | Ar1 |
| 1777 | D26 | Ar1 | D26 | D2 | Ar1 |
| 1778 | D26 | Ar1 | D26 | D3 | Ar1 |
| 1779 | D26 | Ar1 | D26 | D4 | Ar1 |
| 1780 | D26 | Ar1 | D26 | D5 | Ar1 |
| 1781 | D26 | Ar1 | D26 | D6 | Ar1 |
| 1782 | D26 | Ar1 | D26 | D7 | Ar1 |
| 1783 | D26 | Ar1 | D26 | D8 | Ar1 |
| 1784 | D26 | Ar1 | D26 | D9 | Ar1 |
| 1785 | D26 | Ar1 | D26 | D10 | Ar1 |
| 1788 | D26 | Ar1 | D26 | D11 | Ar1 |
| 1787 | D26 | Ar1 | D26 | D12 | Ar1 |
| 1788 | D26 | Ar1 | D26 | D13 | Ar1 |
| 1789 | D26 | Ar1 | D26 | D14 | Ar1 |
| 1790 | D26 | Ar1 | D26 | D15 | Ar1 |
| 1791 | D26 | Ar1 | D26 | D16 | Ar1 |
| 1792 | D26 | Ar1 | D26 | D17 | Ar1 |
| 1793 | D26 | Ar1 | D26 | D18 | Ar1 |
| 1794 | D26 | Ar1 | D26 | D19 | Ar1 |
| 1795 | D26 | Ar1 | D26 | D20 | Ar1 |
| 1796 | D26 | Ar1 | D26 | D21 | Ar1 |
| 1797 | D26 | Ar1 | D26 | D22 | Ar1 |
| 1798 | D26 | Ar1 | D26 | D23 | Ar1 |
| 1799 | D26 | Ar1 | D26 | D24 | Ar1 |
| 1800 | D26 | Ar1 | D26 | D25 | Ar1 |
| 1801 | D21 | D1 | Ar2 | D1 | Ar2 |
| 1802 | D22 | D1 | Ar2 | D1 | Ar2 |
| 1803 | D23 | D1 | Ar2 | D1 | Ar2 |
| 1804 | D24 | D1 | Ar2 | D1 | Ar2 |
| 1805 | D25 | D1 | Ar2 | D1 | Ar2 |
| 1806 | D26 | D1 | Ar2 | D1 | Ar2 |
| 1807 | D21 | D2 | Ar2 | D2 | Ar2 |
| 1808 | D22 | D2 | Ar2 | D2 | Ar2 |
| 1809 | D23 | D2 | Ar2 | D2 | Ar2 |
| 1810 | D24 | D2 | Ar2 | D2 | Ar2 |
| 1811 | D25 | D2 | Ar2 | D2 | Ar2 |
| 1812 | D26 | D2 | Ar2 | D2 | Ar2 |
| 1813 | D21 | D3 | Ar2 | D3 | Ar2 |
| 1814 | D22 | D3 | Ar2 | D3 | Ar2 |
| 1815 | D23 | D3 | Ar2 | D3 | Ar2 |
| 1816 | D24 | D3 | Ar2 | D3 | Ar2 |
| 1817 | D25 | D3 | Ar2 | D3 | Ar2 |
| 1818 | D26 | D3 | Ar2 | D3 | Ar2 |
| 1819 | D21 | D8 | Ar2 | D8 | Ar2 |
| 1820 | D22 | D8 | Ar2 | D8 | Ar2 |
| 1821 | D23 | D8 | Ar2 | D8 | Ar2 |
| 1822 | D24 | D8 | Ar2 | D8 | Ar2 |
| 1823 | D25 | D8 | Ar2 | D8 | Ar2 |
| 1824 | D26 | D8 | Ar2 | D8 | Ar2 |
| 1825 | D21 | D9 | Ar2 | D9 | Ar2 |
| 1826 | D22 | D9 | Ar2 | D9 | Ar2 |
| 1827 | D23 | D9 | Ar2 | D9 | Ar2 |
| 1828 | D24 | D9 | Ar2 | D9 | Ar2 |
| 1829 | D25 | D9 | Ar2 | D9 | Ar2 |
| 1830 | D26 | D9 | Ar2 | D9 | Ar2 |
| 1831 | D21 | D9 | Ar2 | D10 | Ar2 |
| 1832 | D22 | D10 | Ar2 | D10 | Ar2 |
| 1833 | D23 | D10 | Ar2 | D10 | Ar2 |
| 1834 | D24 | D10 | Ar2 | D10 | Ar2 |
| 1835 | D25 | D10 | Ar2 | D10 | Ar2 |
| 1836 | D26 | D10 | Ar2 | D10 | Ar2 |
| 1837 | D21 | D11 | Ar2 | D11 | Ar2 |
| 1838 | D22 | D11 | Ar2 | D11 | Ar2 |
| 1839 | D23 | D11 | Ar2 | D11 | Ar2 |
| 1840 | D24 | D11 | Ar2 | D11 | Ar2 |
| 1841 | D25 | D11 | Ar2 | D11 | Ar2 |
| 1842 | D26 | D11 | Ar2 | D11 | Ar2 |
| 1843 | D21 | D12 | Ar2 | D12 | Ar2 |
| 1844 | D22 | D12 | Ar2 | D12 | Ar2 |
| 1845 | D23 | D12 | Ar2 | D12 | Ar2 |
| 1846 | D24 | D12 | Ar2 | D12 | Ar2 |
| 1847 | D25 | D12 | Ar2 | D12 | Ar2 |
| 1848 | D26 | D12 | Ar2 | D12 | Ar2 |
| 1849 | D21 | D1 | Ar3 | D1 | Ar3 |
| 1850 | D22 | D1 | Ar3 | D1 | Ar3 |
| 1851 | D23 | D1 | Ar3 | D1 | Ar3 |
| 1852 | D24 | D1 | Ar3 | D1 | Ar3 |
| 1853 | D25 | D1 | Ar3 | D1 | Ar3 |
| 1854 | D26 | D1 | Ar3 | D1 | Ar3 |
| 1855 | D21 | D2 | Ar3 | D2 | Ar3 |
| 1856 | D22 | D2 | Ar3 | D2 | Ar3 |
| 1857 | D23 | D2 | Ar3 | D2 | Ar3 |
| 1858 | D24 | D2 | Ar3 | D2 | Ar3 |
| 1859 | D25 | D2 | Ar3 | D2 | Ar3 |
| 1860 | D26 | D2 | Ar3 | D2 | Ar3 |
| 1861 | D21 | D3 | Ar3 | D3 | Ar3 |
| 1862 | D22 | D3 | Ar3 | D3 | Ar3 |
| 1863 | D23 | D3 | Ar3 | D3 | Ar3 |
| 1864 | D24 | D3 | Ar3 | D3 | Ar3 |
| 1865 | D25 | D3 | Ar3 | D3 | Ar3 |
| 1866 | D26 | D3 | Ar3 | D3 | Ar3 |
| 1867 | D21 | D8 | Ar3 | D8 | Ar3 |
| 1868 | D22 | D8 | Ar3 | D8 | Ar3 |
| 1869 | D23 | D8 | Ar3 | D8 | Ar3 |
| 1870 | D24 | D8 | Ar3 | D8 | Ar3 |
| 1871 | D25 | D8 | Ar3 | D8 | Ar3 |
| 1872 | D26 | D8 | Ar3 | D8 | Ar3 |
| 1873 | D21 | D9 | Ar3 | D8 | Ar3 |
| 1874 | D22 | D9 | Ar3 | D9 | Ar3 |
| 1875 | D23 | D9 | Ar3 | D9 | Ar3 |
| 1876 | D24 | D9 | Ar3 | D9 | Ar3 |
| 1877 | D25 | D9 | Ar3 | D9 | Ar3 |
| 1878 | D26 | D9 | Ar3 | D9 | Ar3 |
| 1879 | D21 | D10 | Ar3 | D10 | Ar3 |
| 1880 | D22 | D10 | Ar3 | D10 | Ar3 |
| 1881 | D23 | D10 | Ar3 | D10 | Ar3 |
| 1882 | D24 | D10 | Ar3 | D10 | Ar3 |
| 1883 | D25 | D10 | Ar3 | D10 | Ar3 |
| 1884 | D26 | D10 | Ar3 | D10 | Ar3 |
| 1885 | D21 | D11 | Ar3 | D11 | Ar3 |
| 1886 | D22 | D11 | Ar3 | D11 | Ar3 |
| 1887 | D23 | D11 | Ar3 | D11 | Ar3 |
| 1888 | D24 | D11 | Ar3 | D11 | Ar3 |
| 1889 | D25 | D11 | Ar3 | D11 | Ar3 |
| 1890 | D26 | D11 | Ar3 | D11 | Ar3 |
| 1891 | D21 | D12 | Ar3 | D12 | Ar3 |
| 1892 | D22 | D12 | Ar3 | D12 | Ar3 |
| 1893 | D23 | D12 | Ar3 | D12 | Ar3 |
| 1894 | D24 | D12 | Ar3 | D12 | Ar3 |
| 1895 | D25 | D12 | Ar3 | D12 | Ar3 |
| 1896 | D26 | D12 | Ar3 | D12 | Ar3 |
| 1897 | D21 | D1 | Ar4 | D1 | Ar4 |
| 1898 | D22 | D1 | Ar4 | D1 | Ar4 |
| 1899 | D23 | D1 | Ar4 | D1 | Ar4 |
| 1900 | D24 | D1 | Ar4 | D1 | Ar4 |
| 1901 | D25 | D1 | Ar4 | D1 | Ar4 |
| 1902 | D26 | D1 | Ar4 | D1 | Ar4 |
| 1903 | D21 | D2 | Ar4 | D2 | Ar4 |
| 1904 | D22 | D2 | Ar4 | D2 | Ar4 |
| 1905 | D23 | D2 | Ar4 | D2 | Ar4 |
| 1906 | D24 | D2 | Ar4 | D2 | Ar4 |
| 1907 | D25 | D2 | Ar4 | D2 | Ar4 |
| 1908 | D26 | D2 | Ar4 | D2 | Ar4 |
| 1909 | D21 | D3 | Ar4 | D3 | Ar4 |
| 1910 | D22 | D3 | Ar4 | D3 | Ar4 |
| 1911 | D23 | D3 | Ar4 | D3 | Ar4 |
| 1912 | D24 | D3 | Ar4 | D3 | Ar4 |
| 1913 | D25 | D3 | Ar4 | D3 | Ar4 |
| 1914 | D26 | D3 | Ar4 | D3 | Ar4 |
| 1915 | D21 | D8 | Ar4 | D8 | Ar4 |
| 1916 | D22 | D8 | Ar4 | D8 | Ar4 |
| 1917 | D23 | D8 | Ar4 | D8 | Ar4 |
| 1918 | D24 | D8 | Ar4 | D8 | Ar4 |
| 1919 | D25 | D8 | Ar4 | D8 | Ar4 |
| 1920 | D26 | D8 | Ar4 | D8 | Ar4 |
| 1921 | D21 | D9 | Ar4 | D9 | Ar4 |
| 1922 | D22 | D9 | Ar4 | D9 | Ar4 |
| 1923 | D23 | D9 | Ar4 | D9 | Ar4 |
| 1924 | D24 | D9 | Ar4 | D9 | Ar4 |
| 1925 | D25 | D9 | Ar4 | D9 | Ar4 |
| 1926 | D26 | D9 | Ar4 | D9 | Ar4 |
| 1927 | D21 | D10 | Ar4 | D10 | Ar4 |
| 1928 | D22 | D10 | Ar4 | D10 | Ar4 |
| 1929 | D23 | D10 | Ar4 | D10 | Ar4 |
| 1930 | D24 | D10 | Ar4 | D10 | Ar4 |
| 1931 | D25 | D10 | Ar4 | D10 | Ar4 |
| 1932 | D26 | D10 | Ar4 | D10 | Ar4 |
| 1933 | D21 | D11 | Ar4 | D11 | Ar4 |
| 1934 | D22 | D11 | Ar4 | D11 | Ar4 |
| 1935 | D23 | D11 | Ar4 | D11 | Ar4 |
| 1936 | D24 | D11 | Ar4 | D11 | Ar4 |
| 1937 | D25 | D11 | Ar4 | D11 | Ar4 |
| 1938 | D26 | D11 | Ar4 | D11 | Ar4 |
| 1939 | D21 | D12 | Ar4 | D12 | Ar4 |
| 1940 | D22 | D12 | Ar4 | D12 | Ar4 |
| 1941 | D23 | D12 | Ar4 | D12 | Ar4 |
| 1942 | D24 | D12 | Ar4 | D12 | Ar4 |
| 1943 | D25 | D12 | Ar4 | D12 | Ar4 |
| 1944 | D26 | D12 | Ar4 | D12 | Ar4 |
| 1945 | D21 | D1 | Ar5 | D1 | Ar5 |
| 1946 | D22 | D1 | Ar5 | D1 | Ar5 |
| 1947 | D23 | D1 | Ar5 | D1 | Ar5 |
| 1948 | D24 | D1 | Ar5 | D1 | Ar5 |
| 1949 | D25 | D1 | Ar5 | D1 | Ar5 |
| 1950 | D26 | D1 | Ar5 | D1 | Ar5 |
| 1951 | D21 | D2 | Ar5 | D2 | Ar5 |
| 1952 | D22 | D2 | Ar5 | D2 | Ar5 |
| 1953 | D23 | D2 | Ar5 | D2 | Ar5 |
| 1954 | D24 | D2 | Ar5 | D2 | Ar5 |
| 1955 | D25 | D2 | Ar5 | D2 | Ar5 |
| 1956 | D26 | D2 | Ar5 | D2 | Ar5 |
| 1957 | D21 | D3 | Ar5 | D3 | Ar5 |
| 1958 | D22 | D3 | Ar5 | D3 | Ar5 |
| 1959 | D23 | D3 | Ar5 | D3 | Ar5 |
| 1960 | D24 | D3 | Ar5 | D3 | Ar5 |
| 1961 | D25 | D3 | Ar5 | D3 | Ar5 |
| 1962 | D26 | D3 | Ar5 | D3 | Ar5 |
| 1963 | D21 | D8 | Ar5 | D8 | Ar5 |
| 1964 | D22 | D8 | Ar5 | D8 | Ar5 |
| 1965 | D23 | D8 | Ar5 | D8 | Ar5 |
| 1966 | D24 | D8 | Ar5 | D8 | Ar5 |
| 1967 | D25 | D8 | Ar5 | D8 | Ar5 |
| 1968 | D26 | D8 | Ar5 | D8 | Ar5 |
| 1969 | D21 | D9 | Ar5 | D9 | Ar5 |
| 1970 | D22 | D9 | Ar5 | D9 | Ar5 |
| 1971 | D23 | D9 | Ar5 | D9 | Ar5 |
| 1972 | D24 | D9 | Ar5 | D9 | Ar5 |
| 1973 | D25 | D9 | Ar5 | D9 | Ar5 |
| 1974 | D26 | D9 | Ar5 | D9 | Ar5 |
| 1975 | D21 | D10 | Ar5 | D10 | Ar5 |
| 1976 | D22 | D10 | Ar5 | D10 | Ar5 |
| 1977 | D23 | D10 | Ar5 | D10 | Ar5 |
| 1978 | D24 | D10 | Ar5 | D10 | Ar5 |
| 1979 | D25 | D10 | Ar5 | D10 | Ar5 |
| 1980 | D26 | D10 | Ar5 | D10 | Ar5 |
| 1981 | D21 | D11 | Ar5 | D11 | Ar5 |
| 1982 | D22 | D11 | Ar3 | D11 | Ar5 |
| 1983 | D23 | D11 | Ar5 | D11 | Ar5 |
| 1984 | D24 | D11 | Ar5 | D11 | Ar5 |
| 1985 | D25 | D11 | Ar5 | D11 | Ar5 |
| 1986 | D26 | D11 | Ar5 | D11 | Ar5 |
| 1987 | D21 | D12 | Ar5 | D12 | Ar5 |
| 1988 | D22 | D12 | Ar5 | D12 | Ar5 |
| 1989 | D23 | D12 | Ar5 | D12 | Ar5 |
| 1990 | D24 | D12 | Ar5 | D12 | Ar5 |
| 1991 | D25 | D12 | Ar5 | D12 | Ar5 |
| 1992 | D26 | D12 | Ar5 | D12 | Ar5 |
| 1993 | D21 | D1 | Ar6 | D1 | Ar6 |
| 1994 | D22 | D1 | Ar6 | D1 | Ar6 |
| 1995 | D23 | D1 | Ar6 | D1 | Ar6 |
| 1996 | D24 | D1 | Ar6 | D1 | Ar6 |
| 1997 | D25 | D1 | Ar6 | D1 | Ar6 |
| 1998 | D26 | D1 | Ar6 | D1 | Ar6 |
| 1999 | D21 | D2 | Ar6 | D2 | Ar6 |
| 2000 | D22 | D2 | Ar6 | D2 | Ar6 |
| 2001 | D23 | D2 | Ar6 | D2 | Ar6 |
| 2002 | D24 | D2 | Ar6 | D2 | Ar6 |
| 2003 | D25 | D2 | Ar6 | D2 | Ar6 |
| 2004 | D26 | D2 | Ar6 | D2 | Ar6 |
| 2005 | D21 | D3 | Ar6 | D3 | Ar6 |
| 2006 | D22 | D3 | Ar6 | D3 | Ar6 |
| 2007 | D23 | D3 | Ar6 | D3 | Ar6 |
| 2008 | D24 | D3 | Ar6 | D3 | Ar6 |
| 2009 | D25 | D3 | Ar6 | D3 | Ar6 |
| 2010 | D26 | D3 | Ar6 | D3 | Ar6 |
| 2011 | D21 | D8 | Ar6 | D8 | Ar6 |
| 2012 | D22 | D8 | Ar6 | D8 | Ar6 |
| 2013 | D23 | D8 | Ar6 | D8 | Ar6 |
| 2014 | D24 | D8 | Ar6 | D8 | Ar6 |
| 2015 | D25 | D8 | Ar6 | D8 | Ar6 |
| 2016 | D26 | D8 | Ar6 | D8 | Ar6 |
| 2017 | D21 | D9 | Ar6 | D9 | Ar6 |
| 2018 | D22 | D9 | Ar6 | D9 | Ar6 |
| 2019 | D23 | D9 | Ar6 | D9 | Ar6 |
| 2020 | D24 | D9 | Ar6 | D9 | Ar6 |
| 2021 | D25 | D9 | Ar6 | D9 | Ar6 |
| 2022 | D26 | D9 | Ar6 | D9 | Ar6 |
| 2023 | D21 | D10 | Ar6 | D10 | Ar6 |
| 2024 | D22 | D10 | Ar6 | D10 | Ar6 |
| 2025 | D23 | D10 | Ar6 | D10 | Ar6 |
| 2026 | D24 | D10 | Ar6 | D10 | Ar6 |
| 2027 | D25 | D10 | Ar6 | D10 | Ar6 |
| 2028 | D26 | D10 | Ar6 | D10 | Ar6 |
| 2029 | D21 | D11 | Ar6 | D11 | Ar6 |
| 2030 | D22 | D11 | Ar6 | D11 | Ar6 |
| 2031 | D23 | D11 | Ar6 | D11 | Ar6 |
| 2032 | D24 | D11 | Ar6 | D11 | Ar6 |
| 2033 | D25 | D11 | Ar6 | D11 | Ar6 |
| 2034 | D26 | D11 | Ar6 | D11 | Ar6 |
| 2035 | D21 | D12 | Ar6 | D12 | Ar6 |
| 2036 | D22 | D12 | Ar6 | D12 | Ar6 |
| 2037 | D23 | D12 | Ar6 | D12 | Ar6 |
| 2038 | D24 | D12 | Ar6 | D12 | Ar6 |
| 2039 | D25 | D12 | Ar6 | D12 | Ar6 |
| 2040 | D26 | D12 | Ar6 | D12 | Ar6 |
| 2041 | D21 | D1 | Ar7 | D1 | Ar7 |
| 2042 | D22 | D1 | Ar7 | D1 | Ar7 |
| 2043 | D23 | D1 | Ar7 | D1 | Ar7 |
| 2044 | D24 | D1 | Ar7 | D1 | Ar7 |
| 2045 | D25 | D1 | Ar7 | D1 | Ar7 |
| 2046 | D26 | D1 | Ar7 | D1 | Ar7 |
| 2047 | D21 | D2 | Ar7 | D2 | Ar7 |
| 2048 | D22 | D2 | Ar7 | D2 | Ar7 |
| 2049 | D23 | D2 | Ar7 | D2 | Ar7 |
| 2050 | D24 | D2 | Ar7 | D2 | Ar7 |
| 2051 | D25 | D2 | Ar7 | D2 | Ar7 |
| 2052 | D26 | D2 | Ar7 | D2 | Ar7 |
| 2053 | D21 | D3 | Ar7 | D3 | Ar7 |
| 2054 | D22 | D3 | Ar7 | D3 | Ar7 |
| 2055 | D23 | D3 | Ar7 | D3 | Ar7 |
| 2056 | D24 | D3 | Ar7 | D3 | Ar7 |
| 2057 | D25 | D3 | Ar7 | D3 | Ar7 |
| 2058 | D26 | D3 | Ar7 | D3 | Ar7 |
| 2059 | D21 | D8 | Ar7 | D8 | Ar7 |
| 2060 | D22 | D8 | Ar7 | D8 | Ar7 |
| 2061 | D23 | D8 | Ar7 | D8 | Ar7 |
| 2062 | D24 | D8 | Ar7 | D8 | Ar7 |
| 2063 | D25 | D8 | Ar7 | D8 | Ar7 |
| 2064 | D26 | D8 | Ar7 | D8 | Ar7 |
| 2065 | D21 | D9 | Ar7 | D9 | Ar7 |
| 2066 | D22 | D9 | Ar7 | D9 | Ar7 |
| 2067 | D23 | D9 | Ar7 | D9 | Ar7 |
| 2068 | D24 | D9 | Ar7 | D9 | Ar7 |
| 2069 | D25 | D9 | Ar7 | D9 | Ar7 |
| 2070 | D26 | D9 | Ar7 | D9 | Ar7 |
| 2071 | D21 | D10 | Ar7 | D10 | Ar7 |
| 2072 | D22 | D10 | Ar7 | D10 | Ar7 |
| 2073 | D23 | D10 | Ar7 | D10 | Ar7 |
| 2074 | D24 | D10 | Ar7 | D10 | Ar7 |
| 2075 | D25 | D10 | Ar7 | D10 | Ar7 |
| 2076 | D26 | D10 | Ar7 | D10 | Ar7 |
| 2077 | D21 | D11 | Ar7 | D11 | Ar7 |
| 2078 | D22 | D11 | Ar7 | D11 | Ar7 |
| 2079 | D23 | D11 | Ar7 | D11 | Ar7 |
| 2080 | D24 | D11 | Ar7 | D11 | Ar7 |
| 2081 | D25 | D11 | Ar7 | D11 | Ar7 |
| 2082 | D26 | D11 | Ar7 | D11 | Ar7 |
| 2083 | D21 | D12 | Ar7 | D12 | Ar7 |
| 2084 | D22 | D12 | Ar7 | D12 | Ar7 |
| 2085 | D23 | D12 | Ar7 | D12 | Ar7 |
| 2086 | D24 | D12 | A77 | D12 | Ar7 |
| 2087 | D25 | D12 | Ar7 | D12 | Ar7 |
| 2088 | D26 | D12 | Ar7 | D12 | Ar7 |
| 2089 | D21 | D1 | Ar8 | D1 | Ar8 |
| 2090 | D22 | D1 | Ar8 | D1 | Ar8 |
| 2091 | D23 | D1 | Ar8 | D1 | Ar8 |
| 2092 | D24 | D1 | Ar8 | D1 | Ar8 |
| 2093 | D25 | D1 | Ar8 | D1 | Ar8 |
| 2094 | D26 | D1 | Ar8 | D1 | Ar8 |
| 2095 | D21 | D2 | Ar8 | D2 | Ar8 |
| 2096 | D22 | D2 | Ar8 | D2 | Ar8 |
| 2097 | D23 | D2 | Ar8 | D2 | Ar8 |
| 2098 | D24 | D2 | Ar8 | D2 | Ar8 |
| 2099 | D25 | D2 | Ar8 | D2 | Ar8 |
| 2100 | D26 | D2 | Ar8 | D2 | Ar8 |
| 2101 | D21 | D3 | Ar8 | D3 | Ar8 |
| 2102 | D22 | D3 | Ar8 | D3 | Ar8 |
| 2103 | D23 | D3 | Ar8 | D3 | Ar8 |
| 2104 | D24 | D3 | Ar8 | D3 | Ar8 |
| 2105 | D25 | D3 | Ar8 | D3 | Ar8 |
| 2106 | D26 | D3 | Ar8 | D3 | Ar8 |
| 2107 | D21 | D8 | Ar8 | D8 | Ar8 |
| 2108 | D22 | D8 | Ar8 | D8 | Ar8 |
| 2109 | D23 | D8 | Ar8 | D8 | Ar8 |
| 2110 | D24 | D8 | Ar8 | D8 | Ar8 |
| 2111 | D25 | D8 | Ar8 | D9 | Ar8 |
| 2112 | D26 | D8 | Ar8 | D8 | Ar8 |
| 2113 | D21 | D9 | Ar8 | D9 | Ar8 |
| 2114 | D22 | D9 | Ar8 | D9 | Ar8 |
| 2115 | D23 | D9 | Ar8 | D9 | Ar8 |
| 2116 | D24 | D9 | Ar8 | D9 | Ar8 |
| 2117 | D25 | D9 | Ar8 | D9 | Ar8 |
| 2118 | D26 | D9 | Ar8 | D9 | Ar8 |
| 2119 | D21 | D10 | Ar8 | D10 | Ar8 |
| 2120 | D22 | D10 | Ar8 | D10 | Ar8 |
| 2121 | D23 | D10 | Ar8 | D10 | Ar8 |
| 2122 | D24 | D10 | Ar8 | D10 | Ar8 |
| 2123 | D25 | D10 | Ar8 | D10 | Ar8 |
| 2124 | D26 | D10 | Ar8 | D10 | Ar8 |
| 2125 | D21 | D11 | Ar8 | D11 | Ar8 |
| 2126 | D22 | D11 | Ar8 | D11 | Ar8 |
| 2127 | D23 | D11 | Ar8 | D11 | Ar8 |
| 2128 | D24 | D11 | Ar8 | D11 | Ar8 |
| 2129 | D25 | D11 | Ar8 | D11 | Ar8 |
| 2130 | D26 | D11 | Ar8 | D11 | Ar8 |
| 2131 | D21 | D12 | Ar8 | D12 | Ar8 |
| 2132 | D22 | D12 | Ar8 | D12 | Ar8 |
| 2133 | D23 | D12 | Ar8 | D12 | Ar8 |
| 2134 | D24 | D12 | Ar8 | D12 | Ar8 |
| 2135 | D25 | D12 | Ar8 | D12 | Ar8 |
| 2136 | D26 | D12 | Ar8 | D12 | Ar8 |
| 2137 | D21 | D1 | Ar9 | D1 | Ar9 |
| 2138 | D22 | D1 | Ar9 | D1 | Ar9 |
| 2139 | D23 | D1 | Ar9 | D1 | Ar9 |
| 2140 | D24 | D1 | Ar9 | D1 | Ar9 |
| 2141 | D25 | D1 | Ar9 | D1 | Ar9 |
| 2142 | D26 | D1 | Ar9 | D1 | Ar9 |
| 2143 | D21 | D2 | Ar9 | D2 | Ar9 |
| 2144 | D22 | D2 | Ar9 | D2 | Ar9 |
| 2145 | D23 | D2 | Ar9 | D2 | Ar9 |
| 2146 | D24 | D2 | Ar9 | D2 | Ar9 |
| 2147 | D25 | D2 | Ar9 | D2 | Ar9 |
| 2148 | D26 | D2 | Ar9 | D2 | Ar9 |
| 2149 | D21 | D3 | Ar9 | D3 | Ar9 |
| 2150 | D22 | D3 | Ar9 | D3 | Ar9 |
| 2151 | D23 | D3 | Ar9 | D3 | Ar9 |
| 2152 | D24 | D3 | Ar9 | D3 | Ar9 |
| 2153 | D25 | D3 | Ar9 | D3 | Ar9 |
| 2154 | D26 | D3 | Ar9 | D3 | Ar9 |
| 2155 | D21 | D8 | Ar9 | D8 | Ar9 |
| 2156 | D22 | D8 | Ar9 | D8 | Ar9 |
| 2157 | D23 | D8 | Ar9 | D8 | Ar9 |
| 2158 | D24 | D8 | Ar9 | D8 | Ar9 |
| 2159 | D25 | D8 | Ar9 | D8 | Ar9 |
| 2160 | D26 | D8 | Ar9 | D8 | Ar9 |
| 2161 | D21 | D9 | Ar9 | D9 | Ar9 |
| 2162 | D22 | D9 | Ar9 | D9 | Ar9 |
| 2163 | D23 | D9 | Ar9 | D9 | Ar9 |
| 2164 | D24 | D9 | Ar9 | D9 | Ar9 |
| 2165 | D25 | D9 | Ar9 | D9 | Ar9 |
| 2166 | D26 | D9 | Ar9 | D9 | Ar9 |
| 2167 | D21 | D10 | Ar9 | D10 | Ar9 |
| 2168 | D22 | D10 | Ar9 | D10 | Ar9 |
| 2169 | D23 | D10 | Ar9 | D10 | Ar9 |
| 2170 | D24 | D10 | Ar9 | D10 | Ar9 |
| 2171 | D25 | D10 | Ar9 | D10 | Ar9 |
| 2172 | D26 | D10 | Ar9 | D10 | Ar9 |
| 2173 | D21 | D11 | Ar9 | D11 | Ar9 |
| 2174 | D22 | D11 | Ar9 | D11 | Ar9 |
| 2175 | D23 | D11 | Ar9 | D11 | Ar9 |
| 2176 | D24 | D11 | Ar9 | D11 | Ar9 |
| 2177 | D25 | D11 | Ar9 | D11 | Ar9 |
| 2178 | D26 | D11 | Ar9 | D11 | Ar9 |
| 2179 | D21 | D12 | Ar9 | D12 | Ar9 |
| 2180 | D22 | D12 | Ar9 | D12 | Ar9 |
| 2181 | D23 | D12 | Ar9 | D12 | Ar9 |
| 2182 | D24 | D12 | Ar9 | D12 | Ar9 |
| 2183 | D25 | D12 | Ar9 | D12 | Ar9 |
| 2184 | D26 | D12 | Ar9 | D12 | Ar9 |
| 2185 | D21 | D1 | Ar10 | D1 | Ar10 |
| 2186 | D22 | D1 | Ar10 | D1 | Ar10 |
| 2187 | D23 | D1 | Ar10 | D1 | Ar10 |
| 2188 | D24 | D1 | Ar10 | D1 | Ar10 |
| 2189 | D25 | D1 | Ar10 | D1 | Ar10 |
| 2190 | D26 | D1 | Ar10 | D1 | Ar10 |
| 2191 | D21 | D2 | Ar10 | D2 | Ar10 |
| 2192 | D22 | D2 | Ar10 | D2 | Ar10 |
| 2193 | D23 | D2 | Ar10 | D2 | Ar10 |
| 2194 | D24 | D2 | Ar10 | D2 | Ar10 |
| 2195 | D25 | D2 | Ar10 | D2 | Ar10 |
| 2196 | D26 | D2 | Ar10 | D2 | Ar10 |
| 2197 | D21 | D3 | Ar10 | D3 | Ar10 |
| 2198 | D22 | D3 | Ar10 | D3 | Ar10 |
| 2199 | D23 | D3 | Ar10 | D3 | Ar10 |
| 2200 | D24 | D3 | Ar10 | D3 | Ar10 |
| 2201 | D25 | D3 | Ar10 | D3 | Ar10 |
| 2202 | D26 | D3 | Ar10 | D3 | Ar10 |
| 2203 | D21 | D8 | Ar10 | D8 | Ar10 |
| 2204 | D22 | D8 | Ar10 | D8 | Ar10 |
| 2205 | D23 | D8 | Ar10 | D8 | Ar10 |
| 2206 | D24 | D8 | Ar10 | D8 | Ar10 |
| 2207 | D25 | D8 | Ar10 | D8 | Ar10 |
| 2208 | D26 | D8 | Ar10 | D8 | Ar10 |
| 2209 | D21 | D9 | Ar10 | D9 | Ar10 |
| 2210 | D22 | D9 | Ar10 | D9 | Ar10 |
| 2211 | D23 | D9 | Ar10 | D9 | Ar10 |
| 2212 | D24 | D9 | Ar10 | D9 | Ar10 |
| 2213 | D25 | D9 | Ar10 | D9 | Ar10 |
| 2214 | D26 | D9 | Ar10 | D9 | Ar10 |
| 2215 | D21 | D10 | Ar10 | D10 | Ar10 |
| 2216 | D22 | D10 | Ar10 | D10 | Ar10 |
| 2217 | D23 | D10 | Ar10 | D10 | Ar10 |
| 2218 | D24 | D10 | Ar10 | D10 | Ar10 |
| 2219 | D25 | D10 | Ar10 | D10 | Ar10 |
| 2220 | D26 | D10 | Ar10 | D10 | Ar10 |
| 2221 | D21 | D11 | Ar10 | D11 | Ar10 |
| 2222 | D22 | D11 | Ar10 | D11 | Ar10 |
| 2223 | D23 | D11 | Ar10 | D11 | Ar10 |
| 2224 | D24 | D11 | Ar10 | D11 | Ar10 |
| 2225 | D25 | D11 | Ar10 | D11 | Ar10 |
| 2226 | D26 | D11 | Ar10 | D11 | Ar10 |
| 2227 | D21 | D12 | Ar10 | D12 | Ar10 |
| 2228 | D22 | D12 | Ar10 | D12 | Ar10 |
| 2229 | D23 | D12 | Ar10 | D12 | Ar10 |
| 2230 | D24 | D12 | Ar10 | D12 | Ar10 |
| 2231 | D25 | D12 | Ar10 | D12 | Ar10 |
| 2232 | D26 | D12 | Ar10 | D12 | Ar10 |
| 2233 | D21 | D1 | Ar11 | D1 | Ar11 |
| 2234 | D22 | D1 | Ar11 | D1 | Ar11 |
| 2235 | D23 | D1 | Ar11 | D1 | Ar11 |
| 2236 | D24 | D1 | Ar11 | D1 | Ar11 |
| 2237 | D25 | D1 | Ar11 | D1 | Ar11 |
| 2238 | D26 | D1 | Ar11 | D1 | Ar11 |
| 2239 | D21 | D2 | Ar11 | D2 | Ar11 |
| 2240 | D22 | D2 | Ar11 | D2 | Ar11 |
| 2241 | D23 | D2 | Ar11 | D2 | Ar11 |
| 2242 | D24 | D2 | Ar11 | D2 | Ar11 |
| 2243 | D25 | D2 | Ar11 | D2 | Ar11 |
| 2244 | D26 | D2 | Ar11 | D2 | Ar11 |
| 2245 | D21 | D3 | Ar11 | D3 | Ar11 |
| 2246 | D22 | D3 | Ar11 | D3 | Ar11 |
| 2247 | D23 | D3 | Ar11 | D3 | Ar11 |
| 2248 | D24 | D3 | Ar11 | D3 | Ar11 |
| 2249 | D25 | D3 | Ar11 | D3 | Ar11 |
| 2250 | D26 | D3 | Ar11 | D3 | Ar11 |
| 2251 | D21 | D8 | Ar11 | D8 | Ar11 |
| 2252 | D22 | D8 | Ar11 | D8 | Ar11 |
| 2253 | D23 | D8 | Ar11 | D8 | Ar11 |
| 2254 | D24 | D8 | Ar11 | D8 | Ar11 |
| 2255 | D25 | D8 | Ar11 | D8 | Ar11 |
| 2258 | D26 | D9 | Ar11 | D8 | Ar11 |
| 2257 | D21 | D9 | Ar11 | D9 | Ar11 |
| 2258 | D22 | D9 | Ar11 | D9 | Ar11 |
| 2259 | D23 | D9 | Ar11 | D9 | Ar11 |
| 2260 | D24 | D9 | Ar11 | D9 | Ar11 |
| 2261 | D25 | D9 | Ar11 | D9 | Ar11 |
| 2262 | D26 | D9 | Ar11 | D9 | Ar11 |
| 2263 | D21 | D10 | Ar11 | D10 | Ar11 |
| 2264 | D22 | D10 | Ar11 | D10 | Ar11 |
| 2265 | D23 | D10 | Ar11 | D10 | Ar11 |
| 2266 | D24 | D10 | Ar11 | D10 | Ar11 |
| 2267 | D25 | D10 | Ar11 | D10 | Ar11 |
| 2268 | D26 | D10 | Ar11 | D10 | Ar11 |
| 2269 | D21 | D11 | Ar11 | D11 | Ar11 |
| 2270 | D22 | D11 | Ar11 | D11 | Ar11 |
| 2271 | D23 | D11 | Ar11 | D11 | Ar11 |
| 2272 | D24 | D11 | Ar11 | D11 | Ar11 |
| 2273 | D25 | D11 | Ar11 | D11 | Ar11 |
| 2274 | D26 | D11 | Ar11 | D11 | Ar11 |
| 2275 | D21 | D12 | Ar11 | D12 | Ar11 |
| 2276 | D22 | D12 | Ar11 | D12 | Ar11 |
| 2277 | D23 | D12 | Ar11 | D12 | Ar11 |
| 2278 | D24 | D12 | Ar11 | D12 | Ar11 |
| 2279 | D25 | D12 | Ar11 | D12 | Ar11 |
| 2280 | D26 | D12 | Ar11 | D12 | Ar11 |
| 2281 | D21 | D1 | Ar12 | D1 | Ar12 |
| 2282 | D22 | D1 | Ar12 | D1 | Ar12 |
| 2283 | D23 | D1 | Ar12 | D1 | Ar12 |
| 2284 | D24 | D1 | Ar12 | D1 | Ar12 |
| 2285 | D25 | D1 | Ar12 | D1 | Ar12 |
| 2286 | D26 | D1 | Ar12 | D1 | Ar12 |
| 2287 | D21 | D2 | Ar12 | D2 | Ar12 |
| 2288 | D22 | D2 | Ar12 | D2 | Ar12 |
| 2289 | D23 | D2 | Ar12 | D2 | Ar12 |
| 2290 | D24 | D2 | Ar12 | D2 | Ar12 |
| 2291 | D25 | D2 | Ar12 | D2 | Ar12 |
| 2292 | D26 | D2 | Ar12 | D2 | Ar12 |
| 2293 | D21 | D3 | Ar12 | D3 | Ar12 |
| 2294 | D22 | D3 | Ar12 | D3 | Ar12 |
| 2295 | D23 | D3 | Ar12 | D3 | Ar12 |
| 2296 | D24 | D3 | Ar12 | D3 | Ar12 |
| 2297 | D25 | D3 | Ar12 | D3 | Ar12 |
| 2298 | D26 | D3 | Ar12 | D3 | Ar12 |
| 2299 | D21 | D8 | Ar12 | D8 | Ar12 |
| 2300 | D22 | D8 | Ar12 | D8 | Ar12 |
| 2301 | D23 | D8 | Ar12 | D8 | Ar12 |
| 2302 | D24 | D8 | Ar12 | D8 | Ar12 |
| 2303 | D25 | D8 | Ar12 | D8 | Ar12 |
| 2304 | D26 | D8 | Ar12 | D8 | Ar12 |
| 2305 | D21 | D9 | Ar12 | D9 | Ar12 |
| 2306 | D22 | D9 | Ar12 | D9 | Ar12 |
| 2307 | D23 | D9 | Ar12 | D9 | Ar12 |
| 2308 | D24 | D9 | Ar12 | D9 | Ar12 |
| 2309 | D25 | D9 | Ar12 | D9 | Ar12 |
| 2310 | D26 | D9 | Ar12 | D9 | Ar12 |
| 2311 | D21 | D10 | Ar12 | D10 | Ar12 |
| 2312 | D22 | D10 | Ar12 | D10 | Ar12 |
| 2313 | D23 | D10 | Ar12 | D10 | Ar12 |
| 2314 | D24 | D10 | Ar12 | D10 | Ar12 |
| 2315 | D25 | D10 | Ar12 | D10 | Ar12 |
| 2316 | D26 | D10 | Ar12 | D10 | Ar12 |
| 2317 | D21 | D11 | Ar12 | D11 | Ar12 |
| 2318 | D22 | D11 | Ar12 | D11 | Ar12 |
| 2319 | D23 | D11 | Ar12 | D11 | Ar12 |
| 2320 | D24 | D11 | Ar12 | D11 | Ar12 |
| 2321 | D25 | D11 | Ar12 | D11 | Ar12 |
| 2322 | D26 | D11 | Ar12 | D11 | Ar12 |
| 2323 | D21 | D12 | Ar12 | D12 | Ar12 |
| 2324 | D22 | D12 | Ar12 | D12 | Ar12 |
| 2325 | D23 | D12 | Ar12 | D12 | Ar12 |
| 2326 | D24 | D12 | Ar12 | D12 | Ar12 |
| 2327 | D25 | D12 | Ar12 | D12 | Ar12 |
| 2328 | D26 | D12 | Ar12 | D12 | Ar12 |
| 2329 | D21 | D1 | Ar13 | D1 | Ar13 |
| 2330 | D22 | D1 | Ar13 | D1 | Ar13 |
| 2331 | D23 | D1 | Ar13 | D1 | Ar13 |
| 2332 | D24 | D1 | Ar13 | D1 | Ar13 |
| 2333 | D25 | D1 | Ar13 | D1 | Ar13 |
| 2334 | D26 | D1 | Ar13 | D1 | Ar13 |
| 2335 | D21 | D2 | Ar13 | D2 | Ar13 |
| 2336 | D22 | D2 | Ar13 | D2 | Ar13 |
| 2337 | D23 | D2 | Ar13 | D2 | Ar13 |
| 2338 | D24 | D2 | Ar13 | D2 | Ar13 |
| 2339 | D25 | D2 | Ar13 | D2 | Ar13 |
| 2340 | D26 | D2 | Ar13 | D2 | Ar13 |
| 2341 | D21 | D3 | Ar13 | D3 | Ar13 |
| 2342 | D22 | D3 | Ar13 | D3 | Ar13 |
| 2343 | D23 | D3 | Ar13 | D3 | Ar13 |
| 2344 | D24 | D3 | Ar13 | D3 | Ar13 |
| 2345 | D25 | D3 | Ar13 | D3 | Ar13 |
| 2346 | D26 | D3 | Ar13 | D3 | Ar13 |
| 2347 | D21 | D8 | Ar13 | D8 | Ar13 |
| 2348 | D22 | D8 | Ar13 | D8 | Ar13 |
| 2349 | D23 | D8 | Ar13 | D8 | Ar13 |
| 2350 | D24 | D8 | Ar13 | D8 | Ar13 |
| 2351 | D25 | D8 | Ar13 | D8 | Ar13 |
| 2352 | D26 | D8 | Ar13 | D8 | Ar13 |
| 2353 | D21 | D9 | Ar13 | D9 | Ar13 |
| 2354 | D22 | D9 | Ar13 | D9 | Ar13 |
| 2355 | D23 | D9 | Ar13 | D9 | Ar13 |
| 2356 | D24 | D9 | Ar13 | D9 | Ar13 |
| 2357 | D25 | D9 | Ar13 | D9 | Ar13 |
| 2358 | D21 | D9 | Ar13 | D9 | Ar13 |
| 2359 | D21 | D10 | Ar13 | D10 | Ar13 |
| 2360 | D22 | D10 | Ar13 | D10 | Ar13 |
| 2361 | D23 | D10 | Ar13 | D10 | Ar13 |
| 2362 | D24 | D10 | Ar13 | D10 | Ar13 |
| 2363 | D25 | D10 | Ar13 | D10 | Ar13 |
| 2364 | D26 | D10 | Ar13 | D10 | Ar13 |
| 2365 | D21 | D11 | Ar13 | D11 | Ar13 |
| 2366 | D22 | D11 | Ar13 | D11 | Ar13 |
| 2367 | D23 | D11 | Ar13 | D11 | Ar13 |
| 2368 | D24 | D11 | Ar13 | D11 | Ar13 |
| 2369 | D25 | D11 | Ar13 | D11 | Ar13 |
| 2370 | D26 | D11 | Ar13 | D11 | Ar13 |
| 2371 | D21 | D12 | Ar13 | D12 | Ar13 |
| 2372 | D22 | D12 | Ar13 | D12 | Ar13 |
| 2373 | D23 | D12 | Ar13 | D12 | Ar13 |
| 2374 | D24 | D12 | Ar13 | D12 | Ar13 |
| 2375 | D25 | D12 | Ar13 | D12 | Ar13 |
| 2376 | D26 | D12 | Ar13 | D12 | Ar13 |
| 2377 | D21 | D1 | Ar14 | D1 | Ar14 |
| 2378 | D22 | D1 | Ar14 | D1 | Ar14 |
| 2379 | D23 | D1 | Ar14 | D1 | Ar14 |
| 2380 | D24 | D1 | Ar14 | D1 | Ar14 |
| 2381 | D25 | D1 | Ar14 | D1 | Ar14 |
| 2382 | D26 | D1 | Ar14 | D1 | Ar14 |
| 2383 | D21 | D2 | Ar14 | D2 | Ar14 |
| 2384 | D22 | D2 | Ar14 | D2 | Ar14 |
| 2385 | D23 | D2 | Ar14 | D2 | Ar14 |
| 2386 | D24 | D2 | Ar14 | D2 | Ar14 |
| 2387 | D25 | D2 | Ar14 | D2 | Ar14 |
| 2388 | D26 | D2 | Ar14 | D2 | Ar14 |
| 2389 | D21 | D3 | Ar14 | D3 | Ar14 |
| 2390 | D22 | D3 | Ar14 | D3 | Ar14 |
| 2391 | D23 | D3 | Ar14 | D3 | Ar14 |
| 2392 | D24 | D3 | Ar14 | D3 | Ar14 |
| 2393 | D25 | D3 | Ar14 | D3 | Ar14 |
| 2394 | D26 | D3 | Ar14 | D3 | Ar14 |
| 2395 | D21 | D3 | Ar14 | D3 | Ar14 |
| 2396 | D29 | D8 | Ar14 | D8 | Ar14 |
| 2397 | D23 | D8 | Ar14 | D8 | Ar14 |
| 2398 | D24 | D8 | Ar14 | D8 | Ar14 |
| 2399 | D25 | D8 | Ar14 | D8 | Ar14 |
| 2400 | D26 | D8 | Ar14 | D8 | Ar14 |
| 2401 | D21 | D9 | Ar14 | D9 | Ar14 |
| 2402 | D22 | D9 | Ar14 | D9 | Ar14 |
| 2403 | D23 | D9 | Ar14 | D9 | Ar14 |
| 2404 | D24 | D9 | Ar14 | D9 | Ar14 |
| 2405 | D25 | D9 | Ar14 | D9 | Ar14 |
| 2406 | D26 | D9 | Ar14 | D9 | Ar14 |
| 2407 | D21 | D10 | Ar14 | D10 | Ar14 |
| 2408 | D22 | D10 | Ar14 | D10 | Ar14 |
| 2409 | D23 | D10 | Ar14 | D10 | Ar14 |
| 2410 | D24 | D10 | Ar14 | D10 | Ar14 |
| 2411 | D25 | D10 | Ar14 | D10 | Ar14 |
| 2412 | D26 | D10 | Ar14 | D10 | Ar14 |
| 2413 | D21 | D11 | Ar14 | D11 | Ar14 |
| 2414 | D22 | D11 | Ar14 | D11 | Ar14 |
| 2415 | D23 | D11 | Ar14 | D11 | Ar14 |
| 2416 | D24 | D11 | Ar14 | D11 | Ar14 |
| 2417 | D25 | D11 | Ar14 | D11 | Ar14 |
| 2418 | D26 | D11 | Ar14 | D11 | Ar14 |
| 2419 | D21 | D12 | Ar14 | D12 | Ar14 |
| 2420 | D22 | D12 | Ar14 | D12 | Ar14 |
| 2421 | D23 | D12 | Ar14 | D12 | Ar14 |
| 2422 | D24 | D12 | Ar14 | D12 | Ar14 |
| 2423 | D25 | D12 | Ar14 | D12 | Ar14 |
| 2424 | D26 | D12 | Ar14 | D12 | Ar14 |
| 2425 | D21 | D1 | Ar15 | D1 | Ar15 |
| 2426 | D22 | D1 | Ar15 | D1 | Ar15 |
| 2427 | D23 | D1 | Ar15 | D1 | Ar15 |
| 2428 | D24 | D1 | Ar15 | D1 | Ar15 |
| 2429 | D25 | D1 | Ar15 | D1 | Ar15 |
| 2430 | D26 | D1 | Ar15 | D1 | Ar15 |
| 2431 | D21 | D2 | Ar15 | D2 | Ar15 |
| 2432 | D22 | D2 | Ar15 | D2 | Ar15 |
| 2433 | D23 | D2 | Ar15 | D2 | Ar15 |
| 2434 | D24 | D2 | Ar15 | D2 | Ar15 |
| 2435 | D25 | D2 | Ar15 | D2 | Ar15 |
| 2436 | D26 | D2 | Ar15 | D2 | Ar15 |
| 2437 | D21 | D3 | Ar15 | D3 | Ar15 |
| 2438 | D22 | D3 | Ar15 | D3 | Ar15 |
| 2439 | D23 | D3 | Ar15 | D3 | Ar15 |
| 2440 | D24 | D3 | Ar15 | D3 | Ar15 |
| 2441 | D25 | D3 | Ar15 | D3 | Ar15 |
| 2442 | D26 | D3 | Ar15 | D3 | Ar15 |
| 2443 | D21 | D8 | Ar15 | D8 | Ar15 |
| 2444 | D22 | D8 | Ar15 | D8 | Ar15 |
| 2445 | D23 | D8 | Ar15 | D8 | Ar15 |
| 2446 | D24 | D8 | Ar15 | D8 | Ar15 |
| 2447 | D25 | D8 | Ar15 | D8 | Ar15 |
| 2448 | D26 | D8 | Ar15 | D8 | Ar15 |
| 2449 | D21 | D9 | Ar15 | D9 | Ar15 |
| 2450 | D22 | D9 | Ar15 | D9 | Ar15 |
| 2451 | D23 | D9 | Ar15 | D9 | Ar15 |
| 2452 | D24 | D9 | Ar15 | D9 | Ar15 |
| 2453 | D25 | D9 | Ar15 | D9 | Ar15 |
| 2454 | D26 | D9 | Ar15 | D9 | Ar15 |
| 2455 | D21 | D10 | Ar15 | D10 | Ar15 |
| 2456 | D22 | D10 | Ar15 | D10 | Ar15 |
| 2457 | D23 | D10 | Ar15 | D10 | Ar15 |
| 2458 | D24 | D10 | Ar15 | D10 | Ar15 |
| 2459 | D25 | D10 | Ar15 | D10 | Ar15 |
| 2460 | D26 | D10 | Ar15 | D10 | Ar15 |
| 2461 | D21 | D11 | Ar15 | D11 | Ar15 |
| 2462 | D22 | D11 | Ar15 | D11 | Ar15 |
| 2463 | D23 | D11 | Ar15 | D11 | Ar15 |
| 2464 | D24 | D11 | Ar15 | D11 | Ar15 |
| 2465 | D25 | D11 | Ar15 | D11 | Ar15 |
| 2466 | D26 | D11 | Ar15 | D11 | Ar15 |
| 2467 | D21 | D12 | Ar15 | D12 | Ar15 |
| 2468 | D22 | D12 | Ar15 | D12 | Ar15 |
| 2469 | D23 | D12 | Ar15 | D12 | Ar15 |
| 2470 | D24 | D12 | Ar15 | D12 | Ar15 |
| 2471 | D25 | D12 | Ar15 | D12 | Ar15 |
| 2472 | D26 | D12 | Ar15 | D12 | Ar15 |
| 2473 | D21 | D1 | Ar16 | D1 | Ar16 |
| 2474 | D22 | D1 | Ar16 | D1 | Ar16 |
| 2475 | D23 | D1 | Ar16 | D1 | Ar16 |
| 2476 | D24 | D1 | Ar16 | D1 | Ar16 |
| 2477 | D25 | D1 | Ar16 | D1 | Ar16 |
| 2478 | D26 | D1 | Ar16 | D1 | Ar16 |
| 2479 | D21 | D2 | Ar16 | D2 | Ar16 |
| 2480 | D22 | D2 | Ar16 | D2 | Ar16 |
| 2481 | D23 | D2 | Ar16 | D2 | Ar16 |
| 2482 | D24 | D2 | Ar16 | D2 | Ar16 |
| 2483 | D25 | D2 | Ar16 | D2 | Ar16 |
| 2484 | D26 | D2 | Ar16 | D2 | Ar16 |
| 2485 | D21 | D3 | Ar16 | D3 | Ar16 |
| 2486 | D22 | D3 | Ar16 | D3 | Ar16 |
| 2487 | D23 | D3 | Ar16 | D3 | Ar16 |
| 2488 | D24 | D3 | Ar16 | D3 | Ar16 |
| 2489 | D25 | D3 | Ar16 | D3 | Ar16 |
| 2490 | D26 | D3 | Ar16 | D3 | Ar16 |
| 2491 | D21 | D8 | Ar16 | D8 | Ar16 |
| 2492 | D22 | D8 | Ar16 | D8 | Ar16 |
| 2493 | D23 | D8 | Ar16 | D8 | Ar16 |
| 2494 | D24 | D8 | Ar16 | D8 | Ar16 |
| 2495 | D25 | D8 | Ar16 | D8 | Ar16 |
| 2496 | D26 | D8 | Ar16 | D8 | Ar16 |
| 2497 | D21 | D9 | Ar16 | D9 | Ar16 |
| 2498 | D22 | D9 | Ar16 | D9 | Ar16 |
| 2499 | D23 | D9 | Ar16 | D9 | Ar16 |
| 2500 | D24 | D9 | Ar16 | D9 | Ar16 |
| 2501 | D25 | D9 | Ar16 | D9 | Ar16 |
| 2502 | D26 | D9 | Ar16 | D9 | Ar16 |
| 2503 | D21 | D10 | Ar16 | D10 | Ar16 |
| 2504 | D22 | D10 | Ar16 | D10 | Ar16 |
| 2505 | D23 | D10 | Ar16 | D10 | Ar16 |
| 2506 | D24 | D10 | Ar16 | D10 | Ar16 |
| 2507 | D25 | D10 | Ar16 | D10 | Ar16 |
| 2508 | D26 | D10 | Ar16 | D10 | Ar16 |
| 2509 | D21 | D11 | Ar16 | D11 | Ar16 |
| 2510 | D22 | D11 | Ar16 | D11 | Ar16 |
| 2511 | D23 | D11 | Ar16 | D11 | Ar16 |
| 2512 | D24 | D11 | Ar16 | D11 | Ar16 |
| 2513 | D25 | D11 | Ar16 | D11 | Ar16 |
| 2514 | D26 | D11 | Ar16 | D11 | Ar16 |
| 2515 | D21 | D12 | Ar16 | D12 | Ar16 |
| 2516 | D22 | D12 | Ar16 | D12 | Ar16 |
| 2517 | D23 | D12 | Ar16 | D12 | Ar16 |
| 2518 | D24 | D12 | Ar16 | D12 | Ar16 |
| 2519 | D25 | D12 | Ar16 | D12 | Ar16 |
| 2520 | D26 | D12 | Ar16 | D12 | Ar16 |
| 2521 | D21 | D1 | Ar17 | D1 | Ar17 |
| 2522 | D22 | D1 | Ar17 | D1 | Ar17 |
| 2523 | D23 | D1 | Ar17 | D1 | Ar17 |
| 2524 | D24 | D1 | Ar17 | D1 | Ar17 |
| 2525 | D25 | D1 | Ar17 | D1 | Ar17 |
| 2526 | D26 | D1 | Ar17 | D1 | Ar17 |
| 2527 | D21 | D2 | Ar17 | D2 | Ar17 |
| 2528 | D22 | D2 | Ar17 | D2 | Ar17 |
| 2529 | D23 | D2 | Ar17 | D2 | Ar17 |
| 2530 | D24 | D2 | Ar17 | D2 | Ar17 |
| 2531 | D25 | D2 | Ar17 | D2 | Ar17 |
| 2532 | D26 | D2 | Ar17 | D2 | Ar17 |
| 2533 | D21 | D3 | Ar17 | D3 | Ar17 |
| 2534 | D22 | D3 | Ar17 | D3 | Ar17 |
| 2535 | D23 | D3 | Ar17 | D3 | Ar17 |
| 2536 | D24 | D3 | Ar17 | D3 | Ar17 |
| 2537 | D25 | D3 | Ar17 | D3 | Ar17 |
| 2538 | D26 | D3 | Ar17 | D3 | Ar17 |
| 2539 | D21 | D8 | Ar17 | D8 | Ar17 |
| 2540 | D22 | D8 | Ar17 | D8 | Ar17 |
| 2541 | D23 | D8 | Ar17 | D8 | Ar17 |
| 2542 | D24 | D8 | Ar17 | D8 | Ar17 |
| 2543 | D25 | D8 | Ar17 | D8 | Ar17 |
| 2544 | D26 | D8 | Ar17 | D8 | Ar17 |
| 2545 | D21 | D9 | Ar17 | D9 | Ar17 |
| 2546 | D22 | D9 | Ar17 | D9 | Ar17 |
| 2547 | D23 | D9 | Ar17 | D9 | Ar17 |
| 2548 | D24 | D9 | Ar17 | D9 | Ar17 |
| 2549 | D25 | D9 | Ar17 | D9 | Ar17 |
| 2550 | D26 | D9 | Ar17 | D9 | Ar17 |
| 2551 | D21 | D10 | Ar17 | D10 | Ar17 |
| 2552 | D22 | D10 | Ar17 | D10 | Ar17 |
| 2553 | D23 | D10 | Ar17 | D10 | Ar17 |
| 2554 | D24 | D10 | Ar17 | D10 | Ar17 |
| 2555 | D25 | D10 | Ar17 | D10 | Ar17 |
| 2556 | D26 | D10 | Ar17 | D10 | Ar17 |
| 2557 | D21 | D11 | Ar17 | D11 | Ar17 |
| 2558 | D22 | D11 | Ar17 | D11 | Ar17 |
| 2559 | D23 | D11 | Ar17 | D11 | Ar17 |
| 2560 | D24 | D11 | Ar17 | D11 | Ar17 |
| 2561 | D25 | D11 | Ar17 | D11 | Ar17 |
| 2562 | D26 | D11 | Ar17 | D11 | Ar17 |
| 2563 | D21 | D12 | Ar17 | D12 | Ar17 |
| 2564 | D22 | D12 | Ar17 | D12 | Ar17 |
| 2565 | D23 | D12 | Ar17 | D12 | Ar17 |
| 2566 | D24 | D12 | Ar17 | D12 | Ar17 |
| 2567 | D25 | D12 | Ar17 | D12 | Ar17 |
| 2568 | D26 | D12 | Ar17 | D12 | Ar17 |
| 2569 | D1 | Ar2 | D1 | D21 | Ar2 |
| 2570 | D1 | Ar2 | D1 | D22 | Ar2 |
| 2571 | D1 | Ar2 | D1 | D23 | Ar2 |
| 2572 | D1 | Ar2 | D1 | D24 | Ar2 |
| 2573 | D1 | Ar2 | D1 | D25 | Ar2 |
| 2574 | D1 | Ar2 | D1 | D26 | Ar2 |
| 2575 | D2 | Ar2 | D2 | D21 | Ar2 |
| 2576 | D2 | Ar2 | D2 | D22 | Ar2 |
| 2577 | D2 | Ar2 | D2 | D23 | Ar2 |
| 2578 | D2 | Ar2 | D2 | D24 | Ar2 |
| 2579 | D2 | Ar2 | D2 | D25 | Ar2 |
| 2580 | D2 | Ar2 | D2 | D26 | Ar2 |
| 2581 | D3 | Ar2 | D3 | D21 | Ar2 |
| 2582 | D3 | Ar2 | D3 | D22 | Ar2 |
| 2583 | D3 | Ar2 | D3 | D23 | Ar2 |
| 2584 | D3 | Ar2 | D3 | D24 | Ar2 |
| 2585 | D3 | Ar2 | D3 | D25 | Ar2 |
| 2586 | D3 | Ar2 | D3 | D26 | Ar2 |
| 2587 | D8 | Ar2 | D8 | D21 | Ar2 |
| 2588 | D8 | Ar2 | D8 | D22 | Ar2 |
| 2589 | D8 | Ar2 | D8 | D23 | Ar2 |
| 2590 | D8 | Ar2 | D8 | D24 | Ar2 |
| 2591 | D8 | Ar2 | D8 | D25 | Ar2 |
| 2592 | D8 | Ar2 | D8 | D26 | Ar2 |
| 2593 | D9 | Ar2 | D9 | D21 | Ar2 |
| 2594 | D9 | Ar2 | D9 | D22 | Ar2 |
| 2595 | D9 | Ar2 | D9 | D23 | Ar2 |
| 2596 | D9 | Ar2 | D9 | D24 | Ar2 |
| 2597 | D9 | Ar2 | D9 | D25 | Ar2 |
| 2598 | D9 | Ar2 | D9 | D26 | Ar2 |
| 2599 | D10 | Ar2 | D10 | D21 | Ar2 |
| 2600 | D10 | Ar2 | D10 | D22 | Ar2 |
| 2601 | D10 | Ar2 | D10 | D23 | Ar2 |
| 2602 | D10 | Ar2 | D10 | D24 | Ar2 |
| 2603 | D10 | Ar2 | D10 | D25 | Ar2 |
| 2604 | D10 | Ar2 | D10 | D26 | Ar2 |
| 2605 | D11 | Ar2 | D11 | D21 | Ar2 |
| 2606 | D11 | Ar2 | D11 | D22 | Ar2 |
| 2607 | D11 | Ar2 | D11 | D23 | Ar2 |
| 2608 | D11 | Ar2 | D11 | D24 | Ar2 |
| 2609 | D11 | Ar2 | D11 | D25 | Ar2 |
| 2610 | D11 | Ar2 | D11 | D26 | Ar2 |
| 2611 | D12 | Ar2 | D12 | D21 | Ar2 |
| 2612 | D12 | Ar2 | D12 | D22 | Ar2 |
| 2613 | D12 | Ar2 | D12 | D23 | Ar2 |
| 2614 | D12 | Ar2 | D12 | D24 | Ar2 |
| 2615 | D12 | Ar2 | D12 | D25 | Ar2 |
| 2616 | D12 | Ar2 | D12 | D26 | Ar2 |
| 2617 | D1 | Ar3 | D1 | D21 | Ar3 |
| 2618 | D1 | Ar3 | D1 | D22 | Ar3 |
| 2619 | D1 | Ar3 | D1 | D23 | Ar3 |
| 2620 | D1 | Ar3 | D1 | D24 | Ar3 |
| 2621 | D1 | Ar3 | D1 | D25 | Ar3 |
| 2622 | D1 | Ar3 | D1 | D26 | Ar3 |
| 2623 | D2 | Ar3 | D2 | D21 | Ar3 |
| 2624 | D2 | Ar3 | D2 | D22 | Ar3 |
| 2625 | D2 | Ar3 | D2 | D23 | Ar3 |
| 2626 | D2 | Ar3 | D2 | D24 | Ar3 |
| 2627 | D2 | Ar3 | D2 | D25 | Ar3 |
| 2628 | D2 | Ar3 | D2 | D26 | Ar3 |
| 2629 | D3 | Ar3 | D3 | D21 | Ar3 |
| 2630 | D3 | Ar3 | D3 | D22 | Ar3 |
| 2631 | D3 | Ar3 | D3 | D23 | Ar3 |
| 2632 | D3 | Ar3 | D3 | D24 | Ar3 |
| 2633 | D3 | Ar3 | D3 | D25 | Ar3 |
| 2634 | D3 | Ar3 | D3 | D26 | Ar3 |
| 2635 | D8 | Ar3 | D8 | D21 | Ar3 |
| 2636 | D8 | Ar3 | D8 | D22 | Ar3 |
| 2637 | D8 | Ar3 | D8 | D23 | Ar3 |
| 2638 | D8 | Ar3 | D8 | D24 | Ar3 |
| 2639 | D8 | Ar3 | D8 | D25 | Ar3 |
| 2640 | D8 | Ar3 | D8 | D26 | Ar3 |
| 2641 | D9 | Ar3 | D9 | D21 | Ar3 |
| 2642 | D9 | Ar3 | D9 | D22 | Ar3 |
| 2643 | D9 | Ar3 | D9 | D23 | Ar3 |
| 2644 | D9 | Ar3 | D9 | D24 | Ar3 |
| 2645 | D9 | Ar3 | D9 | D25 | Ar3 |
| 2646 | D9 | Ar3 | D9 | D26 | Ar3 |
| 2647 | D10 | Ar3 | D10 | D21 | Ar3 |
| 2648 | D10 | Ar3 | D10 | D22 | Ar3 |
| 2649 | D10 | Ar3 | D10 | D23 | Ar3 |
| 2650 | D10 | Ar3 | D10 | D24 | Ar3 |
| 2651 | D10 | Ar3 | D10 | D25 | Ar3 |
| 2652 | D10 | Ar3 | D10 | D26 | Ar3 |
| 2653 | D11 | Ar3 | D11 | D21 | Ar3 |
| 2654 | D11 | Ar3 | D11 | D22 | Ar3 |
| 2855 | D11 | Ar3 | D11 | D23 | Ar3 |
| 2656 | D11 | Ar3 | D11 | D24 | Ar3 |
| 2657 | D11 | Ar3 | D11 | D25 | Ar3 |
| 2658 | D11 | Ar3 | D11 | D26 | Ar3 |
| 2659 | D12 | Ar3 | D12 | D21 | Ar3 |
| 2660 | D12 | Ar3 | D12 | D22 | Ar3 |
| 2661 | D12 | Ar3 | D12 | D23 | Ar3 |
| 2662 | D12 | Ar3 | D12 | D24 | Ar3 |
| 2663 | D12 | Ar3 | D12 | D25 | Ar3 |
| 2664 | D12 | Ar3 | D12 | D26 | Ar3 |
| 2665 | D1 | Ar4 | D1 | D21 | Ar4 |
| 2666 | D1 | Ar4 | D1 | D22 | Ar4 |
| 2667 | D1 | Ar4 | D1 | D23 | Ar4 |
| 2668 | D1 | Ar4 | D1 | D24 | Ar4 |
| 2669 | D1 | Ar4 | D1 | D25 | Ar4 |
| 2670 | D1 | Ar4 | D1 | D26 | Ar4 |
| 2671 | D2 | Ar4 | D2 | D21 | Ar4 |
| 2672 | D2 | Ar4 | D2 | D22 | Ar4 |
| 2673 | D2 | Ar4 | D2 | D23 | Ar4 |
| 2674 | D2 | Ar4 | D2 | D24 | Ar4 |
| 2675 | D2 | Ar4 | D2 | D25 | Ar4 |
| 2676 | D2 | Ar4 | D2 | D26 | Ar4 |
| 2677 | D3 | Ar4 | D3 | D21 | Ar4 |
| 2678 | D3 | Ar4 | D3 | D22 | Ar4 |
| 2679 | D3 | Ar4 | D3 | D23 | Ar4 |
| 2680 | D3 | Ar4 | D3 | D24 | Ar4 |
| 2681 | D3 | Ar4 | D3 | D25 | Ar4 |
| 2682 | D3 | Ar4 | D3 | D26 | Ar4 |
| 2683 | D8 | Ar4 | D8 | D21 | Ar4 |
| 2684 | D8 | Ar4 | D8 | D22 | Ar4 |
| 2685 | D8 | Ar4 | D8 | D23 | Ar4 |
| 2686 | D8 | Ar4 | D8 | D24 | Ar4 |
| 2687 | D8 | Ar4 | D8 | D25 | Ar4 |
| 2688 | D8 | Ar4 | D8 | D26 | Ar4 |
| 2689 | D9 | Ar4 | D9 | D21 | Ar4 |
| 2690 | D9 | Ar4 | D9 | D22 | Ar4 |
| 2691 | D9 | Ar4 | D9 | D23 | Ar4 |
| 2692 | D9 | Ar4 | D9 | D24 | Ar4 |
| 2693 | D9 | Ar4 | D9 | D25 | Ar4 |
| 2694 | D9 | Ar4 | D9 | D26 | Ar4 |
| 2695 | D10 | Ar4 | D10 | D21 | Ar4 |
| 2696 | D10 | Ar4 | D10 | D22 | Ar4 |
| 2697 | D10 | Ar4 | D10 | D23 | Ar4 |
| 2698 | D10 | Ar4 | D10 | D24 | Ar4 |
| 2699 | D10 | Ar4 | D10 | D25 | Ar4 |
| 2700 | D10 | Ar4 | D10 | D26 | Ar4 |
| 2701 | D11 | Ar4 | D11 | D21 | Ar4 |
| 2702 | D11 | Ar4 | D11 | D22 | Ar4 |
| 2703 | D11 | Ar4 | D11 | D23 | Ar4 |
| 2704 | D11 | Ar4 | D11 | D24 | Ar4 |
| 2705 | D11 | Ar4 | D11 | D25 | Ar4 |
| 2706 | D11 | Ar4 | D11 | D26 | Ar4 |
| 2707 | D12 | Ar4 | D12 | D21 | Ar4 |
| 2708 | D12 | Ar4 | D12 | D22 | Ar4 |
| 2709 | D12 | Ar4 | D12 | D23 | Ar4 |
| 2710 | D12 | Ar4 | D12 | D24 | Ar4 |
| 2711 | D12 | Ar4 | D12 | D25 | Ar4 |
| 2712 | D12 | Ar4 | D12 | D26 | Ar4 |
| 2713 | D1 | Ar5 | D1 | D21 | Ar5 |
| 2714 | D1 | Ar5 | D1 | D22 | Ar5 |
| 2715 | D1 | Ar5 | D1 | D23 | Ar5 |
| 2716 | D1 | Ar5 | D1 | D24 | Ar5 |
| 2717 | D1 | Ar5 | D1 | D25 | Ar5 |
| 2718 | D1 | Ar5 | D1 | D26 | Ar5 |
| 2719 | D2 | Ar5 | D2 | D21 | Ar5 |
| 2720 | D2 | Ar5 | D2 | D22 | Ar5 |
| 2721 | D2 | Ar5 | D2 | D23 | Ar5 |
| 2722 | D2 | Ar5 | D2 | D24 | Ar5 |
| 2723 | D2 | Ar5 | D2 | D25 | Ar5 |
| 2724 | D2 | Ar5 | D2 | D26 | Ar5 |
| 2725 | D3 | Ar5 | D3 | D21 | Ar5 |
| 2726 | D3 | Ar5 | D3 | D22 | Ar5 |
| 2727 | D3 | Ar5 | D3 | D23 | Ar5 |
| 2728 | D3 | Ar5 | D3 | D24 | Ar5 |
| 2729 | D3 | Ar5 | D3 | D25 | Ar5 |
| 2730 | D3 | Ar5 | D3 | D26 | Ar5 |
| 2731 | D8 | Ar5 | D8 | D21 | Ar5 |
| 2732 | D8 | Ar5 | D8 | D22 | Ar5 |
| 2733 | D8 | Ar5 | D8 | D23 | Ar5 |
| 2734 | D8 | Ar5 | D8 | D24 | Ar5 |
| 2735 | D8 | Ar5 | D8 | D25 | Ar5 |
| 2736 | D8 | Ar5 | D8 | D26 | Ar5 |
| 2737 | D9 | Ar5 | D9 | D21 | Ar5 |
| 2738 | D9 | Ar5 | D9 | D22 | Ar5 |
| 2739 | D9 | Ar5 | D9 | D23 | Ar5 |
| 2740 | D9 | Ar5 | D9 | D24 | Ar5 |
| 2741 | D9 | Ar5 | D9 | D25 | Ar5 |
| 2742 | D9 | Ar5 | D9 | D26 | Ar5 |
| 2743 | D10 | Ar5 | D10 | D21 | Ar5 |
| 2744 | D10 | Ar5 | D10 | D22 | Ar5 |
| 2745 | D10 | Ar5 | D10 | D23 | Ar5 |
| 2746 | D10 | Ar5 | D10 | D24 | Ar5 |
| 2747 | D10 | Ar5 | D10 | D25 | Ar5 |
| 2748 | D10 | Ar5 | D10 | D26 | Ar5 |
| 2749 | D11 | Ar5 | D11 | D21 | Ar5 |
| 2750 | D11 | Ar5 | D11 | D22 | Ar5 |
| 2751 | D11 | Ar5 | D11 | D23 | Ar5 |
| 2752 | D11 | Ar5 | D11 | D24 | Ar5 |
| 2753 | D11 | Ar5 | D11 | D25 | Ar5 |
| 2754 | D11 | Ar5 | D11 | D26 | Ar5 |
| 2755 | D12 | Ar5 | D12 | D21 | Ar5 |
| 2756 | D12 | Ar5 | D12 | D22 | Ar5 |
| 2757 | D12 | Ar5 | D12 | D23 | Ar5 |
| 2758 | D12 | Ar5 | D12 | D24 | Ar5 |
| 2759 | D12 | Ar5 | D12 | D25 | Ar5 |
| 2760 | D12 | Ar5 | D12 | D26 | Ar5 |
| 2761 | D1 | Ar6 | D1 | D21 | Ar6 |
| 2762 | D1 | Ar6 | D1 | D22 | Ar6 |
| 2763 | D1 | Ar6 | D1 | D23 | Ar6 |
| 2764 | D1 | Ar6 | D1 | D24 | Ar6 |
| 2765 | D1 | Ar6 | D1 | D25 | Ar6 |
| 2766 | D1 | Ar6 | D1 | D26 | Ar6 |
| 2767 | D2 | Ar6 | D2 | D21 | Ar6 |
| 2768 | D2 | Ar6 | D2 | D22 | Ar6 |
| 2769 | D2 | Ar6 | D2 | D23 | Ar6 |
| 2770 | D2 | Ar6 | D2 | D24 | Ar6 |
| 2771 | D2 | Ar6 | D2 | D25 | Ar6 |
| 2772 | D2 | Ar6 | D2 | D26 | Ar6 |
| 2773 | D3 | Ar6 | D3 | D21 | Ar6 |
| 2774 | D3 | Ar6 | D3 | D22 | Ar6 |
| 2775 | D3 | Ar6 | D3 | D23 | Ar6 |
| 2776 | D3 | Ar6 | D3 | D24 | Ar6 |
| 2777 | D3 | Ar6 | D3 | D25 | Ar6 |
| 2778 | D3 | Ar6 | D3 | D26 | Ar6 |
| 2779 | D8 | Ar6 | D8 | D21 | Ar6 |
| 2780 | D8 | Ar6 | D8 | D22 | Ar6 |
| 2781 | D8 | Ar6 | D8 | D23 | Ar6 |
| 2782 | D8 | Ar6 | D8 | D24 | Ar6 |
| 2783 | D8 | Ar6 | D8 | D25 | Ar6 |
| 2784 | D8 | Ar6 | D8 | D26 | Ar6 |
| 2785 | D9 | Ar6 | D9 | D21 | Ar6 |
| 2786 | D9 | Ar6 | D9 | D22 | Ar6 |
| 2787 | D9 | Ar6 | D9 | D23 | Ar6 |
| 2788 | D9 | Ar6 | D9 | D24 | Ar6 |
| 2788 | D9 | Ar6 | D9 | D25 | Ar6 |
| 2790 | D9 | Ar6 | D9 | D26 | Ar6 |
| 2791 | D10 | Ar6 | D10 | D21 | Ar6 |
| 2792 | D10 | Ar6 | D10 | D22 | Ar6 |
| 2793 | D10 | Ar6 | D10 | D23 | Ar6 |
| 2794 | D10 | Ar6 | D10 | D24 | Ar6 |
| 2795 | D10 | Ar6 | D10 | D25 | Ar6 |
| 2796 | D10 | Ar6 | D10 | D26 | Ar6 |
| 2797 | D11 | Ar6 | D11 | D21 | Ar6 |
| 2798 | D11 | Ar6 | D11 | D22 | Ar6 |
| 2799 | D11 | Ar6 | D11 | D23 | Ar6 |
| 2800 | D11 | Ar6 | D11 | D24 | Ar6 |
| 2801 | D11 | Ar6 | D11 | D25 | Ar6 |
| 2802 | D11 | Ar6 | D11 | D26 | Ar6 |
| 2803 | D12 | Ar6 | D12 | D21 | Ar6 |
| 2804 | D12 | Ar6 | D12 | D22 | Ar6 |
| 2805 | D12 | Ar6 | D12 | D23 | Ar6 |
| 2806 | D12 | Ar6 | D12 | D24 | Ar6 |
| 2807 | D12 | Ar6 | D12 | D25 | Ar6 |
| 2808 | D12 | Ar6 | D12 | D26 | Ar6 |
| 2809 | D1 | Ar7 | D1 | D21 | Ar7 |
| 2810 | D1 | Ar7 | D1 | D22 | Ar7 |
| 2811 | D1 | Ar7 | D1 | D23 | Ar7 |
| 2812 | D1 | Ar7 | D1 | D24 | Ar7 |
| 2813 | D1 | Ar7 | D1 | D25 | Ar7 |
| 2814 | D1 | Ar7 | D1 | D26 | Ar7 |
| 2815 | D2 | Ar7 | D2 | D21 | Ar7 |
| 2816 | D2 | Ar7 | D2 | D22 | Ar7 |
| 2817 | D2 | Ar7 | D2 | D23 | Ar7 |
| 2818 | D2 | Ar7 | D2 | D24 | Ar7 |
| 2819 | D2 | Ar7 | D2 | D25 | Ar7 |
| 2820 | D2 | Ar7 | D2 | D26 | Ar7 |
| 2821 | D3 | Ar7 | D3 | D21 | Ar7 |
| 2822 | D3 | Ar7 | D3 | D22 | Ar7 |
| 2823 | D3 | Ar7 | D3 | D23 | Ar7 |
| 2824 | D3 | Ar7 | D3 | D24 | Ar7 |
| 2825 | D3 | Ar7 | D3 | D25 | Ar7 |
| 2826 | D3 | Ar7 | D3 | D26 | Ar7 |
| 2827 | D8 | Ar7 | D8 | D21 | Ar7 |
| 2828 | D8 | Ar7 | D8 | D22 | Ar7 |
| 2829 | D8 | Ar7 | D8 | D23 | Ar7 |
| 2830 | D8 | Ar7 | D8 | D24 | Ar7 |
| 2831 | D8 | Ar7 | D8 | D25 | Ar7 |
| 2832 | D8 | Ar7 | D8 | D26 | Ar7 |
| 2833 | D9 | Ar7 | D9 | D21 | Ar7 |
| 2834 | D9 | Ar7 | D9 | D22 | Ar7 |
| 2835 | D9 | Ar7 | D9 | D23 | Ar7 |
| 2836 | D9 | Ar7 | D9 | D24 | Ar7 |
| 2837 | D9 | Ar7 | D9 | D25 | Ar7 |
| 2838 | D9 | Ar7 | D9 | D26 | Ar7 |
| 2839 | D10 | Ar7 | D10 | D21 | Ar7 |
| 2840 | D10 | Ar7 | D10 | D22 | Ar7 |
| 2841 | D10 | Ar7 | D10 | D23 | Ar7 |
| 2842 | D10 | Ar7 | D10 | D24 | Ar7 |
| 2843 | D10 | Ar7 | D10 | D25 | Ar7 |
| 2844 | D10 | Ar7 | D10 | D26 | Ar7 |
| 2845 | D11 | Ar7 | D11 | D21 | Ar7 |
| 2846 | D11 | Ar7 | D11 | D22 | Ar7 |
| 2847 | D11 | Ar7 | D11 | D23 | Ar7 |
| 2848 | D11 | Ar7 | D11 | D24 | Ar7 |
| 2849 | D11 | Ar7 | D11 | D25 | Ar7 |
| 2850 | D11 | Ar7 | D11 | D26 | Ar7 |
| 2851 | D12 | Ar7 | D12 | D21 | Ar7 |
| 2852 | D12 | Ar7 | D12 | D22 | Ar7 |
| 2853 | D12 | Ar7 | D12 | D23 | Ar7 |
| 2854 | D12 | Ar7 | D12 | D24 | Ar7 |
| 2855 | D12 | Ar7 | D12 | D25 | Ar7 |
| 2856 | D12 | Ar7 | D12 | D26 | Ar7 |
| 2857 | D1 | Ar8 | D1 | D21 | Ar8 |
| 2858 | D1 | Ar8 | D1 | D22 | Ar8 |
| 2859 | D1 | Ar8 | D1 | D23 | Ar8 |
| 2860 | D1 | Ar8 | D1 | D24 | Ar8 |
| 2861 | D1 | Ar8 | D1 | D25 | Ar8 |
| 2862 | D1 | Ar8 | D1 | D26 | Ar8 |
| 2863 | D2 | Ar8 | D1 | D21 | Ar8 |
| 2864 | D2 | Ar8 | D2 | D22 | Ar8 |
| 2865 | D2 | Ar8 | D2 | D23 | Ar8 |
| 2866 | D2 | Ar8 | D2 | D24 | Ar8 |
| 2867 | D2 | Ar8 | D2 | D25 | Ar8 |
| 2868 | D2 | Ar8 | D2 | D26 | Ar8 |
| 2869 | D3 | Ar8 | D3 | D21 | Ar8 |
| 2870 | D3 | Ar8 | D3 | D22 | Ar8 |
| 2871 | D3 | Ar8 | D3 | D23 | Ar8 |
| 2872 | D3 | Ar8 | D3 | D24 | Ar8 |
| 2873 | D3 | Ar8 | D3 | D25 | Ar8 |
| 2874 | D3 | Ar8 | D3 | D26 | Ar8 |
| 2875 | D8 | Ar8 | D8 | D21 | Ar8 |
| 2876 | D8 | Ar8 | D8 | D22 | Ar8 |
| 2877 | D8 | Ar8 | D8 | D23 | Ar8 |
| 2878 | D8 | Ar8 | D8 | D24 | Ar8 |
| 2879 | D8 | Ar8 | D8 | D25 | Ar8 |
| 2880 | D8 | Ar8 | D8 | D26 | Ar8 |
| 2881 | D9 | Ar8 | D9 | D21 | Ar8 |
| 2882 | D9 | Ar8 | D9 | D22 | Ar8 |
| 2883 | D9 | Ar8 | D9 | D23 | Ar8 |
| 2884 | D9 | Ar8 | D9 | D24 | Ar8 |
| 2885 | D9 | Ar8 | D9 | D25 | Ar8 |
| 2886 | D9 | Ar8 | D9 | D26 | Ar8 |
| 2887 | D10 | Ar8 | D10 | D21 | Ar8 |
| 2888 | D10 | Ar8 | D10 | D22 | Ar8 |
| 2889 | D10 | Ar8 | D10 | D23 | Ar8 |
| 2890 | D10 | Ar8 | D10 | D24 | Ar8 |
| 2891 | D10 | Ar8 | D10 | D25 | Ar8 |
| 2892 | D10 | Ar8 | D10 | D26 | Ar8 |
| 2893 | D11 | Ar8 | D11 | D21 | Ar8 |
| 2894 | D11 | Ar8 | D11 | D22 | Ar8 |
| 2895 | D11 | Ar8 | D11 | D23 | Ar8 |
| 2896 | D11 | Ar8 | D11 | D24 | Ar8 |
| 2897 | D11 | Ar8 | D11 | D25 | Ar8 |
| 2898 | D11 | Ar8 | D11 | D26 | Ar8 |
| 2899 | D12 | Ar8 | D12 | D21 | Ar8 |
| 2900 | D12 | Ar8 | D12 | D22 | Ar8 |
| 2901 | D12 | Ar8 | D12 | D23 | Ar8 |
| 2902 | D12 | Ar8 | D12 | D24 | Ar8 |
| 2903 | D12 | Ar8 | D12 | D25 | Ar8 |
| 2904 | D12 | Ar8 | D12 | D26 | Ar8 |
| 2905 | D1 | Ar9 | D1 | D21 | Ar9 |
| 2906 | D1 | Ar9 | D1 | D22 | Ar9 |
| 2907 | D1 | Ar9 | D1 | D23 | Ar9 |
| 2908 | D1 | Ar9 | D1 | D24 | Ar9 |
| 2909 | D1 | Ar9 | D1 | D25 | Ar9 |
| 2910 | D1 | Ar9 | D1 | D26 | Ar9 |
| 2911 | D2 | Ar9 | D2 | D21 | Ar9 |
| 2912 | D2 | Ar9 | D2 | D22 | Ar9 |
| 2913 | D2 | Ar9 | D2 | D23 | Ar9 |
| 2914 | D2 | Ar9 | D2 | D24 | Ar9 |
| 2915 | D2 | Ar9 | D2 | D25 | Ar9 |
| 2916 | D2 | Ar9 | D2 | D26 | Ar9 |
| 2917 | D3 | Ar9 | D3 | D21 | Ar9 |
| 2918 | D3 | Ar9 | D3 | D22 | Ar9 |
| 2919 | D3 | Ar9 | D3 | D23 | Ar9 |
| 2920 | D3 | Ar9 | D3 | D24 | Ar9 |
| 2921 | D3 | Ar9 | D3 | D25 | Ar9 |
| 2922 | D3 | Ar9 | D3 | D26 | Ar9 |
| 2923 | D8 | Ar9 | D8 | D21 | Ar9 |
| 2924 | D8 | Ar9 | D8 | D22 | Ar9 |
| 2825 | D8 | Ar9 | D8 | D23 | Ar9 |
| 2926 | D8 | Ar9 | D8 | D24 | Ar9 |
| 2927 | D8 | Ar9 | D8 | D25 | Ar9 |
| 2928 | D8 | Ar9 | D8 | D26 | Ar9 |
| 2929 | D9 | Ar9 | D9 | D21 | Ar9 |
| 2930 | D9 | Ar9 | D9 | D22 | Ar9 |
| 2931 | D9 | Ar9 | D9 | D23 | Ar9 |
| 2932 | D9 | Ar9 | D9 | D24 | Ar9 |
| 2933 | D9 | Ar9 | D9 | D25 | Ar9 |
| 2934 | D9 | Ar9 | D9 | D26 | Ar9 |
| 2935 | D10 | Ar9 | D10 | D21 | Ar9 |
| 2936 | D10 | Ar9 | D10 | D22 | Ar9 |
| 2937 | D10 | Ar9 | D10 | D23 | Ar9 |
| 2938 | D10 | Ar9 | D10 | D24 | Ar9 |
| 2939 | D10 | Ar9 | D10 | D25 | Ar9 |
| 2940 | D10 | Ar9 | D10 | D26 | Ar9 |
| 2941 | D11 | Ar9 | D11 | D21 | Ar9 |
| 2942 | D11 | Ar9 | D11 | D22 | Ar9 |
| 2943 | D11 | Ar9 | D11 | D23 | Ar9 |
| 2944 | D11 | Ar9 | D11 | D24 | Ar9 |
| 2945 | D11 | Ar9 | D11 | D25 | Ar9 |
| 2346 | D11 | Ar9 | D11 | D26 | Ar9 |
| 2947 | D12 | Ar9 | D12 | D21 | Ar9 |
| 2948 | D12 | Ar9 | D12 | D22 | Ar9 |
| 2949 | D12 | Ar9 | D12 | D23 | Ar9 |
| 2950 | D12 | Ar9 | D12 | D24 | Ar9 |
| 2951 | D12 | Ar9 | D12 | D25 | Ar9 |
| 2952 | D12 | Ar9 | D12 | D26 | Ar9 |
| 2953 | D1 | Ar10 | D1 | D21 | Ar10 |
| 2954 | D1 | Ar10 | D1 | D22 | Ar10 |
| 2955 | D1 | Ar10 | D1 | D23 | Ar10 |
| 2956 | D1 | Ar10 | D1 | D24 | Ar10 |
| 2957 | D1 | Ar10 | D1 | D25 | Ar10 |
| 2958 | D1 | Ar10 | D1 | D26 | Ar10 |
| 2959 | D2 | Ar10 | D2 | D21 | Ar10 |
| 2980 | D2 | Ar10 | D2 | D22 | Ar10 |
| 2961 | D2 | Ar10 | D2 | D23 | Ar10 |
| 2962 | D2 | Ar10 | D2 | D24 | Ar10 |
| 2963 | D2 | Ar10 | D2 | D25 | Ar10 |
| 2964 | D2 | Ar10 | D2 | D26 | Ar10 |
| 2965 | D3 | Ar10 | D3 | D21 | Ar10 |
| 2966 | D3 | Ar10 | D3 | D22 | Ar10 |
| 2967 | D3 | Ar10 | D3 | D23 | Ar10 |
| 2968 | D3 | Ar10 | D3 | D24 | Ar10 |
| 2969 | D3 | Ar10 | D3 | D25 | Ar10 |
| 2970 | D3 | Ar10 | D3 | D26 | Ar10 |
| 2971 | D8 | Ar10 | D8 | D21 | Ar10 |
| 2972 | D8 | Ar10 | D8 | D22 | Ar10 |
| 2973 | D8 | Ar10 | D8 | D23 | Ar10 |
| 2974 | D8 | Ar10 | D8 | D24 | Ar10 |
| 2975 | D8 | Ar10 | D8 | D25 | Ar10 |
| 2976 | D8 | Ar10 | D8 | D26 | Ar10 |
| 2977 | D9 | Ar10 | D9 | D21 | Ar10 |
| 2978 | D9 | Ar10 | D9 | D22 | Ar10 |
| 2979 | D9 | Ar10 | D9 | D23 | Ar10 |
| 2980 | D9 | Ar10 | D9 | D24 | Ar10 |
| 2981 | D9 | Ar10 | D9 | D25 | Ar10 |
| 2982 | D9 | Ar10 | D9 | D26 | Ar10 |
| 2983 | D10 | Ar10 | D10 | D21 | Ar10 |
| 2984 | D10 | Ar10 | D10 | D22 | Ar10 |
| 2985 | D10 | Ar10 | D10 | D23 | Ar10 |
| 2986 | D10 | Ar10 | D10 | D24 | Ar10 |
| 2987 | D10 | Ar10 | D10 | D25 | Ar10 |
| 2988 | D10 | Ar10 | D10 | D26 | Ar10 |
| 2989 | D11 | Ar10 | D11 | D21 | Ar10 |
| 2990 | D11 | Ar10 | D11 | D22 | Ar10 |
| 2991 | D11 | Ar10 | D11 | D23 | Ar10 |
| 2992 | D11 | Ar10 | D11 | D24 | Ar10 |
| 2993 | D11 | Ar10 | D11 | D25 | Ar10 |
| 2994 | D11 | Ar10 | D11 | D26 | Ar10 |
| 2995 | D12 | Ar10 | 012 | D21 | Ar10 |
| 2996 | D12 | Ar10 | D12 | D22 | Ar10 |
| 2997 | D12 | Ar10 | D12 | D23 | Ar10 |
| 2998 | D12 | Ar10 | D12 | D24 | Ar10 |
| 2999 | D12 | Ar10 | D12 | D25 | Ar10 |
| 3000 | D12 | Ar10 | D12 | D26 | Ar10 |
| 3001 | D1 | Ar11 | D1 | D21 | Ar11 |
| 3002 | D1 | Ar11 | D1 | D22 | Ar11 |
| 3003 | D1 | Ar11 | D1 | D23 | Ar11 |
| 3004 | D1 | Ar11 | D1 | D24 | Ar11 |
| 3005 | D1 | Ar11 | D1 | D25 | Ar11 |
| 3006 | D1 | Ar11 | D1 | D26 | Ar11 |
| 3007 | D2 | Ar11 | D2 | D21 | Ar11 |
| 3008 | D2 | Ar11 | D2 | D22 | Ar11 |
| 3009 | D2 | Ar11 | D2 | D23 | Ar11 |
| 3010 | D2 | Ar11 | D2 | D24 | Ar11 |
| 3011 | D2 | Ar11 | D2 | D25 | Ar11 |
| 3012 | D2 | Ar11 | D2 | D26 | Ar11 |
| 3013 | D3 | Ar11 | D3 | D21 | Ar11 |
| 3014 | D3 | Ar11 | D3 | D22 | Ar11 |
| 3015 | D3 | Ar11 | D3 | D23 | Ar11 |
| 3016 | D3 | Ar11 | D3 | D24 | Ar11 |
| 3017 | D3 | Ar11 | D3 | D25 | Ar11 |
| 3018 | D3 | Ar11 | D3 | D26 | Ar11 |
| 3019 | D8 | Ar11 | D8 | D21 | Ar11 |
| 3020 | D8 | Ar11 | D8 | D22 | Ar11 |
| 3021 | D8 | Ar11 | D8 | D23 | Ar11 |
| 3022 | D8 | Ar11 | D8 | D24 | Ar11 |
| 3023 | D8 | Ar11 | D8 | D25 | Ar11 |
| 3024 | D8 | Ar11 | D8 | D26 | Ar11 |
| 3025 | D9 | Ar11 | D9 | D21 | Ar11 |
| 3026 | D9 | Ar11 | D9 | D22 | Ar11 |
| 3027 | D9 | Ar11 | D9 | D23 | Ar11 |
| 3028 | D9 | Ar11 | D9 | D24 | Ar11 |
| 3029 | D9 | Ar11 | D9 | D25 | Ar11 |
| 3030 | D9 | Ar11 | D9 | D26 | Ar11 |
| 3031 | D10 | Ar11 | D10 | D21 | Ar11 |
| 3032 | D10 | Ar11 | D10 | D22 | Ar11 |
| 3033 | D10 | Ar11 | D10 | D23 | Ar11 |
| 3034 | D10 | Ar11 | D10 | D24 | Ar11 |
| 3035 | D10 | Ar11 | D10 | D25 | Ar11 |
| 3036 | D10 | Ar11 | D10 | D26 | Ar11 |
| 3037 | D11 | Ar11 | D11 | D21 | Ar11 |
| 3038 | D11 | Ar11 | D11 | D22 | Ar11 |
| 3039 | D11 | Ar11 | D11 | D23 | Ar11 |
| 3040 | D11 | Ar11 | D11 | D24 | Ar11 |
| 3041 | D11 | Ar11 | D11 | D25 | Ar11 |
| 3042 | D11 | Ar11 | D11 | D26 | Ar11 |
| 3043 | D12 | Ar11 | D12 | D21 | Ar11 |
| 3044 | D12 | Ar11 | D12 | D22 | Ar11 |
| 3045 | D12 | Ar11 | D12 | D23 | Ar11 |
| 3046 | D12 | Ar11 | D12 | D24 | Ar11 |
| 3047 | D12 | Ar11 | D12 | D25 | Ar11 |
| 3048 | D12 | Ar11 | D12 | D26 | Ar11 |
| 3049 | D1 | Ar12 | D1 | D21 | Ar12 |
| 3050 | D1 | Ar12 | D1 | D22 | Ar12 |
| 3051 | D1 | Ar12 | D1 | D23 | Ar12 |
| 3052 | D1 | Ar12 | D1 | D24 | Ar12 |
| 3053 | D1 | Ar12 | D1 | D25 | Ar12 |
| 3054 | D1 | Ar12 | D1 | D26 | Ar12 |
| 3055 | D2 | Ar12 | D2 | D21 | Ar12 |
| 3056 | D2 | Ar12 | D2 | D22 | Ar12 |
| 3057 | D2 | Ar12 | D2 | D23 | Ar12 |
| 3058 | D2 | Ar12 | D2 | D24 | Ar12 |
| 3059 | D2 | Ar12 | D2 | D25 | Ar12 |
| 3060 | D2 | Ar12 | D2 | D26 | Ar12 |
| 3061 | D3 | Ar12 | D3 | D21 | Ar12 |
| 3062 | D3 | Ar12 | D3 | D22 | Ar12 |
| 3063 | D3 | Ar12 | D3 | D23 | Ar12 |
| 3064 | D3 | Ar12 | D3 | D24 | Ar12 |
| 3065 | D3 | Ar12 | D3 | D25 | Ar12 |
| 3066 | D3 | Ar12 | D3 | D26 | Ar12 |
| 3067 | D8 | Ar12 | D8 | D21 | Ar12 |
| 3068 | D8 | Ar12 | D8 | D22 | Ar12 |
| 3069 | D8 | Ar12 | D8 | D23 | Ar12 |
| 3070 | D8 | Ar12 | D8 | D24 | Ar12 |
| 3071 | D8 | Ar12 | D8 | D25 | Ar12 |
| 3072 | D8 | Ar12 | D8 | D26 | Ar12 |
| 3073 | D9 | Ar12 | D9 | D21 | Ar12 |
| 3074 | D9 | Ar12 | D9 | D22 | Ar12 |
| 3075 | D9 | Ar12 | D9 | D23 | Ar12 |
| 3076 | D9 | Ar12 | D9 | D24 | Ar12 |
| 3077 | D9 | Ar12 | D9 | D25 | Ar12 |
| 3078 | D9 | Ar12 | D9 | D26 | Ar12 |
| 3079 | D10 | Ar12 | D10 | D21 | Ar12 |
| 3080 | D10 | Ar12 | D10 | D22 | Ar12 |
| 3081 | D10 | Ar12 | D10 | D23 | Ar12 |
| 3082 | D10 | Ar12 | D10 | D24 | Ar12 |
| 3083 | D10 | Ar12 | D10 | D25 | Ar12 |
| 3084 | D10 | Ar12 | D10 | D26 | Ar12 |
| 3085 | D11 | Ar12 | D11 | D21 | Ar12 |
| 3086 | D11 | Ar12 | D11 | D22 | Ar12 |
| 3087 | D11 | Ar12 | D11 | D23 | Ar12 |
| 3088 | D11 | Ar12 | D11 | D24 | Ar12 |
| 3089 | D11 | Ar12 | D11 | D25 | Ar12 |
| 3090 | D11 | Ar12 | D11 | D26 | Ar12 |
| 3091 | D12 | Ar12 | D12 | D21 | Ar12 |
| 3092 | D12 | Ar12 | D12 | D22 | Ar12 |
| 3093 | D12 | Ar12 | D12 | D23 | Ar12 |
| 3094 | D12 | Ar12 | D12 | D24 | Ar12 |
| 3095 | D12 | Ar12 | D12 | D25 | Ar12 |
| 3096 | D12 | Ar12 | D12 | D26 | Ar12 |
| 3097 | D1 | Ar13 | D1 | D21 | Ar13 |
| 3098 | D1 | Ar13 | D1 | D22 | Ar13 |
| 3099 | D1 | Ar13 | D1 | D23 | Ar13 |
| 3100 | D1 | Ar13 | D1 | D24 | Ar13 |
| 3101 | D1 | Ar13 | D1 | D25 | Ar13 |
| 3102 | D1 | Ar13 | D1 | D26 | Ar13 |
| 3103 | D2 | Ar13 | D2 | D21 | Ar13 |
| 3104 | D2 | Ar13 | D2 | D22 | Ar13 |
| 3105 | D2 | Ar13 | D2 | D23 | Ar13 |
| 3106 | D2 | Ar13 | D2 | D24 | Ar13 |
| 3107 | D2 | Ar13 | D2 | D25 | Ar13 |
| 3108 | D2 | Ar13 | D2 | D26 | Ar13 |
| 3109 | D3 | Ar13 | D3 | D21 | Ar13 |
| 3110 | D3 | Ar13 | D3 | D22 | Ar13 |
| 3111 | D3 | Ar13 | D3 | D23 | Ar13 |
| 3112 | D3 | Ar13 | D3 | D24 | Ar13 |
| 3113 | D3 | Ar13 | D3 | D25 | Ar13 |
| 3114 | D3 | Ar13 | D3 | D26 | Ar13 |
| 3115 | D8 | Ar13 | D8 | D21 | Ar13 |
| 3116 | D8 | Ar13 | D8 | D22 | Ar13 |
| 3117 | D8 | Ar13 | D8 | D23 | Ar1a |
| 3118 | D8 | Ar13 | D8 | D24 | Ar13 |
| 3119 | D8 | Ar13 | D8 | D25 | Ar13 |
| 3120 | D8 | Ar13 | D8 | D26 | Ar13 |
| 3121 | D9 | Ar13 | D9 | D21 | Ar13 |
| 3122 | D9 | Ar13 | D9 | D22 | Ar13 |
| 3123 | D9 | Ar13 | D9 | D23 | Ar13 |
| 3124 | D9 | Ar13 | D9 | D24 | Ar13 |
| 3125 | D9 | Ar13 | D9 | D25 | Ar13 |
| 3126 | D9 | Ar13 | D9 | D26 | Ar13 |
| 3127 | D10 | Ar13 | D10 | D21 | Ar13 |
| 3128 | D10 | Ar13 | D10 | D22 | Ar13 |
| 3129 | D10 | Ar13 | D10 | D23 | Ar13 |
| 3130 | D10 | Ar13 | D10 | D24 | Ar13 |
| 3131 | D10 | Ar13 | D10 | D25 | Ar13 |
| 3132 | D10 | Ar13 | D10 | D26 | Ar13 |
| 3133 | D11 | Ar13 | D11 | D21 | Ar13 |
| 3134 | D11 | Ar13 | D11 | D22 | Ar13 |
| 3135 | D11 | Ar13 | D11 | D23 | Ar13 |
| 3136 | D11 | Ar13 | D11 | D24 | Ar13 |
| 3137 | D11 | Ar13 | D11 | D25 | Ar13 |
| 3138 | D11 | Ar13 | D11 | D26 | Ar13 |
| 3139 | D12 | Ar13 | D12 | D21 | Ar13 |
| 3140 | D12 | Ar13 | D12 | D22 | Ar13 |
| 3141 | D12 | Ar13 | D12 | D23 | Ar13 |
| 3142 | D12 | Ar13 | D12 | D24 | Ar13 |
| 3143 | D12 | Ar13 | D12 | D25 | Ar13 |
| 3144 | D12 | Ar13 | D12 | D26 | Ar13 |
| 3145 | D1 | Ar14 | D1 | D21 | Ar14 |
| 3146 | D1 | Ar14 | D1 | D22 | Ar14 |
| 3147 | D1 | Ar14 | D1 | D23 | Ar14 |
| 3148 | D1 | Ar14 | D1 | D24 | Ar14 |
| 3149 | D1 | Ar14 | D1 | D25 | Ar14 |
| 3150 | D1 | Ar14 | D1 | D26 | Ar14 |
| 3151 | D2 | Ar14 | D2 | D21 | Ar14 |
| 3152 | D2 | Ar14 | D2 | D22 | Ar14 |
| 3153 | D2 | Ar14 | D2 | D23 | Ar14 |
| 3154 | D2 | Ar14 | D2 | D24 | Ar14 |
| 3155 | D2 | Ar14 | D2 | D25 | Ar11 |
| 3156 | D2 | Ar14 | D2 | D26 | Ar14 |
| 3157 | D3 | Ar14 | D3 | D21 | Ar14 |
| 3158 | D3 | Ar14 | D3 | D22 | Ar14 |
| 3159 | D3 | Ar14 | D3 | D23 | Ar14 |
| 3160 | D3 | Ar14 | D3 | D24 | Ar14 |
| 3161 | D3 | Ar14 | D3 | D25 | Ar14 |
| 3162 | D3 | Ar14 | D3 | D26 | Ar14 |
| 3163 | D8 | Ar14 | D8 | D21 | Ar14 |
| 3164 | D8 | Ar14 | D8 | D22 | Ar14 |
| 3165 | D8 | Ar14 | D8 | D23 | Ar14 |
| 3166 | D8 | Ar14 | D8 | D24 | Ar14 |
| 3167 | D8 | Ar14 | D8 | D25 | Ar14 |
| 3168 | D8 | Ar14 | D8 | D26 | Ar14 |
| 3169 | D9 | Ar14 | D9 | D21 | Ar14 |
| 3170 | D9 | Ar14 | D9 | D22 | Ar14 |
| 3171 | D9 | Ar14 | D9 | D23 | Ar14 |
| 3172 | D9 | Ar14 | D9 | D24 | Ar14 |
| 3173 | D9 | Ar14 | D9 | D25 | Ar14 |
| 3174 | D9 | Ar14 | D9 | D26 | Ar14 |
| 3175 | D10 | Ar14 | D10 | D21 | Ar14 |
| 3176 | D10 | Ar14 | D10 | D22 | Ar14 |
| 3177 | D10 | Ar14 | D10 | D23 | Ar14 |
| 3178 | D10 | Ar14 | D10 | D24 | Ar14 |
| 3179 | D10 | Ar14 | D10 | D25 | Ar14 |
| 3180 | D10 | Ar14 | D10 | D26 | Ar14 |
| 3181 | D11 | Ar14 | D11 | D21 | Ar14 |
| 3182 | D11 | Ar14 | D11 | D22 | Ar14 |
| 3183 | D11 | Ar14 | D11 | D23 | Ar14 |
| 3184 | D11 | Ar14 | D11 | D24 | Ar14 |
| 3185 | D11 | Ar14 | D11 | D25 | Ar14 |
| 3186 | D11 | Ar14 | D11 | D26 | Ar14 |
| 3187 | D12 | Ar14 | D12 | D21 | Ar14 |
| 3188 | D12 | Ar14 | D12 | D22 | Ar14 |
| 3189 | D12 | Ar14 | D12 | D23 | Ar14 |
| 3180 | D12 | Ar14 | D12 | D24 | Ar14 |
| 3191 | D12 | Ar14 | D12 | D25 | Ar14 |
| 3192 | D12 | Ar14 | D12 | D26 | Ar14 |
| 3193 | D1 | Ar15 | D1 | D21 | Ar15 |
| 3194 | D1 | Ar15 | D1 | D22 | Ar15 |
| 3195 | D1 | Ar15 | D1 | D23 | Ar15 |
| 3196 | D1 | Ar15 | D1 | D24 | Ar15 |
| 3197 | D1 | Ar15 | D1 | D25 | Ar15 |
| 3198 | D1 | Ar15 | D1 | D26 | Ar15 |
| 3199 | D2 | Ar15 | D2 | D21 | Ar15 |
| 3200 | D2 | Ar15 | D2 | D22 | Ar15 |
| 3201 | D2 | Ar15 | D2 | D23 | Ar15 |
| 3202 | D2 | Ar15 | D2 | D24 | Ar15 |
| 3293 | D2 | Ar15 | D2 | D25 | Ar15 |
| 3204 | D2 | Ar15 | D2 | D26 | Ar15 |
| 3205 | D3 | Ar15 | D3 | D21 | Ar15 |
| 3206 | D3 | Ar15 | D3 | D22 | Ar15 |
| 3207 | D3 | Ar15 | D3 | D23 | Ar15 |
| 3208 | D3 | Ar15 | D3 | D24 | Ar15 |
| 3209 | D3 | Ar15 | D3 | D25 | Ar15 |
| 3210 | D3 | Ar15 | D3 | D26 | Ar15 |
| 3211 | D8 | Ar15 | D8 | D21 | Ar15 |
| 3212 | D8 | Ar15 | D8 | D22 | Ar15 |
| 3213 | D8 | Ar15 | D8 | D23 | Ar15 |
| 3214 | D8 | Ar15 | D8 | D24 | Ar15 |
| 3215 | D8 | Ar15 | D8 | D25 | Ar15 |
| 3216 | D8 | Ar15 | D8 | D26 | Ar15 |
| 3217 | D9 | Ar15 | D9 | D21 | Ar15 |
| 3218 | D9 | Ar15 | D9 | D22 | Ar15 |
| 3219 | D9 | Ar15 | D9 | D23 | Ar15 |
| 3220 | D9 | Ar15 | D9 | D24 | Ar15 |
| 3221 | D9 | Ar15 | D9 | D25 | Ar15 |
| 3222 | D9 | Ar15 | D9 | D26 | Ar15 |
| 3223 | D10 | Ar15 | D10 | D21 | Ar15 |
| 3224 | D10 | Ar15 | D10 | D22 | Ar15 |
| 3225 | D10 | Ar15 | D10 | D23 | Ar15 |
| 3226 | D10 | Ar15 | D10 | D24 | Ar15 |
| 3227 | D10 | Ar15 | D10 | D25 | Ar15 |
| 3228 | D10 | Ar15 | D10 | D26 | Ar15 |
| 3229 | D11 | Ar15 | D11 | D21 | Ar15 |
| 3230 | D11 | Ar15 | D11 | D22 | Ar15 |
| 3231 | D11 | Ar15 | D11 | D23 | Ar15 |
| 3232 | D11 | Ar15 | D11 | D24 | Ar15 |
| 3233 | D11 | Ar15 | D11 | D25 | Ar15 |
| 3234 | D11 | Ar15 | D11 | D26 | Ar15 |
| 3235 | D12 | Ar15 | D12 | D21 | Ar15 |
| 3236 | D12 | Ar15 | D12 | D22 | Ar15 |
| 3237 | D12 | Ar15 | D12 | D23 | Ar15 |
| 3238 | D12 | Ar15 | D12 | D24 | Ar15 |
| 3239 | D12 | Ar15 | D12 | D25 | Ar15 |
| 3240 | D12 | Ar15 | D12 | D26 | Ar15 |
| 3241 | D1 | Ar16 | D1 | D21 | Ar16 |
| 3242 | D1 | Ar16 | D1 | D22 | Ar16 |
| 3243 | D1 | Ar16 | D1 | D23 | Ar16 |
| 3244 | D1 | Ar16 | D1 | D24 | Ar16 |
| 3245 | D1 | Ar16 | D1 | D25 | Ar16 |
| 3246 | D1 | Ar16 | D1 | D26 | Ar16 |
| 3247 | D2 | Ar16 | D2 | D21 | Ar16 |
| 3248 | D2 | Ar16 | D2 | D22 | Ar16 |
| 3249 | D2 | Ar16 | D2 | D23 | Ar16 |
| 3250 | D2 | Ar16 | D2 | D24 | Ar16 |
| 3251 | D2 | Ar16 | D2 | D25 | Ar16 |
| 3252 | D2 | Ar16 | D2 | D26 | Ar16 |
| 3253 | D3 | Ar16 | D3 | D21 | Ar16 |
| 3254 | D3 | Ar16 | D3 | D22 | Ar16 |
| 3255 | D3 | Ar16 | D3 | D23 | Ar16 |
| 3256 | D3 | Ar16 | D3 | D24 | Ar16 |
| 3257 | D3 | Ar16 | D3 | D25 | Ar16 |
| 3258 | D3 | Ar16 | D3 | D26 | Ar16 |
| 3259 | D8 | Ar16 | D8 | D21 | Ar16 |
| 3260 | D8 | Ar16 | D8 | D22 | Ar16 |
| 3261 | D8 | Ar16 | D8 | D23 | Ar16 |
| 3262 | D8 | Ar16 | D8 | D24 | Ar16 |
| 3263 | D8 | Ar16 | D8 | D25 | Ar16 |
| 3264 | D8 | Ar16 | D8 | D26 | Ar16 |
| 3265 | D9 | Ar16 | D9 | D21 | Ar16 |
| 3266 | D9 | Ar16 | D9 | D22 | Ar16 |
| 3267 | D9 | Ar16 | D9 | D23 | Ar16 |
| 3268 | D9 | Ar16 | D9 | D24 | Ar16 |
| 3269 | D9 | Ar16 | D9 | D25 | Ar16 |
| 3270 | D9 | Ar16 | D9 | D26 | Ar16 |
| 3271 | D10 | Ar16 | D10 | D21 | Ar16 |
| 3272 | D10 | Ar16 | D10 | D22 | Ar10 |
| 3273 | D10 | Ar16 | D10 | D23 | Ar16 |
| 3274 | D10 | Ar16 | D10 | D24 | Ar16 |
| 3275 | D10 | Ar16 | D10 | D25 | Ar16 |
| 3276 | D10 | Ar16 | D10 | D26 | Ar16 |
| 3277 | D11 | Ar16 | D11 | D21 | Ar16 |
| 3278 | D11 | Ar16 | D11 | D22 | Ar16 |
| 3279 | D11 | Ar16 | D11 | D23 | Ar16 |
| 3280 | D11 | Ar16 | D11 | D24 | Ar16 |
| 3281 | D11 | Ar16 | D11 | D25 | Ar16 |
| 3282 | D11 | Ar16 | D11 | D26 | Ar16 |
| 3283 | D12 | Ar16 | D12 | D21 | Ar16 |
| 3284 | D12 | Ar16 | D12 | D22 | Ar16 |
| 3285 | D12 | Ar16 | D12 | D23 | Ar16 |
| 3286 | D12 | Ar16 | D12 | D24 | Ar16 |
| 3287 | D12 | Ar16 | D12 | D25 | Ar16 |
| 3288 | D12 | Ar16 | D12 | D26 | Ar16 |
| 3289 | D1 | Ar17 | D1 | D21 | Ar17 |
| 3290 | D1 | Ar17 | D1 | D22 | Ar17 |
| 3291 | D1 | Ar17 | D1 | D23 | Ar17 |
| 3292 | D1 | Ar17 | D1 | D24 | Ar17 |
| 3293 | D1 | Ar17 | D1 | D25 | Ar17 |
| 3294 | D1 | Ar17 | D1 | D26 | Ar17 |
| 3295 | D2 | Ar17 | D2 | D21 | Ar17 |
| 3296 | D2 | Ar17 | D2 | D22 | Ar17 |
| 3297 | D2 | Ar17 | D2 | D23 | Ar17 |
| 3298 | D2 | Ar17 | D2 | D24 | Ar17 |
| 3299 | D2 | Ar17 | D2 | D25 | Ar17 |
| 3300 | D2 | Ar17 | D2 | D26 | Ar17 |
| 3301 | D3 | Ar1 | D3 | D21 | Ar17 |
| 3302 | D3 | Ar1 | D3 | D22 | Ar17 |
| 3303 | D3 | Ar1 | D3 | D23 | Ar17 |
| 3304 | D3 | Ar1 | D3 | D24 | Ar17 |
| 3305 | D3 | Ar1 | D3 | D25 | Ar17 |
| 3306 | D3 | Ar1 | D3 | D26 | Ar17 |
| 3307 | D8 | Ar1 | D8 | D21 | Ar17 |
| 3308 | D8 | Ar1 | D8 | D22 | Ar17 |
| 3309 | D8 | Ar1 | D8 | D23 | Ar17 |
| 3310 | D8 | Ar1 | D8 | D24 | Ar17 |
| 3311 | D8 | Ar1 | D8 | D25 | Ar17 |
| 3312 | D8 | Ar1 | D8 | D26 | Ar17 |
| 3313 | D9 | Ar1 | D9 | D21 | Ar17 |
| 3314 | D9 | Ar1 | D9 | D22 | Ar17 |
| 3315 | D9 | Ar1 | D9 | D23 | Ar17 |
| 3316 | D9 | Ar1 | D9 | D24 | Ar17 |
| 3317 | D9 | Ar1 | D9 | D25 | Ar17 |
| 3318 | D9 | Ar1 | D9 | D26 | Ar17 |
| 3319 | D10 | Ar1 | D10 | D21 | Ar17 |
| 3320 | D10 | Ar1 | D10 | D22 | Ar17 |
| 3321 | D10 | Ar1 | D10 | D23 | Ar17 |
| 3322 | D10 | Ar1 | D10 | D24 | Ar17 |
| 3323 | D10 | Ar1 | D10 | D25 | Ar17 |
| 3324 | D10 | Ar1 | D10 | D26 | Ar17 |
| 3325 | D11 | Ar1 | D11 | D21 | Ar17 |
| 3326 | D11 | Ar1 | D11 | D22 | Ar17 |
| 3327 | D11 | Ar1 | D11 | D23 | Ar17 |
| 3328 | D11 | Ar1 | D11 | D24 | Ar17 |
| 3329 | D11 | Ar1 | D11 | D25 | Ar17 |
| 3330 | D11 | Ar1 | D11 | D26 | Ar17 |
| 3331 | D12 | Ar1 | D12 | D21 | Ar17 |
| 3332 | D12 | Ar1 | D12 | D22 | Ar17 |
| 3333 | D12 | Ar1 | D12 | D23 | Ar17 |
| 3334 | D12 | Ar1 | D12 | D24 | Ar17 |
| 3335 | D12 | Ar1 | D12 | D25 | Ar17 |
| 3336 | D12 | Ar1 | D12 | D26 | Ar17 |
| 3337 | Ar1 | D1 | D21 | D1 | Ar1 |
| 3338 | Ar1 | D2 | D21 | D2 | Ar1 |
| 3339 | Ar1 | D3 | D21 | D3 | Ar1 |
| 3340 | Ar1 | D4 | D21 | D4 | Ar1 |
| 3341 | Ar1 | D5 | D21 | D5 | Ar1 |
| 3342 | Ar1 | D6 | D21 | D6 | Ar1 |
| 3343 | Ar1 | D7 | D21 | D7 | Ar1 |
| 3344 | Ar1 | D8 | D21 | D8 | Ar1 |
| 3345 | Ar1 | D9 | D21 | D9 | Ar1 |
| 3346 | Ar1 | D10 | D21 | D10 | Ar1 |
| 3347 | Ar1 | D11 | D21 | D11 | Ar1 |
| 3348 | Ar1 | D12 | D21 | D12 | Ar1 |
| 3349 | Ar1 | D13 | D21 | D13 | Ar1 |
| 3350 | Ar1 | D14 | D21 | D14 | Ar1 |
| 3351 | Ar1 | D15 | D21 | D15 | Ar1 |
| 3352 | Ar1 | D16 | D21 | D16 | Ar1 |
| 3353 | Ar1 | D17 | D21 | D17 | Ar1 |
| 3354 | Ar1 | D18 | D21 | D18 | Ar1 |
| 3355 | Ar1 | D19 | D21 | D19 | Ar1 |
| 3356 | Ar1 | D20 | D21 | D20 | Ar1 |
| 3357 | Ar1 | D22 | D21 | D22 | Ar1 |
| 3358 | Ar1 | D23 | D21 | D23 | Ar1 |
| 3359 | Ar1 | D24 | D21 | D24 | Ar1 |
| 3360 | Ar1 | D25 | D21 | D25 | Ar1 |
| 3361 | Ar1 | D26 | D21 | D26 | Ar1 |
| 3362 | Ar1 | D1 | D22 | D1 | Ar1 |
| 3363 | Ar1 | D2 | D22 | D2 | Ar1 |
| 3364 | Ar1 | D3 | D22 | D3 | Ar1 |
| 3365 | Ar1 | D4 | D22 | D4 | Ar1 |
| 3366 | Ar1 | D5 | D22 | D5 | Ar1 |
| 3367 | Ar1 | D6 | D22 | D6 | Ar1 |
| 3368 | Ar1 | D7 | D22 | D7 | Ar1 |
| 3369 | Ar1 | D8 | D22 | D8 | Ar1 |
| 3370 | Ar1 | D9 | D22 | D9 | Ar1 |
| 3371 | Ar1 | D10 | D22 | D10 | Ar1 |
| 3372 | Ar1 | D11 | D22 | D11 | Ar1 |
| 3373 | Ar1 | D12 | D22 | D12 | Ar1 |
| 3374 | Ar1 | D13 | D22 | D13 | Ar1 |
| 3375 | Ar1 | D14 | D22 | D14 | Ar1 |
| 3376 | Ar1 | D15 | D22 | D15 | Ar1 |
| 3377 | Ar1 | D16 | D22 | D16 | Ar1 |
| 3378 | Ar1 | D17 | D22 | D17 | Ar1 |
| 3379 | Ar1 | D18 | D22 | D18 | Ar1 |
| 3380 | Ar1 | D19 | D22 | D19 | Ar1 |
| 3381 | Ar1 | D20 | D22 | D20 | Ar1 |
| 3382 | Ar1 | D21 | D22 | D21 | Ar1 |
| 3383 | Ar1 | D23 | D22 | D23 | Ar1 |
| 3384 | Ar1 | D24 | D22 | D24 | Ar1 |
| 3385 | Ar1 | D25 | D22 | D25 | Ar1 |
| 3386 | Ar1 | D26 | D22 | D26 | Ar1 |
| 3387 | Ar1 | D1 | D23 | D1 | Ar1 |
| 3388 | Ar1 | D2 | D23 | D2 | Ar1 |
| 3389 | Ar1 | D3 | D23 | D3 | Ar1 |
| 3390 | Ar1 | D4 | D23 | D4 | Ar1 |
| 3391 | Ar1 | D5 | D23 | D5 | Ar1 |
| 3392 | Ar1 | D6 | D23 | D6 | Ar1 |
| 3393 | Ar1 | D7 | D23 | D7 | Ar1 |
| 3394 | Ar1 | D8 | D23 | D8 | Ar1 |
| 3395 | Ar1 | D9 | D23 | D9 | Ar1 |
| 3396 | Ar1 | D10 | D23 | D10 | Ar1 |
| 3397 | Ar1 | D11 | D23 | D11 | Ar1 |
| 3398 | Ar1 | D12 | D23 | D12 | Ar1 |
| 3399 | Ar1 | D13 | D23 | D13 | Ar1 |
| 3400 | Ar1 | D14 | D23 | D14 | Ar1 |
| 3401 | Ar1 | D15 | D23 | D15 | Ar1 |
| 3402 | Ar1 | D16 | D23 | D16 | Ar1 |
| 3403 | Ar1 | D17 | D23 | D17 | Ar1 |
| 3404 | Ar1 | D18 | D23 | D18 | Ar1 |
| 3405 | Ar1 | D19 | D23 | D19 | Ar1 |
| 3406 | Ar1 | D20 | D23 | D20 | Ar1 |
| 3407 | Ar1 | D21 | D23 | D21 | Ar1 |
| 3408 | Ar1 | D22 | D23 | D22 | Ar1 |
| 3409 | Ar1 | D24 | D23 | D24 | Ar1 |
| 3410 | Ar1 | D25 | D23 | D25 | Ar1 |
| 3411 | Ar1 | D26 | D23 | D26 | Ar1 |
| 3412 | Ar1 | D1 | D24 | D1 | Ar1 |
| 3413 | Ar1 | D2 | D24 | D2 | Ar1 |
| 3414 | Ar1 | D3 | D24 | D3 | Ar1 |
| 3415 | Ar1 | D4 | D24 | D4 | Ar1 |
| 3416 | Ar1 | D5 | D24 | D5 | Ar1 |
| 3417 | Ar1 | D6 | D24 | D6 | Ar1 |
| 3418 | Ar1 | D7 | D24 | D7 | Ar1 |
| 3419 | Ar1 | D8 | D24 | D8 | Ar1 |
| 3420 | Ar1 | D9 | D24 | D9 | Ar1 |
| 3421 | Ar1 | D10 | D24 | D10 | Ar1 |
| 3422 | Ar1 | D11 | D24 | D11 | Ar1 |
| 3423 | Ar1 | D12 | D24 | D12 | Ar1 |
| 3424 | Ar1 | D13 | D24 | D13 | Ar1 |
| 3425 | Ar1 | D14 | D24 | D14 | Ar1 |
| 3426 | Ar1 | D15 | D24 | D15 | Ar1 |
| 3427 | Ar1 | D16 | D24 | D16 | Ar1 |
| 3428 | Ar1 | D17 | D24 | D17 | Ar1 |
| 3429 | Ar1 | D18 | D24 | D18 | Ar1 |
| 3430 | Ar1 | D19 | D24 | D19 | Ar1 |
| 3431 | Ar1 | D20 | D24 | D20 | Ar1 |
| 3432 | Ar1 | D21 | D24 | D21 | Ar1 |
| 3433 | Ar1 | D22 | D24 | D22 | Ar1 |
| 3434 | Ar1 | D23 | D24 | D23 | Ar1 |
| 3435 | Ar1 | D25 | D24 | D25 | Ar1 |
| 3436 | Ar1 | D26 | D24 | D26 | Ar1 |
| 3437 | Ar1 | D1 | D25 | D1 | Ar1 |
| 3438 | Ar1 | D2 | D25 | D2 | Ar1 |
| 3439 | Ar1 | D3 | D25 | D3 | Ar1 |
| 3440 | Ar1 | D4 | D25 | D4 | Ar1 |
| 3441 | Ar1 | D5 | D25 | D5 | Ar1 |
| 3442 | Ar1 | D6 | D25 | D6 | Ar1 |
| 3443 | Ar1 | D7 | D25 | D7 | Ar1 |
| 3444 | Ar1 | D8 | D25 | D8 | Ar1 |
| 3445 | Ar1 | D9 | D25 | D9 | Ar1 |
| 3446 | Ar1 | D10 | D25 | D10 | Ar1 |
| 3447 | Ar1 | D11 | D25 | D11 | Ar1 |
| 3448 | Ar1 | D12 | D25 | D12 | Ar1 |
| 3449 | Ar1 | D13 | D25 | D13 | Ar1 |
| 3450 | Ar1 | D14 | D25 | D14 | Ar1 |
| 3451 | Ar1 | D15 | D25 | D15 | Ar1 |
| 3452 | Ar1 | D16 | D25 | D16 | Ar1 |
| 3453 | Ar1 | D17 | D25 | D17 | Ar1 |
| 3454 | Ar1 | D18 | D25 | D18 | Ar1 |
| 3455 | Ar1 | D19 | D25 | D19 | Ar1 |
| 3456 | Ar1 | D20 | D25 | D20 | Ar1 |
| 3457 | Ar1 | D21 | D25 | D21 | Ar1 |
| 3458 | Ar1 | D22 | D25 | D22 | Ar1 |
| 3459 | Ar1 | D23 | D25 | D23 | Ar1 |
| 3460 | Ar1 | D24 | D25 | D24 | Ar1 |
| 3461 | Ar1 | D26 | D25 | D26 | Ar1 |
| 3462 | Ar1 | D1 | D26 | D1 | Ar1 |
| 3463 | Ar1 | D2 | D26 | D2 | Ar1 |
| 3464 | Ar1 | D3 | D26 | D3 | Ar1 |
| 3465 | Ar1 | D4 | D26 | D4 | Ar1 |
| 3465 | Ar1 | D5 | D26 | D5 | Ar1 |
| 3467 | Ar1 | D8 | D26 | D6 | Ar1 |
| 3468 | Ar1 | D7 | D26 | D7 | Ar1 |
| 3469 | Ar1 | D8 | D26 | D8 | Ar1 |
| 3470 | Ar1 | D9 | D26 | D9 | Ar1 |
| 3471 | Ar1 | D10 | D26 | D10 | Ar1 |
| 3472 | Ar1 | D11 | D26 | D11 | Ar1 |
| 3473 | Ar1 | D12 | D26 | D12 | Ar1 |
| 3474 | Ar1 | D13 | D26 | D13 | Ar1 |
| 3475 | Ar1 | D14 | D26 | D14 | Ar1 |
| 3476 | Ar1 | D15 | D26 | D15 | Ar1 |
| 3477 | Ar1 | D16 | D26 | D16 | Ar1 |
| 3478 | Ar1 | D17 | D26 | D17 | Ar1 |
| 3479 | Ar1 | D18 | D26 | D18 | Ar1 |
| 3480 | Ar1 | D19 | D26 | D19 | Ar1 |
| 3481 | Ar1 | D20 | D26 | D20 | Ar1 |
| 3482 | Ar1 | D21 | D26 | D21 | Ar1 |
| 3483 | Ar1 | D22 | D26 | D22 | Ar1 |
| 3484 | Ar1 | D23 | D26 | D23 | Ar1 |
| 3485 | Ar1 | D24 | D26 | D24 | Ar1 |
| 3486 | Ar1 | D25 | D26 | D25 | Ar1 |
| 3487 | Ar1 | D21 | D1 | D21 | Ar1 |
| 3483 | Ar1 | D21 | D2 | D21 | Ar1 |
| 3489 | Ar1 | D21 | D3 | D21 | Ar1 |
| 3490 | Ar1 | D21 | D4 | D21 | Ar1 |
| 3491 | Ar1 | D21 | D5 | D21 | Ar1 |
| 3492 | Ar1 | D21 | D6 | D21 | Ar1 |
| 3493 | Ar1 | D21 | D7 | D21 | Ar1 |
| 3494 | Ar1 | D21 | D8 | D21 | Ar1 |
| 3495 | Ar1 | D21 | D9 | D21 | Ar1 |
| 3496 | Ar1 | D21 | D10 | D21 | Ar1 |
| 3497 | Ar1 | D21 | D11 | D21 | Ar1 |
| 3498 | Ar1 | D21 | D12 | D21 | Ar1 |
| 3499 | Ar1 | D21 | D13 | D21 | Ar1 |
| 3500 | Ar1 | D21 | D14 | D21 | Ar1 |
| 3501 | Ar1 | D21 | D15 | D21 | Ar1 |
| 3502 | Ar1 | D21 | D16 | D21 | Ar1 |
| 3503 | Ar1 | D21 | D17 | D21 | Ar1 |
| 3504 | Ar1 | D21 | D18 | D21 | Ar1 |
| 3505 | Ar1 | D21 | D19 | D21 | Ar1 |
| 3506 | Ar1 | D21 | D20 | D21 | Ar1 |
| 3507 | Ar1 | D21 | D22 | D21 | Ar1 |
| 3508 | Ar1 | D21 | D23 | D21 | Ar1 |
| 3509 | Ar1 | D21 | D24 | D21 | Ar1 |
| 3510 | Ar1 | D21 | D25 | D21 | Ar1 |
| 3511 | Ar1 | D21 | D26 | D21 | Ar1 |
| 3512 | Ar1 | D22 | D1 | D22 | Ar1 |
| 3513 | Ar1 | D22 | D2 | D22 | Ar1 |
| 3514 | Ar1 | D22 | D3 | D22 | Ar1 |
| 3515 | Ar1 | D22 | D4 | D22 | Ar1 |
| 3516 | Ar1 | D22 | D5 | D22 | Ar1 |
| 3517 | Ar1 | D22 | D6 | D22 | Ar1 |
| 3518 | Ar1 | D22 | D7 | D22 | Ar1 |
| 3519 | Ar1 | D22 | D8 | D22 | Ar1 |
| 3520 | Ar1 | D22 | D9 | D22 | Ar1 |
| 3521 | Ar1 | D22 | D10 | D22 | Ar1 |
| 3522 | Ar1 | D22 | D11 | D22 | Ar1 |
| 3523 | Ar1 | D22 | D12 | D22 | Ar1 |
| 3524 | Ar1 | D22 | D13 | D22 | Ar1 |
| 3525 | Ar1 | D22 | D14 | D22 | Ar1 |
| 3526 | Ar1 | D22 | D15 | D22 | Ar1 |
| 3527 | Ar1 | D22 | D16 | D22 | Ar1 |
| 3528 | Ar1 | D22 | D17 | D22 | Ar1 |
| 3529 | Ar1 | D22 | D18 | 022 | Ar1 |
| 3530 | Ar1 | D22 | D19 | D22 | Ar1 |
| 3531 | Ar1 | D22 | D20 | D22 | Ar1 |
| 3532 | Ar1 | D22 | D21 | D22 | Ar1 |
| 3533 | Ar1 | D22 | D23 | D22 | Ar1 |
| 3534 | Ar1 | D22 | D24 | D22 | Ar1 |
| 3535 | Ar1 | D22 | D25 | D22 | Ar1 |
| 3536 | Ar1 | D22 | D26 | D22 | Ar1 |
| 3537 | Ar1 | D23 | D1 | D23 | Ar1 |
| 3538 | Ar1 | D23 | D2 | D23 | Ar1 |
| 3539 | Ar1 | D23 | D3 | D23 | Ar1 |
| 3540 | Ar1 | D23 | D4 | D23 | Ar1 |
| 3541 | Ar1 | D23 | D5 | D23 | Ar1 |
| 3542 | Ar1 | D23 | D6 | D23 | Ar1 |
| 3543 | Ar1 | D23 | D7 | D23 | Ar1 |
| 3544 | Ar1 | D23 | D8 | D23 | Ar1 |
| 3545 | Ar1 | D23 | D9 | D23 | Ar1 |
| 3546 | Ar1 | D23 | D10 | D23 | Ar1 |
| 3547 | Ar1 | D23 | D11 | D23 | Ar1 |
| 3548 | Ar1 | D23 | D12 | D23 | Ar1 |
| 3549 | Ar1 | D23 | D13 | D23 | Ar1 |
| 3550 | Ar1 | D23 | D14 | D23 | Ar1 |
| 3551 | Ar1 | D23 | D15 | D23 | Ar1 |
| 3552 | Ar1 | D23 | D16 | D23 | Ar1 |
| 3553 | Ar1 | D23 | D17 | D23 | Ar1 |
| 3554 | Ar1 | D23 | D18 | D23 | Ar1 |
| 3555 | Ar1 | D23 | D19 | D23 | Ar1 |
| 3556 | Ar1 | D23 | D20 | D23 | Ar1 |
| 3557 | Ar1 | D23 | D21 | D23 | Ar1 |
| 3558 | Ar1 | D23 | D22 | D23 | Ar1 |
| 3559 | Ar1 | D23 | D24 | D23 | Ar1 |
| 3560 | Ar1 | D23 | D25 | D23 | Ar1 |
| 3561 | Ar1 | D23 | D26 | D23 | Ar1 |
| 3562 | Ar1 | D24 | D1 | D24 | Ar1 |
| 3563 | Ar1 | D24 | D2 | D24 | Ar1 |
| 3564 | Ar1 | D24 | D3 | D24 | Ar1 |
| 3565 | Ar1 | D24 | D4 | D24 | Ar1 |
| 3566 | Ar1 | D24 | Do | D24 | Ar1 |
| 3567 | Ar1 | D24 | D6 | D24 | Ar1 |
| 3568 | Ar1 | D24 | D7 | D24 | Ar1 |
| 3569 | Ar1 | D24 | D8 | D24 | Ar1 |
| 3570 | Ar1 | D24 | D9 | D24 | Ar1 |
| 3571 | Ar1 | D24 | D10 | D24 | Ar1 |
| 3572 | Ar1 | D24 | D11 | D24 | Ar1 |
| 3573 | Ar1 | D24 | D12 | D24 | Ar1 |
| 3574 | Ar1 | D24 | D13 | D24 | Ar1 |
| 3575 | Ar1 | D24 | D14 | D24 | Ar1 |
| 3576 | Ar1 | D24 | D15 | D24 | Ar1 |
| 3577 | Ar1 | D24 | D16 | D24 | Ar1 |
| 3578 | Ar1 | D24 | D17 | D24 | Ar1 |
| 3579 | Ar1 | D24 | D18 | D24 | Ar1 |
| 3580 | Ar1 | D24 | D19 | D24 | Ar1 |
| 3581 | Ar1 | D24 | D20 | D24 | Ar1 |
| 3582 | Ar1 | D24 | D21 | 024 | Ar1 |
| 3583 | Ar1 | D24 | D22 | D24 | Ar1 |
| 3584 | Ar1 | D24 | D23 | D24 | Ar1 |
| 3585 | Ar1 | D24 | D25 | D24 | Ar1 |
| 3586 | Ar1 | D24 | D26 | D24 | Ar1 |
| 3587 | Ar1 | D25 | D1 | D25 | Ar1 |
| 3588 | Ar1 | D25 | D2 | D25 | Ar1 |
| 3589 | Ar1 | D25 | D3 | D25 | Ar1 |
| 3590 | Ar1 | D25 | D4 | D25 | Ar1 |
| 3591 | Ar1 | D25 | D5 | D25 | Ar1 |
| 3592 | Ar1 | D25 | D6 | D25 | Ar1 |
| 3593 | Ar1 | D25 | D7 | D25 | Ar1 |
| 3594 | Ar1 | D25 | D8 | D26 | Ar1 |
| 3595 | Ar1 | D25 | D9 | D25 | Ar1 |
| 3596 | Ar1 | D25 | D10 | D25 | Ar1 |
| 3597 | Ar1 | D25 | D11 | D25 | Ar1 |
| 3598 | Ar1 | D25 | D12 | D25 | Ar1 |
| 3599 | Ar1 | D25 | D13 | D25 | Ar1 |
| 3600 | Ar1 | D25 | D14 | D25 | Ar1 |
| 3601 | Ar1 | D25 | D15 | D25 | Ar1 |
| 3602 | Ar1 | D25 | D16 | D25 | Ar1 |
| 3603 | Ar1 | D25 | D17 | D25 | Ar1 |
| 3604 | Ar1 | D25 | D18 | D25 | Ar1 |
| 3605 | Ar1 | D25 | D19 | D25 | Ar1 |
| 3606 | Ar1 | D25 | D20 | D25 | Ar1 |
| 3607 | Ar1 | D25 | D21 | D25 | Ar1 |
| 3608 | Ar1 | D25 | D22 | D25 | Ar1 |
| 3609 | Ar1 | D25 | D23 | D25 | Ar1 |
| 3610 | Ar1 | D25 | D24 | D25 | Ar1 |
| 3611 | Ar1 | D25 | D26 | D25 | Ar1 |
| 3612 | Ar1 | D26 | D1 | D26 | Ar1 |
| 3613 | Ar1 | D26 | D2 | D26 | Ar1 |
| 3614 | Ar1 | D26 | D3 | D26 | Ar1 |
| 3615 | Ar1 | D26 | D4 | D26 | Ar1 |
| 3616 | Ar1 | D26 | D5 | D26 | Ar1 |
| 3617 | Ar1 | D26 | D6 | D26 | Ar1 |
| 3618 | Ar1 | D26 | D7 | D26 | Ar1 |
| 3619 | Ar1 | D26 | D8 | D26 | Ar1 |
| 3620 | Ar1 | D26 | D9 | D26 | Ar1 |
| 3621 | Ar1 | D26 | D10 | D26 | Ar1 |
| 3622 | Ar1 | D26 | D11 | D26 | Ar1 |
| 3623 | Ar1 | D26 | D12 | D26 | Ar1 |
| 3624 | Ar1 | D26 | D13 | D26 | Ar1 |
| 3625 | Ar1 | D26 | D14 | D26 | Ar1 |
| 3626 | Ar1 | D26 | D15 | D26 | Ar1 |
| 3627 | Ar1 | D26 | D16 | D26 | Ar1 |
| 3628 | Ar1 | D26 | D17 | D26 | Ar1 |
| 3629 | Ar1 | D26 | D18 | D26 | Ar1 |
| 3630 | Ar1 | D26 | D19 | D26 | Ar1 |
| 3631 | Ar1 | D26 | D20 | D26 | Ar1 |
| 3632 | Ar1 | D26 | D21 | D26 | Ar1 |
| 3633 | Ar1 | D26 | D22 | D26 | Ar1 |
| 3634 | Ar1 | D26 | D23 | D26 | Ar1 |
| 3635 | Ar1 | D26 | D24 | D26 | Ar1 |
| 3636 | Ar1 | D26 | D25 | D26 | Ar1 |
| 3637 | Ar1 | D21 | D1 | D1 | Ar1 |
| 3638 | Ar1 | D21 | D2 | D2 | Ar1 |
| 3639 | Ar1 | D21 | D3 | D3 | Ar1 |
| 3640 | Ar1 | D21 | D4 | D4 | Ar1 |
| 3641 | Ar1 | D21 | D5 | D5 | Ar1 |
| 3642 | Ar1 | D21 | D6 | D6 | Ar1 |
| 3643 | Ar1 | D21 | D7 | D7 | Ar1 |
| 3644 | Ar1 | D21 | D8 | D8 | Ar1 |
| 3645 | Ar1 | D21 | D9 | D9 | Ar1 |
| 3646 | Ar1 | D21 | D10 | D10 | Ar1 |
| 3647 | Ar1 | D21 | D11 | D11 | Ar1 |
| 3648 | Ar1 | D21 | D12 | D12 | Ar1 |
| 3649 | Ar1 | D21 | D13 | D13 | Ar1 |
| 3650 | Ar1 | D21 | D14 | D14 | Ar1 |
| 3651 | Ar1 | D21 | D15 | D15 | Ar1 |
| 3652 | Ar1 | D21 | D16 | D16 | Ar1 |
| 3653 | Ar1 | D21 | D17 | D17 | Ar1 |
| 3654 | Ar1 | D21 | D18 | D18 | Ar1 |
| 3655 | Ar1 | D21 | D19 | D19 | Ar1 |
| 3656 | Ar1 | D21 | D20 | D20 | Ar1 |
| 3657 | Ar1 | D21 | D22 | D22 | Ar1 |
| 3658 | Ar1 | D21 | D23 | D23 | Ar1 |
| 3659 | Ar1 | D21 | D24 | D24 | Ar1 |
| 3660 | Ar1 | D21 | D25 | D25 | Ar1 |
| 3661 | Ar1 | D21 | D26 | D26 | Ar1 |
| 3662 | Ar1 | D22 | D1 | D1 | Ar1 |
| 3663 | Ar1 | D22 | D2 | D2 | Ar1 |
| 3664 | Ar1 | D22 | D3 | D3 | Ar1 |
| 3665 | Ar1 | D22 | D4 | D4 | Ar1 |
| 3666 | Ar1 | D22 | D5 | D5 | Ar1 |
| 3667 | Ar1 | D22 | D6 | D6 | Ar1 |
| 3668 | Ar1 | D22 | D7 | D7 | Ar1 |
| 3669 | Ar1 | D22 | D8 | D8 | Ar1 |
| 3670 | Ar1 | D22 | D9 | D9 | Ar1 |
| 3671 | Ar1 | D22 | D10 | D10 | Ar1 |
| 3672 | Ar1 | D22 | D11 | D11 | Ar1 |
| 3673 | Ar1 | D22 | D12 | D12 | Ar1 |
| 3674 | Ar1 | D22 | D13 | D13 | Ar1 |
| 3675 | Ar1 | D22 | D14 | D14 | Ar1 |
| 3676 | Ar1 | D22 | D15 | D15 | Ar1 |
| 3677 | Ar1 | D22 | D16 | D16 | Ar1 |
| 3678 | Ar1 | D22 | D17 | D17 | Ar1 |
| 3679 | Ar1 | D22 | D18 | D18 | Ar1 |
| 3680 | Ar1 | D22 | D19 | D19 | Ar1 |
| 3681 | Ar1 | D22 | D20 | D20 | Ar1 |
| 3682 | Ar1 | D22 | D21 | D21 | Ar1 |
| 3683 | Ar1 | D22 | D23 | D23 | Ar1 |
| 3684 | Ar1 | D22 | D24 | D24 | Ar1 |
| 3685 | Ar1 | D22 | D25 | D25 | Ar1 |
| 3686 | Ar1 | D22 | D26 | D26 | Ar1 |
| 3687 | Ar1 | D23 | D1 | D1 | Ar1 |
| 3688 | Ar1 | D23 | D2 | D2 | Ar1 |
| 3689 | Ar1 | D23 | D3 | D3 | Ar1 |
| 3690 | Ar1 | D23 | D4 | D4 | Ar1 |
| 3691 | Ar1 | D23 | D5 | D5 | Ar1 |
| 3692 | Ar1 | D23 | D6 | D6 | Ar1 |
| 3693 | Ar1 | D23 | D7 | D7 | Ar1 |
| 3694 | Ar1 | D23 | D8 | D8 | Ar1 |
| 3695 | Ar1 | D23 | D9 | D9 | Ar1 |
| 3696 | Ar1 | D23 | D10 | D10 | Ar1 |
| 3697 | Ar1 | D23 | D11 | D11 | Ar1 |
| 3698 | Ar1 | D23 | D12 | D12 | Ar1 |
| 3699 | Ar1 | D23 | D13 | D13 | Ar1 |
| 3700 | Ar1 | D23 | D14 | D14 | Ar1 |
| 3701 | Ar1 | D23 | D15 | D15 | Ar1 |
| 3702 | Ar1 | D23 | D16 | D16 | Ar1 |
| 3703 | Ar1 | D23 | D17 | D17 | Ar1 |
| 3704 | Ar1 | D23 | D18 | D18 | Ar1 |
| 3705 | Ar1 | D23 | D19 | D19 | Ar1 |
| 3706 | Ar1 | D23 | D20 | D20 | Ar1 |
| 3707 | Ar1 | D23 | D21 | D21 | Ar1 |
| 3708 | Ar1 | D23 | D22 | D22 | Ar1 |
| 3709 | Ar1 | D23 | D24 | D24 | Ar1 |
| 3710 | Ar1 | D23 | D25 | D25 | Ar1 |
| 3711 | Ar1 | D23 | D26 | D26 | Ar1 |
| 3712 | Ar1 | D24 | D1 | D1 | Ar1 |
| 3713 | Ar1 | D24 | D2 | D2 | Ar1 |
| 3714 | Ar1 | D24 | D3 | D3 | Ar1 |
| 3715 | Ar1 | D24 | D4 | D4 | Ar1 |
| 3716 | Ar1 | D24 | D5 | D5 | Ar1 |
| 3717 | Ar1 | D24 | D6 | D6 | Ar1 |
| 3718 | Ar1 | D24 | D7 | D7 | Ar1 |
| 3719 | Ar1 | D24 | D8 | D8 | Ar1 |
| 3720 | Ar1 | D24 | D9 | D9 | Ar1 |
| 3721 | Ar1 | D24 | D10 | D10 | Ar1 |
| 3722 | Ar1 | D24 | D11 | D11 | Ar1 |
| 3723 | Ar1 | D24 | D12 | D12 | Ar1 |
| 3724 | Ar1 | D24 | D13 | D13 | Ar1 |
| 3725 | Ar1 | D24 | D14 | D14 | Ar1 |
| 3726 | Ar1 | D24 | D15 | D15 | Ar1 |
| 3727 | Ar1 | D24 | D16 | D16 | Ar1 |
| 3728 | Ar1 | D24 | D17 | D17 | Ar1 |
| 3729 | Ar1 | D24 | D18 | D18 | Ar1 |
| 3730 | Ar1 | D24 | D19 | D19 | Ar1 |
| 3731 | Ar1 | D24 | D20 | D20 | Ar1 |
| 3732 | Ar1 | D24 | D21 | D21 | Ar1 |
| 3733 | Ar1 | D24 | D22 | D22 | Ar1 |
| 3734 | Ar1 | D24 | D23 | D23 | Ar1 |
| 3735 | Ar1 | D24 | D25 | D25 | Ar1 |
| 3736 | Ar1 | D24 | D26 | D26 | Ar1 |
| 3737 | Ar1 | D25 | D1 | D1 | Ar1 |
| 3738 | Ar1 | D25 | D2 | D2 | Ar1 |
| 3739 | Ar1 | D25 | D3 | D3 | Ar1 |
| 3740 | Ar1 | D25 | D4 | D4 | Ar1 |
| 3741 | Ar1 | D25 | D5 | D5 | Ar1 |
| 3742 | Ar1 | D25 | D6 | D6 | Ar1 |
| 3743 | Ar1 | D25 | D7 | D7 | Ar1 |
| 3744 | Ar1 | D25 | D8 | D8 | Ar1 |
| 3745 | Ar1 | D25 | D9 | D9 | Ar1 |
| 3746 | Ar1 | D25 | D10 | D10 | Ar1 |
| 3747 | Ar1 | D25 | D11 | D11 | Ar1 |
| 3748 | Ar1 | D25 | D12 | D12 | Ar1 |
| 3749 | Ar1 | D25 | D13 | D13 | Ar1 |
| 3750 | Ar1 | D25 | D14 | D14 | Ar1 |
| 3751 | Ar1 | D25 | D15 | D15 | Ar1 |
| 3752 | Ar1 | D25 | D16 | D16 | Ar1 |
| 3753 | Ar1 | D25 | D17 | D17 | Ar1 |
| 3754 | Ar1 | D25 | D18 | D18 | Ar1 |
| 3755 | Ar1 | D25 | D19 | D19 | Ar1 |
| 3756 | Ar1 | D25 | D20 | D20 | Ar1 |
| 3757 | Ar1 | D25 | D21 | D21 | Ar1 |
| 3758 | Ar1 | D25 | D22 | D22 | Ar1 |
| 3759 | Ar1 | D25 | D23 | D23 | Ar1 |
| 3760 | Ar1 | D25 | D24 | D24 | Ar1 |
| 3761 | Ar1 | D25 | D26 | D26 | Ar1 |
| 3762 | Ar1 | D26 | D1 | D1 | Ar1 |
| 3763 | Ar1 | D26 | D2 | D2 | Ar1 |
| 3764 | Ar1 | D26 | D3 | D3 | Ar1 |
| 3765 | Ar1 | D26 | D4 | D4 | Ar1 |
| 3766 | Ar1 | D26 | D5 | D5 | Ar1 |
| 3767 | Ar1 | D26 | D6 | D6 | Ar1 |
| 3768 | Ar1 | D26 | D7 | D7 | Ar1 |
| 3769 | Ar1 | D26 | D8 | D8 | Ar1 |
| 3770 | Ar1 | D26 | D9 | D9 | Ar1 |
| 3771 | Ar1 | D26 | D10 | D10 | Ar1 |
| 3772 | Ar1 | D26 | D11 | D11 | Ar1 |
| 3773 | Ar1 | D26 | D12 | D12 | Ar1 |
| 3774 | Ar1 | D26 | D13 | D13 | Ar1 |
| 3775 | Ar1 | D26 | D14 | D14 | Ar1 |
| 3776 | Ar1 | D26 | D15 | D15 | Ar1 |
| 3777 | Ar1 | D26 | D16 | D16 | Ar1 |
| 3778 | Ar1 | D26 | D17 | D17 | Ar1 |
| 3779 | Ar1 | D26 | D18 | D18 | Ar1 |
| 3730 | Ar1 | D26 | D19 | D19 | Ar1 |
| 3781 | Ar1 | D26 | D20 | D20 | Ar1 |
| 3782 | Ar1 | D26 | D21 | D21 | Ar1 |
| 3783 | Ar1 | D26 | D22 | D22 | Ar1 |
| 3784 | Ar1 | D26 | D23 | D23 | Ar1 |
| 3785 | Ar1 | D26 | D24 | D24 | Ar1 |
| 3786 | Ar1 | D26 | D25 | D25 | Ar1 |
| 3787 | Ar1 | D1 | D21 | D21 | Ar1 |
| 3788 | Ar1 | D2 | D21 | D21 | Ar1 |
| 3789 | Ar1 | D3 | D21 | D21 | Ar1 |
| 3790 | Ar1 | D4 | D21 | D21 | Ar1 |
| 3791 | Ar1 | D5 | D21 | D21 | Ar1 |
| 3792 | Ar1 | D6 | D21 | D21 | Ar1 |
| 3793 | Ar1 | D7 | D21 | D21 | Ar1 |
| 3794 | Ar1 | D8 | D21 | D21 | Ar1 |
| 3795 | Ar1 | D9 | D21 | D21 | Ar1 |
| 3796 | Ar1 | D10 | D21 | D21 | Ar1 |
| 3797 | Ar1 | D11 | D21 | D21 | Ar1 |
| 3798 | Ar1 | D12 | D21 | D21 | Ar1 |
| 3799 | Ar1 | D13 | D21 | D21 | Ar1 |
| 3800 | Ar1 | D14 | D21 | D21 | Ar1 |
| 3801 | Ar1 | D15 | D21 | D21 | Ar1 |
| 3802 | Ar1 | D16 | D21 | D21 | Ar1 |
| 3803 | Ar1 | D17 | D21 | D21 | Ar1 |
| 3804 | Ar1 | D18 | D21 | D21 | Ar1 |
| 3805 | Ar1 | D19 | D21 | D21 | Ar1 |
| 3806 | Ar1 | D20 | D21 | D21 | Ar1 |
| 3807 | Ar1 | D22 | D21 | D21 | Ar1 |
| 3808 | Ar1 | D23 | D21 | D21 | Ar1 |
| 3809 | Ar1 | D24 | D21 | D21 | Ar1 |
| 3810 | Ar1 | D25 | D21 | D21 | Ar1 |
| 3811 | Ar1 | D26 | D21 | D21 | Ar1 |
| 3812 | Ar1 | D1 | D22 | D22 | Ar1 |
| 3813 | Ar1 | D2 | D22 | D22 | Ar1 |
| 3814 | Ar1 | D3 | D22 | D22 | Ar1 |
| 3815 | Ar1 | D4 | D22 | D22 | Ar1 |
| 3816 | Ar1 | D5 | D22 | D22 | Ar1 |
| 3817 | Ar1 | D6 | D22 | D22 | Ar1 |
| 3818 | Ar1 | D7 | D22 | D22 | Ar1 |
| 3819 | Ar1 | D8 | D22 | D22 | Ar1 |
| 3820 | Ar1 | D9 | D22 | D22 | Ar1 |
| 3821 | Ar1 | D10 | D22 | D22 | Ar1 |
| 3822 | Ar1 | D11 | D22 | D22 | Ar1 |
| 3823 | Ar1 | D12 | D22 | D22 | Ar1 |
| 3824 | Ar1 | D13 | D22 | D22 | Ar1 |
| 3825 | Ar1 | D14 | D22 | D22 | Ar1 |
| 3826 | Ar1 | D15 | D22 | D22 | Ar1 |
| 3827 | Ar1 | D16 | D22 | D22 | Ar1 |
| 3828 | Ar1 | D17 | D22 | D22 | Ar1 |
| 3829 | Ar1 | D18 | D22 | D22 | Ar1 |
| 3830 | Ar1 | D19 | D22 | D22 | Ar1 |
| 3831 | Ar1 | D20 | D22 | D22 | Ar1 |
| 3832 | Ar1 | D21 | D22 | D22 | Ar1 |
| 3833 | Ar1 | D23 | D22 | D22 | Ar1 |
| 3834 | Ar1 | D24 | D22 | D22 | Ar1 |
| 3835 | Ar1 | D25 | D22 | D22 | Ar1 |
| 3836 | Ar1 | D26 | D22 | D22 | Ar1 |
| 3837 | Ar1 | D1 | D23 | D23 | Ar1 |
| 3838 | Ar1 | D2 | D23 | D23 | Ar1 |
| 3839 | Ar1 | D3 | D23 | D23 | Ar1 |
| 3840 | Ar1 | D4 | D23 | D23 | Ar1 |
| 3841 | Ar1 | D5 | D23 | D23 | Ar1 |
| 3842 | Ar1 | D6 | D23 | D23 | Ar1 |
| 3843 | Ar1 | D7 | D23 | D23 | Ar1 |
| 3844 | Ar1 | D8 | D23 | D23 | Ar1 |
| 3845 | Ar1 | D9 | D23 | D23 | Ar1 |
| 3846 | Ar1 | D10 | D23 | D23 | Ar1 |
| 3847 | Ar1 | D11 | D23 | D23 | Ar1 |
| 3848 | Ar1 | D12 | D23 | D23 | Ar1 |
| 3849 | Ar1 | D13 | D23 | D23 | Ar1 |
| 3850 | Ar1 | D14 | D23 | D23 | Ar1 |
| 3851 | Ar1 | D15 | D23 | D23 | Ar1 |
| 3852 | Ar1 | D16 | D23 | D23 | Ar1 |
| 3853 | Ar1 | D17 | D23 | D23 | Ar1 |
| 3854 | Ar1 | D18 | D23 | D23 | Ar1 |
| 3855 | Ar1 | D19 | D23 | D23 | Ar1 |
| 3856 | Ar1 | D20 | D23 | D23 | Ar1 |
| 3857 | Ar1 | D21 | D23 | D23 | Ar1 |
| 3858 | Ar1 | D22 | D23 | D23 | Ar1 |
| 3859 | Ar1 | D24 | D23 | D23 | Ar1 |
| 3860 | Ar1 | D25 | D23 | D23 | Ar1 |
| 3861 | Ar1 | D26 | D23 | D23 | Ar1 |
| 3862 | Ar1 | D1 | D24 | D24 | Ar1 |
| 3863 | Ar1 | D2 | D24 | D24 | Ar1 |
| 3864 | Ar1 | D3 | D24 | D24 | Ar1 |
| 3865 | Ar1 | D4 | D24 | D24 | Ar1 |
| 3866 | Ar1 | D5 | D24 | D24 | Ar1 |
| 3867 | Ar1 | D6 | D24 | D24 | Ar1 |
| 3868 | Ar1 | D7 | D24 | D24 | Ar1 |
| 3869 | Ar1 | D8 | D24 | D24 | Ar1 |
| 3870 | Ar1 | D9 | D24 | D24 | Ar1 |
| 3871 | Ar1 | D10 | D24 | D24 | Ar1 |
| 3872 | Ar1 | D11 | D24 | D24 | Ar1 |
| 3873 | Ar1 | D12 | D24 | D24 | Ar1 |
| 3874 | Ar1 | D13 | D24 | D24 | Ar1 |
| 3875 | Ar1 | D14 | D24 | D24 | Ar1 |
| 3876 | Ar1 | D15 | D24 | D24 | Ar1 |
| 3877 | Ar1 | D16 | D24 | D24 | Ar1 |
| 3878 | Ar1 | D17 | D24 | D24 | Ar1 |
| 3879 | Ar1 | D18 | D24 | D24 | Ar1 |
| 3880 | Ar1 | D19 | D24 | D24 | Ar1 |
| 3881 | Ar1 | D20 | D24 | D24 | Ar1 |
| 3882 | Ar1 | D21 | D24 | D24 | Ar1 |
| 3883 | Ar1 | D22 | D24 | D24 | Ar1 |
| 3884 | Ar1 | D23 | D24 | D24 | Ar1 |
| 3885 | Ar1 | D25 | D24 | D24 | Ar1 |
| 3886 | Ar1 | D26 | D24 | D24 | Ar1 |
| 3887 | Ar1 | D1 | D25 | D25 | Ar1 |
| 3888 | Ar1 | D2 | D25 | D25 | Ar1 |
| 3889 | Ar1 | D3 | D25 | D25 | Ar1 |
| 3890 | Ar1 | D4 | D25 | D25 | Ar1 |
| 3891 | Ar1 | D5 | D25 | D25 | Ar1 |
| 3892 | Ar1 | D6 | D25 | D25 | Ar1 |
| 3893 | Ar1 | D7 | D25 | D25 | Ar1 |
| 3894 | Ar1 | D8 | D25 | D25 | Ar1 |
| 3895 | Ar1 | D9 | D25 | D25 | Ar1 |
| 3896 | Ar1 | D10 | D25 | D25 | Ar1 |
| 3897 | Ar1 | D11 | D25 | D25 | Ar1 |
| 3898 | Ar1 | D12 | D25 | D25 | Ar1 |
| 3899 | Ar1 | D13 | D25 | D25 | Ar1 |
| 3900 | Ar1 | D14 | D25 | D25 | Ar1 |
| 3901 | Ar1 | D15 | D25 | D25 | Ar1 |
| 3902 | Ar1 | D16 | D25 | D25 | Ar1 |
| 3903 | Ar1 | D17 | D25 | D25 | Ar1 |
| 3904 | Ar1 | D18 | D25 | D26 | Ar1 |
| 3905 | Ar1 | D19 | D25 | D25 | Ar1 |
| 3906 | Ar1 | D20 | D25 | D25 | Ar1 |
| 3907 | Ar1 | D21 | D25 | D25 | Ar1 |
| 3908 | Ar1 | D22 | D25 | D25 | Ar1 |
| 3909 | Ar1 | D23 | D25 | D25 | Ar1 |
| 3910 | Ar1 | D24 | D25 | D25 | Ar1 |
| 3911 | Ar1 | D26 | D25 | D25 | Ar1 |
| 3912 | Ar1 | D1 | D26 | D26 | Ar1 |
| 3913 | Ar1 | D2 | D26 | D26 | Ar1 |
| 3914 | Ar1 | D3 | D26 | D26 | Ar1 |
| 3915 | Ar1 | D4 | D26 | D26 | Ar1 |
| 3916 | Ar1 | D5 | D26 | D26 | Ar1 |
| 3917 | Ar1 | D6 | D26 | D26 | Ar1 |
| 3918 | Ar1 | D7 | D26 | D26 | Ar1 |
| 3919 | Ar1 | D8 | D26 | D26 | Ar1 |
| 3920 | Ar1 | D9 | D26 | D26 | Ar1 |
| 3921 | Ar1 | D10 | D26 | D26 | Ar1 |
| 3922 | Ar1 | D11 | D26 | D26 | Ar1 |
| 3923 | Ar1 | D12 | D26 | D26 | Ar1 |
| 3924 | Ar1 | D13 | D26 | D26 | Ar1 |
| 3925 | Ar1 | D14 | D26 | D26 | Ar1 |
| 3926 | Ar1 | D15 | D26 | D26 | Ar1 |
| 3927 | Ar1 | D16 | D26 | D26 | Ar1 |
| 3928 | Ar1 | D17 | D26 | D26 | Ar1 |
| 3929 | Ar1 | D18 | D26 | D26 | Ar1 |
| 3930 | Ar1 | D19 | D26 | D26 | Ar1 |
| 3931 | Ar1 | D20 | D26 | D26 | Ar1 |
| 3932 | Ar1 | D21 | D26 | D26 | Ar1 |
| 3933 | Ar1 | D22 | D26 | D26 | Ar1 |
| 3934 | Ar1 | D23 | D26 | D26 | Ar1 |
| 3935 | Ar1 | D24 | D26 | D26 | Ar1 |
| 3936 | Ar1 | D25 | D26 | D26 | Ar1 |
| 3937 | D1 | Ar1 | D21 | Ar1 | D1 |
| 3938 | D2 | Ar1 | D21 | Ar1 | D2 |
| 3939 | D3 | Ar1 | D21 | Ar1 | D3 |
| 3340 | D4 | Ar1 | D21 | Ar1 | D4 |
| 3941 | D5 | Ar1 | D21 | Ar1 | D5 |
| 3942 | D6 | Ar1 | D21 | Ar1 | D6 |
| 3943 | D7 | Ar1 | D21 | Ar1 | D7 |
| 3944 | D8 | Ar1 | D21 | Ar1 | D8 |
| 3945 | D9 | Ar1 | D21 | Ar1 | D9 |
| 3946 | D10 | Ar1 | D21 | Ar1 | D10 |
| 3947 | D11 | Ar1 | D21 | Ar1 | D11 |
| 3948 | D12 | Ar1 | D21 | Ar1 | D12 |
| 3949 | D13 | Ar1 | D21 | Ar1 | D13 |
| 3950 | D14 | Ar1 | D21 | Ar1 | D14 |
| 3951 | D15 | Ar1 | D21 | Ar1 | D15 |
| 3952 | D16 | Ar1 | D21 | Ar1 | D16 |
| 3953 | D17 | Ar1 | D21 | Ar1 | D17 |
| 3954 | D18 | Ar1 | D21 | Ar1 | D18 |
| 3955 | D19 | Ar1 | D21 | Ar1 | D19 |
| 3956 | D20 | Ar1 | D21 | Ar1 | D20 |
| 3957 | D22 | Ar1 | D21 | Ar1 | D22 |
| 3958 | D23 | Ar1 | D21 | Ar1 | D23 |
| 3959 | D24 | Ar1 | D21 | Ar1 | D24 |
| 3960 | D25 | Ar1 | D21 | Ar1 | D25 |
| 3961 | D26 | Ar1 | D21 | Ar1 | D26 |
| 3962 | D1 | Ar1 | D22 | Ar1 | D1 |
| 3963 | D2 | Ar1 | D22 | Ar1 | D2 |
| 3364 | D3 | Ar1 | D22 | Ar1 | D3 |
| 3965 | D4 | Ar1 | D22 | Ar1 | D4 |
| 3966 | D5 | Ar1 | D22 | Ar1 | D5 |
| 3967 | D6 | Ar1 | D22 | Ar1 | D6 |
| 3968 | D7 | Ar1 | D22 | Ar1 | D7 |
| 3969 | D8 | Ar1 | D22 | Ar1 | D8 |
| 3970 | D9 | Ar1 | D22 | Ar1 | D9 |
| 3971 | D10 | Ar1 | D22 | Ar1 | D10 |
| 3972 | D11 | Ar1 | D22 | Ar1 | D11 |
| 3973 | D12 | Ar1 | D22 | Ar1 | D12 |
| 3974 | D13 | Ar1 | D22 | Ar1 | D13 |
| 3975 | D14 | Ar1 | D22 | Ar1 | D14 |
| 3976 | D15 | Ar1 | D27 | Ar1 | D15 |
| 3977 | D16 | Ar1 | D22 | Ar1 | D16 |
| 3978 | D17 | Ar1 | D22 | Ar1 | D17 |
| 3979 | D18 | Ar1 | D22 | Ar1 | D18 |
| 3980 | D19 | Ar1 | D22 | Ar1 | D19 |
| 3981 | D20 | Ar1 | D22 | Ar1 | D20 |
| 3982 | D21 | Ar1 | D22 | Ar1 | D21 |
| 3983 | D23 | Ar1 | D22 | Ar1 | D23 |
| 3984 | D24 | Ar1 | D22 | Ar1 | D24 |
| 3985 | D25 | Ar1 | D22 | Ar1 | D25 |
| 3986 | D26 | Ar1 | D22 | Ar1 | D26 |
| 3987 | D1 | Ar1 | D23 | Ar1 | D1 |
| 3988 | D2 | Ar1 | D23 | Ar1 | D2 |
| 3989 | D3 | Ar1 | D23 | Ar1 | D3 |
| 3990 | D4 | Ar1 | D23 | Ar1 | D4 |
| 3991 | D5 | Ar1 | D22 | Ar1 | D5 |
| 3992 | D6 | Ar1 | D23 | Ar1 | D6 |
| 3993 | D7 | Ar1 | D23 | Ar1 | D7 |
| 3994 | D8 | Ar1 | D23 | Ar1 | D8 |
| 3995 | D9 | Ar1 | D23 | Ar1 | D9 |
| 3996 | D10 | Ar1 | D23 | Ar1 | D10 |
| 3997 | D11 | Ar1 | D23 | Ar1 | D11 |
| 3998 | D12 | Ar1 | D23 | Ar1 | D12 |
| 3999 | D13 | Ar1 | D23 | Ar1 | D13 |
| 4000 | D14 | Ar1 | D23 | Ar1 | D14 |
| 4001 | D15 | Ar1 | D23 | Ar1 | D15 |
| 4002 | D16 | Ar1 | D23 | Ar1 | D16 |
| 4003 | D17 | Ar1 | D23 | Ar1 | D17 |
| 4004 | D18 | Ar1 | D23 | Ar1 | D18 |
| 4005 | D19 | Ar1 | D23 | Ar1 | D19 |
| 4006 | D20 | Ar1 | D23 | Ar1 | D20 |
| 4007 | D21 | Ar1 | D23 | Ar1 | D21 |
| 4008 | D22 | Ar1 | D23 | Ar1 | D22 |
| 4009 | D24 | Ar1 | D23 | Ar1 | D24 |
| 4010 | D25 | Ar1 | D23 | Ar1 | D25 |
| 4011 | D26 | Ar1 | D23 | Ar1 | D26 |
| 4012 | D1 | Ar1 | D24 | Ar1 | D1 |
| 4013 | D2 | Ar1 | D24 | Ar1 | D2 |
| 4014 | D3 | Ar1 | D24 | Ar1 | D3 |
| 4015 | D4 | Ar1 | D24 | Ar1 | D4 |
| 4016 | D5 | Ar1 | D24 | Ar1 | D5 |
| 4017 | D6 | Ar1 | D24 | Ar1 | D6 |
| 4018 | D7 | Ar1 | D24 | Ar1 | D7 |
| 4019 | D8 | Ar1 | D24 | Ar1 | D8 |
| 4020 | D9 | Ar1 | D24 | Ar1 | D9 |
| 4021 | D10 | Ar1 | D24 | Ar1 | D10 |
| 4022 | D11 | Ar1 | D24 | Ar1 | D11 |
| 4023 | D12 | Ar1 | D24 | Ar1 | D12 |
| 4024 | D13 | Ar1 | D24 | Ar1 | D13 |
| 4025 | D14 | Ar1 | D24 | Ar1 | D14 |
| 4026 | D15 | Ar1 | D24 | Ar1 | D15 |
| 4027 | D16 | Ar1 | D24 | Ar1 | D16 |
| 4028 | D17 | Ar1 | D24 | Ar1 | D17 |
| 4029 | D18 | Ar1 | D24 | Ar1 | D18 |
| 4030 | D19 | Ar1 | D24 | Ar1 | D19 |
| 4031 | D20 | Ar1 | D24 | Ar1 | D20 |
| 4032 | D21 | Ar1 | D24 | Ar1 | D21 |
| 4033 | D22 | Ar1 | D24 | Ar1 | D22 |
| 4034 | D23 | Ar1 | D24 | Ar1 | D23 |
| 4035 | D25 | Ar1 | D24 | Ar1 | D25 |
| 4036 | D26 | Ar1 | D24 | Ar1 | D26 |
| 4037 | D1 | Ar1 | D25 | Ar1 | D1 |
| 4038 | D2 | Ar1 | D25 | Ar1 | D2 |
| 4039 | D3 | Ar1 | D25 | Ar1 | D3 |
| 4040 | D4 | Ar1 | D25 | Ar1 | D4 |
| 4041 | D5 | Ar1 | D25 | Ar1 | D5 |
| 4042 | D6 | Ar1 | D25 | Ar1 | D6 |
| 4043 | D7 | Ar1 | D25 | Ar1 | D7 |
| 4044 | D8 | Ar1 | D25 | Ar1 | D8 |
| 4045 | D9 | Ar1 | D25 | Ar1 | D9 |
| 4046 | D10 | Ar1 | D25 | Ar1 | D10 |
| 4047 | D11 | Ar1 | D25 | Ar1 | D11 |
| 4048 | D12 | Ar1 | D25 | Ar1 | D12 |
| 4049 | D13 | Ar1 | D25 | Ar1 | D13 |
| 4050 | D14 | Ar1 | D25 | Ar1 | D14 |
| 4051 | D15 | Ar1 | D25 | Ar1 | D15 |
| 4052 | D16 | Ar1 | D25 | Ar1 | D16 |
| 4053 | D17 | Ar1 | D25 | Ar1 | D17 |
| 4054 | D18 | Ar1 | D25 | Ar1 | D18 |
| 4055 | D19 | Ar1 | D25 | Ar1 | D19 |
| 4056 | D20 | Ar1 | D25 | Ar1 | D20 |
| 4057 | D21 | Ar1 | D25 | Ar1 | D21 |
| 4058 | D22 | Ar1 | D25 | Ar1 | D22 |
| 4059 | D23 | Ar1 | D25 | Ar1 | D23 |
| 4060 | D24 | Ar1 | D25 | Ar1 | D24 |
| 4061 | D26 | Ar1 | D25 | Ar1 | D26 |
| 4062 | D1 | Ar1 | D26 | Ar1 | D1 |
| 4063 | D2 | Ar1 | D26 | Ar1 | D2 |
| 4064 | D3 | Ar1 | D26 | Ar1 | D3 |
| 4065 | D4 | Ar1 | D26 | Ar1 | D4 |
| 4066 | D5 | Ar1 | D26 | Ar1 | D5 |
| 4067 | D6 | Ar1 | D26 | Ar1 | D6 |
| 4068 | D7 | Ar1 | D26 | Ar1 | D7 |
| 4069 | D8 | Ar1 | D26 | Ar1 | D8 |
| 4070 | D9 | Ar1 | D26 | Ar1 | D9 |
| 4071 | D10 | Ar1 | D26 | Ar1 | D10 |
| 4072 | D11 | Ar1 | D26 | Ar1 | D11 |
| 4073 | D12 | Ar1 | D26 | Ar1 | D12 |
| 4074 | D13 | Ar1 | D26 | Ar1 | D13 |
| 4075 | D14 | Ar1 | D26 | Ar1 | D14 |
| 4076 | D15 | Ar1 | D26 | Ar1 | D15 |
| 4077 | D16 | Ar1 | D26 | Ar1 | D16 |
| 4078 | D17 | Ar1 | D26 | Ar1 | D17 |
| 4079 | D18 | Ar1 | D26 | Ar1 | D18 |
| 4080 | D19 | Ar1 | D26 | Ar1 | D19 |
| 4081 | D20 | Ar1 | D26 | Ar1 | D20 |
| 4082 | D21 | Ar1 | D26 | Ar1 | D21 |
| 4083 | D22 | Ar1 | D26 | Ar1 | D22 |
| 4084 | D23 | Ar1 | D26 | Ar1 | D23 |
| 4085 | D24 | Ar1 | D26 | Ar1 | D24 |
| 4086 | D25 | Ar1 | D26 | Ar1 | D25 |
| 4087 | D21 | Ar1 | D1 | Ar1 | D21 |
| 4088 | D21 | Ar1 | D2 | Ar1 | D21 |
| 4089 | D21 | Ar1 | D3 | Ar1 | D21 |
| 4090 | D21 | Ar1 | D4 | Ar1 | D21 |
| 4091 | D21 | Ar1 | D5 | Ar1 | D21 |
| 4092 | D21 | Ar1 | D6 | Ar1 | D21 |
| 4093 | D21 | Ar1 | D7 | Ar1 | D21 |
| 4094 | D21 | Ar1 | D8 | Ar1 | D21 |
| 4085 | D21 | Ar1 | D9 | Ar1 | D21 |
| 4096 | D21 | Ar1 | D10 | Ar1 | D21 |
| 4097 | D21 | Ar1 | D11 | Ar1 | D21 |
| 4098 | D21 | Ar1 | D12 | Ar1 | D21 |
| 4099 | D21 | Ar1 | D13 | Ar1 | D21 |
| 4100 | D21 | Ar1 | D14 | Ar1 | D21 |
| 4101 | D21 | Ar1 | D15 | Ar1 | D21 |
| 4102 | D21 | Ar1 | D16 | Ar1 | D21 |
| 4103 | D21 | Ar1 | D17 | Ar1 | D21 |
| 4104 | D21 | Ar1 | D18 | Ar1 | D21 |
| 4105 | D21 | Ar1 | D19 | Ar1 | D21 |
| 4106 | D21 | Ar1 | D20 | Ar1 | D21 |
| 4107 | D21 | Ar1 | D22 | Ar1 | D21 |
| 4108 | D21 | Ar1 | D23 | Ar1 | D21 |
| 4109 | D21 | Ar1 | D24 | Ar1 | D21 |
| 4110 | D21 | Ar1 | D25 | Ar1 | D21 |
| 4111 | D21 | Ar1 | D26 | Ar1 | D21 |
| 4112 | D22 | Ar1 | D1 | Ar1 | D22 |
| 4113 | D22 | Ar1 | D2 | Ar1 | D22 |
| 4114 | D22 | Ar1 | D3 | Ar1 | D22 |
| 4115 | D22 | Ar1 | D4 | Ar1 | D22 |
| 4116 | D22 | Ar1 | D5 | Ar1 | D22 |
| 4117 | D22 | Ar1 | D6 | Ar1 | D22 |
| 4118 | D22 | Ar1 | D7 | Ar1 | D22 |
| 4119 | D22 | Ar1 | D8 | Ar1 | D22 |
| 4120 | D22 | Ar1 | D9 | Ar1 | D22 |
| 4121 | D22 | Ar1 | D10 | Ar1 | D22 |
| 4122 | D22 | Ar1 | D11 | Ar1 | D22 |
| 4123 | D22 | Ar1 | D12 | Ar1 | D22 |
| 4124 | D22 | Ar1 | D13 | Ar1 | D22 |
| 4125 | D22 | Ar1 | D14 | Ar1 | D22 |
| 4126 | D22 | Ar1 | D15 | Ar1 | D22 |
| 4127 | D22 | Ar1 | D16 | Ar1 | D22 |
| 4128 | D22 | Ar1 | D17 | Ar1 | D22 |
| 4129 | D22 | Ar1 | D18 | Ar1 | D22 |
| 4130 | D22 | Ar1 | D19 | Ar1 | D22 |
| 4131 | D22 | Ar1 | D26 | Ar1 | D22 |
| 4132 | D22 | Ar1 | D21 | Ar1 | D22 |
| 4133 | D22 | Ar1 | D23 | Ar1 | D22 |
| 4134 | D22 | Ar1 | D24 | Ar1 | D22 |
| 4135 | D22 | Ar1 | D25 | Ar1 | D22 |
| 4136 | D22 | Ar1 | D26 | Ar1 | D22 |
| 4137 | D23 | Ar1 | D1 | Ar1 | D23 |
| 4138 | D23 | Ar1 | D2 | Ar1 | D23 |
| 4139 | D23 | Ar1 | D3 | Ar1 | D23 |
| 4140 | D23 | Ar1 | D4 | Ar1 | D23 |
| 4141 | D23 | Ar1 | D5 | Ar1 | D23 |
| 4142 | D23 | Ar1 | D6 | Ar1 | D23 |
| 4143 | D23 | Ar1 | D7 | Ar1 | D23 |
| 4144 | D23 | Ar1 | D8 | Ar1 | D23 |
| 4145 | D23 | Ar1 | D9 | Ar1 | D23 |
| 4146 | D23 | Ar1 | D10 | Ar1 | D23 |
| 4147 | D23 | Ar1 | D11 | Ar1 | D23 |
| 4148 | D23 | Ar1 | D12 | Ar1 | D23 |
| 4149 | D23 | Ar1 | D13 | Ar1 | D23 |
| 4150 | D23 | Ar1 | D14 | Ar1 | D23 |
| 4151 | D23 | Ar1 | D15 | Ar1 | D23 |
| 4152 | D23 | Ar1 | D16 | Ar1 | D23 |
| 4153 | D23 | Ar1 | D17 | Ar1 | D23 |
| 4154 | D23 | Ar1 | D18 | Ar1 | D23 |
| 4155 | D23 | Ar1 | D19 | Ar1 | D23 |
| 4156 | D23 | Ar1 | D20 | Ar1 | D23 |
| 4157 | D23 | Ar1 | D21 | Ar1 | D23 |
| 4158 | D23 | Ar1 | D22 | Ar1 | D23 |
| 4159 | D23 | Ar1 | D24 | Ar1 | D23 |
| 4160 | D23 | Ar1 | D25 | Ar1 | D23 |
| 4161 | D23 | Ar1 | D26 | Ar1 | D23 |
| 4162 | D24 | Ar1 | D1 | Ar1 | D24 |
| 4163 | D24 | Ar1 | D2 | Ar1 | D24 |
| 4164 | D24 | Ar1 | D3 | Ar1 | D24 |
| 4165 | D24 | Ar1 | D4 | Ar1 | D24 |
| 4166 | D24 | Ar1 | D5 | Ar1 | D24 |
| 4167 | D24 | Ar1 | D6 | Ar1 | D24 |
| 4168 | D24 | Ar1 | D7 | Ar1 | D24 |
| 4169 | D24 | Ar1 | D8 | Ar1 | D24 |
| 4170 | D24 | Ar1 | D9 | Ar1 | D24 |
| 4171 | D24 | Ar1 | D10 | Ar1 | D24 |
| 4172 | D24 | Ar1 | D11 | Ar1 | D24 |
| 4173 | D24 | Ar1 | D12 | Ar1 | D24 |
| 4174 | D24 | Ar1 | D13 | Ar1 | D24 |
| 4175 | D24 | Ar1 | D14 | Ar1 | D24 |
| 4176 | D24 | Ar1 | D15 | Ar1 | D24 |
| 4177 | D24 | Ar1 | D16 | Ar1 | D24 |
| 4178 | D24 | Ar1 | D17 | Ar1 | D24 |
| 4179 | D24 | Ar1 | D18 | Ar1 | D24 |
| 4180 | D24 | Ar1 | D19 | Ar1 | D24 |
| 4181 | D24 | Ar1 | D20 | Ar1 | D24 |
| 4182 | D24 | Ar1 | D21 | Ar1 | D24 |
| 4183 | D24 | Ar1 | D22 | Ar1 | D24 |
| 4184 | D24 | Ar1 | D23 | Ar1 | D24 |
| 4185 | D24 | Ar1 | D25 | Ar1 | D24 |
| 4186 | D24 | Ar1 | D26 | Ar1 | D24 |
| 4187 | D25 | Ar1 | D1 | Ar1 | D25 |
| 4188 | D25 | Ar1 | D2 | Ar1 | D25 |
| 4189 | D25 | Ar1 | D3 | Ar1 | D25 |
| 4190 | D25 | Ar1 | D4 | Ar1 | D25 |
| 4191 | D25 | Ar1 | D5 | Ar1 | D25 |
| 4192 | D25 | Ar1 | D6 | Ar1 | D25 |
| 4193 | D25 | Ar1 | D7 | Ar1 | D25 |
| 4194 | D25 | Ar1 | D8 | Ar1 | D25 |
| 4195 | D25 | Ar1 | D9 | Ar1 | D25 |
| 4196 | D25 | Ar1 | D10 | Ar1 | D25 |
| 4197 | D25 | Ar1 | D11 | Ar1 | D25 |
| 4198 | D25 | Ar1 | D12 | Ar1 | D25 |
| 4199 | D25 | Ar1 | D13 | Ar1 | D25 |
| 4200 | D25 | Ar1 | D14 | Ar1 | D25 |
| 4201 | D25 | Ar1 | D15 | Ar1 | D25 |
| 4202 | D25 | Ar1 | D16 | Ar1 | D25 |
| 4203 | D25 | Ar1 | D17 | Ar1 | D25 |
| 4204 | D25 | Ar1 | D18 | Ar1 | D25 |
| 4205 | D25 | Ar1 | D19 | Ar1 | D25 |
| 4206 | D25 | Ar1 | D20 | Ar1 | D25 |
| 4207 | D25 | Ar1 | D21 | Ar1 | D25 |
| 4208 | D25 | Ar1 | D22 | Ar1 | D25 |
| 4209 | D25 | Ar1 | D23 | Ar1 | D25 |
| 4210 | D25 | Ar1 | D24 | Ar1 | D25 |
| 4211 | D25 | Ar1 | D26 | Ar1 | D25 |
| 4212 | D26 | Ar1 | D1 | Ar1 | D26 |
| 4213 | D26 | Ar1 | D2 | Ar1 | D26 |
| 4214 | D26 | Ar1 | D3 | Ar1 | D26 |
| 4215 | D26 | Ar1 | D4 | Ar1 | D26 |
| 4216 | D26 | Ar1 | D5 | Ar1 | D26 |
| 4217 | D26 | Ar1 | D6 | Ar1 | D26 |
| 4218 | D26 | Ar1 | D7 | Ar1 | D26 |
| 4219 | D26 | Ar1 | D8 | Ar1 | D26 |
| 4220 | D26 | Ar1 | D9 | Ar1 | D26 |
| 4221 | D26 | Ar1 | D10 | Ar1 | D26 |
| 4222 | D26 | Ar1 | D11 | Ar1 | D26 |
| 4223 | D26 | Ar1 | D12 | Ar1 | D26 |
| 4224 | D26 | Ar1 | D13 | Ar1 | D26 |
| 4225 | D26 | Ar1 | D14 | Ar1 | D26 |
| 4226 | D26 | Ar1 | D15 | Ar1 | D26 |
| 4227 | D26 | Ar1 | D16 | Ar1 | D26 |
| 4228 | D26 | Ar1 | D17 | Ar1 | D26 |
| 4229 | D26 | Ar1 | D18 | Ar1 | D26 |
| 4230 | D26 | Ar1 | D19 | Ar1 | D26 |
| 4231 | D26 | Ar1 | D20 | Ar1 | D26 |
| 4232 | D26 | Ar1 | D21 | Ar1 | D26 |
| 4233 | D26 | Ar1 | D22 | Ar1 | D26 |
| 4234 | D26 | Ar1 | D23 | Ar1 | D26 |
| 4235 | D26 | Ar1 | D24 | Ar1 | D26 |
| 4236 | D26 | Ar1 | D25 | Ar1 | D26 |
| 4237 | D21 | Ar1 | D1 | Ar1 | D1 |
| 4238 | D21 | Ar1 | D2 | Ar1 | D2 |
| 4239 | D21 | Ar1 | D3 | Ar1 | D3 |
| 4240 | D21 | Ar1 | D4 | Ar1 | D4 |
| 4241 | D21 | Ar1 | D5 | Ar1 | D5 |
| 4242 | D21 | Ar1 | D6 | Ar1 | D6 |
| 4243 | D21 | Ar1 | D7 | Ar1 | D7 |
| 4244 | D21 | Ar1 | D8 | Ar1 | D8 |
| 4245 | D21 | Ar1 | D9 | Ar1 | D9 |
| 4246 | D21 | Ar1 | D10 | Ar1 | D10 |
| 4247 | D21 | Ar1 | D11 | Ar1 | D11 |
| 4248 | D21 | Ar1 | D12 | Ar1 | D12 |
| 4249 | D21 | Ar1 | D13 | Ar1 | D13 |
| 4250 | D21 | Ar1 | D14 | Ar1 | D14 |
| 4251 | D21 | Ar1 | D15 | Ar1 | D15 |
| 4252 | D21 | Ar1 | D16 | Ar1 | D16 |
| 4253 | D21 | Ar1 | D17 | Ar1 | D17 |
| 4254 | D21 | Ar1 | D18 | Ar1 | D18 |
| 4255 | D21 | Ar1 | D19 | Ar1 | D19 |
| 4256 | D21 | Ar1 | D20 | Ar1 | D20 |
| 4257 | D21 | Ar1 | D22 | Ar1 | D22 |
| 4258 | D21 | Ar1 | D23 | Ar1 | D23 |
| 4259 | D21 | Ar1 | D24 | Ar1 | D24 |
| 4260 | D21 | Ar1 | D25 | Ar1 | D25 |
| 4261 | D21 | Ar1 | D26 | Ar1 | D26 |
| 4262 | D22 | Ar1 | D1 | Ar1 | D1 |
| 4263 | D22 | Ar1 | D2 | Ar1 | D2 |
| 4264 | D22 | Ar1 | D3 | Ar1 | D3 |
| 4265 | D22 | Ar1 | D4 | Ar1 | D4 |
| 4266 | D22 | Ar1 | D5 | Ar1 | D5 |
| 4267 | D22 | Ar1 | D6 | Ar1 | D6 |
| 4268 | D22 | Ar1 | D7 | Ar1 | D7 |
| 4269 | D22 | Ar1 | D8 | Ar1 | D8 |
| 4270 | D22 | Ar1 | D9 | Ar1 | D9 |
| 4271 | D22 | Ar1 | D10 | Ar1 | D10 |
| 4272 | D22 | Ar1 | D11 | Ar1 | D11 |
| 4273 | D22 | Ar1 | D12 | Ar1 | D12 |
| 4274 | D22 | Ar1 | D13 | Ar1 | D13 |
| 4275 | D22 | Ar1 | D14 | Ar1 | D14 |
| 4276 | D22 | Ar1 | D15 | Ar1 | D15 |
| 4277 | D22 | Ar1 | D16 | Ar1 | D16 |
| 4278 | D22 | Ar1 | D17 | Ar1 | D17 |
| 4279 | D22 | Ar1 | D18 | Ar1 | D18 |
| 4280 | D22 | Ar1 | D19 | Ar1 | D19 |
| 4281 | D22 | Ar1 | D20 | Ar1 | D20 |
| 4282 | D22 | Ar1 | D21 | Ar1 | D21 |
| 4283 | D22 | Ar1 | D23 | Ar1 | D23 |
| 4284 | D22 | Ar1 | D24 | Ar1 | D24 |
| 4285 | D22 | Ar1 | D25 | Ar1 | D25 |
| 4286 | D22 | Ar1 | D26 | Ar1 | D26 |
| 4287 | D23 | Ar1 | D1 | Ar1 | D1 |
| 4288 | D23 | Ar1 | D2 | Ar1 | D2 |
| 4289 | D23 | Ar1 | D3 | Ar1 | D3 |
| 4290 | D23 | Ar1 | D4 | Ar1 | D4 |
| 4291 | D23 | Ar1 | D5 | Ar1 | D5 |
| 4292 | D23 | Ar1 | D6 | Ar1 | D6 |
| 4293 | D23 | Ar1 | D7 | Ar1 | D7 |
| 4294 | D23 | Ar1 | D8 | Ar1 | D8 |
| 4295 | D23 | Ar1 | D9 | Ar1 | D9 |
| 4296 | D23 | Ar1 | D10 | Ar1 | D10 |
| 4297 | D23 | Ar1 | D11 | Ar1 | D11 |
| 4298 | D23 | Ar1 | D12 | Ar1 | D12 |
| 4299 | D23 | Ar1 | D13 | Ar1 | D13 |
| 4300 | D23 | Ar1 | D14 | Ar1 | D14 |
| 4301 | D23 | Ar1 | D15 | Ar1 | D15 |
| 4302 | D23 | Ar1 | D16 | Ar1 | D16 |
| 4303 | D23 | Ar1 | D17 | Ar1 | D17 |
| 4304 | D23 | Ar1 | D18 | Ar1 | D18 |
| 4305 | D23 | Ar1 | D19 | Ar1 | D19 |
| 4306 | D23 | Ar1 | D20 | Ar1 | D20 |
| 4307 | D23 | Ar1 | D21 | Ar1 | D21 |
| 4308 | D23 | Ar1 | D22 | Ar1 | D22 |
| 4309 | D23 | Ar1 | D24 | Ar1 | D24 |
| 4310 | D23 | Ar1 | D25 | Ar1 | D25 |
| 4311 | D23 | Ar1 | D26 | Ar1 | D26 |
| 4312 | D24 | Ar1 | D1 | Ar1 | D1 |
| 4313 | D24 | Ar1 | D2 | Ar1 | D2 |
| 4314 | D24 | Ar1 | D3 | Ar1 | D3 |
| 4315 | D24 | Ar1 | D4 | Ar1 | D4 |
| 4316 | D24 | Ar1 | D5 | Ar1 | D5 |
| 4317 | D24 | Ar1 | D6 | Ar1 | D6 |
| 4318 | D24 | Ar1 | D7 | Ar1 | D7 |
| 4319 | D24 | Ar1 | D8 | Ar1 | D8 |
| 4320 | D24 | Ar1 | D9 | Ar1 | D9 |
| 4321 | D24 | Ar1 | D10 | Ar1 | D10 |
| 4322 | D24 | Ar1 | D11 | Ar1 | D11 |
| 4323 | D24 | Ar1 | D12 | Ar1 | D12 |
| 4324 | D24 | Ar1 | D13 | Ar1 | D13 |
| 4325 | D24 | Ar1 | D14 | Ar1 | D14 |
| 4326 | D24 | Ar1 | D15 | Ar1 | D15 |
| 4327 | D24 | Ar1 | D16 | Ar1 | D16 |
| 4328 | D24 | Ar1 | D17 | Ar1 | D17 |
| 4329 | D24 | Ar1 | D18 | Ar1 | D18 |
| 4330 | D24 | Ar1 | D19 | Ar1 | D19 |
| 4331 | D24 | Ar1 | D20 | Ar1 | D20 |
| 4332 | D24 | Ar1 | D21 | Ar1 | D21 |
| 4333 | D24 | Ar1 | D22 | Ar1 | D22 |
| 4334 | D24 | Ar1 | D23 | Ar1 | D23 |
| 4335 | D24 | Ar1 | D25 | Ar1 | D25 |
| 4336 | D24 | Ar1 | D26 | Ar1 | D26 |
| 4337 | D25 | Ar1 | D1 | Ar1 | D1 |
| 4338 | D25 | Ar1 | D2 | Ar1 | D2 |
| 4339 | D25 | Ar1 | D3 | Ar1 | D3 |
| 4340 | D25 | Ar1 | D4 | Ar1 | D4 |
| 4341 | D25 | Ar1 | D5 | Ar1 | D5 |
| 4342 | D25 | Ar1 | D6 | Ar1 | D6 |
| 4343 | D25 | Ar1 | D7 | Ar1 | D7 |
| 4344 | D25 | Ar1 | D8 | Ar1 | D8 |
| 4345 | D25 | Ar1 | D9 | Ar1 | D9 |
| 4346 | D25 | Ar1 | D10 | Ar1 | D10 |
| 4347 | D25 | Ar1 | D11 | Ar1 | D11 |
| 4348 | D25 | Ar1 | D12 | Ar1 | D12 |
| 4349 | D25 | Ar1 | D13 | Ar1 | D13 |
| 4350 | D25 | Ar1 | D14 | Ar1 | D14 |
| 4351 | D25 | Ar1 | D15 | Ar1 | D15 |
| 4352 | D25 | Ar1 | D16 | Ar1 | D16 |
| 4353 | D25 | Ar1 | D17 | Ar1 | D17 |
| 4354 | D25 | Ar1 | D18 | Ar1 | D18 |
| 4355 | D25 | Ar1 | D19 | Ar1 | D19 |
| 4356 | D25 | Ar1 | D20 | Ar1 | D20 |
| 4357 | D25 | Ar1 | D21 | Ar1 | D21 |
| 4358 | D25 | Ar1 | D22 | Ar1 | D22 |
| 4359 | D25 | Ar1 | D23 | Ar1 | D23 |
| 4360 | D25 | Ar1 | D24 | Ar1 | D24 |
| 4361 | D25 | Ar1 | D26 | Ar1 | D26 |
| 4362 | D26 | Ar1 | D1 | Ar1 | D1 |
| 4363 | D26 | Ar1 | D2 | Ar1 | D2 |
| 4364 | D26 | Ar1 | D3 | Ar1 | D3 |
| 4365 | D26 | Ar1 | D4 | Ar1 | D4 |
| 4366 | D26 | Ar1 | D5 | Ar1 | D5 |
| 4367 | D26 | Ar1 | D6 | Ar1 | D6 |
| 4368 | D26 | Ar1 | D7 | Ar1 | D7 |
| 4369 | D26 | Ar1 | D8 | Ar1 | D8 |
| 4370 | D26 | Ar1 | D9 | Ar1 | D9 |
| 4371 | D26 | Ar1 | D10 | Ar1 | D10 |
| 4372 | D26 | Ar1 | D11 | Ar1 | D11 |
| 4373 | D26 | Ar1 | D12 | Ar1 | D12 |
| 4374 | D26 | Ar1 | D13 | Ar1 | D13 |
| 4375 | D26 | Ar1 | D14 | Ar1 | D14 |
| 4376 | D26 | Ar1 | D15 | Ar1 | D15 |
| 4377 | D26 | Ar1 | D16 | Ar1 | D16 |
| 4378 | D26 | Ar1 | D17 | Ar1 | D17 |
| 4379 | D26 | Ar1 | D18 | Ar1 | D18 |
| 4380 | D26 | Ar1 | D19 | Ar1 | D19 |
| 4381 | D26 | Ar1 | D20 | Ar1 | D20 |
| 4382 | D26 | Ar1 | D21 | Ar1 | D21 |
| 4383 | D26 | Ar1 | D22 | Ar1 | D22 |
| 4384 | D26 | Ar1 | D23 | Ar1 | D23 |
| 4385 | D26 | Ar1 | D24 | Ar1 | D24 |
| 4386 | D26 | Ar1 | D25 | Ar1 | D25 |
| 4387 | D1 | Ar1 | D21 | Ar1 | D21 |
| 4388 | D2 | Ar1 | D21 | Ar1 | D21 |
| 4389 | D3 | Ar1 | D21 | Ar1 | D21 |
| 4390 | D4 | Ar1 | D21 | Ar1 | D21 |
| 4391 | D5 | Ar1 | D21 | Ar1 | D21 |
| 4392 | D6 | Ar1 | D21 | Ar1 | D21 |
| 4393 | D7 | Ar1 | D21 | Ar1 | D21 |
| 4394 | D8 | Ar1 | D21 | Ar1 | D21 |
| 4395 | D9 | Ar1 | D21 | Ar1 | D21 |
| 4396 | D10 | Ar1 | D21 | Ar1 | D21 |
| 4397 | D11 | Ar1 | D21 | Ar1 | D21 |
| 4398 | D12 | Ar1 | D21 | Ar1 | D21 |
| 4399 | D13 | Ar1 | D21 | Ar1 | D21 |
| 4400 | D14 | Ar1 | D21 | Ar1 | D21 |
| 4401 | D15 | Ar1 | D21 | Ar1 | D21 |
| 4402 | D16 | Ar1 | D21 | Ar1 | D21 |
| 4403 | D17 | Ar1 | D21 | Ar1 | D21 |
| 4404 | D18 | Ar1 | D21 | Ar1 | D21 |
| 4405 | D19 | Ar1 | D21 | Ar1 | D21 |
| 4406 | D20 | Ar1 | D21 | Ar1 | D21 |
| 4407 | D22 | Ar1 | D21 | Ar1 | D21 |
| 4408 | D23 | Ar1 | D21 | Ar1 | D21 |
| 4409 | D24 | Ar1 | D21 | Ar1 | D21 |
| 4410 | D25 | Ar1 | D21 | Ar1 | D21 |
| 4411 | D26 | Ar1 | D21 | Ar1 | D21 |
| 4412 | D1 | Ar1 | D22 | Ar1 | D22 |
| 4413 | D2 | Ar1 | D22 | Ar1 | D22 |
| 4414 | D3 | Ar1 | D22 | Ar1 | D22 |
| 4415 | D4 | Ar1 | D22 | Ar1 | D22 |
| 4416 | D5 | Ar1 | D22 | Ar1 | D22 |
| 4417 | D6 | Ar1 | D22 | Ar1 | D22 |
| 4418 | D7 | Ar1 | D22 | Ar1 | D22 |
| 4419 | D8 | Ar1 | D22 | Ar1 | D22 |
| 4420 | D9 | Ar1 | D22 | Ar1 | D22 |
| 4421 | D10 | Ar1 | D22 | Ar1 | D22 |
| 4422 | D11 | Ar1 | D22 | Ar1 | D22 |
| 4423 | D12 | Ar1 | D22 | Ar1 | D22 |
| 4424 | D13 | Ar1 | D22 | Ar1 | D22 |
| 4425 | D14 | Ar1 | D22 | Ar1 | D22 |
| 4426 | D15 | Ar1 | D22 | Ar1 | D22 |
| 4427 | D16 | Ar1 | D22 | Ar1 | D22 |
| 4428 | D17 | Ar1 | D22 | Ar1 | D22 |
| 4429 | D18 | Ar1 | D22 | Ar1 | D22 |
| 4430 | D19 | Ar1 | D22 | Ar1 | D22 |
| 4431 | D20 | Ar1 | D22 | Ar1 | D22 |
| 4432 | D21 | Ar1 | D22 | Ar1 | D22 |
| 4433 | D23 | Ar1 | D22 | Ar1 | D22 |
| 4434 | D24 | Ar1 | D22 | Ar1 | D22 |
| 4435 | D25 | Ar1 | D22 | Ar1 | D22 |
| 4436 | D26 | Ar1 | D22 | Ar1 | D22 |
| 4437 | D1 | Ar1 | 023 | Ar1 | D23 |
| 4438 | D2 | Ar1 | D23 | Ar1 | D23 |
| 4439 | D3 | Ar1 | D23 | Ar1 | D23 |
| 4440 | D4 | Ar1 | D23 | Ar1 | D23 |
| 4441 | D5 | Ar1 | D23 | Ar1 | D23 |
| 4442 | D6 | Ar1 | D23 | Ar1 | D23 |
| 4443 | D7 | Ar1 | D23 | Ar1 | D23 |
| 4444 | D8 | Ar1 | D23 | Ar1 | D23 |
| 4445 | D9 | Ar1 | D23 | Ar1 | D23 |
| 4446 | D10 | Ar1 | D23 | Ar1 | D23 |
| 4447 | D11 | Ar1 | D23 | Ar1 | D23 |
| 4448 | D12 | Ar1 | D23 | Ar1 | D23 |
| 4449 | D13 | Ar1 | D23 | Ar1 | D23 |
| 4450 | D14 | Ar1 | D23 | Ar1 | D23 |
| 4451 | D15 | Ar1 | D23 | Ar1 | D23 |
| 4452 | D1e | Ar1 | D23 | Ar1 | D23 |
| 4453 | D17 | Ar1 | D23 | Ar1 | D23 |
| 4454 | D18 | Ar1 | D23 | Ar1 | D23 |
| 4455 | D19 | Ar1 | D23 | Ar1 | D23 |
| 4456 | D20 | Ar1 | D23 | Ar1 | D23 |
| 4457 | D21 | Ar1 | D23 | Ar1 | D23 |
| 4458 | D22 | Ar1 | D23 | Ar1 | D23 |
| 4459 | D24 | Ar1 | D23 | Ar1 | D23 |
| 4460 | D25 | Ar1 | D23 | Ar1 | D23 |
| 4461 | D26 | Ar1 | D23 | Ar1 | D23 |
| 4462 | D1 | Ar1 | D24 | Ar1 | D24 |
| 4463 | D2 | Ar1 | D24 | Ar1 | D24 |
| 4464 | D3 | Ar1 | D24 | Ar1 | D24 |
| 4465 | D4 | Ar1 | D24 | Ar1 | D24 |
| 4466 | D5 | Ar1 | D24 | Ar1 | D24 |
| 4467 | D6 | Ar1 | D24 | Ar1 | D24 |
| 4468 | D7 | Ar1 | D24 | Ar1 | D24 |
| 4469 | D8 | Ar1 | D24 | Ar1 | D24 |
| 4470 | D9 | Ar1 | D24 | Ar1 | D24 |
| 4471 | D10 | Ar1 | D24 | Ar1 | D24 |
| 4472 | D11 | Ar1 | D24 | Ar1 | D24 |
| 4473 | D12 | Ar1 | D24 | Ar1 | D24 |
| 4474 | D13 | Ar1 | D24 | Ar1 | D24 |
| 4475 | D14 | Ar1 | D24 | Ar1 | D24 |
| 4476 | D15 | Ar1 | D24 | Ar1 | D24 |
| 4477 | D16 | Ar1 | D24 | Ar1 | D24 |
| 4478 | D17 | Ar1 | D24 | Ar1 | D24 |
| 4479 | D18 | Ar1 | D24 | Ar1 | D24 |
| 4480 | D19 | Ar1 | D24 | Ar1 | D24 |
| 4481 | D20 | Ar1 | D24 | Ar1 | D24 |
| 4482 | D21 | Ar1 | D24 | Ar1 | D24 |
| 4483 | D22 | Ar1 | D24 | Ar1 | D24 |
| 4484 | D23 | Ar1 | D24 | Ar1 | D24 |
| 4485 | D25 | Ar1 | D24 | Ar1 | D24 |
| 4486 | D26 | Ar1 | D24 | Ar1 | D24 |
| 4487 | D1 | Ar1 | D25 | Ar1 | D25 |
| 4488 | D2 | Ar1 | D25 | Ar1 | D25 |
| 4489 | D3 | Ar1 | D25 | Ar1 | D25 |
| 4490 | D4 | Ar1 | D25 | Ar1 | D25 |
| 4491 | D5 | Ar1 | D25 | Ar1 | D25 |
| 4492 | D6 | Ar1 | D25 | Ar1 | D25 |
| 4493 | D7 | Ar1 | D25 | Ar1 | D25 |
| 4494 | D8 | Ar1 | D25 | Ar1 | D25 |
| 4495 | D9 | Ar1 | D25 | Ar1 | D25 |
| 4496 | D10 | Ar1 | D25 | Ar1 | D25 |
| 4497 | D11 | Ar1 | D25 | Ar1 | D25 |
| 4498 | D12 | Ar1 | D25 | Ar1 | D25 |
| 4499 | D13 | Ar1 | D25 | Ar1 | D25 |
| 4500 | D14 | Ar1 | D25 | Ar1 | D25 |
| 4501 | D15 | Ar1 | D25 | Ar1 | D25 |
| 4502 | D16 | Ar1 | D25 | Ar1 | D25 |
| 4503 | D17 | Ar1 | D25 | Ar1 | D25 |
| 4504 | D18 | Ar1 | D25 | Ar1 | D25 |
| 4505 | D19 | Ar1 | D25 | Ar1 | D25 |
| 4506 | D20 | Ar1 | D25 | Ar1 | D25 |
| 4507 | D21 | Ar1 | D25 | Ar1 | D25 |
| 4508 | D22 | Ar1 | D25 | Ar1 | D25 |
| 4509 | D23 | Ar1 | D25 | Ar1 | D25 |
| 4510 | D24 | Ar1 | D25 | Ar1 | D25 |
| 4511 | D26 | Ar1 | D25 | Ar1 | D25 |
| 4512 | D1 | Ar1 | D26 | Ar1 | D26 |
| 4513 | D2 | Ar1 | D26 | Ar1 | D26 |
| 4514 | D3 | Ar1 | D26 | Ar1 | D26 |
| 4515 | D4 | Ar1 | D26 | Ar1 | D26 |
| 4516 | D5 | Ar1 | D26 | Ar1 | D26 |
| 4517 | D6 | Ar1 | D26 | Ar1 | D26 |
| 4518 | D7 | Ar1 | D26 | Ar1 | D26 |
| 4519 | D8 | Ar1 | D26 | Ar1 | D26 |
| 4520 | D9 | Ar1 | D26 | Ar1 | D26 |
| 4521 | D10 | Ar1 | D26 | Ar1 | D26 |
| 4522 | D11 | Ar1 | D26 | Ar1 | D26 |
| 4523 | D12 | Ar1 | D26 | Ar1 | D26 |
| 4524 | D13 | Ar1 | D26 | Ar1 | D26 |
| 4525 | D14 | Ar1 | D26 | Ar1 | D26 |
| 4526 | D15 | Ar1 | D26 | Ar1 | D26 |
| 4527 | D16 | Ar1 | D26 | Ar1 | D26 |
| 4528 | D17 | Ar1 | D26 | Ar1 | D26 |
| 4529 | D18 | Ar1 | D26 | Ar1 | D26 |
| 4530 | D19 | Ar1 | D26 | Ar1 | D26 |
| 4531 | D20 | Ar1 | D26 | Ar1 | D26 |
| 4532 | D21 | Ar1 | D26 | Ar1 | D26 |
| 4533 | D22 | Ar1 | D26 | Ar1 | D26 |
| 4534 | D23 | Ar1 | D26 | Ar1 | D26 |
| 4535 | D24 | Ar1 | D26 | Ar1 | D26 |
| 4536 | D25 | Ar1 | D26 | Ar1 | D26 |

TABLE 2

| No. | R¹ | R² | R³ | R⁴ | R⁵ |
|---|---|---|---|---|---|
| 4537~4572 | D27~D62 | D1 | Ar1 | D1 | Ar1 |
| 4573~5868 | D1 | D27~D62 | Ar1 | D27~D62 | Ar1 |
| 5869~5904 | D1 | D1 | Ar1 | D27~D62 | Ar1 |
| 5905~7200 | D27~D62 | D1 | Ar1 | D27~D62 | Ar1 |
| 7201~8496 | D27~D62 | Ar1 | D1 | D27~D62 | Ar1 |
| 8497~9792 | D27~D62 | Ar1 | D27~D62 | D1 | Ar1 |
| 9793~9828 | D27~D62 | Ar1 | D1 | D1 | Ar1 |
| 9829~11124 | D1 | Ar1 | D27~D62 | D27~D62 | Ar1 |
| 11125~12420 | D27~D62 | Ar1 | D1 | Ar1 | D27~D62 |
| 12421~13716 | D1 | Ar1 | D27~D62 | Ar1 | D27~D62 |
| 13717~13752 | D1 | Ar1 | D1 | Ar1 | D27~D62 |
| 13753~13788 | D1 | Ar1 | D27~D62 | Ar1 | D1 |
| 13789~15084 | Ar1 | D27~D62 | D27~D62 | D1 | Ar1 |
| 15085~16380 | Ar1 | D1 | D27~D62 | D27~D62 | Ar1 |
| 16381~16416 | Ar1 | D1 | D1 | D27~D62 | Ar1 |
| 16417~16452 | Ar1 | D27~D62 | D1 | D1 | Ar1 |

The molecular weight of the compound represented by the general formula (1) is, for example, when an organic layer containing the compound represented by the general formula (1) is intended to be formed by an evaporation method and used, preferably 1500 or less, more preferably 1200 or less, even more preferably 1000 or less, further more preferably 900 or less. The lower limit of the molecular weight is a molecular weight of the smallest compound represented by the general formula (1).

The compound represented by the general formula (1) can be formed into a layer by a coating method, irrespective of the molecular weight thereof. According to a coating method, the compound having a relatively large molecular weight can be formed into a layer. The compound represented by the general formula (1) has an advantage that, among cyanobenzene compounds, the compound is readily soluble in an organic compound. Consequently, a coating method is readily applicable to the compound represented by the general formula (1) and, in addition, the compound can be purified to have an increased purity.

By applying the present invention, it is considered that a compound containing plural number of structures represented by the general formula (1) in the molecule can be used as a light emitting material.

For example, it is considered that a polymerizable group is previously introduced into the structure represented by the general formula (1), and the polymer formed by polymerizing the polymerizable group is used as a light emitting material. Specifically, it is considered that a monomer containing a polymerizable functional group in any of R¹ to R⁵ in the general formula (1) is prepared, and this is homo-polymerized, or is copolymerized with any other monomer to give a polymer having a repeating unit, and the polymer is used as a light emitting material. Or it is also considered that compounds each having the structure represented by the general formula (1) are coupled to give a dimer or a trimer, and these are used as a light emitting material.

Examples of the polymer having a repeating unit that contains the structure represented by the general formula (1) include polymers having a structure represented by the following general formula (3) or (4).

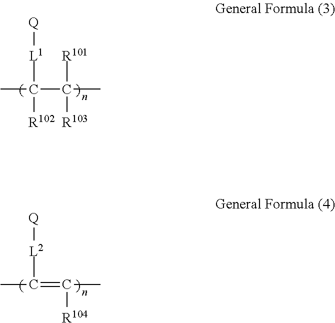

In the general formula (3) or (4), Q represents a group containing the structure represented by the general formula (1), L¹ and L² each represent a linking group. The carbon number of the linking group is preferably 0 to 20, more preferably 1 to 15, even more preferably 2 to 10. The linking group preferably has a structure represented by —X¹¹-L¹¹-. Here, X¹¹ represents an oxygen atom or a sulfur atom, and is preferably an oxygen atom. L¹¹ represents a linking group, and is preferably a substituted or unsubstituted alkylene group, or a substituted or unsubstituted arylene group, more preferably a substituted or unsubstituted alkylene group having 1 to 10 carbon atoms, or a substituted or unsubstituted phenylene group.

In the general formula (3) or (4), R¹⁰¹, R¹⁰², R¹⁰³ and R¹⁰⁴ each independently represent a substituent. Preferably, they each are a substituted or unsubstituted alkyl group having 1 to 6 carbon atoms, a substituted or unsubstituted alkoxy group having 1 to 6 carbon atoms, or a halogen atom, more preferably an unsubstituted alkyl group having 1 to 3 carbon atoms, an unsubstituted alkoxy group having 1 to 3 carbon atoms, a fluorine atom or a chlorine atom, even more preferably an unsubstituted alkyl group having 1 to 3 carbon atoms or an unsubstituted alkoxy group having 1 to 3 carbon atoms.

The linking group represented by L¹ and L² bonds to any of R¹ to R⁵ in formula (1) that constitutes Q. Two or more linking groups can bond to one Q to form a crosslinked structure or a network structure.

Examples of specific structures of the repeating unit include structures represented by the following formulae (5) to (8).

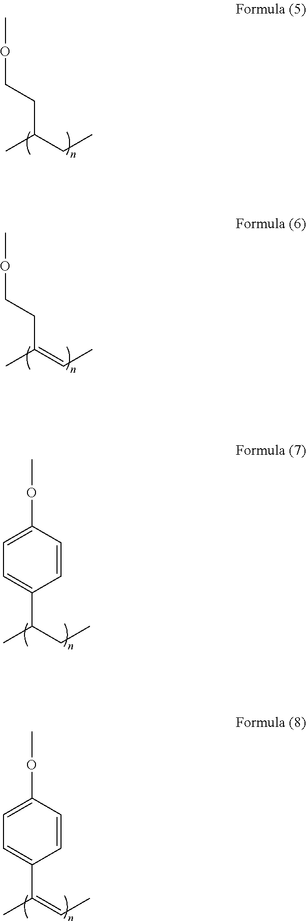

Polymers having a repeating unit that contains any of these formulae (5) to (8) can be synthesized by previously introducing a hydroxy group into any of R₁ to R⁵ in the general formula (1), then reacting the group serving as a linker with the following compound to thereby introduce a polymerizable group, and polymerizing the polymerizable group.

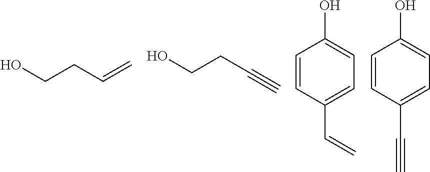

The polymer having a structure represented by the general formula (1) in the molecule can be a polymer having only a repeating unit that has the structure represented by the general formula (1), or can be a polymer containing a repeating unit that has any other structure. The repeating unit having the structure represented by the general formula (1) to be contained in the polymer may be a single kind or two or more kinds. The repeating unit not having the structure of the general formula (1) includes those derived from monomers used in general copolymerization. For example, it includes repeating units derived from monomers having an ethylenically unsaturated bond, such as ethylene or styrene.

### [Synthesis Method for Compound Represented by General Formula (1)]

The compound represented by the general formula (1) is a novel compound.

The compound represented by the general formula (1) can be synthesized by combining known reactions. For example, a starting substance of cyanobenzene trifluoride is reacted with a halide of an aromatic hydrocarbon in the presence of a catalyst to give a derivative having two aromatic hydrocarbon cyclic groups introduced in place of hydrogen atoms. The resultant derivative is reacted with carbazole in the presence of a catalyst to substitute a part of fluorine atoms with a carbazol-9-yl group, and is further reacted with a benzofuran ring-condensed carbazole to thereby substitute the remaining fluorine atoms with a benzofuran ring-condensed carbazol-9-yl group. In this process, the intended compound represented by the general formula (1) can be synthesized. For the specific conditions and the reaction process, reference can be made to Synthesis Examples given hereinunder. Also the other compounds represented by the general formula (1) can be synthesized according to the same process or using a known synthesis method.

### [Organic Light Emitting Device]

The compound represented by the general formula (1) of the present invention is useful as a light emitting material of organic light emitting devices. Therefore, the compound represented by the general formula (1) of the present invention can be effectively used as a light emitting material of organic light emitting devices. Also the compound represented by the general formula (1) of the present invention can be used as a host or an assist dopant.

The compound represented by the general formula (1) includes a delayed fluorescent material that emits delayed fluorescence. Specifically, the present invention also provides an invention of a delayed fluorescent material having a structure represented by the general formula (1), an invention using the compound represented by the general formula (1) as a delayed fluorescent material, and an invention of using the compound represented by the general formula (1) to emit delayed fluorescence. The organic light emitting device using a compound of the type as a light emitting material is characterized in that it emits delayed fluorescence and has a high emission efficiency. The principle is described with reference to an organic electroluminescent device as one example.

In an organic electroluminescent device, carriers are injected to the light-emitting material from both the positive and negative electrodes to form an excited light-emitting material, and the thus-excited light-emitting material emits light. In general, in the case of a carrier-injection-type organic electroluminescent device, among the formed excitons, 25% are excited to an excited singlet state, and the remaining 75% are excited to an excited triplet state. Accordingly, use of phosphorescence from the excited triplet state for light emission enjoys a high energy utilization efficiency. However, the excited triplet state has a long lifetime, and therefore may cause energy deactivation owing to saturation of the excited state or the interaction with the excitons in the excited triplet state, and accordingly, in general, the quantum yield of phosphorescence is often not high. On the other hand, a delayed fluorescent material is, after having undergone energy transition to an excited triplet state through reverse intersystem crossing, again subjected to reverse intersystem crossing to an excited singlet state by triplet-triplet annihilation or thermal energy absorption to thereby emit fluorescence. For an organic electroluminescent device, in particular, a thermally activated delayed fluorescent material with thermal energy absorption is considered to be especially useful. In the case where a delayed fluorescent material is used in an organic electroluminescent device, an excited singlet state exciton emits fluorescence as usual. On the other hand, an excited triplet state exciton undergoes reverse intersystem crossing to an excited singlet state by absorption of heat from the device to emit fluorescence. At that time, the emission is from an excited singlet state and is therefore at the same wavelength as that in fluorescence, but the lifetime of light generated by the reverse intersystem crossing from the excited triplet state to the excited singlet state (emission lifetime) is longer than that of ordinary fluorescence and is therefore observed as fluorescence delayed from ordinary fluorescence. This is defined as delayed fluorescence. Using such a thermally activated reverse intersystem crossing mechanism, fluorescence generation by a compound in an excited singlet state that can generally generate up to 25% fluorescence via thermal energy absorption after carrier injection can be increased 25% or more. When a compound capable of emitting strong fluorescence and delayed fluorescence even at a low temperature of lower than 100° C. is used, there can occur sufficient reverse intersystem crossing from an excited triplet state to an excited singlet state by the heat of a device to realize delayed fluorescence emission, and therefore in the case, the emission efficiency can be dramatically increased.

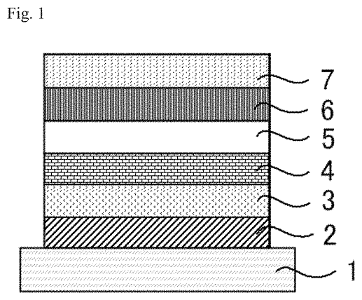

Using the compound represented by the general formula (1) of the present invention as a light emitting material in a light emitting layer, there can be provided an excellent organic light emitting device such as an organic photoluminescent device (organic PL device) and an organic electroluminescent device (organic EL device). An organic photoluminescent device is so configured as to have at least a light emitting layer formed on a substrate. Also an organic electroluminescent device is so configured as to have at least an anode, a cathode and an organic layer formed between the anode and the cathode. The organic layer includes at least a light emitting layer, and can be formed of a light emitting layer alone, or can have any other one or more organic layers than a light emitting layer. The other organic layers include a hole transport layer, a hole injection layer, an electron blocking layer, a hole blocking layer, an electron injection layer, an electron transport layer, and an exciton blocking layer. The hole transport layer may be a hole injection transport layer having a hole injection function, and the electron transport layer may be an electron injection transport layer having an electron injection function. A specific configuration example of an organic electroluminescent device is shown in FIG. 1. In FIG. 1, 1 is a substrate, 2 is an anode, 3 is a hole injection layer, 4 is a hole transport layer, 5 is a light emitting layer, 6 is an electron transport layer, and 7 is a cathode.

In the following, the constituent members and the layers of the organic electroluminescent device are described. The description of the substrate and the light emitting layer given below may apply to the substrate and the light emitting layer of an organic photoluminescent device.

### (Substrate)

The organic electroluminescent device of the invention is preferably supported by a substrate. The substrate is not particularly limited and may be those that have been commonly used in an organic electroluminescent device, and examples thereof used include those formed of glass, transparent plastics, quartz and silicon.

### (Anode)

The anode of the organic electroluminescent device used is preferably formed of, as an electrode material, a metal, an alloy, or an electroconductive compound each having a large work function (4 eV or more), or a mixture thereof. Specific examples of the electrode material include a metal, such as Au, and an electroconductive transparent material, such as CuI, indium tin oxide (ITO), SnO₂ and ZnO. A material that is amorphous and is capable of forming a transparent electroconductive film, such as IDIXO (In₂O—ZnO), may also be used. The anode may be formed in such a manner that the electrode material is formed into a thin film by such a method as vapor deposition or sputtering, and the film is patterned into a desired pattern by a photolithography method, or in the case where the pattern may not require high accuracy (for example, approximately 100 μm or more), the pattern may be formed with a mask having a desired shape on vapor deposition or sputtering of the electrode material. In alternative, in the case where a material capable of being coated, such as an organic electroconductive compound, is used, a wet film forming method, such as a printing method and a coating method, may be used. In the case where emitted light is to be taken out through the anode, the anode preferably has a transmittance of more than 10%, and the anode preferably has a sheet resistance of several hundred Ω/sq or less. The thickness of the anode may be generally selected from a range of from 10 to 1,000 nm, and preferably from 10 to 200 nm, while depending on the material used.

### (Cathode)

The cathode is preferably formed of as an electrode material a metal (which is referred to as an electron injection metal), an alloy, or an electroconductive compound, having a small work function (4 eV or less), or a mixture thereof. Specific examples of the electrode material include sodium, a sodium-potassium alloy, magnesium, lithium, a magnesium-cupper mixture, a magnesium-silver mixture, a magnesium-aluminum mixture, a magnesium-indium mixture, an aluminum-aluminum oxide (Al₂O₃) mixture, indium, a lithium-aluminum mixture, and a rare earth metal. Among these, a mixture of an electron injection metal and a second metal that is a stable metal having a larger work function than the electron injection metal, for example, a magnesium-silver mixture, a magnesium-aluminum mixture, a magnesium-indium mixture, an aluminum-aluminum oxide (Al₂O₃) mixture, a lithium-aluminum mixture, and aluminum, is preferred from the standpoint of the electron injection property and the durability against oxidation and the like. The cathode may be produced by forming the electrode material into a thin film by such a method as vapor deposition or sputtering. The cathode preferably has a sheet resistance of several hundred Ω/sq or less, and the thickness thereof may be generally selected from a range of from 10 nm to 5 μm, and preferably from 50 to 200 nm. For transmitting the emitted light, any one of the anode and the cathode of the organic electroluminescent device is preferably transparent or translucent, thereby enhancing the light emission luminance.

The cathode may be formed with the electroconductive transparent materials described for the anode, thereby forming a transparent or translucent cathode, and by applying the cathode, a device having an anode and a cathode, both of which have transmittance, may be produced.

### (Light Emitting Layer)

The light emitting layer is a layer in which holes and electrons injected from an anode and a cathode are recombined to give excitons for light emission, and a light emitting material can be used alone as the light emitting layer, but the light emitting layer contains alight emitting material and a host material. As the light emitting material, one or more selected from the compound group of the present invention represented by the general formula (1) can be used. In order that the organic electroluminescent device and the organic photoluminescent device of the present invention can express a high emission efficiency, it is important that the singlet exciton and the triplet exciton formed in the light emitting material can be confined in the light emitting material. Accordingly, it is preferable to add a host material to the light emitting layer in addition to the light emitting material therein. As the host material, an organic compound, of which at least any one of the excited singlet energy and the excited triplet energy is higher than that of the light emitting material in the present invention, can be used. As a result, the singlet exciton and the triplet exciton formed in the light emitting material in the present invention can be confined in the molecules of the light emitting material and the emission efficiency thereof can be sufficiently drawn. Needless-to-say, even though the singlet exciton and the triplet exciton could not be sufficiently confined, a high emission efficiency can be attained in some cases, and therefore, any host material capable of realizing a high emission efficiency can be used in the present invention with no specific limitation. In the organic light emitting device or the organic electroluminescent device of the present invention, emission occurs from the light emitting material of the present invention contained in the light emitting layer. The emission includes both fluorescent emission and delayed fluorescent emission. However, a part of emission can partially include emission from a host material.

The content of the compound represented by the general formula (1) in the light emitting layer is preferably less than 50% by weight. Further, the upper limit of the content of the compound represented by the general formula (1) is preferably less than 30% by weight, and the upper limit of the content can be, for example, less than 20% by weight, less than 10% by weight, less than 5% by weight, less than 3% by weight, less than 1% by weight, or less than 0.5% by weight. The lower limit is preferably 0.001% by weight or more, and can be, for example, more than 0.01% by weight, more than 0.1% by weight, more than 0.5% by weight or more than 1% by weight.

The host material in the light emitting layer is preferably an organic compound having a hole transporting capability or an electron transporting capability, capable of preventing the wavelength of light emission from being prolonged, and having a high glass transition temperature.

The compound represented by the general formula (1) can be used as a host material in the light emitting layer.

### (Injection Layer)

The injection layer is a layer that is provided between the electrode and the organic layer, for decreasing the driving voltage and enhancing the light emission luminance, and includes a hole injection layer and an electron injection layer, which may be provided between the anode and the light-emitting layer or the hole transport layer and between the cathode and the light emitting layer or the electron transport layer. The injection layer may be provided depending on necessity.

### (Blocking Layer)

The blocking layer is a layer that is capable of inhibiting charges (electrons or holes) and/or excitons present in the light-emitting layer from being diffused outside the light-emitting layer. The electron blocking layer may be disposed between the light-emitting layer and the hole transport layer, and inhibits electrons from passing through the light-emitting layer toward the hole transport layer. Similarly, the hole blocking layer may be disposed between the light-emitting layer and the electron transport layer, and inhibits holes from passing through the light-emitting layer toward the electron transport layer. The blocking layer may also be used for inhibiting excitons from being diffused outside the light-emitting layer. Thus, the electron blocking layer and the hole blocking layer each may also have a function as an exciton blocking layer. The term “the electron blocking layer” or “the exciton blocking layer” referred to herein is intended to include a layer that has both the functions of an electron blocking layer and an exciton blocking layer by one layer.

### (Hole Blocking Layer)

The hole blocking layer has the function of an electron transport layer in a broad sense. The hole blocking layer has a function of inhibiting holes from reaching the electron transport layer w % bile transporting electrons, and thereby enhances the recombination probability of electrons and holes in the light-emitting layer. As the material for the hole blocking layer, the material for the electron transport layer to be mentioned below may be used optionally.

### (Electron Blocking Layer)

The electron blocking layer has the function of transporting holes in a broad sense. The electron blocking layer has a function of inhibiting electrons from reaching the hole transport layer while transporting holes, and thereby enhances the recombination probability of electrons and holes in the light-emitting layer.

### (Exciton Blocking Layer)

The exciton blocking layer is a layer for inhibiting excitons generated through recombination of holes and electrons in the light-emitting layer from being diffused to the charge transporting layer, and the use of the layer inserted enables effective confinement of excitons in the light-emitting layer, and thereby enhances the light emission efficiency of the device. The exciton blocking layer may be inserted adjacent to the light-emitting layer on any of the side of the anode and the side of the cathode, and on both the sides. Specifically, in the case where the exciton blocking layer is present on the side of the anode, the layer may be inserted between the hole transport layer and the light-emitting layer and adjacent to the light-emitting layer, and in the case where the layer is inserted on the side of the cathode, the layer may be inserted between the light-emitting layer and the cathode and adjacent to the light-emitting layer. Between the anode and the exciton blocking layer that is adjacent to the light-emitting layer on the side of the anode, a hole injection layer, an electron blocking layer and the like may be provided, and between the cathode and the exciton blocking layer that is adjacent to the light-emitting layer on the side of the cathode, an electron injection layer, an electron transport layer, a hole blocking layer and the like may be provided. In the case where the blocking layer is provided, preferably, at least one of the excited singlet energy and the excited triplet energy of the material used as the blocking layer is higher than the excited singlet energy and the excited triplet energy of the light-emitting layer, respectively, of the light-emitting material.

### (Hole Transport Layer)

The hole transport layer is formed of a hole transport material having a function of transporting holes, and the hole transport layer may be provided as a single layer or plural layers.

The hole transport material has one of injection or transporting property of holes and blocking property of electrons, and may be any of an organic material and an inorganic material. Examples of known hole transport materials that may be used herein include a triazole derivative, an oxadiazole derivative, an imidazole derivative, a carbazole derivative, an indolocarbazole derivative, a polyarylalkane derivative, a pyrazoline derivative, a pyrazolone derivative, a phenylenediamine derivative, an arylamine derivative, an amino-substituted chalcone derivative, an oxazole derivative, a styrylanthracene derivative, a fluorenone derivative, a hydrazone derivative, a stilbene derivative, a silazane derivative, an aniline copolymer and an electroconductive polymer oligomer, particularly a thiophene oligomer. Among these, a porphyrin compound, an aromatic tertiary amine compound and a styrylamine compound are preferably used, and an aromatic tertiary amine compound is more preferably used.

### (Electron Transport Layer)

The electron transport layer is formed of a material having a function of transporting electrons, and the electron transport layer may be a single layer or may be formed of plural layers.

The electron transport material (often also acting as a hole blocking material) may have a function of transmitting the electrons injected from a cathode to a light-emitting layer. The electron transport layer usable here includes, for example, nitro-substituted fluorene derivatives, diphenylquinone derivatives, thiopyran dioxide derivatives, carbodiimides, fluorenylidenemethane derivatives, anthraquinodimethane and anthrone derivatives, oxadiazole derivatives, etc. Further, thiadiazole derivatives derived from the above-mentioned oxadiazole derivatives by substituting the oxygen atom in the oxadiazole ring with a sulfur atom, and quinoxaline derivatives having a quinoxaline ring known as an electron-attractive group are also usable as the electron transport material. Further, polymer materials prepared by introducing these materials into the polymer chain, or having these material in the polymer main chain are also usable.

In producing the organic electroluminescent device, the compound represented by the general formula (1) can be used not only in one organic layer (for example, electron transporting layer) but also in plural organic layers. In that case, the compound represented by the general formula (1) used in each organic layer can be the same as or different from each other. For example, the compound represented by the general formula (1) can be used in the above-mentioned injection layer, blocking layer, hole blocking layer, electron broking layer, exciton blocking layer and hole transport layer, in addition to the electron transport layer and the light emitting layer. The method for forming these layers is not specifically limited, and the layers can be formed in any of a dry process or a wet process.

Preferred materials for use for the organic electroluminescent device are concretely exemplified below. However, the materials for use in the present invention are not limitatively interpreted by the following exemplary compounds. Compounds, even though exemplified as materials having a specific function, can also be used as other materials having any other function.

First, preferred compounds that can be used also as a host material in the light emitting layer are mentioned below.

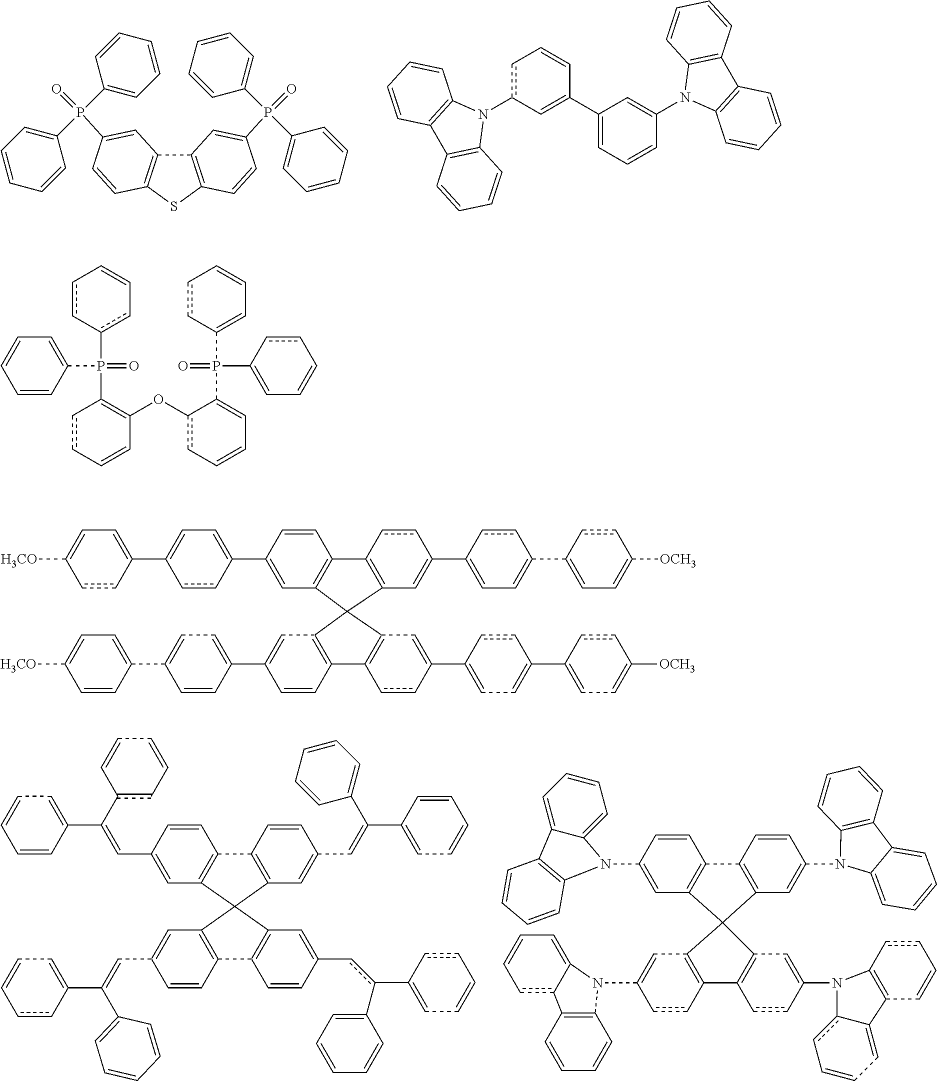

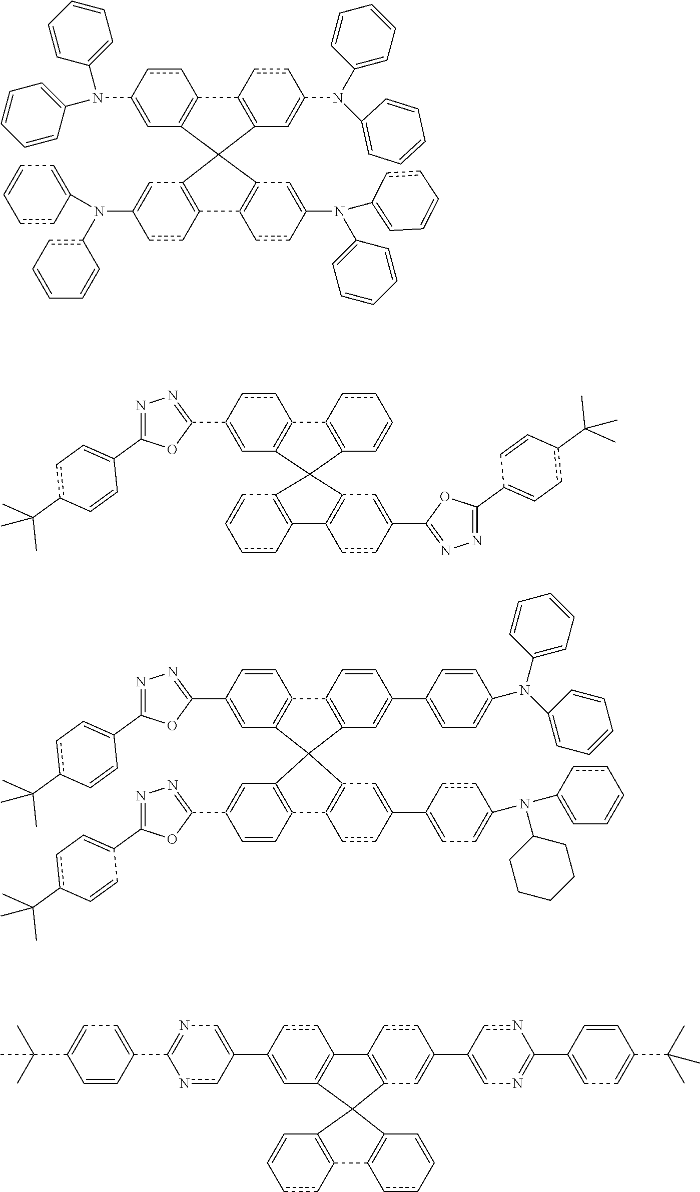

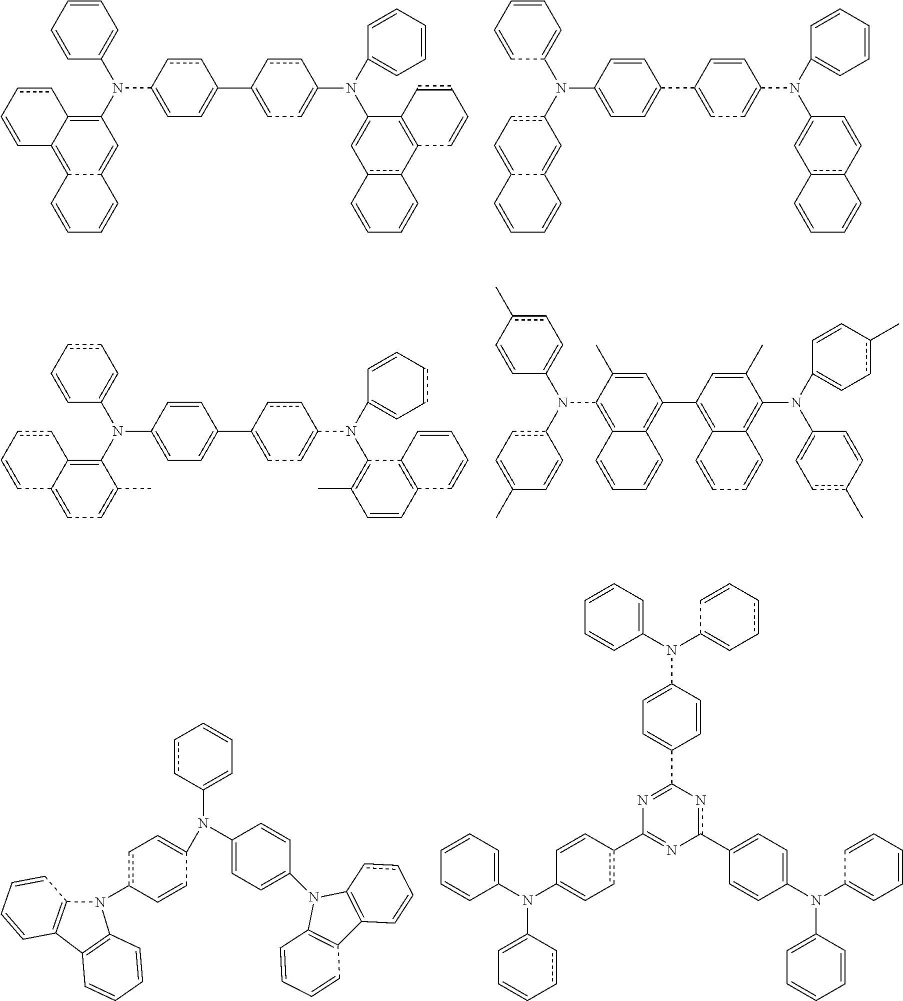

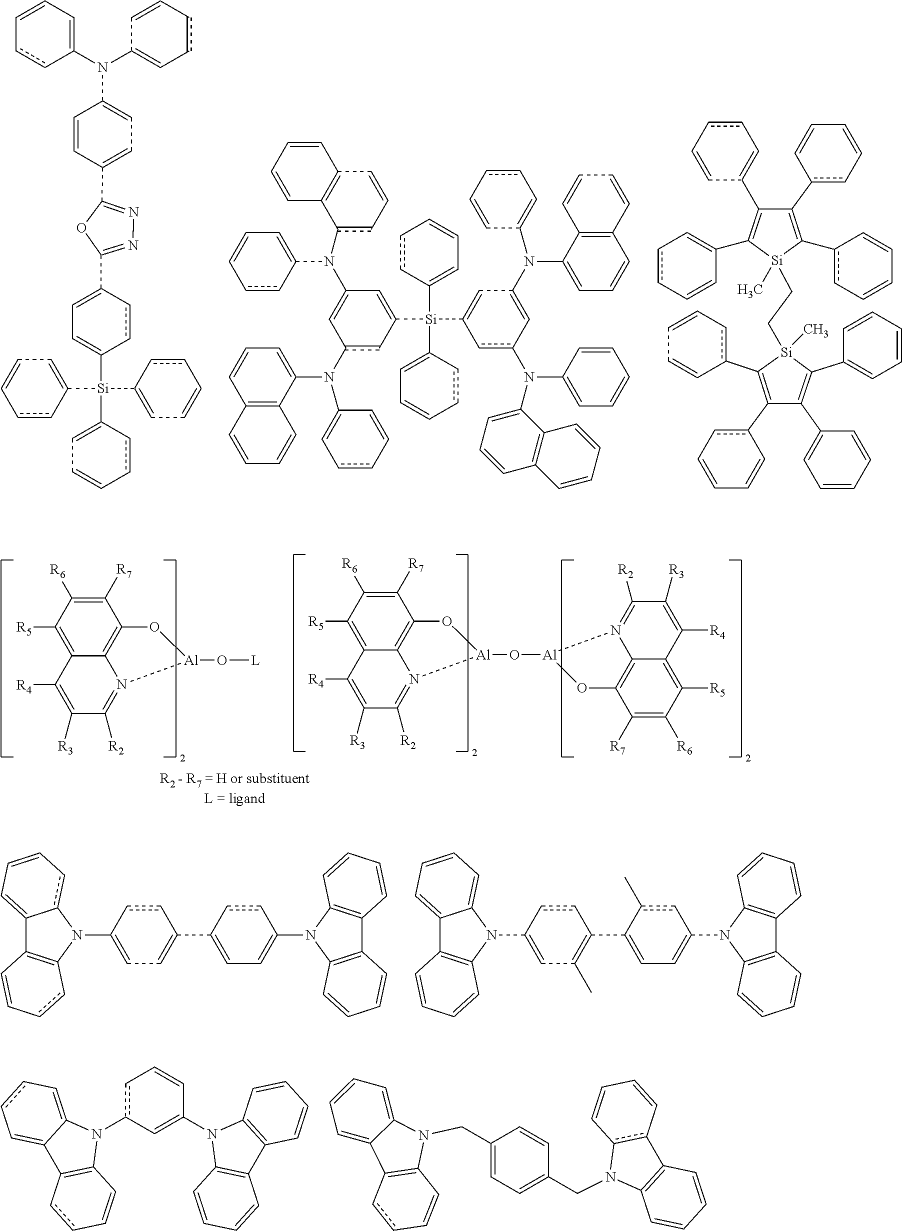

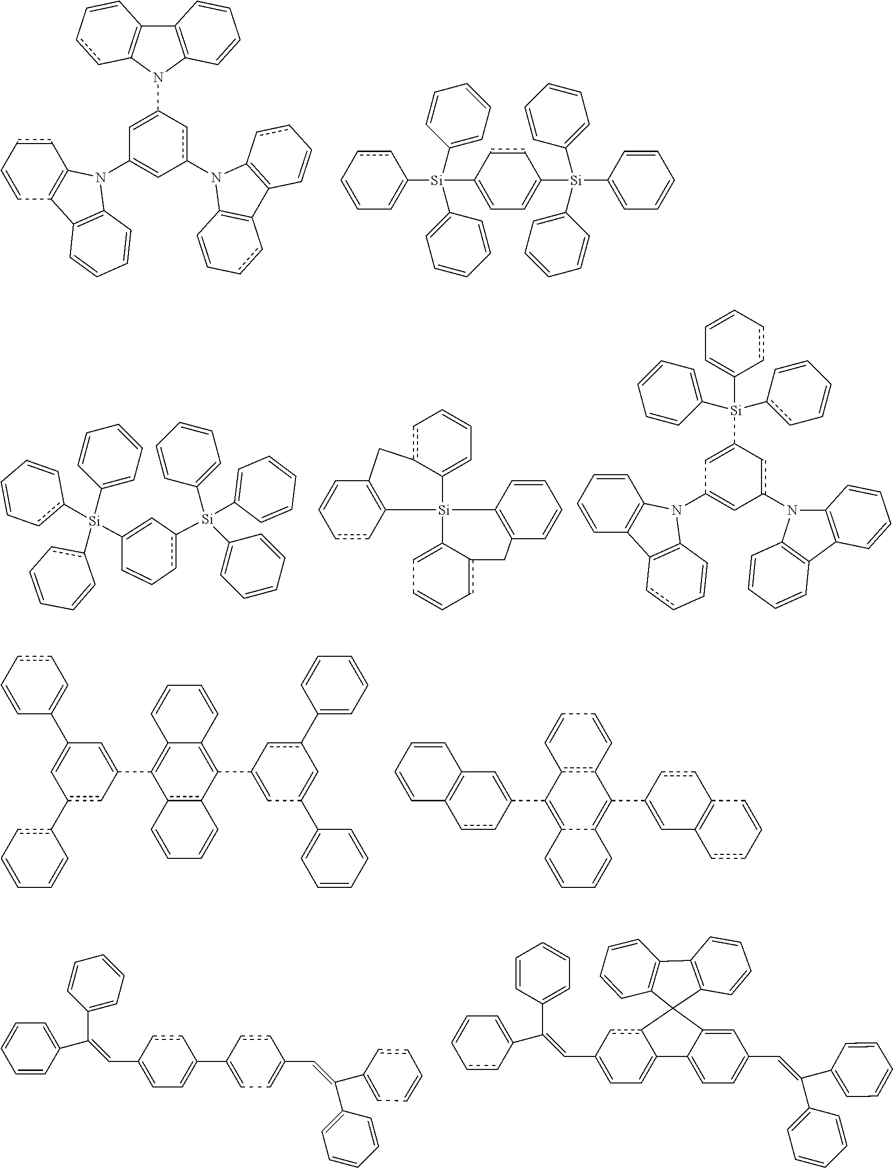

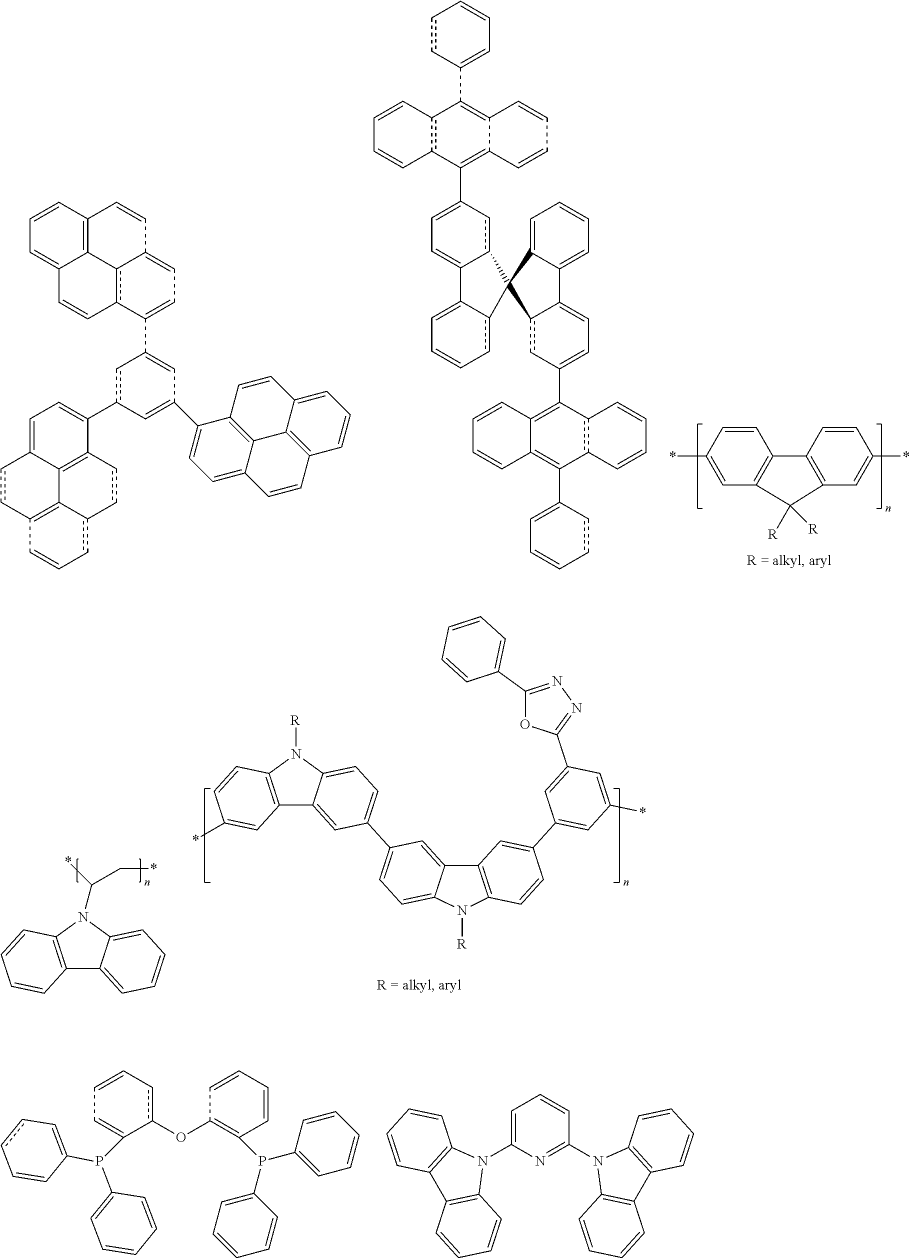

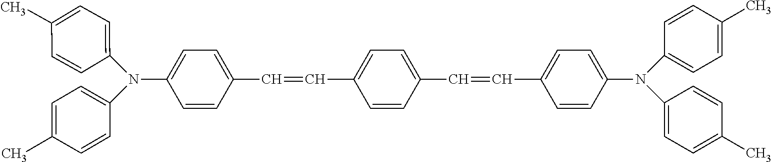

Next, examples of preferred compounds for use as a hole injection material are mentioned below.

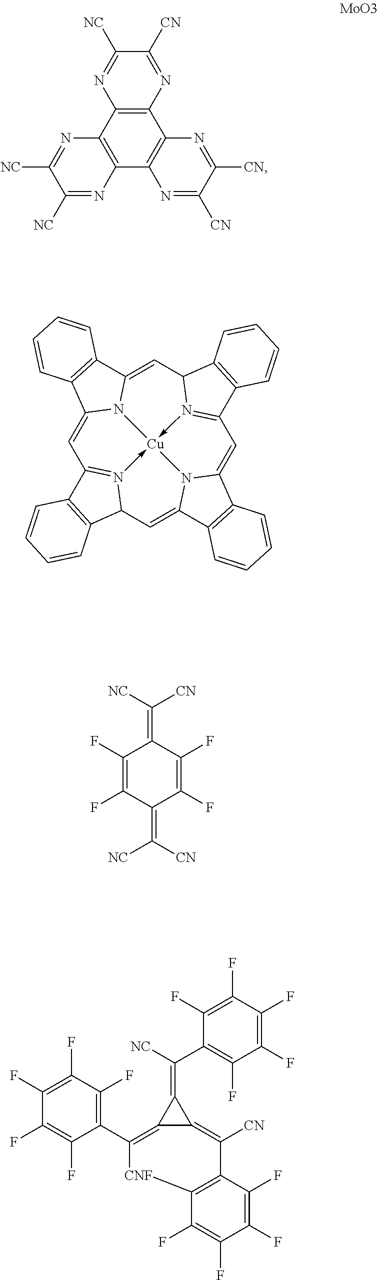

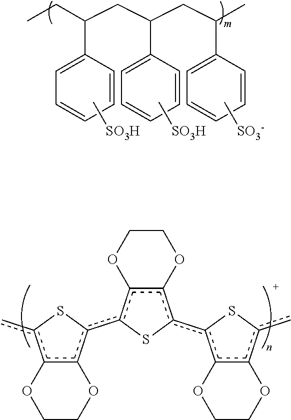

Next, examples of preferred compounds for use as a hole transport material are mentioned below.

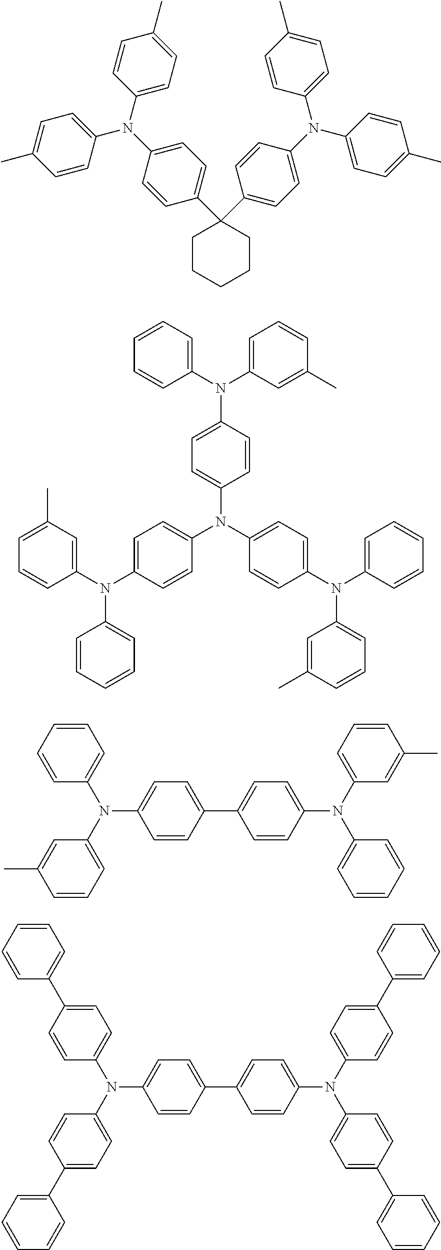

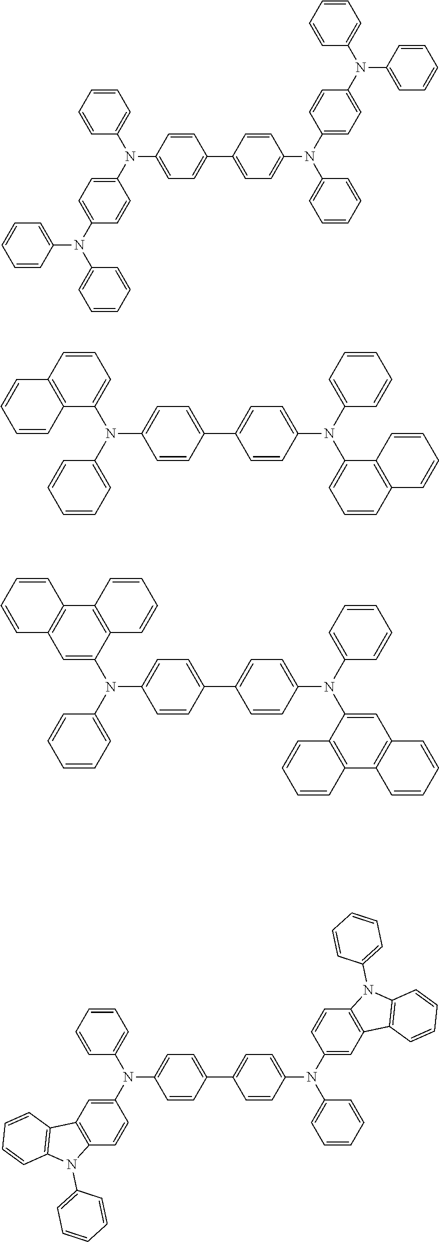 [SMILES: c1ccc(N(c2ccccc2)c2ccc(N(c3ccccc3)c3ccc(-c4ccc(N(c5ccccc5)c5ccc(N(c6ccccc6)c6ccccc6)cc5)cc4)cc3)cc2)cc1 ; c1ccc(N(c2ccc(-c3ccc(N(c4ccccc4)c4cccc5ccccc45)cc3)cc2)c2cccc3ccccc23)cc1 ; c1ccc(N(c2ccc(-c3ccc(N(c4ccccc4)c4cc5ccccc5c5ccccc45)cc3)cc2)c2cc3ccccc3c3ccccc23)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

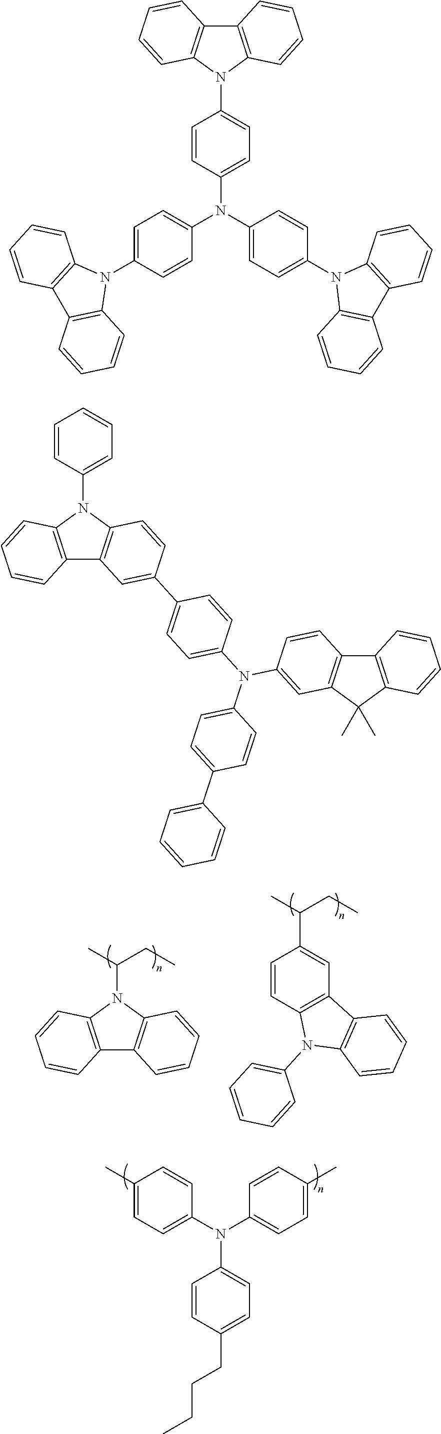

Next, examples of preferred compounds for use as an electron blocking material are mentioned below.

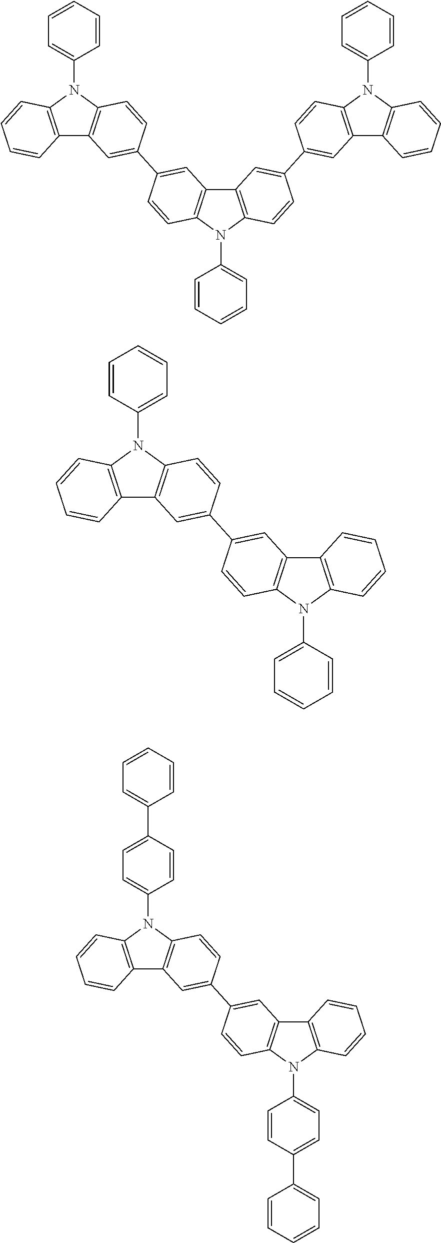 [SMILES: c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1 ; c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

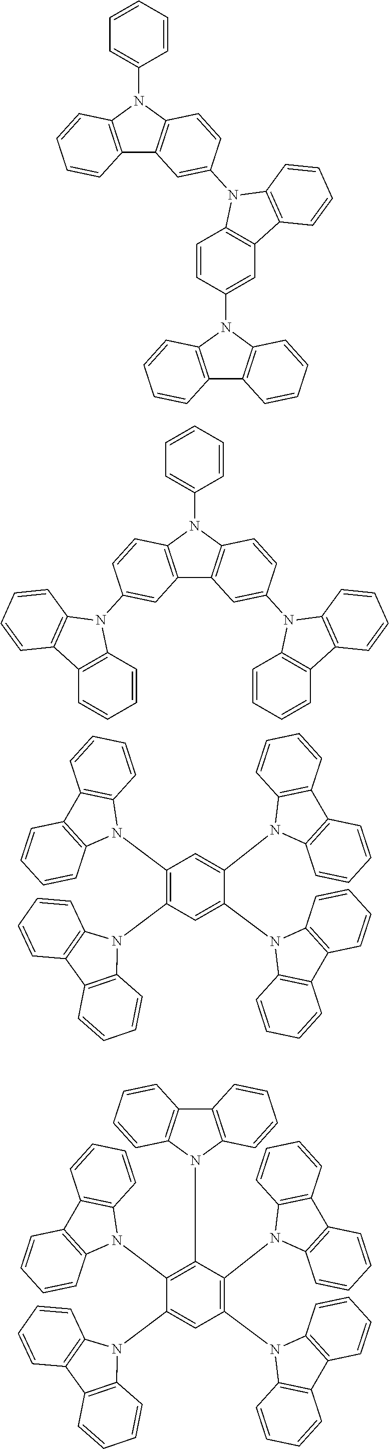

Next, examples of preferred compounds for use as a hole blocking material are mentioned below.

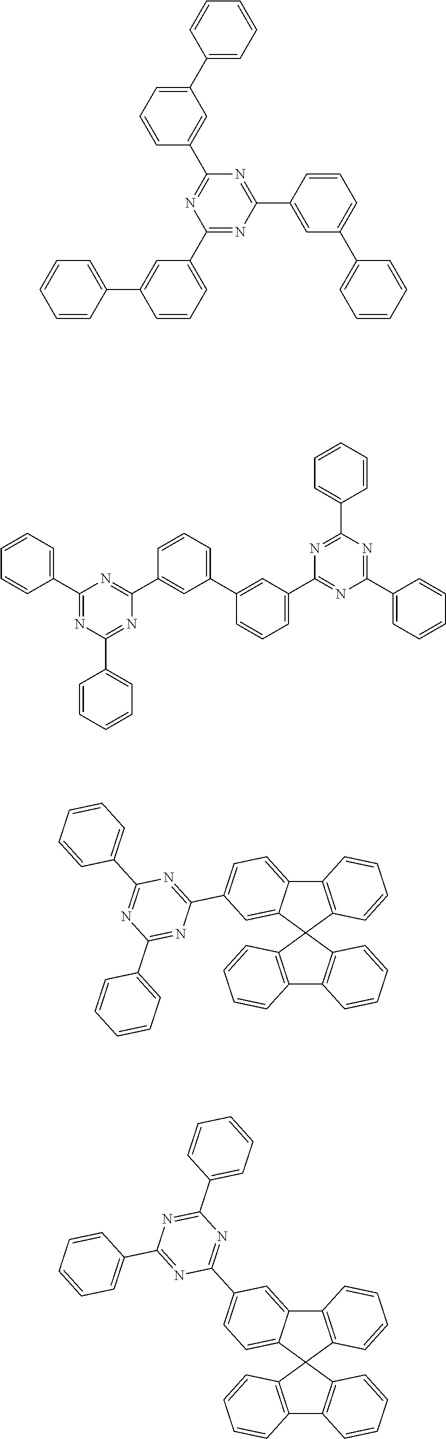 [SMILES: c1ccc(-c2cccc(-c3nc(-c4cccc(-c5ccccc5)c4)nc(-c4cccc(-c5ccccc5)c4)n3)c2)cc1 ; c1ccc(-c2nc(-c3ccccc3)nc(-c3cccc(-c4cccc(-c5nc(-c6ccccc6)nc(-c6ccccc6)n5)c4)c3)n2)cc1 ; c1ccc(-c2nc(-c3ccccc3)nc(-c3ccc4c(c3)C3(c5ccccc5-c5ccccc53)c3ccccc3-4)n2)cc1 ; c1ccc(-c2nc(-c3ccccc3)nc(-c3ccc4c(c3)-c3ccccc3C43c4ccccc4-c4ccccc43)n2)cc1]

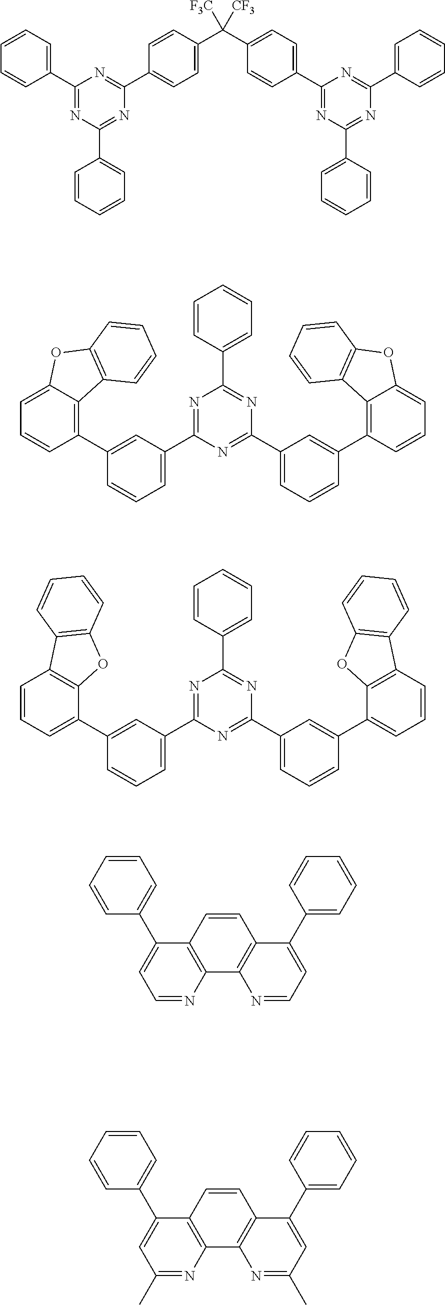 [SMILES: FC(F)(F)C(c1ccc(-c2nc(-c3ccccc3)nc(-c3ccccc3)n2)cc1)(c1ccc(-c2nc(-c3ccccc3)nc(-c3ccccc3)n2)cc1)C(F)(F)F ; c1ccc(-c2nc(-c3cccc(-c4cccc5oc6ccccc6c45)c3)nc(-c3cccc(-c4cccc5oc6ccccc6c45)c3)n2)cc1 ; c1ccc(-c2nc(-c3cccc(-c4cccc5c4oc4ccccc45)c3)nc(-c3cccc(-c4cccc5c4oc4ccccc45)c3)n2)cc1 ; c1ccc(-c2ccnc3c2ccc2c(-c4ccccc4)ccnc23)cc1 ; Cc1cc(-c2ccccc2)c2ccc3c(-c4ccccc4)cc(C)nc3c2n1]

Next, examples of preferred compounds for use as an electron transport material are mentioned below.

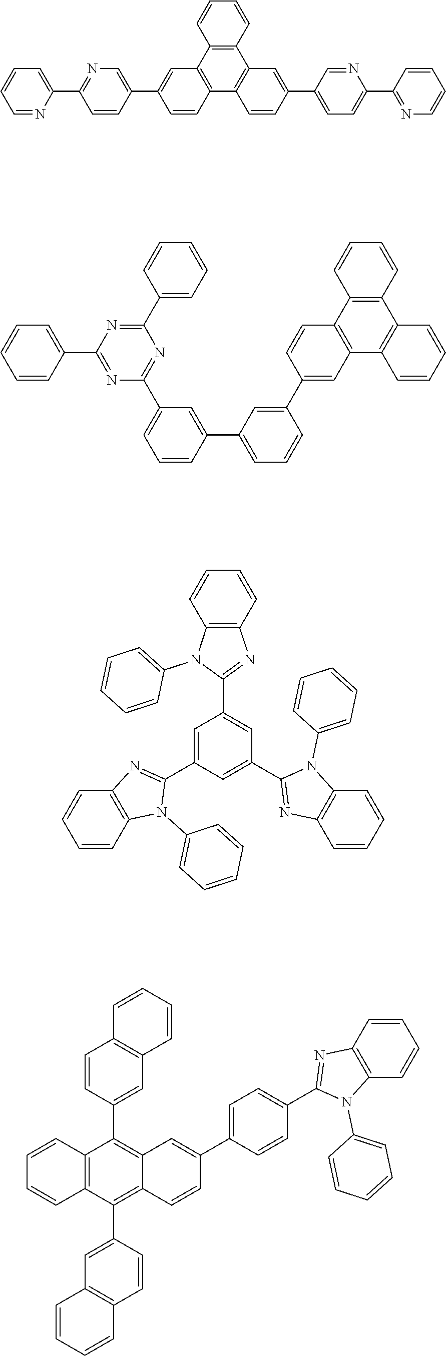 [SMILES: c1ccc(-c2ccc(-c3ccc4c5ccc(-c6ccc(-c7ccccn7)nc6)cc5c5ccccc5c4c3)cn2)nc1 ; c1ccc(-c2nc(-c3ccccc3)nc(-c3cccc(-c4cccc(-c5ccc6c7ccccc7c7ccccc7c6c5)c4)c3)n2)cc1 ; c1ccc(-n2c(-c3cc(-c4nc5ccccc5n4-c4ccccc4)cc(-c4nc5ccccc5n4-c4ccccc4)c3)nc3ccccc32)cc1 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1]

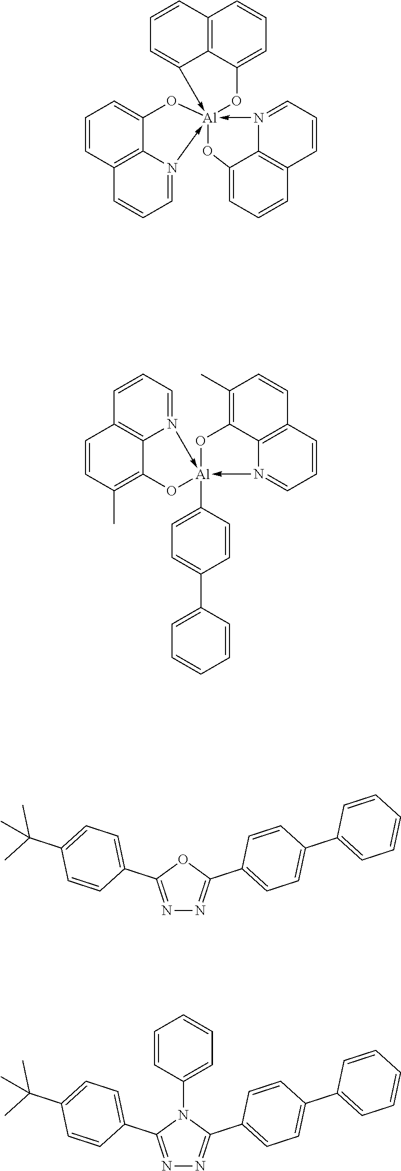

Next, examples of preferred compounds for use as an electron injection material are mentioned below.

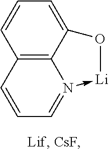

Further, preferred compounds for use as additional materials are mentioned below. For example, these are considered to be added as a stabilization material.

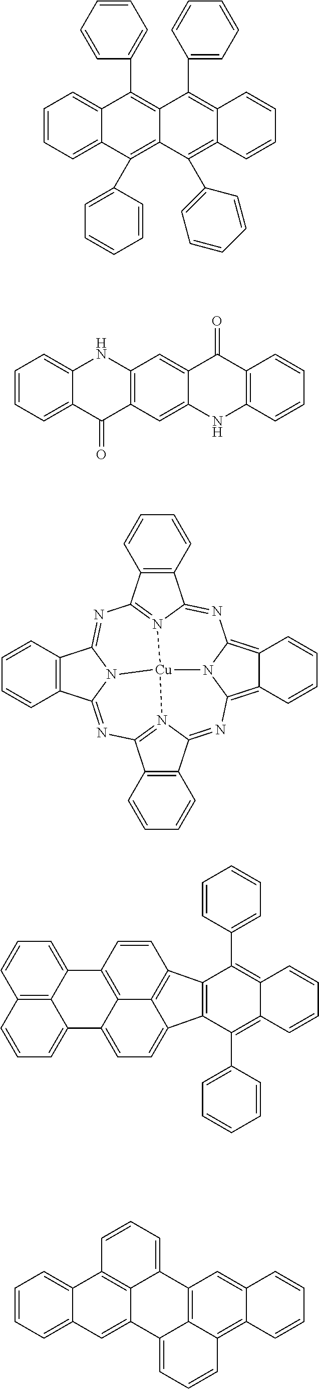

The organic electroluminescent device produced according to the above-mentioned method emits light on application of an electric field between the anode and the cathode of the device. In this case, when the light emission is caused by the excited singlet energy, light having a wavelength that corresponds to the energy level thereof may be confirmed as fluorescent light and delayed fluorescent light. When the light emission is caused by the excited triplet energy, light having a wavelength that corresponds to the energy level thereof may be confirmed as phosphorescent light. The normal fluorescent light has a shorter light emission lifetime than the delayed fluorescent light, and thus the light emission lifetime may be distinguished between the fluorescent light and the delayed fluorescent light.

On the other hand, regarding phosphorescence, the excited triplet energy of ordinary organic compounds like the compound of the present invention is unstable, the rate constant of thermal deactivation thereof is large, and the rate constant of light emission thereof is small, and therefore such ordinary organic compounds immediately deactivate and could emit little phosphorescence at room temperature. For measuring the excited triplet energy of ordinary organic compounds, measurement is possible by observing the emission from the compounds under the condition of extreme low temperatures.

The organic electroluminescent device of the invention may be applied to any of a single device, a structure with plural devices disposed in an array, and a structure having anodes and cathodes disposed in an X-Y matrix. According to the present invention in which the compound represented by the general formula (1) is incorporated in the light emitting layer, an organic light emitting device having a greatly improved light emission efficiency can be provided. The organic light emitting device such as the organic electroluminescent device of the present invention may be applied to a further wide range of purposes. For example, an organic electroluminescent display apparatus may be produced with the organic electroluminescent device of the invention, and for the details thereof, reference may be made to S. Tokito, C. Adachi and H. Murata, “Yuki EL Display” (Organic EL Display) (Ohmsha, Ltd.). In particular, the organic electroluminescent device of the invention may be applied to organic electroluminescent illumination and backlight which are highly demanded.

### EXAMPLES

The features of the present invention will be described more specifically with reference to Synthesis Examples and Examples given below. The materials, processes, procedures and the like shown below may be appropriately modified unless they deviate from the substance of the invention. Accordingly, the scope of the invention is not construed as being limited to the specific examples shown below. Hereinunder the light emission characteristics were evaluated using a source meter (available from Keithley Corporation, Keithley 2400), a semiconductor parameter analyzer (available from Agilent Technology Corporation, E5273A), a light power meter apparatus (available from Newport Corporation, 1930C), an optical spectroscope (available from Ocean Optics Corporation, USB 2000), a spectroradiometer (available from Topcon Corporation, SR-3) and a streak camera (available from Hamamatsu Photonics K.K., C4334).

### (Synthesis Example 1) Synthesis of Compound 26

### (1-1) Synthesis of Intermediate 1

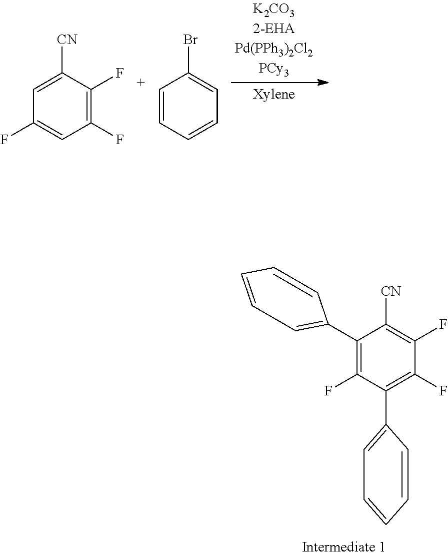

In a nitrogen stream atmosphere, 0.29 g (2.00 mmol) of 2-ethylhexanoic acid and 6.28 g (40.0 mmol) of bromobenzene were added to a xylene (30 mL) solution of 1.57 g (10.0 mmol) of 2,3,5-trifluorobenzonitrile, 4.15 g (30.0 mmol) of potassium carbonate, 0.25 g (0.03 mmol) of Pd(PPh₃)₂Cl₂ and 0.25 g (0.09 mmol) of tricyclohexyl phosphine, and stirred at 80° C. for 24 hours and at 100° C. for 24 hours. The reaction solution was restored to room temperature, an aqueous saturated ammonium chloride solution was added and extracted with ethyl acetate. The solvent was evaporated away, and the residue was purified by silica gel column chromatography (hexane/chloroform=9/1 to 7/3) to give 2.20 g (7.13 mmol, yield 73%) of a while solid of Intermediate 1.

¹H NMR (500 MHz, CDCl₃, δ): 7.60-7.45 (m, 10H)

ASAP MS spectrometry: C₁₉H₁₀F₃N: theoretical 309.08, found 310.10

### (1-2) Synthesis of Intermediate 2

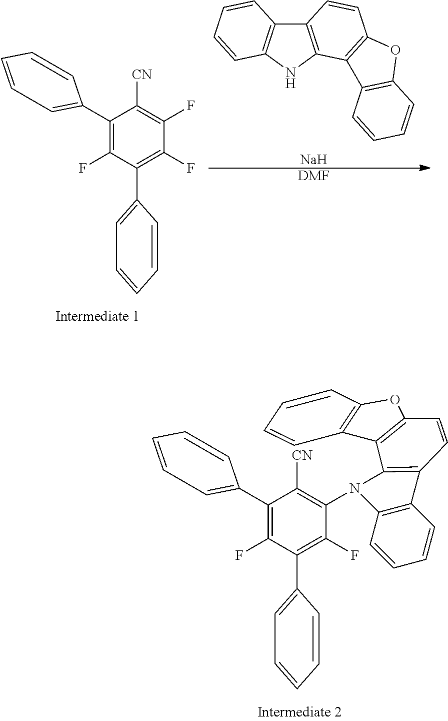

In a nitrogen stream atmosphere, dimethylformamide (40 mL) was added to 0.35 g (14.7 mmol) of sodium hydride and 1.26 g (4.90 mmol) of 12H-[3,2-a]-benzofluorocarbazole, and stirred at room temperature for 1 hour. The reaction mixture was added to a dimethylformamide (20 mL) solution of the Intermediate 1 (2.17 g, 7.00 mmol) at 0′C, and stirred for 10 hours. Subsequently, this was heated up to 10° C. and stirred for 6 hours. The reaction solution was quenched with water added thereto, then extracted with ethyl acetate, and the organic layer was washed with water and saturated saline water, dried with anhydrous magnesium sulfate, and then the solvent was evaporated away. The crude product was purified by silica gel column chromatography (hexane/toluene=2.19) to give 0.93 g (1.70 mmol, yield 35%) of a pale yellow solid of Intermediate 2.

¹H NMR (500 MHz, CDCl₃, δ): 8.23 (d, J=8.7 Hz, 1H), 8.17 (d, J=6.9 Hz, 1H), 7.65 (d, J=8.7 Hz, 1H), 7.63 (d, J=8.7 Hz, 1H), 7.57-7.38 (m, 13H), 7.25-7.14 (m, 1H), 7.10 (t, J=7.8 Hz, 1H), 6.13 (d, J=7.8 Hz, 1H)

ASAP MS spectrometry: C₃₇H₂₀F₂N₂O: theoretical 546.15, found 547.65

### (1-3) Synthesis of Compound 26

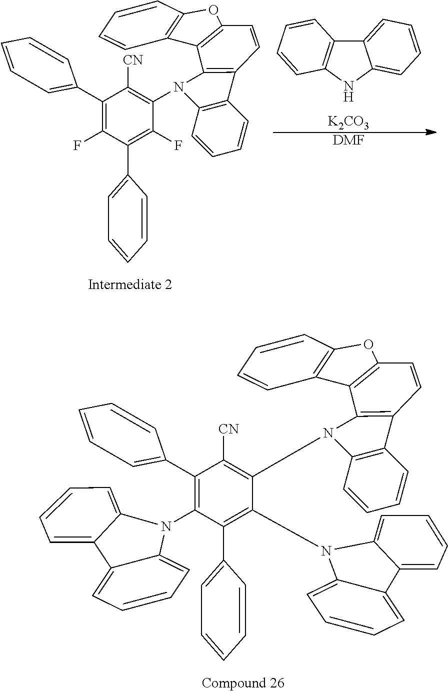

In a nitrogen stream atmosphere, dimethylformamide (26 mL) was added to 1.04 g (6.24 mmol) of carbazole and 1.08 g (7.80 mmol) of potassium carbonate, and stirred at room temperature for 2 hours. 1.42 g (2.60 mmol) of Intermediate 2 was added to the reaction mixture, and stirred at 100° C. for 16 hours. The reaction solution was restored to room temperature, water was added and the precipitate was filtered out. The residue on the filter was washed with methanol, and dried in vacuum. The residue was purified by silica gel column chromatography (hexane/toluene=2/5) to give 1.71 g (2.03 mmol, yield 76%) of a pale yellow solid of Compound 26.

¹H NMR (500 MHz, CDCl₃, δ): 7.87 (t, J=8.7 Hz, 2H), 7.81 (d, J=8.7 Hz, 1H), 7.72-7.65 (m, 2H), 7.58-7.50 (m, 3H), 7.47-7.42 (m, 2H), 7.35 (d, J=8.2 Hz, 2H), 7.33-7.31 (m, 2H), 7.27-7.08 (m, 7H), 7.06-6.87 (m, 7H), 6.69 (t, J=7.6 Hz, 1H), 6.69-6.58 (br, 2H), 6.55 (d. J=8.2 Hz, 1H), 6.46 (t, J=7.6 Hz, 1H), 6.39-6.29 (br, 2H), 6.27 (t, J=8.2 Hz, 1H)

ASAP MS spectrometry. C₆₁1H₃₆N₄O theoretical 840.29, found 841.51

### (Synthesis Example 2) Synthesis of Compound 1626

### (2-1) Synthesis of Intermediate 3

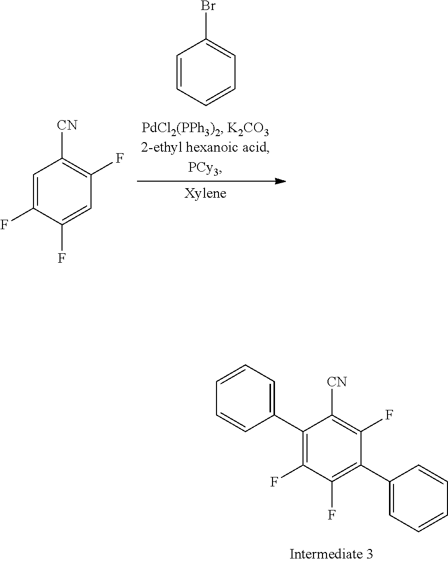 [SMILES: Brc1ccccc1 ; N#Cc1cc(F)c(F)cc1F ; N#Cc1c(F)c(-c2ccccc2)c(F)c(F)c1-c1ccccc1]

In a nitrogen stream atmosphere, 0.40 g (0.57 mmol) of dichlorobis(triphenylphosphine)palladium(II) was added to a xylene solution (10 mL) of 3.0 g (19.1 mmol) of 2,4,5-trifluorobenzonitrile, 8.99 g (57.3 mmol) of bromobenzene, 7.92 g (57.3 mmol) of potassium carbonate, 0.28 g (1.91 mmol) of 2-ethylhexanoic acid, and 0.48 g (1.72 mmol) of tricyclohexyl phosphine, and stirred overnight at 100° C. The reaction solution was restored to room temperature, then quenched with water added thereto, and extracted with chloroform. The solvent was evaporated away with an evaporator, and the residue was purified by silica gel column chromatography (hexane/chloroform) to give 4.99 g (16.13 mmol, yield 84.5%) of a white solid of Intermediate 3.

¹H NMR (400 MHz, CDCl₃, 6): 7.51-7.54 (m, 4H), 7.56-7.61 (m, 6H)

ASAP MS spectrometry: C₁₉H₁₀F₃N: theoretical 309.1, found 310.1

### (2-2) Synthesis of Intermediate 4

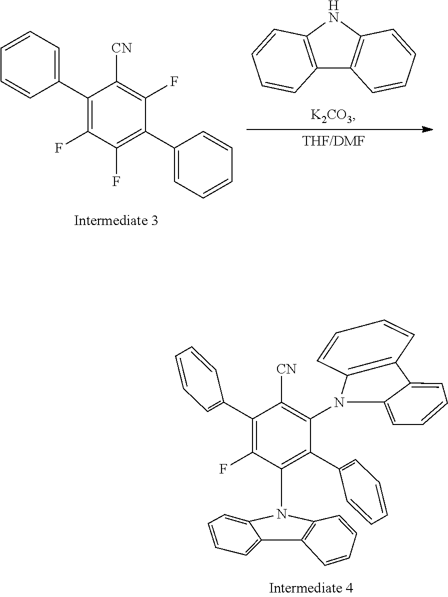 [SMILES: N#Cc1c(F)c(-c2ccccc2)c(F)c(F)c1-c1ccccc1 ; c1ccc2c(c1)[nH]c1ccccc12 ; N#Cc1c(-c2ccccc2)c(F)c(-n2c3ccccc3c3ccccc32)c(-c2ccccc2)c1-n1c2ccccc2c2ccccc21]

In a nitrogen stream atmosphere, 3.24 g (19.4 mmol) of 9H-carbazole, 2.68 g (19.4 mmol) of potassium carbonate, and a solution of dimethylformamide (25 mL) and tetrahydrofuran (25 mL) in which 3.00 g (9.7 mmol) of Intermediate 3 had been dissolved were stirred overnight at 70° C. The mixture was restored to room temperature, then quenched with a saturated ammonium chloride solution added thereto, and extracted with chloroform. The solvent was evaporated away with an evaporator, and the residue was purified by silica gel column chromatography (hexane/ethyl acetate=1/9) to give 0.50 g (0.83 mmol, yield 8.5%) of Intermediate 4.

¹H NMR (400 MHz, CDCl₃, δ): 6.46 (t, J=8.4 Hz, 2H), 6.57 (d, J=6.4 Hz, 4H), 7.17 (t, J=6.8 Hz, 4H), 7.23 (t, J=7.2 Hz, 4H), 7.36 (dd. J=8.8 Hz, 4H), 7.54-7.57 (m, 2H), 7.74 (d, J=6.8 Hz, 2H), 7.99-8.02 (m, 4H),

ASAP MS spectrometry: C₄₃H₂₆N₃: theoretical 603.2, found 604.2

### (2-3) Synthesis of Compound 1626

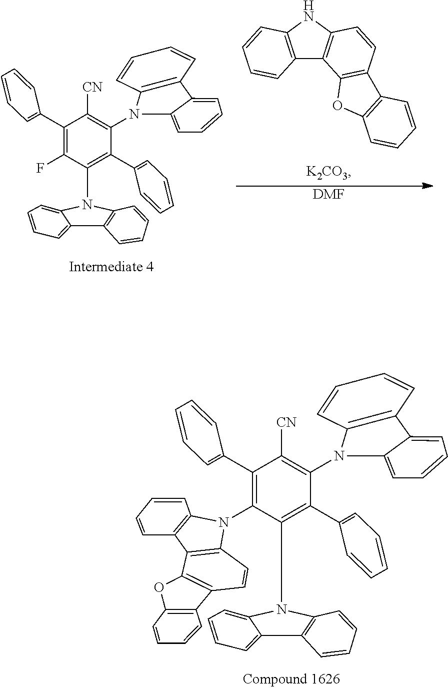

In a nitrogen stream atmosphere, a dimethylformamide solution (10 mL), in which 0.51 g (1.99 mmol) of 5H-benzofuro[3,2-c]carbazole, 0.27 g (1.99 mmol) of potassium carbonate and 0.80 g (1.33 mmol) of Intermediate 4 had been dissolved, was stirred overnight at 120° C. The mixture was restored to room temperature, then quenched with a saturated ammonium chloride solution added thereto, and extracted with chloroform. The solvent was evaporated away with an evaporator, and the residue was purified by silica gel column chromatography (hexane/ethyl acetate=1/9) to give 0.95 g (1.13 mmol, yield 85.0%) of Compound 1626.

¹H NMR (400 MHz, CDCl₃, δ): 6.39 (t, J=8.0 Hz, 2H), 6.52 (t, J=8.0 Hz, 1H), 6.60 (d, J=8.0 Hz, 2H), 6.81-6.89 (m, 3H), 6.91-6.98 (m, 4H), 7.01-7.16 (m, 6H), 7.23-7.47 (m, 12H), 7.54 (t. J=6.8 Hz, 2H), 7.85 (d, J=6.8 Hz, 1H), 7.99-8.05 (m, 3H)

ASAP MS spectrometry: C₆₁H₃₆N₄O: theoretical 840.3. found 841.3

### (Synthesis Example 3) Synthesis of Compound 3387

### (3-1) Synthesis of Intermediate 5

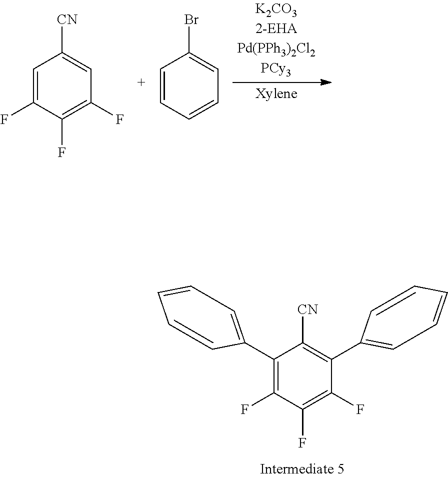

In a nitrogen stream atmosphere, 0.55 g (3.82 mmol) of 2-ethylhexanoic acid and 12.0 g (76.4 mmol) of bromobenzene were added to a xylene (57 mL) solution of 3.00 g (19.1 mmol) of 3,4,5-trifluorobenzonitrile, 7.92 g (57.0 mmol) of potassium carbonate, 0.42 g (0.57 mmol) of Pd(PPh₃)₂Cl₂ and 0.48 g (1.72 mmol) of tricyclohexyl phosphine, and stirred at 80° C. for 7 hours and at 10° C. for 16 hours. The reaction solution was restored to room temperature, an aqueous saturated ammonium chloride solution was added and extracted with ethyl acetate. The solvent was evaporated away and the residue was purified by silica gel column chromatography (hexane/dichloromethane=1/1, hexane/ethyl acetate=3/17) to give 4.34 g (14.0 mmol, yield 73%) of a white solid of Intermediate 5.

¹H NMR (500 MHz, CDCl₃, δ): 7.53-7.43 (m, 10H)

ASAP MS spectrometry: C₁₉H₁₀F₃N: theoretical 309.08, found 310.04

### (3-2) Synthesis of Intermediate 6

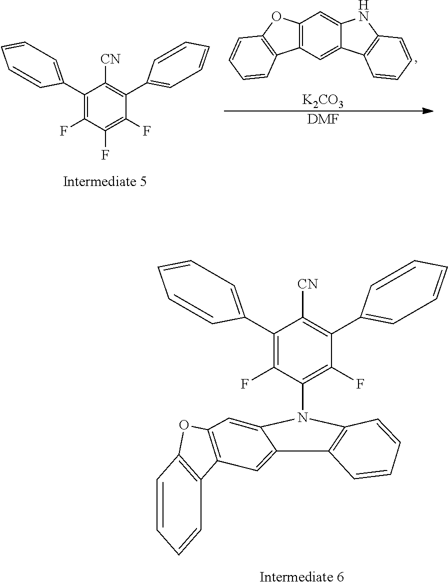

In a nitrogen stream atmosphere, dimethylformamide (15 mL) was added to 1.00 g (3.88 mmol) of 7H-benzofuro[2,3-b]carbazole, 1.50 g (4.85 mmol) of Intermediate 5 and 1.01 g (7.28 mmol) of potassium carbonate, and stirred at 100° C. for 13 hours. The reaction solution was quenched with water added thereto, and the suspension was filtered. The crude product was purified by silica gel column chromatography (hexane/dichloromethane=2/2) to give 2.07 g (3.79 mmol, yield 78%) of Intermediate 6.

¹H NMR (500) MHz, CDCl₃, δ): 8.64 (s, 1H), 8.23 (d, J=7.5 Hz, 1H), 8.06 (d, J=7.5 Hz, 1H), 7.64-7.35 (m, 16H), 7.25-7.24 (m, 1H)

ASAP MS spectrometry: C₃₇H₂₀F₂N₂O theoretical 546.15, found 547.24

### (3-3) Synthesis of Compound 3387

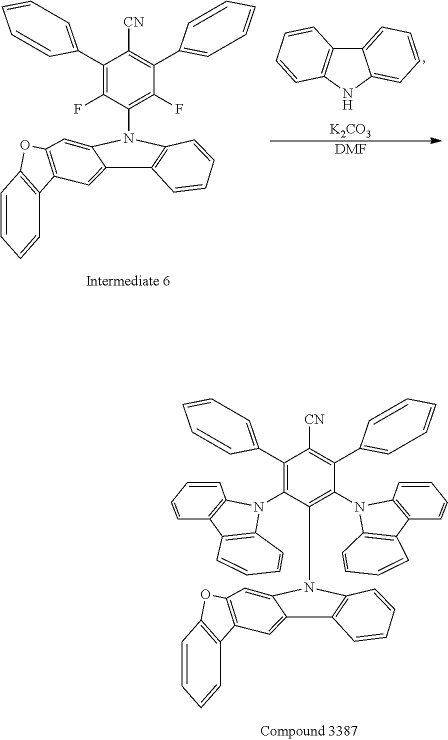

In a nitrogen stream atmosphere, dimethylformamide (19 mL) was added to 1.10 g (6.58 mmol) of 9H-carbazole, 1.50 g (2.74 mmol) of Intermediate 6, and 1.33 g (9.59 mmol) of potassium carbonate, and stirred at 130° C. for 15 hours. The reaction solution was quenched with water added thereto, and the suspension was filtered. The crude product was purified by silica gel column chromatography (o-dichlorobenzene), then dissolved in o-dichlorobenzene, and methanol was added to give a precipitate, and the resultant solid was filtered out and purified to give 2.05 g (2.44 mmol, yield 89%) of Compound 3387.

¹H NMR (400 MHz, CDCl₃, δ): 7.75-7.72 (m, 2H), 7.51-7.39 (m, 6H), 7.34-7.11 (m, 11H), 7.07-6.98 (m, 7H), 6.95-6.87 (m, 6H), 6.81 (t, J=7.5 Hz, 2H), 6.74 (t, J=7.5 Hz, 1H), 6.59 (t, J=7.5 Hz, 1H).

ASAP MS spectrometry: C₆₁H₃₆N₄O theoretical 840.29, found 841.47

### (Synthesis Example 4) Synthesis of Comparative Compound 3

### (4-1) Synthesis of Intermediate 7

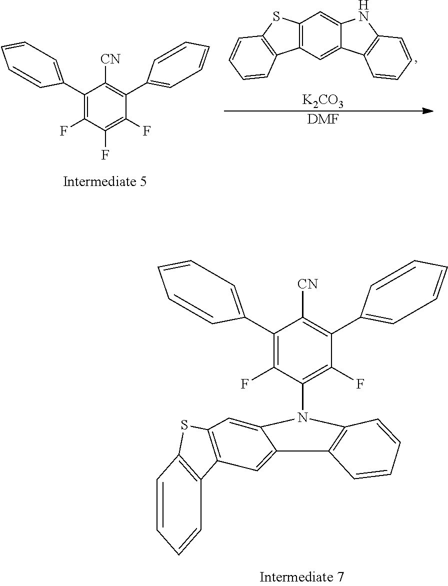

In a nitrogen stream atmosphere, dimethylformamide (13 mL) was added to 0.92 g (3.36 mmol) of 7H-benzothio[2,3-b]carbazole, 1.30 g (4.20 mmol) of Intermediate 5 and 0.87 g (6.30 mmol) of potassium carbonate, and stirred at 100° C. for 13 hours. The reaction solution was quenched with water added thereto, and the suspension was filtered. The crude product was purified by silica gel column chromatography (hexane/dichloromethane=2/3) to give 1.56 g (2.77 mmol, yield 66%) of Intermediate 7.

¹H NMR (500 MHz, CDCl₃, δ): 8.66 (s, 1H), 8.30-8.26 (m, 2H), 7.84 (d, J=8.0 Hz, 1H), 7.63-7.49 (m, 13H), 7.45 (d, J=8.0 Hz, 1H), 7.40 (t, J=7.5 Hz, 1H), 7.25-7.24 (m, 1H)

ASAP MS spectrometry: C₃₇H₂₀F₂N₂S theoretical 562.64, found 563.24

### (4-2) Synthesis of Comparative Compound 3

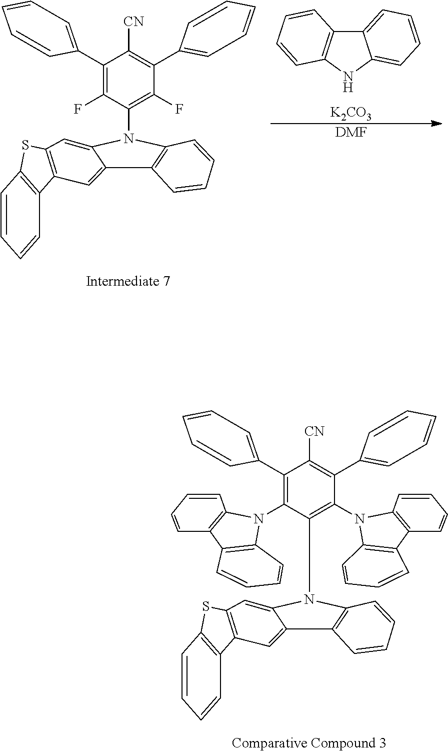

In a nitrogen stream atmosphere, dimethylformamide (20 mL) was added to 1.07 g (6.41 mmol) of 9H-carbazole, 1.50 g (2.67 mmol) of Intermediate 7 and 1.29 g (9.35 mmol) of potassium carbonate, and stirred at 130° C. for 15 hours. The reaction solution was quenched with water added thereto, and the suspension was filtered. The crude product was purified by silica gel column chromatography (o-dichlorobenzene), then dissolved in o-dichlorobenzene and purified by reprecipitation with methanol to give 2.07 g (2.42 mmol, yield 90%) of Comparative Compound 3.

¹H NMR (4(0) MHz, CDCl₃, δ): 7.99 (s, 1H), 7.92-7.88 (m, J=6.0, 4.3 Hz, 1H), 7.68-7.65 (m, J=7.1, 2.9 Hz, 1H), 7.51-7.49 (m, 5H), 7.40-7.26 (m, 15H), 7.19 (d, J=8.0 Hz, 2H), 7.13 (d, J=8.0 Hz, 2H), 7.08-7.00 (m, 6H), 6.96-6.87 (m, 5H), 6.83 (t, J=7.5 Hz, 2H), 6.74 (t, J=7.5 Hz, 1H), 6.58 (t, J=8.0 Hz, 1H).

ASAP MS spectrometry: C₆₁H₃₆N₄S theoretical 856.27, found 857.44

### Examples 1 to 2, Comparative Example 1 to 2

### Production and Evaluation of Thin Film

On a quartz substrate according to a vacuum evaporation method, Compound 26 and PYD2Cz were evaporated from different evaporation sources under the condition of a vacuum degree of lower than 1×10⁻³ Pa to form a thin film having a thickness of 100 nm in which the concentration of Compound 26 was 20% by weight, and this is a thin film of Example 1.

Compound 1626, Comparative Compound 1 and Comparative Compound 2 were individually used in place of Compound 26 to produce thin films of Example 2, Comparative Example 1 and Comparative Example 2, respectively.

The resultant thin films were individually irradiated with 300-nm excitation light to observe emission spectra, in which the peak wavelength (λₘₐₓ) was read. In addition, the lifetime (τ_{d}) of the delayed fluorescence was derived from the transient decay curve of emission observed with the same excitation light. Further, according to the following measurement method, ΔE_{ST} was measured, and the photoluminescence quantum yield (PLQY) was measured with a 300-nm excitation light in a nitrogen atmosphere. The measurement results are as shown in Table 3.

ΔE_{ST} is a value calculated by E_{S1}-E_{T1} in which the lowest excited singlet energy (E_{S1}) and the lowest excited triplet energy (E_{T1}) of the compound targeted for measurement are determined according to the following process.

### (1) Lowest Excited Singlet Energy (E_{S1})

At room temperature (300 K), a fluorescent spectrum of a thin film of the targeted compound was measured (vertical axis: emission intensity, horizontal axis: wavelength). A tangent line was drawn to the rising on the short wavelength side of the fluorescent spectrum, and the wavelength value λedge [nm] at the intersection of the tangent line and the horizontal axis was read. The wavelength value was converted into an energy value according to the following conversion expression to be E_{S1}.

Conversion Expression: E_{S1} [eV]=1239.85/λedge

(2) Lowest excited triplet energy (E_{T1})

The same thin film as above was cooled to 77 [K] with liquid nitrogen, the sample for phosphorescence measurement was irradiated with an excitation light (300 nm), and the phosphorescence was measured with a detector. The light emission after 100 millisecond from irradiation with the excitation light provided a phosphorescence spectrum. A tangent line was drawn to the rising on the short wavelength side of the phosphorescent spectrum, and the wavelength value λedge [nm] at the intersection of the tangent line and the horizontal axis was read. The wavelength value was converted into an energy value according to the above-mentioned conversion expression to be E_{T1}.

The tangent line to the rising of the phosphorescent spectrum on the short wavelength side was drawn as follows. While moving on the spectral curve from the short wavelength side of the phosphorescent spectrum toward the maximum value on the shortest wavelength side among the maximum values of the spectrum, a tangent line at each point on the curve toward the long wavelength side was taken into consideration. With rising thereof (that is, with increase in the vertical axis), the inclination of the tangent line increases. The tangent line drawn at the point at which the inclination value has a maximum value is referred to as the tangent line to the rising on the short wavelength side of the phosphorescent spectrum.

The maximum point having a peak intensity of 10% or less of the maximum peak intensity of the spectrum was not included in the maximum value on the above-mentioned shortest wavelength side, and the tangent line drawn at the point which was closest to the maximum value on the shortest wavelength side and at which the inclination value had a maximum value was referred to as the tangent line to the rising on the short wavelength side of the phosphorescent spectrum.

TABLE 3

| | Light Emitting | ΔE_{ST} | λmax | τ_{d} | PLQY |
|---|---|---|---|---|---|
| | Material | (eV) | (nm) | (μs) | (%) |
| Comparative | Comparative | 0.159 | 464 | 13.6 | 64 |
| Example 1 | Compound 1 | | | | |
| Example 1 | Compound 26 | 0.136 | 468 | 7.3 | 67 |
| Comparative | Comparative | 0.225 | 470 | 22.7 | 57 |
| Example 2 | Compound 2 | | | | |
| Example 2 | Compound 1626 | 0.150 | 478 | 12.5 | 84 |

The comparative results of Example 1 and Comparative Example 1 and the comparative results of Example 2 and Comparative Example 2 indicate that ΔE_{ST} of the compound of the present invention having, as introduced thereinto, a benzofuran ring-condensed carbazol-9-yl group is small, therefore shortening the delayed fluorescence lifetime (τ_{d}) and increasing the photoluminescence quantum yield (PLQY).

### Examples 3 to 4

### Production and Evaluation of Doped Thin Film Differing in the Host Material Therein

Thin films of Example 3 and Example 4 were produced according to the same process as above, except that the host material in Example 1 and Example 2 was changed from PYD2Cz to PPF.

The resultant thin films were irradiated with an excitation light in the same manner as above, and the films emitted delayed fluorescence. The delayed fluorescence lifetime (Td) of Example 3 and Example 4 was 12.5 μs and 18.8 μs, respectively. The photoluminescence quantum yield (PLQY) of Example 3 and Example 4 was 70% and 81%, respectively.

### Example 5, Comparative Example 3

### Production and Evaluation of Thin Film of Similar Light Emitting Material Differing in Constituting Elements

On a quartz substrate, Compound 3387 was vapor-deposited according to a vacuum evaporation method under a vacuum degree of lower than 1×10⁻³ Pa to form a neat thin film of Example 5 having a thickness of 10 nm. Using Comparative Compound 3 in place of Compound 3387 and according to the same process, a neat thin film of Comparative Example 3 was formed.

In the same manner as in Examples 1 to 3, the resultant thin films were irradiated with an excitation light, and both the two emitted delayed fluorescence. The peak wavelength (λₘₐₓ) was 493 nm in Example 5, and was 499 nm in Comparative Example 3. The photoluminescence quantum yield (PLQY) of Example 5 was 1.1 times that of Comparative Example 3. These results indicate that, in Example 5 using the compound having a benzofuran ring-condensed carbazol-9-yl group, the emission peak wavelength shifted shorter on the short wavelength range than in Comparative Example 3 using the compound having a benzothiophene ring-condensed carbazol-9-yl group, and that the emission efficiency in the former was higher than in the latter.

On the other hand, on a quartz substrate, Compound 3387 and PPF were vapor-deposited according to a vacuum evaporation method under a vacuum degree of lower than 1×10³ Pa from different evaporation sources to form a thin film having a thickness of 100 nm in which the concentration of Compound 3387 was 20% by weight. This is a doped thin film of Example 5. Using Comparative Compound 3 in place of Compound 3387 and according to the same process, a doped thin film of Comparative Example 3 was formed. These were irradiated with a 300-nm excitation light to draw a transient decay curve of emission, from which the delayed fluorescence lifetime (τ_{d}) was determined. As a result, the lifetime was 11.4 μs in Comparative Example 3, and was 7.4 μs in Example 5, that is, shorter by about 30% than in Comparative Example 3. This indicates that the compound having a benzofuran ring-condensed carbazol-9-yl group has a shorter delayed fluorescence lifetime than the compound having a benzothiophene ring-condensed carbazol-9-yl group.

### Examples 6 to 7

### Production and Evaluation of Organic Electroluminescent Device

On a glass substrate having, as formed thereon, an indium-tin oxide (ITO) anode film having a thickness of 100 nm, thin films were layered according to a vacuum evaporation method under a vacuum degree of 1×10⁻⁶ Pa. First, on ITO, a first hole injection layer of a first hole injection material was formed, and on this, a second hole injection layer of a second hole injection material was formed, and on this, a hole transport layer of a hole transport material was formed, and further on this, an electron blocking layer of an electron blocking material was formed. Further on this, Compound 26 and a host material were vapor co-deposited from different evaporation sources to form a light emitting layer in which the concentration of Compound 26 was 30% by weight. Next, a hole blocking layer of a hole blocking material was formed, and on this, an electron transport layer was formed, and further on this, an electrode was formed. According to this process, an organic electroluminescent device of Example 6 was produced.

Using Compound 1626 in place of Compound 26 and according to the same process, an organic electroluminescent device of Example 7 was produced.

The organic electroluminescent devices of Example 6 and Example 7 exhibited a high emission efficiency, took a low driving voltage, and had a long device lifetime (high device durability).

In addition, using the other compounds of the present invention, there can be provided organic electroluminescent devices having a high emission efficiency, taking a low driving voltage and having a long device lifetime (high device durability).

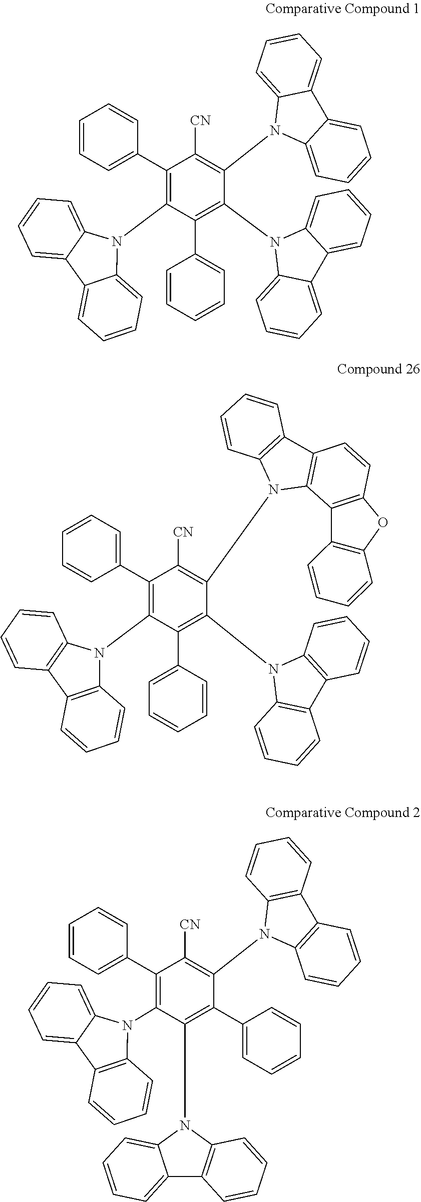 [SMILES: N#Cc1c(-c2ccccc2)c(-n2c3ccccc3c3ccccc32)c(-c2ccccc2)c(-n2c3ccccc3c3ccccc32)c1-n1c2ccccc2c2ccccc21 ; N#Cc1c(-c2ccccc2)c(-n2c3ccccc3c3ccccc32)c(-c2ccccc2)c(-n2c3ccccc3c3ccccc32)c1-n1c2ccccc2c2ccc3oc4ccccc4c3c21 ; N#Cc1c(-c2ccccc2)c(-n2c3ccccc3c3ccccc32)c(-n2c3ccccc3c3ccccc32)c(-c2ccccc2)c1-n1c2ccccc2c2ccccc21]

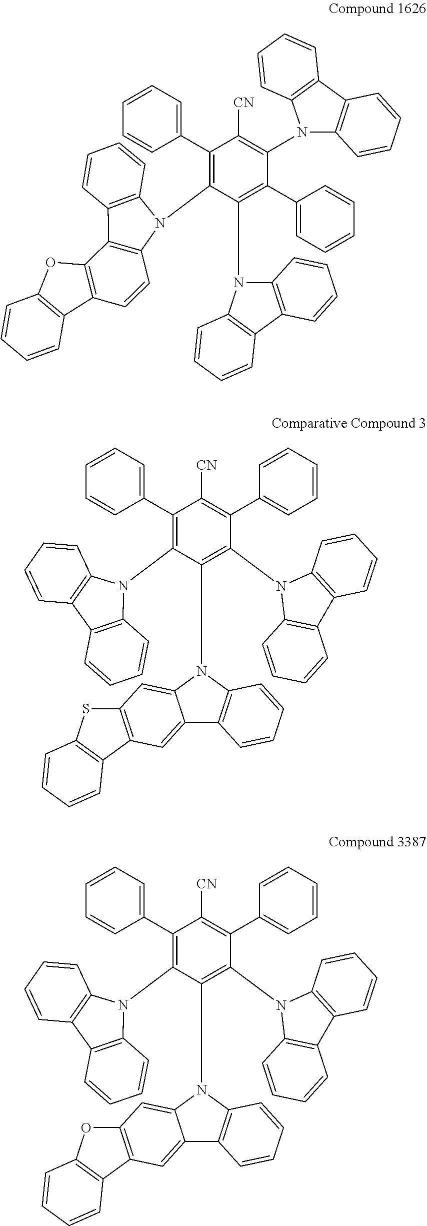 [SMILES: N#Cc1c(-c2ccccc2)c(-n2c3ccccc3c3c4oc5ccccc5c4ccc32)c(-n2c3ccccc3c3ccccc32)c(-c2ccccc2)c1-n1c2ccccc2c2ccccc21 ; N#Cc1c(-c2ccccc2)c(-n2c3ccccc3c3ccccc32)c(-n2c3ccccc3c3cc4c(cc32)sc2ccccc24)c(-n2c3ccccc3c3ccccc32)c1-c1ccccc1 ; N#Cc1c(-c2ccccc2)c(-n2c3ccccc3c3ccccc32)c(-n2c3ccccc3c3cc4c(cc32)oc2ccccc24)c(-n2c3ccccc3c3ccccc32)c1-c1ccccc1]

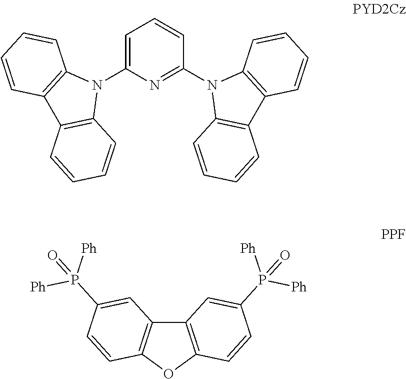 [SMILES: c1cc(-n2c3ccccc3c3ccccc32)nc(-n2c3ccccc3c3ccccc32)c1 ; O=P(c1ccccc1)(c1ccccc1)c1ccc2oc3ccc(P(=O)(c4ccccc4)c4ccccc4)cc3c2c1]

1. A compound represented by the following general formula (1): 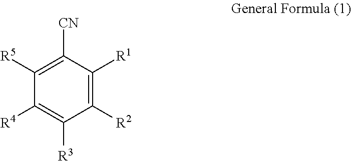
wherein:
two of R¹ to R⁵ are each independently a substituted or unsubstituted aromatic hydrocarbon cyclic group, or a substituted or unsubstituted aromatic heterocyclic group containing a nitrogen atom as a ring skeleton constituting atom,
three of R¹ to R⁵ are each independently a donor group but excepting a substituted or unsubstituted aromatic hydrocarbon cyclic group and a substituted or unsubstituted aromatic heterocyclic group containing a nitrogen atom as a ring skeleton constituting atom, all the three donor groups are not the same, and at least one of the three donor groups is a carbazolyl-9-yl group condensed with a benzofuran ring.

2. The compound according to claim 1, wherein R¹, R² and R⁴ each are independently a donor group.

3. The compound according to claim 1, wherein R¹, R³ and R⁴ each are independently a donor group.

4. The compound according to claim 1, wherein two of the three donor groups are the same as each other.

5. The compound according to claim 1, wherein the carbazolyl-9-yl group condensed with a benzofuran ring has a structure where the benzofuran ring is directly condensed with one benzene ring constituting the carbazolyl-9-yl group.

6. The compound according to claim 5, wherein the carbazolyl-9-yl group condensed with a benzofuran ring has a structure of any of the following: 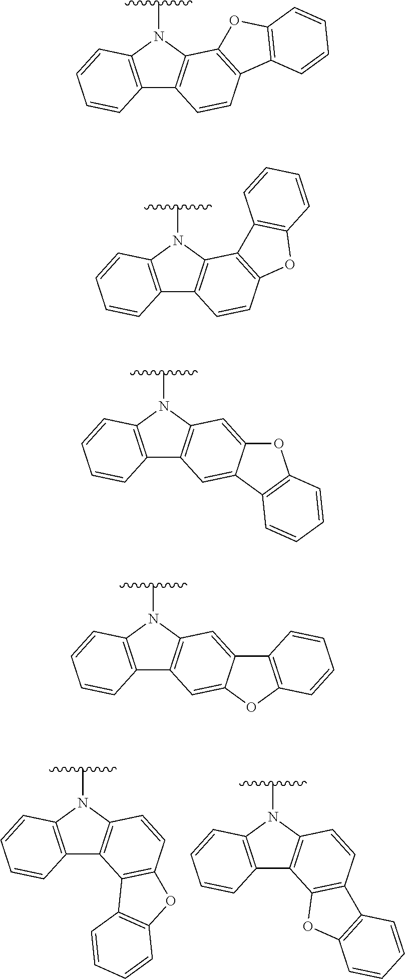
wherein the hydrogen atoms can be substituted.

7. The compound according to claim 5, wherein two of R¹ to R⁵ are a carbazolyl-9-yl group condensed with a benzofuran ring.

8. The compound according to claim 7, wherein the two carbazolyl-9-yl groups condensed with a benzofuran ring are the same as each other.

9. The compound according to claim 5, wherein only one of R¹ to R⁵ is a carbazolyl-9-yl group condensed with a benzofuran ring.

10. The compound according to claim 1, wherein two of R¹ to R⁵ each are independently a substituted or unsubstituted aromatic hydrocarbon cyclic group.

11. The compound according to claim 1, wherein two of R¹ to R⁵ are each independently a substituted or unsubstituted aromatic heterocyclic group containing a nitrogen atom as a ring skeleton constituting atom.

12. The compound according to claim 10, wherein two of R¹ to R⁵ are the same as each other.

13. A light emitting material of a compound of claim 1.

14. A light emitting device containing a compound of claim 1.

15. The light emitting device according to claim 14, wherein the light emitting device has a light emitting layer and the light emitting layer contains the above compound and a host material.

16. The light emitting device according to claim 14, wherein the light emitting device has a light emitting layer, the light emitting layer contains the above compound and a light emitting material, and the light emitting material mainly emits light.
